(19) United States
(12) Patent Application Publication
(10) Pub. No.: US 2023/0122249 A1
(43) Pub. Date: Apr. 20, 2023

(54) 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Yam B. POUDEL, Fremont, CA (US); Matthew COX, San Francisco, CA (US); Liqi HE, San Jose, CA (US); Daniel O'MALLEY, New Hope, PA (US); Ashvinikumar V. GAVAI, Princeton Junction, NJ (US); Sanjeev GANGWAR, Foster City, CA (US); Matthias BROEKEMA, New Hope, PA (US); Prasanna SIVAPRAKASAM, Plainsboro, NJ (US); Christine M. TARBY, Lawrenceville, NJ (US); Murugaiah ANDAPPAN MURUGAIAH SUBBAIAH,Murugaiah, Bangalore (IN)

(73) Assignee: BRISTOL-MYERS SQUIBB COMPANY, Princeton, NJ (US)

(21) Appl. No.: 17/793,162

(22) PCT Filed: Jan. 26, 2021
(86) PCT No.: PCT/US2021/014977

§ 371 Date: Jul. 15, 2022

Related U.S. Application Data
(60) Provisional application No. 62/966,111, filed on Jan. 27, 2020.

(51) Int. Cl. C07D 487/04; C07D 519/00

(52) U.S. Cl. CPC C07D 487/04; C07D 519/00

(57) ABSTRACT

Compounds according to formula I are useful as agonists of Toll-like receptor 7 (TLR7). (I) Such compounds can be used in cancer treatment, especially in combination with an anti-cancer immunotherapy agent, or as a vaccine adjuvant.

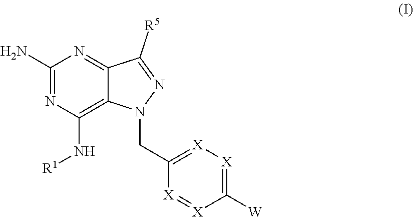

### CROSS-REFERENCE TO RELATED APPLICATIONS

This application claims the benefit under 35 U.S.C. § 119(e) of U.S. Provisional Application Ser. No. 62/966,111, filed Jan. 27, 2020 the disclosure of which is incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghöfer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

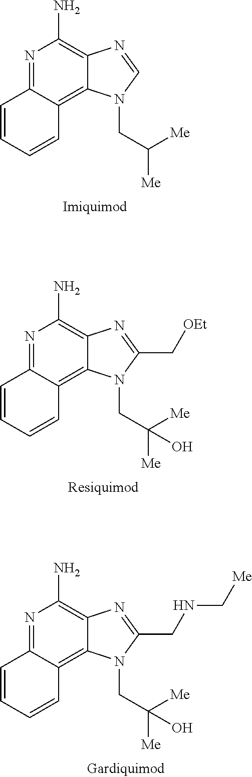 [SMILES: CC(C)Cn1cnc2c(N)nc3ccccc3c21 ; CCOCc1nc2c(N)nc3ccccc3c2n1CC(C)(C)O ; CCNCc1nc2c(N)nc3ccccc3c2n1CC(C)(C)O]

Synthetic TLR7 agonists based on a pteridinone molecular scaffold are also known, as exemplified by vesatolimod (Desai et al. 2015).

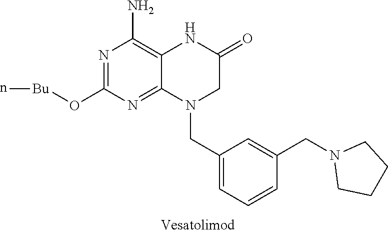

Other synthetic TLR7 agonists based on a purine-like scaffold have been disclosed, frequently according to the general formula (A):

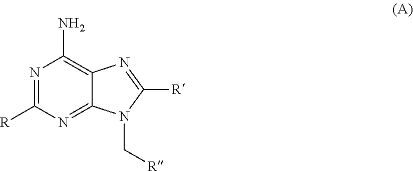

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections 15 include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system, having activity as TLR7 agonists.

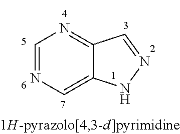

In one aspect, there is provided a compound having a structure according to formula

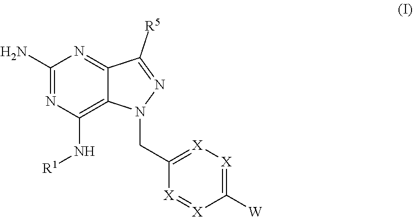

wherein

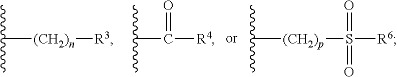

wherein
W is H, halo, C₁-C₃ alkyl, CN, (C₁-C₄ alkanediyl)OH,
each X is independently N or CR²;
R¹ is (C₁-C₅ alkyl),
   (C₂-C₅ alkenyl),
   (C₁-C₈ alkanediyl)₀₋₁(C₃-C₃ cycloalkyl),
   (C₁-C₈ alkanediyl)₀₋₁(C₅-C₁₀ spiroalkyl),
   (C₂-C₈ alkanediyl)OH,
   (C₂-C₈ alkanediyl)O(C₁-C₃ alkyl),
   (C₁-C₄ alkanediyl)₀₋₁(5-6 membered heteroaryl),
   (C₁-C₄ alkanediyl)₀₋₁phenyl,
   (C₁-C₄ alkanediyl)CF₃,
   (C₂-C₈ alkanediyl)N[C(═O)](C₁-C₃ alkyl),
   (C₂-C₈ alkanediyl)₀₋₁(C₃-C₆ cycloalkanediyl)(C₃-C₆ cycloalkyl), or
   (C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN, or [C(═O)]₀₋₁NR^{x}R^{y};
R³ is H, halo, OH, CN,
   NH₂,
   NH[C(═O)]₀₋₁(C₁-C₅ alkyl),
   N(C₁-C₅ alkyl)₂,
   NH[C(═O)]₀₋₁(C₁-C₄ alkanediyl)₀₋₁(C₃-C₈ cycloalkyl),
   NH[C(═O)]₀₋₁(C₁-C₄ alkanediyl)₀₋₁(C₄-C₁₀ bicycloalkyl),
   NH[C(═O)]₀₋₁(C₁-C₄ alkanediyl)₀₋₁(C₅-C₁₀ spiroalkyl),
   N(C₃-C₆ cycloalkyl)₂,
   O(C₁-C₄ alkanediyl)₀₋₁(C₃-C₈ cycloalkyl),
   O(C₁-C₄ alkanediyl)₀₋₁(C₄-C₈ bicycloalkyl),
   O(C₁-C₄ alkanediyl)₀₋₁(C₅-C₁₀ spiroalkyl),
   O(C₁-C₄ alkanediyl)₀₋₁(C₁-C₆ alkyl),
   N[C₁-C₃ alkyl]C(═O)(C₁-C₆ alkyl),
   NH(SO₂)(C₁-C₅ alkyl),
   NH(SO₂)(C₁-C₄ alkanediyl)₀₋₁(C₃-C₈ cycloalkyl),
   NH(SO₂)(C₁-C₄ alkanediyl)₀₋₁(C₄-C₁₀ bicycloalkyl),
   NH(SO₂)(C₁-C₄ alkanediyl)₀₋₁(C₅-C₁₀ spiroalkyl),
   a 6-membered aromatic or heteroaromatic moiety,
   a 5-membered heteroaromatic moiety, or
   a moiety having the structure

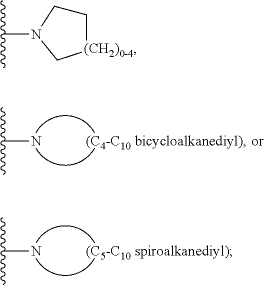

R⁴ is NH₂,
   NH(C₁-C₅ alkyl),
   N(C₁-C₅ alkyl)₂,
   NH(C₁-C₄ alkanediyl)₀₋₁(C₃-C₈ cycloalkyl),
   NH(C₁-C₄ alkanediyl)₀₋₁(C₄-C₁₀ bicycloalkyl),
   NH(C₁-C₄ alkanediyl)₀₋₁(C₅-C₁₀ spiroalkyl),
   N(C₃-C₆ cycloalkyl)₂,
   or
   a moiety having the structure

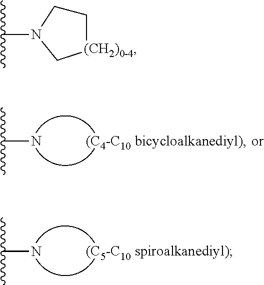

R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl,

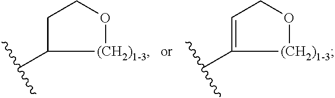

R⁶ is NH₂,
   (NH)₀₋₁(C₁-C₅ alkyl),
   N(C₁-C₅ alkyl)₂,
   (NH)₀₋₁(C₁-C₄ alkanediyl)₀₋₁(C₃-C₅ cycloalkyl),
   (NH)₀₋₁(C₁-C₄ alkanediyl)o 1(C₄-C₁₀ bicycloalkyl),
   (NH)₀₋₁(C₁-C₄ alkanediyl)₀₋₁(C₅-C₁₀ spiroalkyl),
   N(C₃-C₆ cycloalkyl)₂, or
   a moiety having the structure

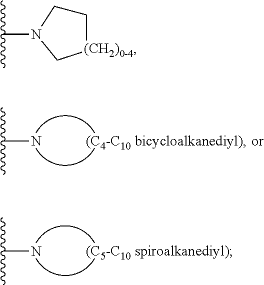

R^{x} and R^{y} are independently H or C₁-C₃ alkyl or R^{x} and R^{y} combine with the nitrogen to which they are bonded to form a 3- to 7-membered heterocycle;
n is 1, 2, or 3;
and
p is 0, 1, 2, or 3;
wherein in R¹, R², R³, R⁴, R⁵, and R⁶
   an alkyl, alkenyl, cycloalkyl, alkanediyl, bicycloalkyl, spiroalkyl, cyclic amine, 6-membered aromatic or heteroaromatic moiety, 5-membered heteroaromatic moiety or a moiety of the formula

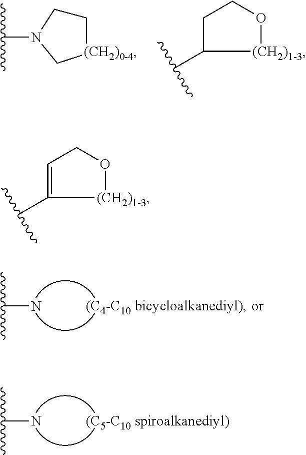

is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl); and
an alkyl, alkenyl, alkanediyl, cycloalkyl, bicycloalkyl, spiroalkyl, or a moiety of the formula

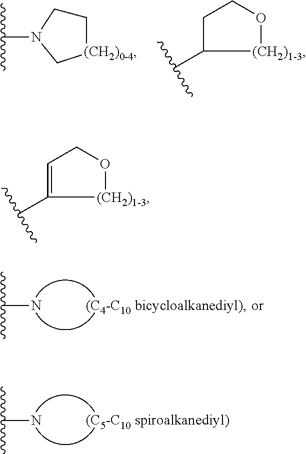

optionally may have a CH₂ group replaced by 0, SO₂, CF₂, C(═O), NH,
N[C(═O)]₀₋₁(C₁-C₅ alkyl),
N[C(═O)]₀₋₁(C₁-C₄ alkanediyl)CF₃,
N[C(═O)]₀₋₁(C₂-C₄ alkanediyl)OH
N(SO₂)(C₁-C₃ alkyl),
N(C₁-C₃ alkanediyl)₀₋₁[C(═O)]NR^{x}R^{y}, or
N[C(═O)]₀₋₁(C₁-C₄ alkanediyl)₀₋₁(C₃-C₅ cycloalkyl);
with the provisos that at least one or R¹ and W comprises a spiroalkyl or spiroalkanediyl moiety and that the compound of formula (I) is other than

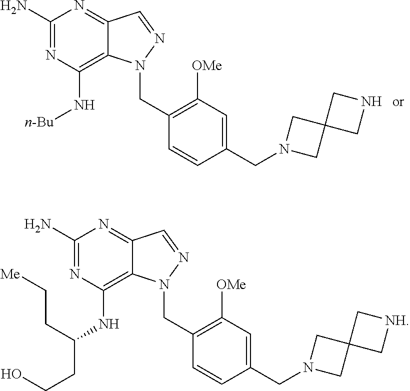

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In one aspect, compounds of this disclosure are according to formula (Ia), wherein R¹, R², R¹, and W are as defined in respect of formula (I):

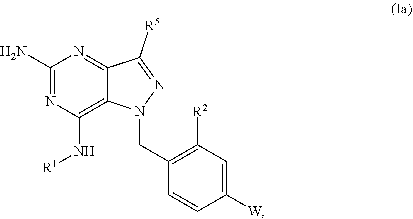

with R² preferably being OMe.

In another aspect, compounds of this disclosure are according to formula (Ib), wherein R¹, R², R³, and R⁵ are as defined in respect of formula (I):

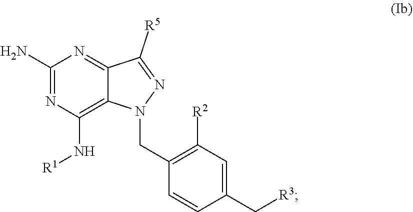

with R² preferably being OMe.

In another aspect, compounds of this disclosure are according to formula (Ic), wherein R¹, R², R⁴, and R⁵ are as defined in respect of formula (I):

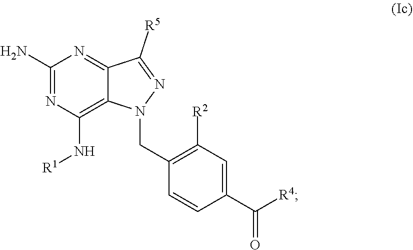

with R² preferably being OMe.

In one aspect, this disclosure provides a compound having a structure according to formula (Id)

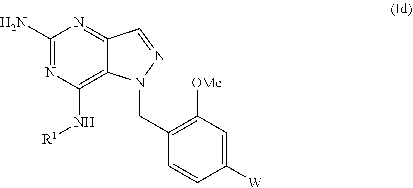

wherein

### R¹ is

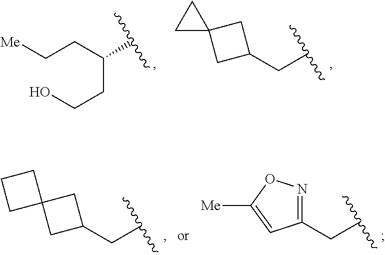

and

### W is

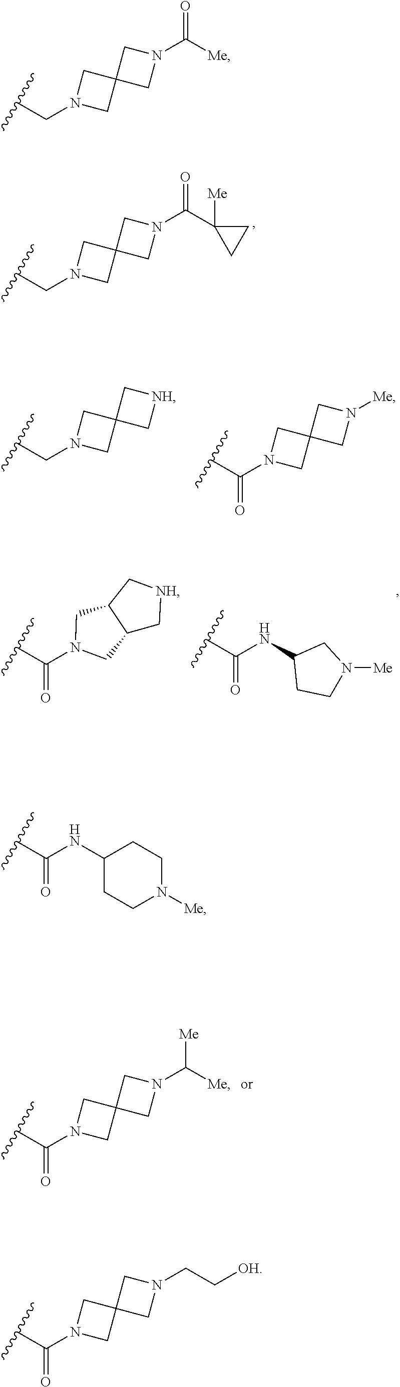

In another aspect, this disclosure provides a compound having a structure according to formula (Ie)

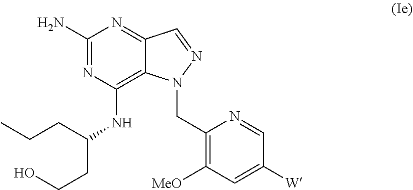

wherein W′ is

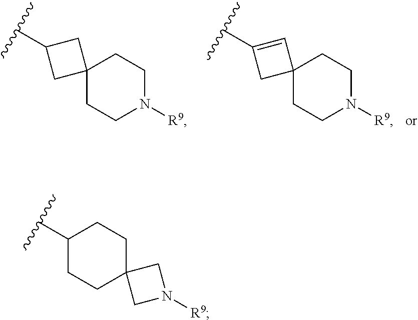

and R⁹ is H, C₁-C₅ alkyl, (CH₂)₁₋₂(C₃-C₅ cycloalkyl), or

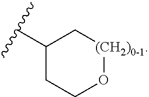

Specific examples of W′ include

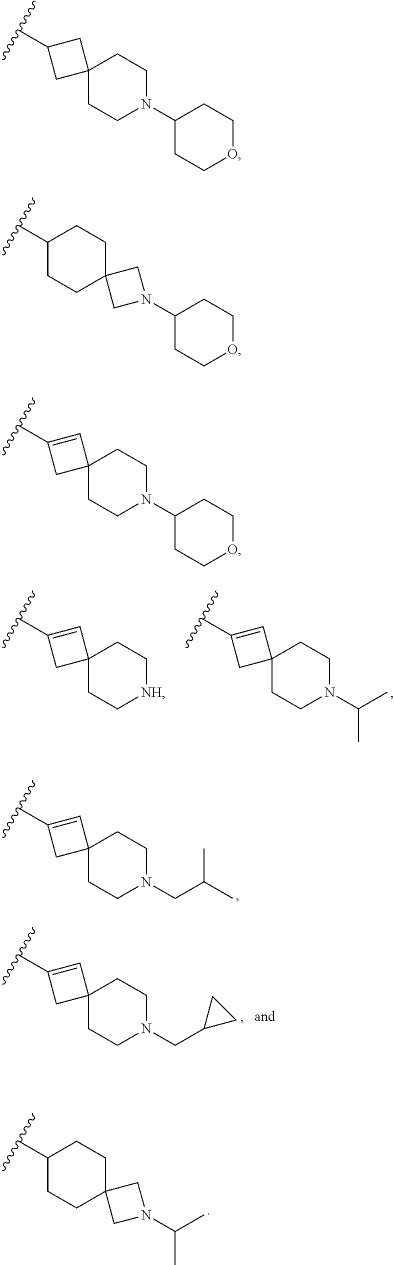

### EXAMPLES OF SUITABLE GROUPS R¹ INCLUDE

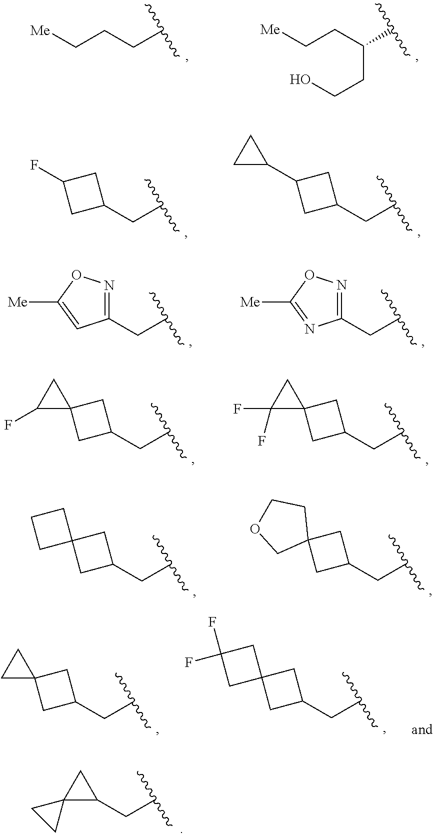

Preferably, R¹ is selected from the group consisting of

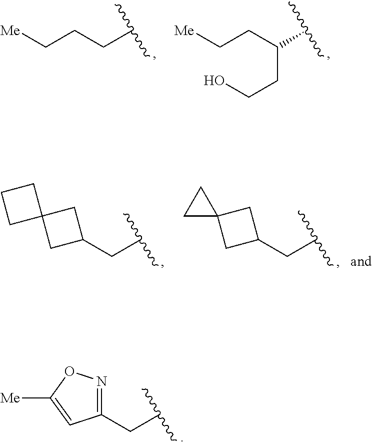

R² preferably is OMe or OCHF₂, more preferably OMe.

R⁵ preferably is H, CH₂OH, or Me, more preferably H.

Examples where W is

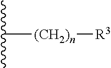

with n equals 1 include:

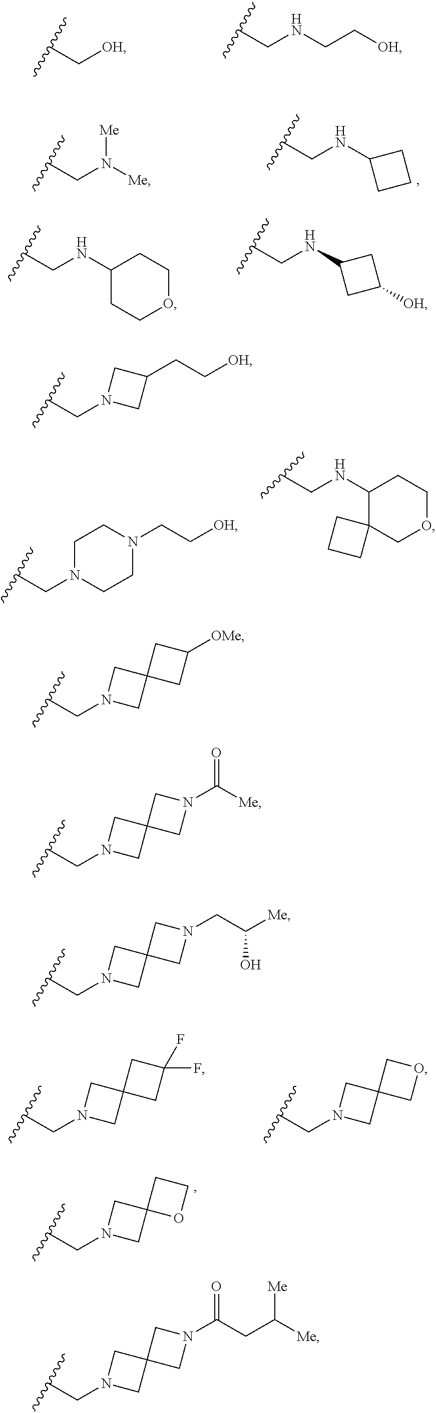

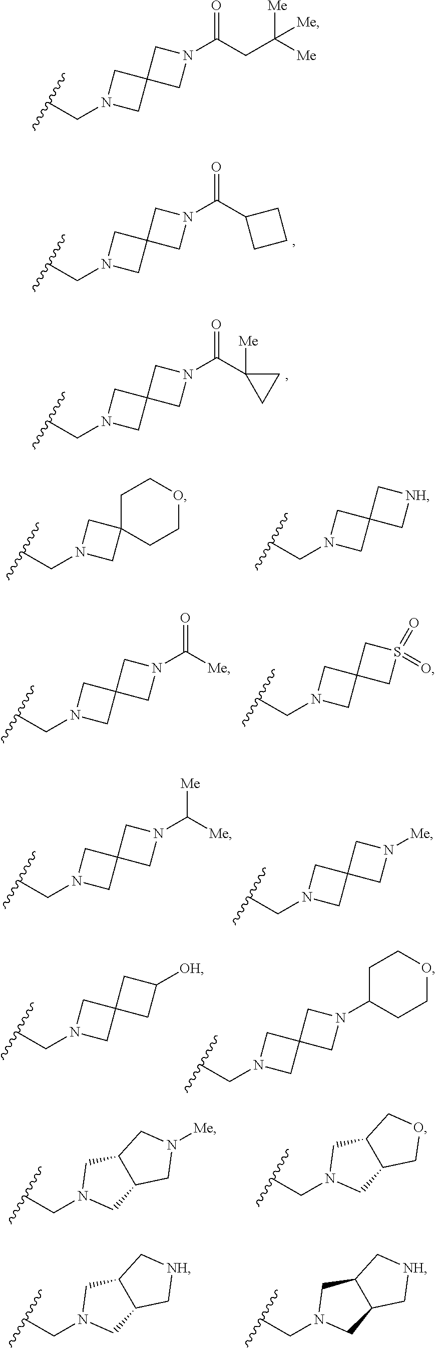

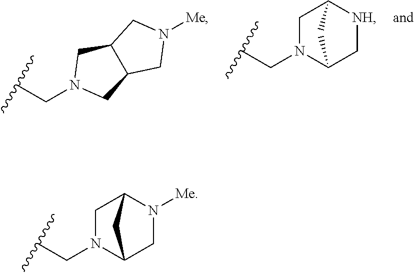 [SMILES: *CN1C[C@H]2CN(C)C[C@H]2C1 ; *CN1C[C@@H]2C[C@H]1CN2 ; *CN1C[C@H]2C[C@@H]1CN2C]

Preferably

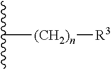

is selected from the group consisting of

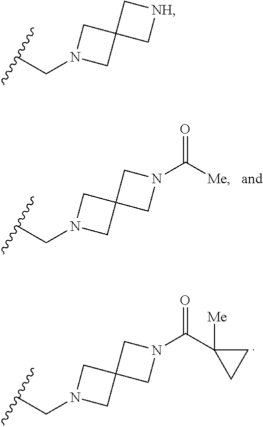 [SMILES: *CN1CC2(CNC2)C1 ; *CN1CC2(C1)CN(C(C)=O)C2 ; *CN1CC2(C1)CN(C(=O)C1(C)CC1)C2]

Examples where W is

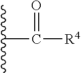

include:

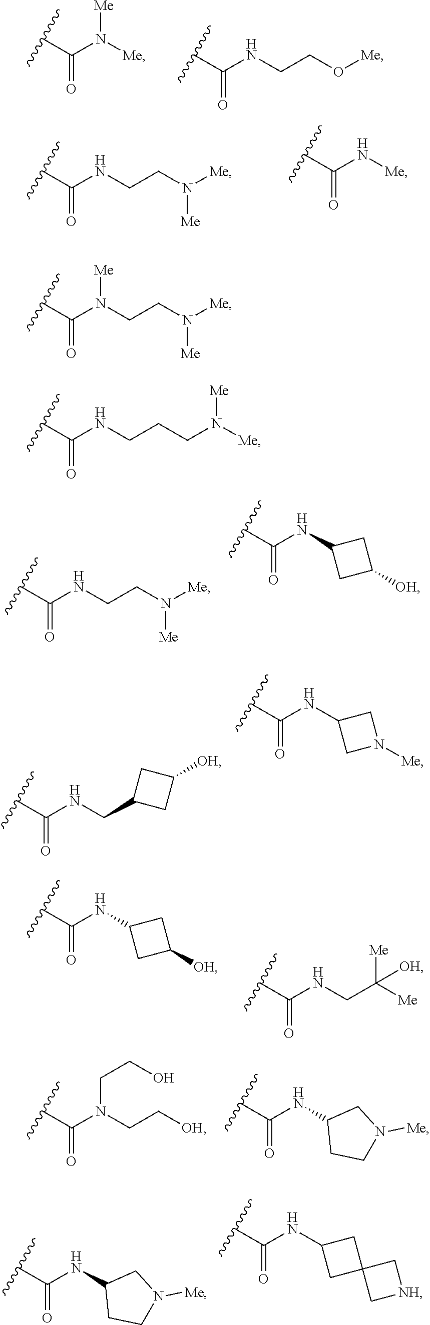

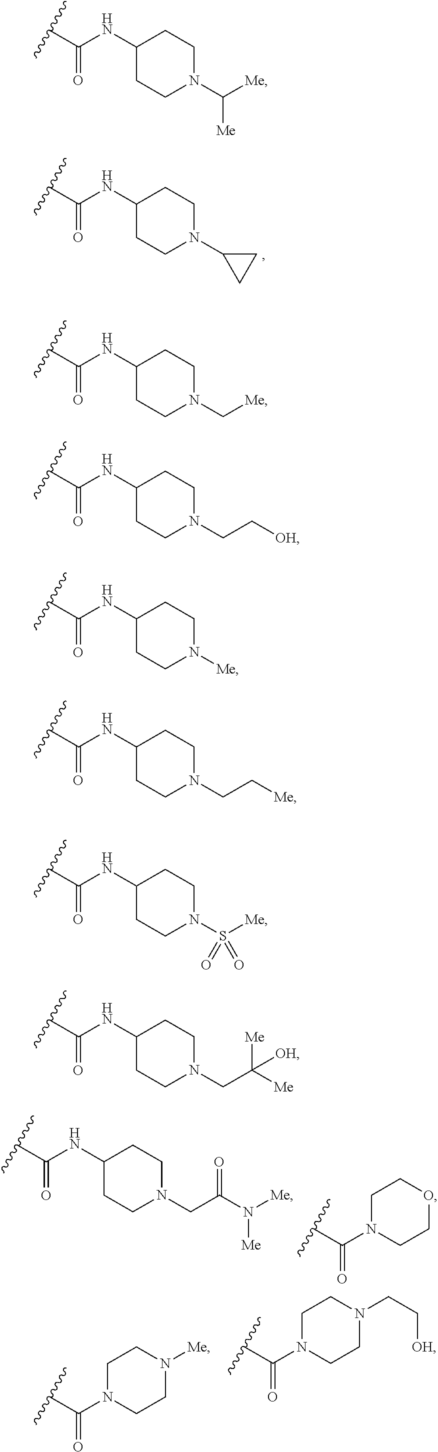

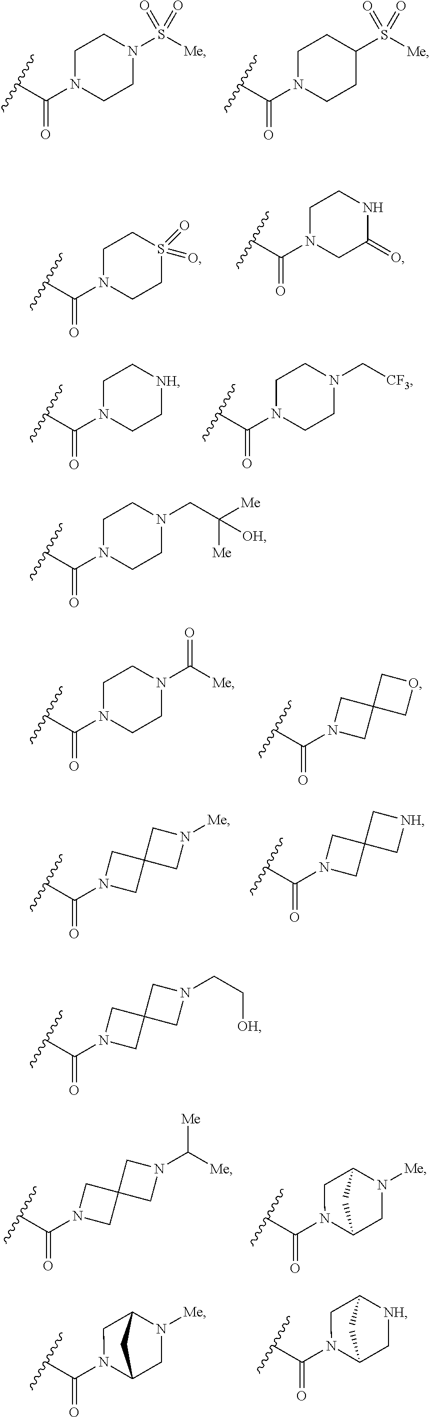

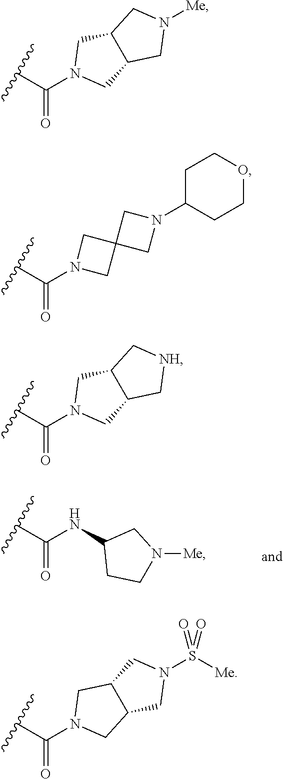

Preferably,

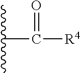

is selected from the group consisting of

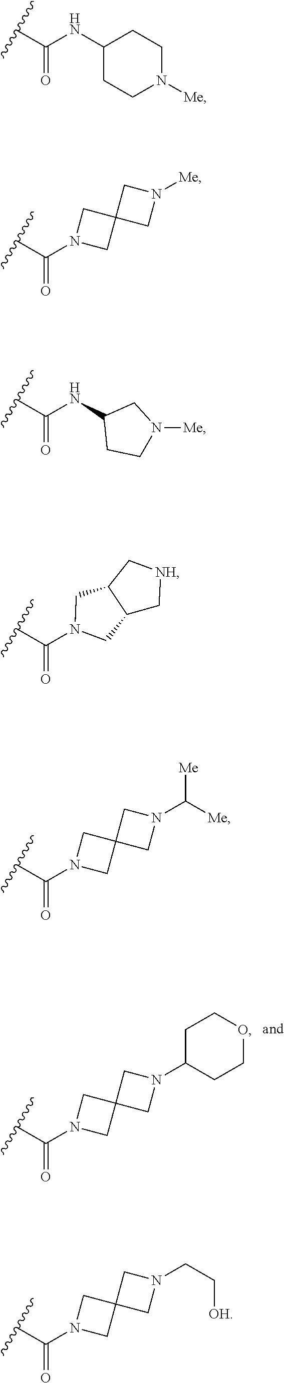

In one aspect, W is

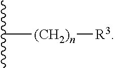

In one aspect, W is

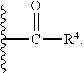

In another aspect,
R³ is H, halo, OH, CN,
   NH₂,
   NH[C(═O)]₀₋₁(C₁-C₅ alkyl),
   N(C₁-C₅ alkyl)₂,
   NH[C(═O)]₀₋₁(C₁-C₄ alkanediyl)₀₋₁(C₃-C₅ cycloalkyl),
   NH[C(═O)]₀₋₁(C₁-C₄ alkanediyl)₀₋₁(C₄-C₁₀ bicycloalkyl),
   NH[C(═O)]₀₋₁(C₁-C₄ alkanediyl)₀₋₁(C₅-C₁₀ spiroalkyl),
   N(C₃-C₆ cycloalkyl)₂,
   N[C₁-C₃ alkyl]C(═O)(C₁-C₆ alkyl),
   a 6-membered aromatic or heteroaromatic moiety,
   a 5-membered heteroaromatic moiety, or
   a moiety having the structure

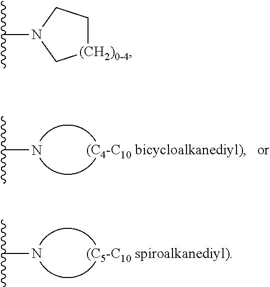

In one aspect, each of R¹ and W comprises a spiroalkyl or spiroalkanediyl moiety.

In one aspect, R¹ comprises a spiroalkyl moiety and W comprises a bicycloalkyl or bicycloalkanediyl moiety.

In one aspect, R¹ comprises a spiroalkyl moiety and W does not comprise a spiroalkyl or spiroalkanediyl moiety.

In one aspect, W comprises a spiroalkyl or spiroalkanediyl moiety and R¹ does not comprise a spiroalkyl moiety.

By way of exemplification and not of limitation, moieties of the formula

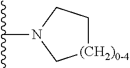 [SMILES: *N1CCCC1]

include

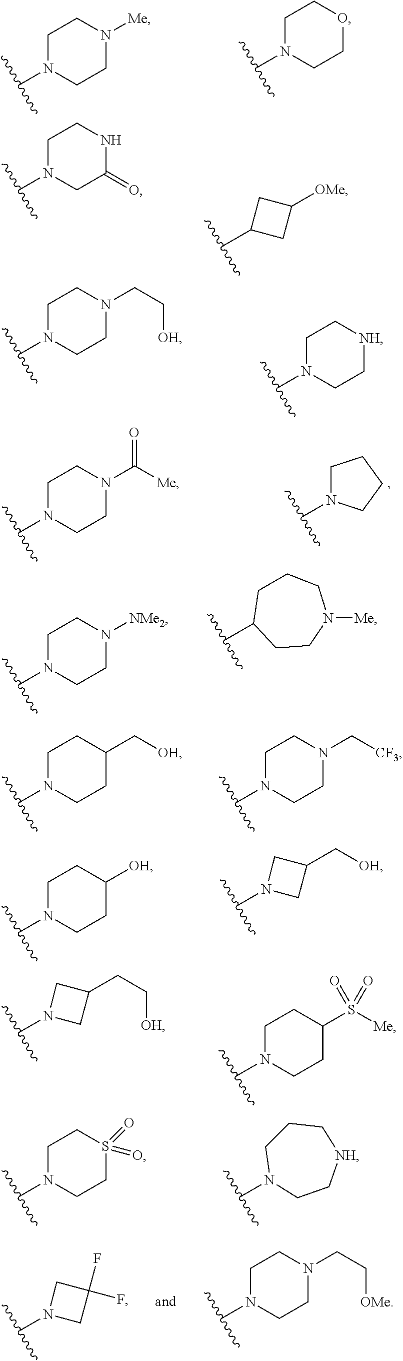

By way of exemplification and not of limitation, spiroalkyl groups include

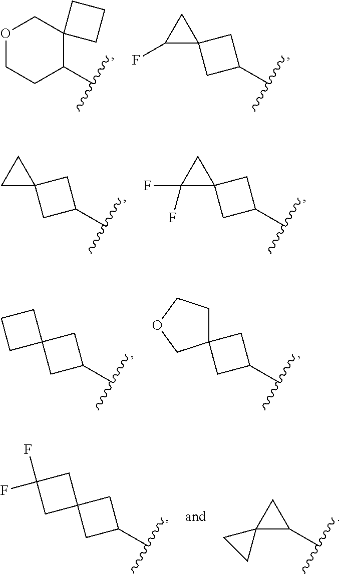

By way of exemplification and not of limitation, moieties of the formula

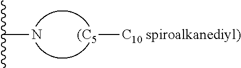

include

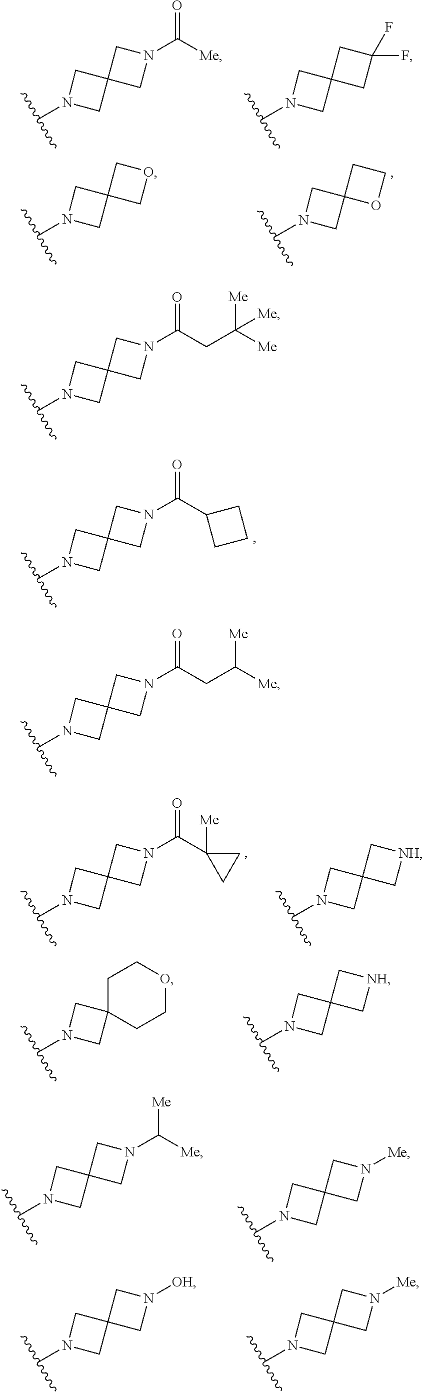

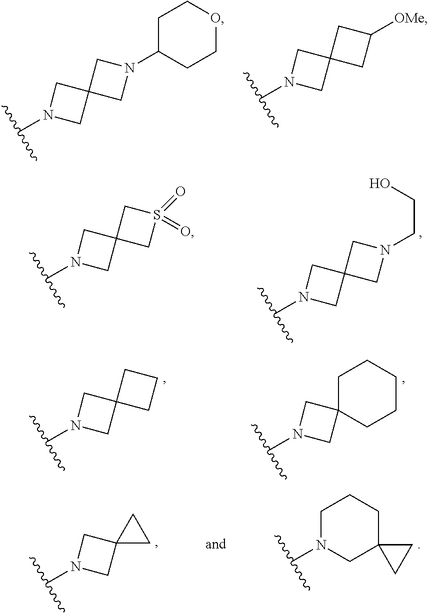

By way of exemplification and not of limitation, bicycloalkyl groups include

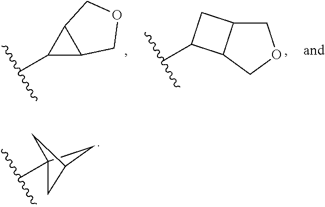 [SMILES: *C1C2COCC12 ; *C1CC2COCC12 ; *C12CC(C1)C2]

By way of exemplification and not of limitation, moieties of the formula

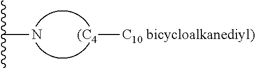

include

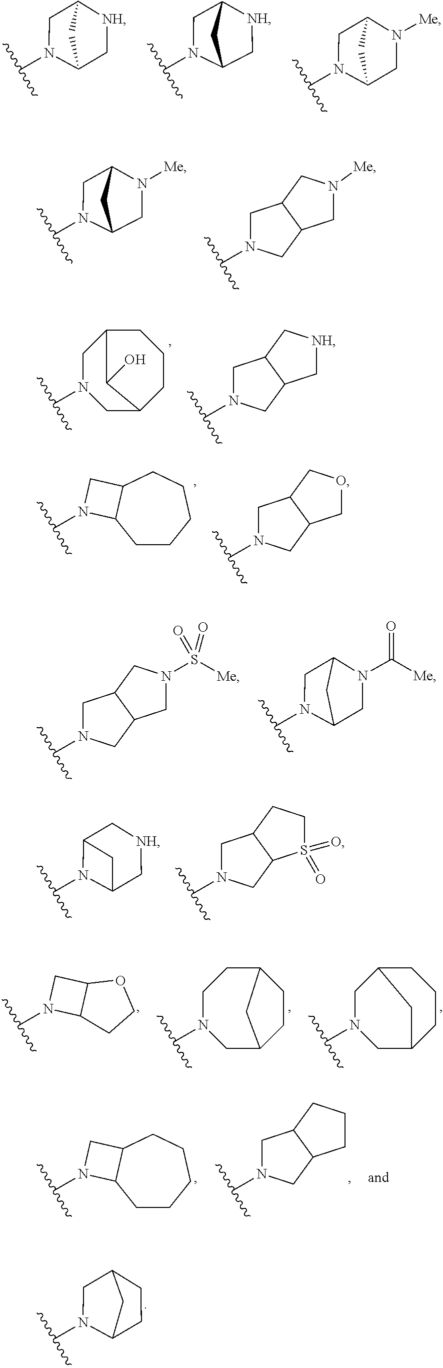

Some of the above exemplary spiroalkyl and bicyloalkyl groups and moieties of the formula

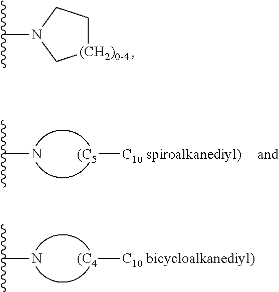

bear optional substituents and/or optionally have one or more CH₂ groups replaced by O, SO₂, etc., as described in the BRIEF SUMMARY OF THE DISCLOSURE above.

Specific examples of compounds disclosed herein are shown in the following Table A. The table also provides data relating to biological activity: human TLR7 reporter assay and/or induction of the CD69 gene in human whole blood, determined per the procedures provided hereinbelow. The right-most column contains analytical data (mass spectrum, LC/MS retention time, and NMR). In one embodiment, a compound of this disclosure has (a) a human TLR7 (hTLR7) Reporter Assay EC₅₀ value of less than 1,000 nM and (b) a human whole blood (hWB) CD69 induction EC₅₀ value of less than 1,000 nM. (Where an assay was performed multiple times, the reported value is an average.)

TABLE A

| | | hTLR7 | hWB | Analytical Data ((Mass spectrum, |
|---|---|---|---|---|
| | | Reporter | CD69 | LC/MS Retention Time, ¹H NMR (500 |
| Cpd | | EC₅₀, | EC₅₀ | MHz, DMSO-d₆ unless noted |
| No. | Structure | nM | (nM) | otherwise)) |
| 101 | 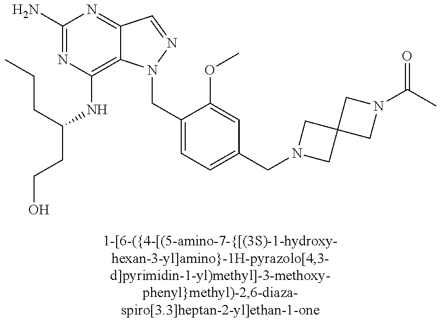 [SMILES: CCC[C@@H](CCO)Nc1nc(N)nc2cnn(Cc3ccc(CN4CC5(C4)CN(C(C)=O)C5)cc3OC)c12] | 32.4 | 4.5 | LC/MS [M + H]⁺ = 523.4 RT (min) 1.04 (LC/MS Procedure B) δ 7.57 (s, 1H), 6.93 (s, 1H), 6.74- 6.68 (m, 1H), 6.36 (d, J = 7.7 Hz, 1H), 5.74-5.62 (m, 3H), 5.55 (d, J = 17.0 Hz, 1H), 4.31 (s, 1H), 4.14 (s, 2H), 3.85 (d, J = 5.0 Hz, 4H), 3.30 (q, J = 6.4, 5.9 Hz, 1H), 1.91 (d, J = 1.1 Hz, 3H), 1.70 (s, 3H), 1.63 (dd, J = 13.2, 6.0 Hz, 1H), 1.54-1.45 (m, 1H), 1.44-1.31 (m, 3H), 1.04 (q, J = 8.7, 8.1 Hz, 2H), 0.76 (t, J = 7.3 Hz, 3H) |
| 102 | 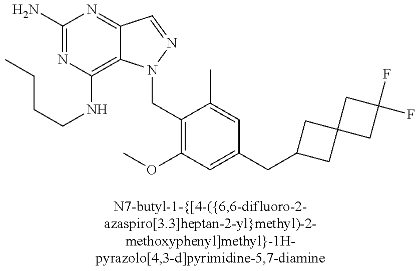 | 95.4 | | LC/MS [M + H]⁺ = 472.2 RT (min) 1.16 (LC/MS Procedure B) δ 7.56 (s, 1H), 6.92 (d, J = 1.5 Hz, 1H), 6.74-6.68 (m, 1H), 6.47 (s, 1H), 6.41 (d, J = 7.7 Hz, 1H), 5.70 (s, 1H), 5.60 (s, 2H), 3.85-3.81 (m, 2H), 3.54 (s, 1H), 3.41-3.35 (m, 1H), 3.21 (s, 3H), 2.70 (s, 1H), 2.65 (s, 1H), 2.55 (s, 2H), 1.92 (s, 2H), 1.46 (p, J = 7.2 Hz, 2H), 1.18 (h, J = 7.4 Hz, 2H), 0.83 (t, J = 7.4 Hz, 3H) |
| 103 | 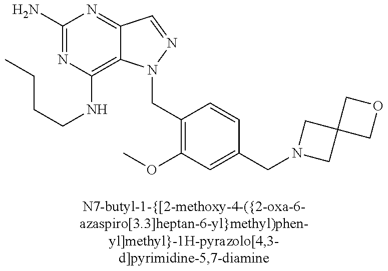 [SMILES: CCCCNc1nc(N)nc2cnn(Cc3ccc(CN4CC5(COC5)C4)cc3OC)c12] | 62.9 | 13.5 | LC/MS [M + H]⁺ = 438.0 RT (min) 0.8 (LC/MS Procedure B) δ 7.57 (s, 1H), 6.93 (s, 1H), 6.72 (d, J = 7.9 Hz, 1H), 6.45 (d, J = 7.8 Hz, 1H), 5.78 (s, 1H), 5.60 (s, 2H), 4.59 (s, 4H), 3.83 (s, 3H), 1.92 (s, 5H), 1.53-1.43 (m, 2H), 1.20 (h, J = 7.4 Hz, 2H), 0.85 (t, J = 7.3 Hz, 3H) |
| 104 | 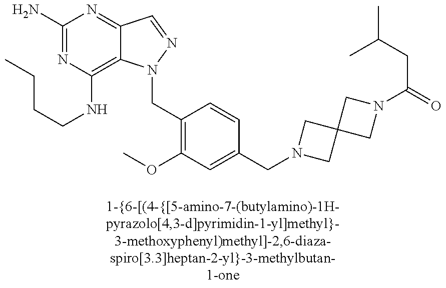 [SMILES: CCCCNc1nc(N)nc2cnn(Cc3ccc(CN4CC5(C4)CN(C(=O)CC(C)C)C5)cc3OC)c12] | 137.9 | | LC/MS [M + H]⁺ = 521.1 RT (min) 1 (LC/MS Procedure B) δ 7.55 (s, 1H), 6.91 (s, 1H), 6.71 (d, J = 7.8 Hz, 1H), 6.42 (d, J = 7.8 Hz, 1H), 5.69 (s, 1H), 5.60 (s, 2H), 4.13 (s, 2H), 3.87 (s, 2H), 3.83 (s, 3H), 1.92 (s, 9H), 1.87 (d, J = 6.7 Hz, 2H), 1.47 (p, J = 7.1 Hz, 2H), 1.19 (q, J = 7.5 Hz, 2H), 0.88-0.81 (m, 9H). |
| 105 | 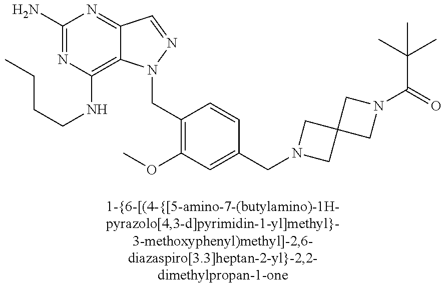 [SMILES: CCCCNc1nc(N)nc2cnn(Cc3ccc(CN4CC5(C4)CN(C(=O)C(C)(C)C)C5)cc3OC)c12] | 60.0 | | LC/MS [M + H]⁺ = 521.1 RT (min) 1.01 (LC/MS Procedure B) δ 7.55 (s, 1H), 6.91 (s, 1H), 6.70 (d, J = 7.7 Hz, 1H), 6.43 (d, J = 7.8 Hz, 1H), 5.61 (d, J = 17.9 Hz, 3H), 4.39 (s, 1H), 3.85-3.81 (m, 3H), 3.38 (t, J = 6.3 Hz, 1H), 3.27-3.21 (m, 7H), 1.89 (s, 4H), 1.46 (p, J = 7.3 Hz, 2H), 1.18 (h, J = 7.4 Hz, 2H), 1.06 (s, 9H), 0.84 (t, J = 7.4 Hz, 3H) |
| 106 | 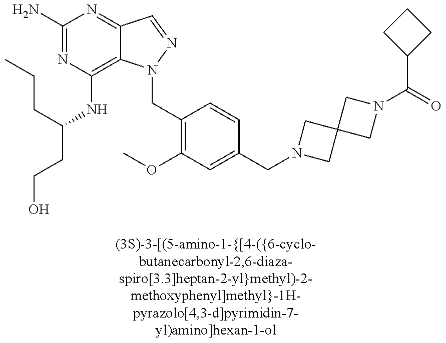 [SMILES: CCC[C@@H](CCO)Nc1nc(N)nc2cnn(Cc3ccc(CN4CC5(C4)CN(C(=O)C4CCC4)C5)cc3OC)c12] | 11.6 | 24.9 | LC/MS [M + H]⁺ = 563.1 RT (min) 0.91 (LC/MS Procedure B) δ 7.64 (s, 1H), 7.01 (s, 1H), 6.80 (d, J = 7.9 Hz, 1H), 6.48 (d, J = 7.7 Hz, 1H), 6.33 (s, 1H), 5.70 (d, J = 16.9 Hz, 1H), 5.62 (d, J = 17.0 Hz, 1H), 4.39 (s, 1H), 4.11 (s, 2H), 3.92 (s, 2H), 3.33 (s, 1H), 3.03 (t, J = 8.6 Hz, 1H), 2.93 (q, J = 7.4 Hz, 3H), 2.06 (q, J = 9.7 Hz, 2H), 1.99 (d, J = 9.5 Hz, 2H), 1.88 (dd, J = 20.0, 10.3 Hz, 2H), 1.77-1.63 (m, 2H), 1.57 (d, J = 6.6 Hz, 1H), 1.44 (p, J = 7.6 Hz, 2H), 1.17 (t, J = 7.3 Hz, 5H), 1.12 (s, 1H), 1.10-1.05 (m, 1H), 0.78 (t, J = 7.3 Hz, 3H) |
| 107 | 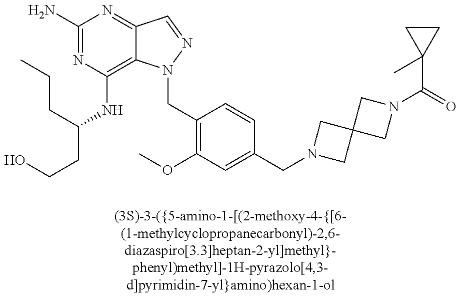 [SMILES: CCC[C@@H](CCO)Nc1nc(N)nc2cnn(Cc3ccc(CN4CC5(C4)CN(C(=O)C4(C)CC4)C5)cc3OC)c12] | 6.6 | 9.9 | LC/MS [M + H]⁺ = 563.2 RT (min) 1.01 (LC/MS Procedure B) δ 7.95 (s, 1H), 7.75 (s, 1H), 7.28 (s, 1H), 7.11 (s, 1H), 6.91 (d, J = 7.9 Hz, 1H), 6.66 (d, J = 7.2 Hz, 1H), 5.80- 5.68 (m, 3H), 4.50 (s, 1H), 4.20 (s, 2H), 3.82 (s, 3H), 3.47 (s, 1H), 3.38 (s, 1H), 3.18 (s, 1H), 2.90 (s, 2H), 1.75-1.67 (m, 3H), 1.51 (t, J = 7.8 Hz, 3H), 1.21 (s, 6H), 1.18-1.11 (m, 3H), 0.88 (d, J = 2.6 Hz, 3H), 0.82 (t, J = 7.3 Hz, 5H), 0.47 (q, J = 3.8 Hz, 3H) |
| 108 | 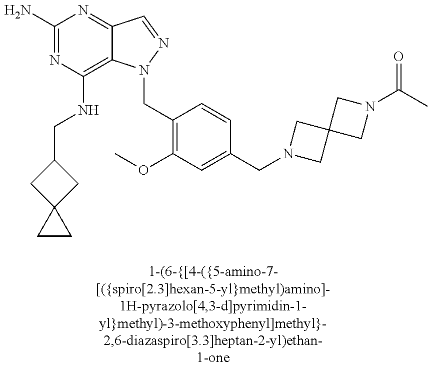 [SMILES: COc1cc(CN2CC3(C2)CN(C(C)=O)C3)ccc1Cn1ncc2nc(N)nc(NCC3CC4(CC4)C3)c21] | 50.1 | 52.9 | LC/MS [M + H]+ = 517.2 RT (min) 1.25 (LC/MS Procedure B) δ 7.57 (s, 1H), 6.92 (s, 1H), 6.71 (d, J = 7.7 Hz, 1H), 6.49 (s, 1H), 6.43 (d, J = 7.7 Hz, 1H), 5.70 (s, 1H), 5.60 (s, 2H), 4.15 (s, 2H), 3.85 (d, J = 17.2 Hz, 4H), 3.48 (s, 1H), 3.25 (s, 3H), 3.01 (s, 0H), 2.64 (d, J = 7.9 Hz, 1H), 1.99 (t, J = 10.0 Hz, 2H), 1.93 (s, 1H), 1.74 (d, J = 18.0 Hz, 5H), 0.35 (s, 4H) |
| 109 | 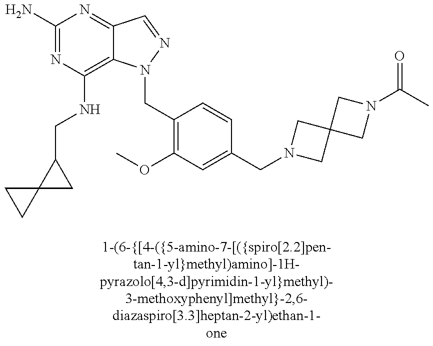 [SMILES: COc1cc(CN2CC3(C2)CN(C(C)=O)C3)ccc1Cn1ncc2nc(N)nc(NCC3CC34CC4)c21] | 1,227.5 | | LC/MS [M + H]+ = 503.1 RT (min) 1.07 (LC/MS Procedure B) δ 8.41 (d, J = 5.9 Hz, 1H), 7.95 (s, 0H), 7.78 (s, 1H), 7.29 (s, 0H), 7.19 (s, 0H), 7.11 (d, J = 15.8 Hz, 1H), 6.98-6.93 (m, 1H), 6.85 (d, J = 7.6 Hz, 1H), 5.75 (s, 2H), 4.32 (s, 2H), 4.25 (s, 7H), 4.18 (s, 1H), 3.98 (s, 1H), 3.79 (s, 3H), 3.64 (dd, J = 12.9, 6.5 Hz, 1H), 1.72 (s, 3H), 1.61-1.55 (m, 1H), 0.95 (dd, J = 7.8, 4.2 Hz, 1H), 0.78-0.72 (m, 3H), 0.68 (t, J = 4.2 Hz, 2H) |
| 110 | 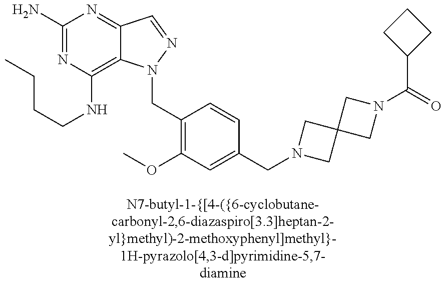 [SMILES: CCCCNc1nc(N)nc2cnn(Cc3ccc(CN4CC5(C4)CN(C(=O)C4CCC4)C5)cc3OC)c12] | 31.9 | | LC/MS [M + H]⁺ = 519.2 RT (min) 0.97 (LC/MS Procedure B) δ 7.55 (s, 1H), 6.91 (s, 1H), 6.71 (d, J = 7.9 Hz, 1H), 6.43 (d, J = 7.6 Hz, 2H), 5.65 (s, 2H), 5.60 (s, 2H), 4.09- 4.05 (m, 2H), 3.89-3.81 (m, 5H), 3.23 (s, 3H), 3.02 (q, J = 8.4 Hz, 1H), 2.07 (dd, J = 10.6, 8.2 Hz, 2H), 1.99 (d, J = 9.5 Hz, 2H), 1.92 (s, 3H), 1.91- 1.83 (m, 1H), 1.74 (t, J = 9.7 Hz, 1H), 1.47 (p, J = 7.1 Hz, 2H), 1.20 (q, J = 7.6 Hz, 2H), 0.85 (t, J = 7.3 Hz, 3H) |
| 111 | 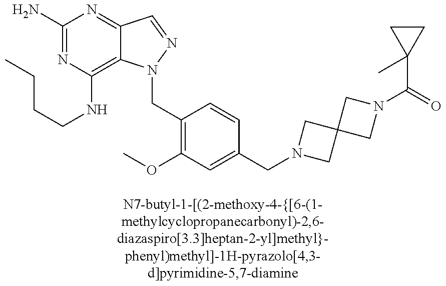 [SMILES: CCCCNc1nc(N)nc2cnn(Cc3ccc(CN4CC5(C4)CN(C(=O)C4(C)CC4)C5)cc3OC)c12] | 52.5 | 24.9 | LC/MS [M + H]+ = 519.2 RT (min) 0.97 (LC/MS Procedure B) δ 7.56 (s, 1H), 6.92 (s, 1H), 6.72 (d, J = 7.8 Hz, 1H), 6.44 (d, J = 7.6 Hz, 2H), 5.66 (s, 2H), 5.60 (s, 2H), 3.83 (s, 3H), 3.52-3.35 (m, 1H), 3.27 (s, 2H), 3.18 (s, 0H), 3.00 (s, 0H), 2.56 (s, 6H), 1.92 (s, 0H), 1.47 (t, J = 7.5 Hz, 2H), 1.20 (d, J = 7.6 Hz, 5H), 1.17 (s, 1H), 0.88-0.81 (m, 5H), 0.44 (q, J = 3.9 Hz, 2H) |
| 112 | 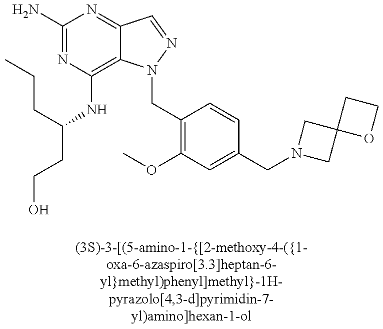 [SMILES: CCC[C@@H](CCO)Nc1nc(N)nc2cnn(Cc3ccc(CN4CC5(CCO5)C4)cc3OC)c12] | 1.5 | 333.3 | LC/MS [M + H]+ = 482.1 RT (min) 0.99 (LC/MS Procedure B) δ 7.57 (s, 1H), 6.94 (s, 1H), 6.72 (d, J = 7.8 Hz, 1H), 6.38 (d, J = 7.8 Hz, 1H), 5.66 (dd, J = 23.8, 7.7 Hz, 3H), 5.55 (d, J = 17.0 Hz, 1H), 4.35 (t, J = 7.5 Hz, 3H), 3.85 (s, 3H), 3.50-3.43 (m, 1H), 3.32 (q, J = 6.9, 6.1 Hz, 1H), 3.09-3.02 (m, 2H), 2.73 (d, J = 15.1 Hz, 2H), 1.91 (s, 1H), 1.64 (dd, J = 13.4, 6.4 Hz, 1H), 1.54-1.48 (m, 1H), 1.45-1.38 (m, 1H), 1.40- 1.31 (m, 1H), 1.05 (d, J = 8.2 Hz, 2H), 0.77 (t, J = 7.3 Hz, 3H) |
| 113 | 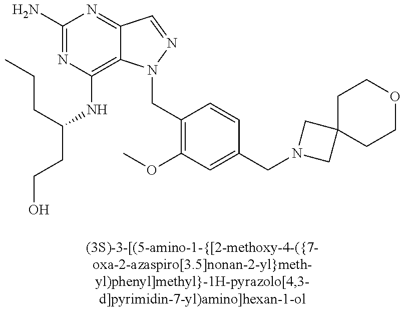 [SMILES: CCC[C@@H](CCO)Nc1nc(N)nc2cnn(Cc3ccc(CN4CC5(CCOCC5)C4)cc3OC)c12] | 4.6 | 0.5 | LC/MS [M + H]+ = 509.9 RT (min) 1.31 (LC/MS Procedure B) δ 7.58 (s, 1H), 6.96 (s, 1H), 6.74 (d, J = 8.0 Hz, 1H), 6.38 (d, J = 7.7 Hz, 1H), 5.71-5.63 (m, 3H), 5.56 (d, J = 17.0 Hz, 1H), 4.33 (s, 1H), 3.86 (s, 3H), 3.54 (s, 1H), 3.33 (d, J = 7.0 Hz, 1H), 2.96 (s, 4H), 2.56 (s, 5H), 1.92 (s, 3H), 1.64 (t, J = 5.4 Hz, 5H), 1.55- 1.48 (m, 1H), 1.42 (s, 1H), 1.36 (t, J = 7.1 Hz, 1H), 1.06 (s, 1H), 0.78 (t, J = 7.2 Hz, 3H) |
| 114 | 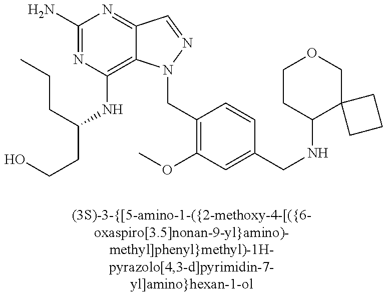 [SMILES: CCC[C@@H](CCO)Nc1nc(N)nc2cnn(Cc3ccc(CNC4CCOCC45CCC5)cc3OC)c12] | 3.4 | | LC/MS [M + H]+ = 524.3 RT (min) 0.93 (LC/MS Procedure B) δ 7.57 (s, 1H), 7.14 (s, 1H), 6.83 (d, J = 7.7 Hz, 1H), 6.41 (d, J = 7.7 Hz, 1H), 5.73-5.62 (m, 3H), 5.55 (dd, J = 16.9, 3.4 Hz, 1H), 4.33 (s, 1H), 3.85 (s, 3H), 3.67 (dd, J = 30.8, 12.5 Hz, 2H), 3.33 (s, 1H), 3.25 (t, J = 11.0 Hz, 0H), 3.16 (d, J = 11.2 Hz, 1H), 2.30 (d, J = 8.1 Hz, 1H), 2.00 (dt, J = 18.2, 9.4 Hz, 2H), 1.92 (s, 1H), 1.81 (q, J = 9.3, 8.9 Hz, 1H), 1.72 (dt, J = 10.7, 5.2 Hz, 1H), 1.67- 1.60 (m, 2H), 1.52 (d, J = 11.1 Hz, 2H), 1.42 (d, J = 7.9 Hz, 2H), 1.35 (td, J = 14.1, 13.4, 6.2 Hz, 2H), 1.23- 1.17 (m, 1H), 1.05 (d, J = 7.5 Hz, 2H), 0.77 (t, J = 7.3 Hz, 3H) |
| 115 | 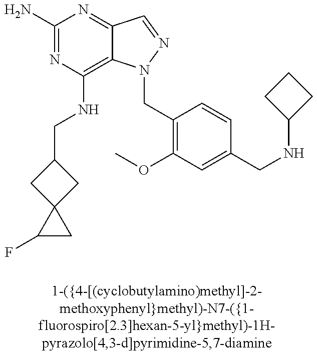 [SMILES: COc1cc(CNC2CCC2)ccc1Cn1ncc2nc(N)nc(NCC3CC4(C3)CC4F)c21] | 84.1 | | LC/MS [M + H]+ = 466.2 RT (min) 1 (LC/MS Procedure B) δ 8.39 (s, 1H), 7.87 (s, 0H), 7.75 (d, J = 1.8 Hz, 1H), 7.17 (s, 1H), 6.97 (d, J = 7.7 Hz, 1H), 6.88 (dd, J = 12.7, 7.7 Hz, 1H), 5.73 (s, 2H), 4.62 (s, 0H), 4.47 (s, 0H), 3.99 (s, 2H), 3.76 (d, J = 3.4 Hz, 3H), 3.64 (s, 1H), 3.52 (s, 1H), 2.88-2.74 (m, 1H), 2.14 (q, J = 8.0 Hz, 5H), 1.98 (dt, J = 18.3, 10.6 Hz, 2H), 1.77 (td, J = 19.4, 18.4, 9.8 Hz, 3H), 0.79 (s, 1H), 0.78-0.68 (m, 2H) |
| 116 | 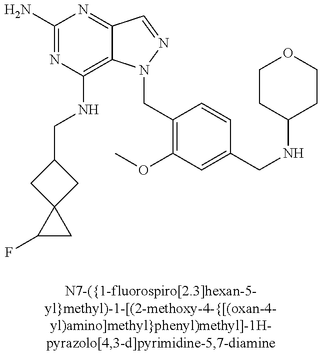 [SMILES: COc1cc(CNC2CCOCC2)ccc1Cn1ncc2nc(N)nc(NCC3CC4(C3)CC4F)c21] | 162.6 | | LC/MS [M + H]+ = 495.9 RT (min) 1.13 (LC/MS Procedure B) δ 7.55 (d, J = 2.1 Hz, 1H), 7.04 (s, 1H), 6.81-6.75 (m, 1H), 6.46 (dd, J = 16.3, 7.7 Hz, 1H), 5.67 (s, 2H), 5.60 (s, 2H), 4.59 (d, J = 5.4 Hz, 1H), 4.46 (d, J = 5.9 Hz, 1H), 3.85-3.77 (m, 5H), 3.68 (s, 1H), 3.27-3.16 (m, 2H), 2.68 (dd, J = 15.4, 7.8 Hz, 1H), 2.11 (q, J = 10.3 Hz, 1H), 1.95 (t, J = 9.7 Hz, 2H), 1.91 (s, 6H), 1.83 (dd, J = 11.7, 6.8 Hz, 1H), 1.32- 1.20 (m, 3H), 0.75-0.62 (m, 2H). |
| 117 | 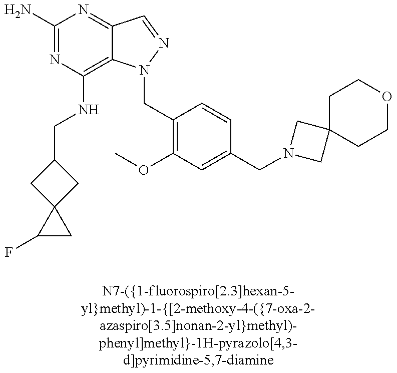 [SMILES: COc1cc(CN2CC3(CCOCC3)C2)ccc1Cn1ncc2nc(N)nc(NCC3CC4(C3)CC4F)c21] | 141.2 | 121.8 | LC/MS [M + H]+ = 522.2 RT (min) 0.97 (LC/MS Procedure B) δ 7.56 (d, J = 2.1 Hz, 1H), 6.93 (s, 1H), 6.72 (d, J = 7.7 Hz, 1H), 6.55- 6.49 (m, 1H), 6.40 (dd, J = 13.1, 7.7 Hz, 1H), 5.67 (s, 2H), 5.60 (s, 2H), 4.58 (s, 0H), 4.44 (d, J = 7.3 Hz, 1H), 3.83 (d, J = 1.7 Hz, 3H), 3.55 (d, J = 6.3 Hz, 1H), 2.96 (s, 3H), 2.67 (d, J = 7.8 Hz, 1H), 2.55 (s, 7H), 2.08 (q, J = 12.1 Hz, 1H), 1.97-1.90 (m, 1H), 1.87-1.76 (m, 1H), 1.68 (s, 1H), 1.63 (t, J = 5.2 Hz, 5H), 0.76-0.62 (m, 2H). |
| 118 | 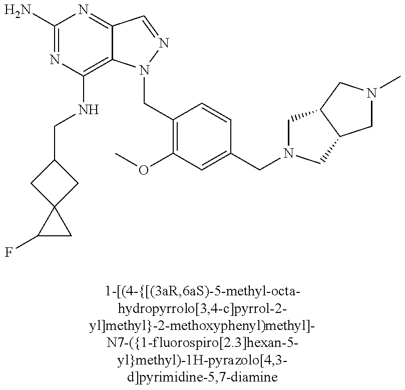 [SMILES: COc1cc(CN2C[C@H]3CN(C)C[C@H]3C2)ccc1Cn1ncc2nc(N)nc(NCC3CC4(C3)CC4F)c21] | 137.0 | | LC/MS [M + H]+ = 521.3 RT (min) 0.83 (LC/MS Procedure B) δ 7.56 (d, J = 1.9 Hz, 1H), 6.98 (s, 1H), 6.77 (d, J = 7.7 Hz, 1H), 6.59- 6.52 (m, 1H), 6.40 (dd, J = 12.5, 7.7 Hz, 1H), 5.69 (s, 1H), 5.61 (s, 2H), 4.59-4.52 (m, 1H), 4.42 (dd, J = 13.9, 5.8 Hz, 1H), 3.82 (d, J = 1.5 Hz, 3H), 3.55 (s, 1H), 3.53 (d, J = 5.8 Hz, 1H), 2.75 (s, 1H), 2.66 (s, 2H), 2.55 (s, 8H), 2.38 (s, 3H), 2.32 (d, J = 9.2 Hz, 2H), 2.07 (q, J = 11.3 Hz, 1H), 1.92 (t, J = 10.2 Hz, 1H), 1.86-1.75 (m, 1H), 1.67 (dt, J = 12.1, 6.7 Hz, 1H), 0.72 (d, J = 15.7 Hz, 1H), 0.68 (s, 1H). |
| 119 | 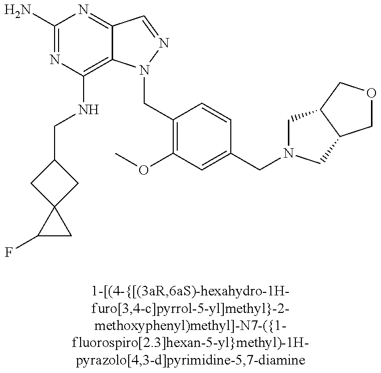 [SMILES: COc1cc(CN2C[C@H]3COC[C@H]3C2)ccc1Cn1ncc2nc(N)nc(NCC3CC4(C3)CC4F)c21] | 144.1 | 257.8 | LC/MS [M + H]+ = 508.2 RT (min) 0.95 (LC/MS Procedure B) δ 7.56 (d, J = 2.0 Hz, 1H), 6.97 (s, 1H), 6.74 (d, J = 7.9 Hz, 1H), 6.51 (d, J = 14.8 Hz, 1H), 6.41 (dd, J = 12.1, 7.8 Hz, 1H), 5.67 (s, 2H), 5.61 (s, 2H), 4.56 (s, 1H), 4.44 (d, J = 5.8 Hz, 1H), 3.83 (s, 3H), 3.71 (t, J = 7.1 Hz, 2H), 3.61-3.53 (m, 1H), 3.37 (d, J = 8.6 Hz, 1H), 2.70 (s, 2H), 2.66 (s, 2H), 2.26 (dd, J = 9.2, 2.9 Hz, 3H), 2.08 (q, J = 11.9 Hz, 2H), 1.87-1.76 (m, 2H), 1.69 (d, J = 11.8 Hz, 1H), 0.76-0.62 (m, 3H) |
| 120 | 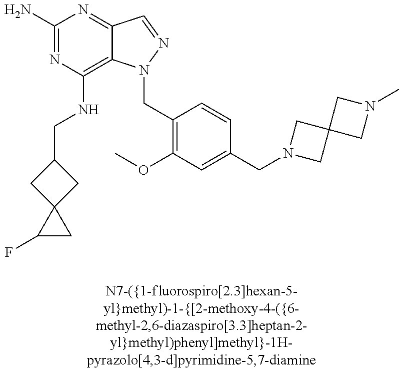 [SMILES: COc1cc(CN2CC3(CN(C)C3)C2)ccc1Cn1ncc2nc(N)nc(NCC3CC4(C3)CC4F)c21] | 261.8 | | LC/MS [M + H]+ = 506.9 RT (min) 1.03 (LC/MS Procedure B) δ 7.55 (d, J = 1.9 Hz, 1H), 6.88 (s, 1H), 6.68 (d, J = 7.7 Hz, 1H), 6.55- 6.49 (m, 1H), 6.39 (dd, J = 14.2, 7.7 Hz, 1H), 5.65 (s, 2H), 5.59 (s, 2H), 4.58 (s, 0H), 4.46-4.38 (m, 1H), 3.81 (d, J = 1.6 Hz, 3H), 3.18 (s, 4H), 3.13 (s, 4H), 2.68 (d, J = 10.4 Hz, 1H), 2.16 (s, 3H), 2.07 (q, J = 10.7 Hz, 2H), 1.91 (t, J = 10.1 Hz, 2H), 1.86-1.75 (m, 2H), 1.66 (s, 1H), 0.77-0.66 (m, 2H), 0.63 (d, J = 6.8 Hz, 1H). |
| 121 | 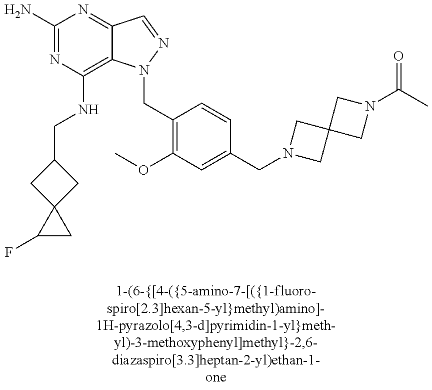 [SMILES: COc1cc(CN2CC3(C2)CN(C(C)=O)C3)ccc1Cn1ncc2nc(N)nc(NCC3CC4(C3)CC4F)c21] | 358.2 | 160.0 | LC/MS [M + H]+ = 535.3 RT (min) 0.9 (LC/MS Procedure B) δ 7.57 (d, J = 2.2 Hz, 1H), 6.92 (s, 1H), 6.69 (dd, J = 22.7, 10.4 Hz, 2H), 6.43 (dd, J = 16.1, 7.7 Hz, 1H), 5.80 (s, 1H), 5.61 (s, 2H), 4.57 (d, J = 14.8 Hz, 1H), 4.43 (dd, J = 15.9, 6.2 Hz, 1H), 4.14 (s, 2H), 3.86 (s, 2H), 3.82 (d, J = 2.2 Hz, 3H), 3.53 (d, J = 14.5 Hz, 2H), 3.32 (s, 1H), 2.71-2.63 (m, 1H), 2.08 (q, J = 9.2 Hz, 1H), 1.93 (t, J = 10.1 Hz, 1H), 1.88-1.77 (m, 1H), 1.71 (s, 4H), 1.68 (d, J = 7.1 Hz, 1H), 0.77-0.62 (m, 2H) |
| 122 | 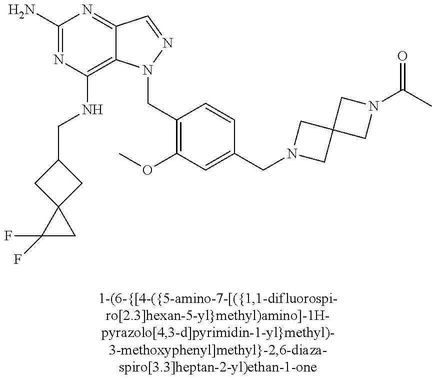 [SMILES: COc1cc(CN2CC3(C2)CN(C(C)=O)C3)ccc1Cn1ncc2nc(N)nc(NCC3CC4(C3)CC4(F)F)c21] | 241.4 | | LC/MS [M + H]+ = 553.1 RT (min) 0.95 (LC/MS Procedure B) δ 8.43-8.38 (m, 1H), 7.76 (d, J = 2.2 Hz, 1H), 7.09 (s, 1H), 6.97-6.91 (m, 1H), 6.86 (dd, J = 15.5, 7.7 Hz, 1H), 5.72 (s, 2H), 4.27 (d, J = 18.4 Hz, 4H), 4.20 (s, 3H), 3.98 (s, 1H), 3.76 (d, J = 4.5 Hz, 1H), 2.82 (s, 0H), 2.70 (q, J = 7.6 Hz, 1H), 2.16 (dt, J = 23.3, 10.7 Hz, 2H), 2.02-1.92 (m, 2H), 1.72 (s, 3H), 1.34 (dt, J = 15.9, 8.3 Hz, 2H) |
| 123 | 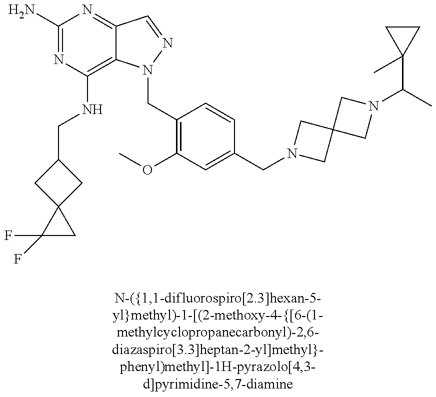 [SMILES: COc1cc(CN2CC3(C2)CN(C(C)C2(C)CC2)C3)ccc1Cn1ncc2nc(N)nc(NCC3CC4(C3)CC4(F)F)c21] | 319.6 | | LC/MS (M + H]+ = 593.2 RT (min) 1.24 (LC/MS Procedure B) δ 7.57 (d, J = 4.0 Hz, 1H), 6.91 (d, J = 2.7 Hz, 1H), 6.71 (d, J = 7.8 Hz, 1H), 6.39 (dd, J = 25.4, 7.7 Hz, 1H), 5.71 (d, J = 9.0 Hz, 2H), 5.61 (d, J = 4.6 Hz, 2H), 3.82 (d, J = 3.9 Hz, 3H), 3.27 (s, 2H), 2.71 (d, J = 13.8 Hz, 1H), 2.10 (dt, J = 20.7, 10.6 Hz, 3H), 1.92 (s, 5H), 1.90-1.77 (m, 3H), 1.30 (dt, J = 23.2, 8.4 Hz, 3H), 1.20 (s, 3H), 0.86 (d, J = 2.6 Hz, 2H), 0.45 (d, J = 2.7 Hz, 2H) |
| 124 | 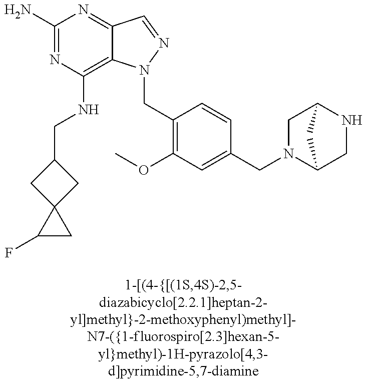 [SMILES: COc1cc(CN2C[C@@H]3C[C@H]2CN3)ccc1Cn1ncc2nc(N)nc(NCC3CC4(C3)CC4F)c21] | 193.1 | | LC/MS [M + H]+ = 493.1 RT (min) 1 (LC/MS Procedure B) δ 7.55 (d, J = 2.0 Hz, 1H), 6.98 (s, 1H), 6.76 (d, J = 7.7 Hz, 1H), 6.42 (dd, J = 13.0, 7.7 Hz, 1H), 5.65 (s, 1H), 5.60 (s, 2H), 4.56 (d, J = 18.4 Hz, 1H), 4.4.6-4.38 (m, 1H), 3.82 (d, J = 1.7 Hz, 3H), 3.70 (s, 1H), 3.63 (t, J = 15.1 Hz, 1H), 3.19-3.10 (m, 1H), 2.79 (d, J = 10.3 Hz, 1H), 2.69 (d, J = 10.2 Hz, 2H), 2.46 (s, 1H), 2.08 (d, J = 10.6 Hz, 1H), 1.92 (t, J = 10.0 Hz, 1H), 1.82 (s, 5H), 1.79 (d, J = 9.8 Hz, 2H), 1.70-1.64 (m, 1H), 1.52 (d, J = 9.9 Hz, 1H), 0.76-0.60 (m, 3H). |
| 125 | 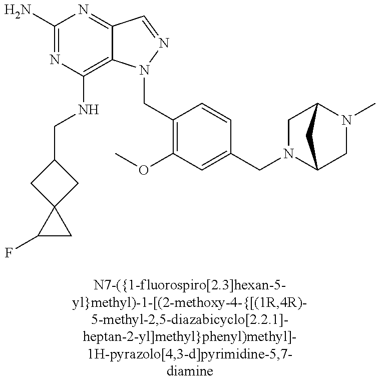 [SMILES: COc1cc(CN2C[C@H]3C[C@@H]2CN3C)ccc1Cn1ncc2nc(N)nc(NCC3CC4(C3)CC4F)c21] | 59.2 | | LC/MS [M + H]+ = 507.1 RT (min) 1.34 (LC/MS Procedure B) δ 7.56 (d, J = 1.8 Hz, 1H), 7.00 (s, 1H), 6.77 (d, J = 7.9 Hz, 1H), 6.50 (s, 0H), 6.41 (dd, J = 10.9, 7.6 Hz, 1H), 5.67 (s, 1H), 5.60 (s, 2H), 4.56 (d, J = 13.7 Hz, 1H), 4.42 (d, J = 13.6 Hz, 1H), 3.83 (s, 3H), 3.61 (t, J = 15.8 Hz, 1H), 3.16 (d, J = 19.8 Hz, 2H), 2.72 (s, 1H), 2.66 (d, J = 7.5 Hz, 1H), 2.27 (s, 3H), 2.11 (d, J = 12.6 Hz, 1H), 2.05 (s, 0H), 1.93 (t, J = 9.8 Hz, 1H), 1.87 (s, 4H), 1.84-1.77 (m, 1H), 1.72-1.65 (m, 1H), 1.60 (s, 2H), 0.68 (dd, J = 21.4, 13.5 Hz, 2H) |
| 126 | 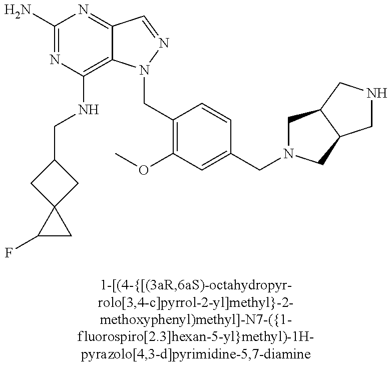 [SMILES: COc1cc(CN2C[C@H]3CNC[C@H]3C2)ccc1Cn1ncc2nc(N)nc(NCC3CC4(C3)CC4F)c21] | 243.8 | | LC/MS [M + H]+ = 507.1 RT (min) 1.32 (LC/MS Procedure B) δ 7.56 (d, J = 2.1 Hz, 1H), 6.97 (s, 1H), 6.77 (d, J = 7.9 Hz, 1H), 6.41 (dd, J = 13.4, 7.6 Hz, 2H), 5.67 (s, 2H), 5.61 (s, 3H), 4.56 (d, J = 15.2 Hz, 1H), 4.46-4.39 (m, 1H), 3.83 (s, 3H), 3.11 (s, 1H), 2.43 (d, J = 9.0 Hz, 3H), 2.35 (s, 2H), 2.07 (dd, J = 12.7, 8.6 Hz, 2H), 1.88 (s, 6H), 1.80 (dd, J = 11.8, 7.3 Hz, 2H), 1.71-1.66 (m, 1H), 0.74-0.61 (m, 3H). |
| 127 | 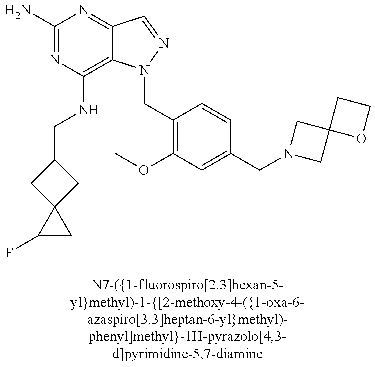 [SMILES: COc1cc(CN2CC3(CCO3)C2)ccc1Cn1ncc2nc(N)nc(NCC3CC4(C3)CC4F)c21] | 213.6 | 413.2 | LC/MS [M + H]+ = 494.1 RT (min) 0.95 (LC/MS Procedure B) δ 8.39 (s, 1H), 7.95 (s, 1H), 7.77 (d, J = 1.9 Hz, 1H), 7.15 (s, 1H), 6.96 (d, J = 7.7 Hz, 1H), 6.81 (dd, J = 12.0, 7.7 Hz, 1H), 5.74 (s, 2H), 4.47 (dd, J = 13.2, 5.3 Hz, 1H), 4.40 (t, J = 7.5 Hz, 2H), 4.31 (d, J = 14.8 Hz, 4H), 4.20 (d, J = 11.5 Hz, 2H), 3.77 (d, J = 3.6 Hz, 3H), 3.73 (t, J = 6.8 Hz, 2H), 3.47 (s, 1H), 2.88-2.77 (m, 3H), 2.15 (s, 1H), 2.01-1.89 (m, 2H), 1.80 (s, 1H), 0.79-0.68 (m, 2H). |
| 128 | 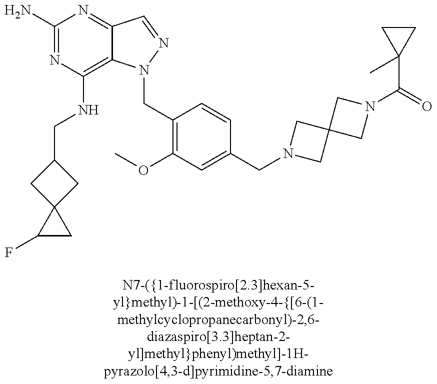 [SMILES: COc1cc(CN2CC3(C2)CN(C(=O)C2(C)CC2)C3)ccc1Cn1ncc2nc(N)nc(NCC3CC4(C3)CC4F)c21] | 348.6 | | LC/MS [M + H]+ = 575.1 RT (min) 1.2 (LC/MS Procedure B) δ 7.56 (d, J = 2.5 Hz, 1H), 6.90 (s, 1H), 6.70 (d, J = 7.8 Hz, 1H), 6.53 (d, J = 16.2 Hz, 1H), 6.39 (dd, J = 15.4, 7.7 Hz, 1H), 5.68 (s, 2H), 5.60 (s, 2H), 4.57 (d, J = 13.4 Hz, 1H), 4.43 (d, J = 13.2 Hz, 1H), 3.83 (d, J = 2.2 Hz, 3H), 3.54 (t, J = 6.6 Hz, 1H), 3.22 (s, 2H), 3.01 (d, J = 1.3 Hz, 2H), 2.72- 2.64 (m, 1H), 2.09 (q, J = 10.2 Hz, 1H), 1.97-1.89 (m, 1H), 1.80 (t, J = 9.4 Hz, 1H), 1.72-1.66 (m, 1H), 1.21 (s, 3H), 0.86 (s, 2H), 0.77- 0.63 (m, 2H), 0.44 (t, J = 3.2 Hz, 2H). |
| 129 | 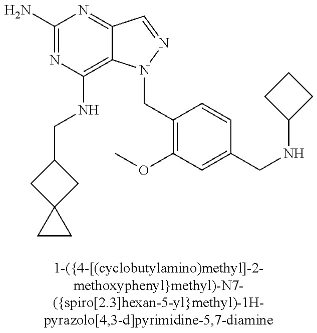 [SMILES: COc1cc(CNC2CCC2)ccc1Cn1ncc2nc(N)nc(NCC3CC4(CC4)C3)c21] | 24.4 | | LC/MS [M + H]+ = 448.2 RT (min) 1.57 (LC/MS Procedure B) δ 8.08 (s, 1H), 7.61 (s, 0H), 7.50 (s, 1H), 6.94 (s, 1H), 6.76-6.70 (m, 1H), 6.61 (d, J = 7.7 Hz, 1H), 5.49 (s, 2H), 3.75 (s, 2H), 3.50 (d, J = 12.8 Hz, 4H), 3.40 (t, J = 8.0 Hz, 1H), 2.93 (s, 0H), 2.56-2.47 (m, 1H), 1.89 (t, J = 8.5 Hz, 4H), 1.82 (dd, J = 11.5, 8.4 Hz, 2H), 1.63 (dd, J = 11.9, 6.1 Hz, 2H), 1.57-1.47 (m, 2H), 0.14 (s, 4H) |
| 130 | 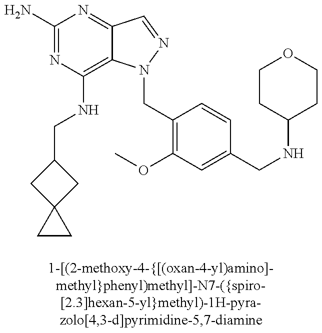 [SMILES: COc1cc(CNC2CCOCC2)ccc1Cn1ncc2nc(N)nc(NCC3CC4(CC4)C3)c21] | 39.7 | | LC/MS [M + H]+ = 478.2 RT (min) 1.21 [LC/MS Procedure B) δ 7.55 (s, 1H), 7.06 (s, 1H), 6.80 (d, J = 7.6 Hz, 1H), 6.47 (d, J = 7.7 Hz, 1H), 5.65 (s, 1H), 5.60 (s, 2H), 3.83 (s, 3H), 3.80 (s, 0H), 3.72 (s, 1H), 3.21 (td, J = 12.5, 10.4 Hz, 2H), 2.64 (d, J = 7.0 Hz, 1H), 2.56 (s, 6H), 2.02 (s, 1H), 1.91 (s, 3H), 1.76 (dd, J = 11.7, 5.3 Hz, 4H), 1.35-1.23 (m, 2H), 0.35 (s, 4H) |
| 131 | 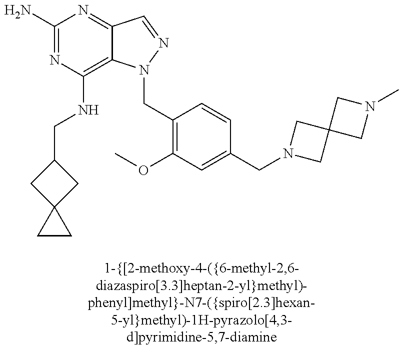 [SMILES: COc1cc(CN2CC3(CN(C)C3)C2)ccc1Cn1ncc2nc(N)nc(NCC3CC4(CC4)C3)c21] | 233.6 | 4.6 | LC/MS [M + H]+ = 489.2 RT (min) 1.31 (LC/MS Procedure B) δ 7.54 (s, 1H), 6.88 (s, 1H), 6.67 (d, J = 7.8 Hz, 1H), 6.45-6.37 (m, 2H), 5.62 (s, 2H), 5.57 (s, 2H), 3.80 (s, 2H), 3.52 (d, J = 6.7 Hz, 1H), 3.42 (s, 1H), 3.29 (s, 3H), 3.14 (s, 3H), 2.98 (d, J = 1.2 Hz, 1H), 2.55 (s, 5H), 2.22 (s, 3H), 1.95 (dd, J = 11.5, 8.5 Hz, 2H), 1.71 (dd, J = 11.8, 6.1 Hz, 2H), 0.33 (s, 4H). |
| 132 | 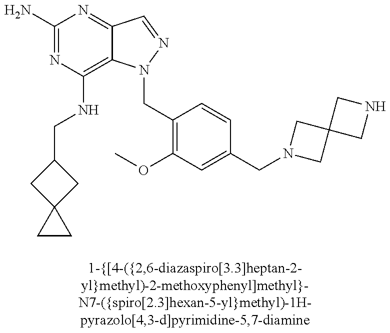 [SMILES: COc1cc(CN2CC3(CNC3)C2)ccc1Cn1ncc2nc(N)nc(NCC3CC4(CC4)C3)c21] | 314.4 | 70.2 | LC/MS [M + H]+ = 473.3 RT (min) 1.29 (LC/MS Procedure B) δ 7.30 (s, 1H), 6.64 (s, 1H), 6.43 (d, J = 7.8 Hz, 1H), 6.20 (s, 0H), 6.13 (d, J = 7.6 Hz, 1H), 5.43 (s, 2H), 5.34 (s, 2H), 3.58 (d, J = 2.1 Hz, 3H), 3.40 (s, 1H), 3.29 (s, 1H), 2.92 (s, 2H), 2.75 (d, J = 2.2 Hz, 1H), 2.30 (s, 7H), 1.72 (dd, J = 11.4, 8.7 Hz, 2H), 1.61 (s, 1H), 1.48 (dd, J = 11.6, 6.5 Hz, 3H), 0.10 (s, 4H) |
| 133 | 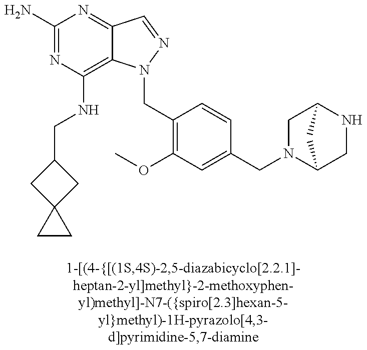 | 39.3 | | LC/MS [M + H]+ = 472. RT (min) 1.1 (LC/MS Procedure B) δ 8.33 (s, 1H), 7.90 (s, 0H), 7.76 (s, 1H), 7.25 (d, J = 51.0 Hz, 0H), 7.10 (s, 1H), 6.95 (d, J = 7.9 Hz, 1H), 6.82 (d, J = 7.6 Hz, 1H), 5.72 (s, 2H), 4.33 (s, 1H), 4.09 (s, 0H), 4.01 (s, 1H), 3.76 (s, 2H), 3.54 (s, 0H), 3.23 (d, J = 12.3 Hz, 1H), 3.10 (s, 1H), 2.77- 2.70 (m, 1H), 2.29 (s, 1H), 2.04 (dd, J = 11.7, 8.5 Hz, 2H), 1.92-1.81 (m, 3H), 0.37 (s, 4H). |
| 134 | 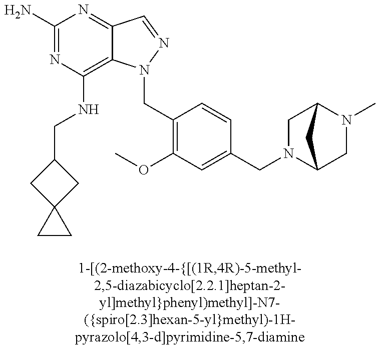 [SMILES: COc1cc(CN2C[C@H]3C[C@@H]2CN3C)ccc1Cn1ncc2nc(N)nc(NCC3CC4(CC4)C3)c21] | 16.6 | | LC/MS [M + H]+ = 489.1 RT (min) 1.1 (LC/MS Procedure B) δ 7.31 (s, 1H), 6.76 (s, 1H), 6.52 (d, J = 7.6 Hz, 1H), 6.15 (d, J = 7.5 Hz, 2H), 5.42 (s, 2H), 5.35 (s, 2H), 3.41- 3.27 (m, 4H), 3.27 (s, 0H), 2.92 (s, 1H), 2.86 (s, 1H), 2.76 (s, 2H), 2.44 (d, J = 9.5 Hz, 1H), 2.01 (s, 3H), 1.73 (dd, J = 11.5, 8.5 Hz, 3H), 1.48 (dd, J = 11.9, 6.1 Hz, 3H), 1.34 (s, 2H), 0.09 (s, 4H) |
| 135 | 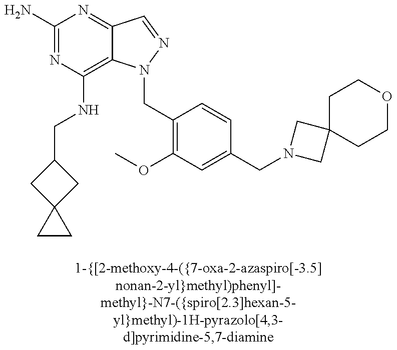 [SMILES: COc1cc(CN2CC3(CCOCC3)C2)ccc1Cn1ncc2nc(N)nc(NCC3CC4(CC4)C3)c21] | 69.8 | | LC/MS [M + H]+ = 503.9 RT (min) 1.57 (LC/MS Procedure B) δ 8.13 (s, 1H), 7.71 (s, 1H), 7.54 (d, J = 1.8 Hz, 1H), 6.95-6.91 (m, 1H), 6.79-6.73 (m, 1H), 6.59 (d, J = 7.7 Hz, 1H), 5.51 (s, 2H), 4.12 (s, 2H), 3.65 (s, 2H), 3.54 (s, 3H), 3.50 (t, J = 6.6 Hz, 2H), 3.25 (s, 1H), 2.70 (s, 0H), 2.56-2.49 (m, 1H), 2.50 (s, 0H), 1.86-1.78 (m, 2H), 1.63 (dd, J = 11.7, 6.2 Hz, 2H), 1.54 (s, 4H), 1.43 (d, J = 7.0 Hz, 1H), 1.32 (s, 0H), 0.94 (t, J = 7.3 Hz, 1H), 0.15 (s, 4H) |
| 136 | 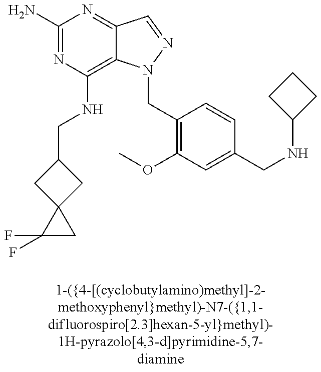 [SMILES: COc1cc(CNC2CCC2)ccc1Cn1ncc2nc(N)nc(NCC3CC4(C3)CC4(F)F)c21] | 68.1 | | LC/MS [M + H]+ = 484.1 RT (min) 1.25 (LC/MS Procedure B) δ 7.55 (d, J = 3.6 Hz, 1H), 7.01 (s, 1H), 6.75 (d, J = 7.5 Hz, 1H), 6.65 (d, J = 5.8 Hz, 1H), 6.60 (s, 0H), 6.41 (dd, J = 23.3, 7.7 Hz, 1H), 5.67 (d, J = 9.3 Hz, 2H), 5.60 (s, 2H), 3.82 (d, J = 5.4 Hz, 3H), 3.17-3.10 (m, 1H), 2.68 (dd, J = 9.7, 2.2 Hz, 1H), 2.09 (ddd, J = 31.7, 20.8, 9.7 Hz, 4H), 1.89 (d, J = 16.5 Hz, 2H), 1.82 (s, 1H), 1.73-1.66 (m, 2H), 1.65- 1.57 (m, 1H), 1.52 (q, J = 9.4 Hz, 1H), 1.37-1.25 (m, 2H). |
| 137 | 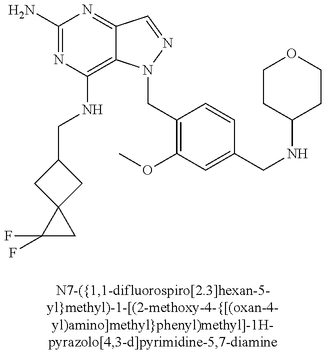 [SMILES: COc1cc(CNC2CCOCC2)ccc1Cn1ncc2nc(N)nc(NCC3CC4(C3)CC4(F)F)c21] | 88.1 | | LC/MS [M + H]+ = 514.3 RT (min) 1.44 (LC/MS Procedure B) δ 7.55 (d, J = 3.8 Hz, 1H), 7.04 (s, 1H), 6.79 (d, J = 7.5 Hz, 1H), 6.68 (s, 0H), 6.62 (s, 0H), 6.44 (dd, J = 24.3, 7.6 Hz, 1H), 5.67 (d, J = 9.5 Hz, 2H), 5.60 (d, J = 3.5 Hz, 2H), 3.82 (dd, J = 3.0, 1.3 Hz, 4H), 3.80 (s, 1H), 3.70 (s, 2H), 3.23 (t, J = 11.4 Hz, 2H), 2.70 (d, J = 16.6 Hz, 1H), 2.11 (dt, J = 20.8, 10.3 Hz, 2H), 1.92-1.82 (m, 2H), 1.75 (d, J = 12.9 Hz, 2H), 1.34 (d, J = 8.2 Hz, 1H), 1.28 (td, J = 18.3, 16.5, 7.5 Hz, 4H) |
| 138 | 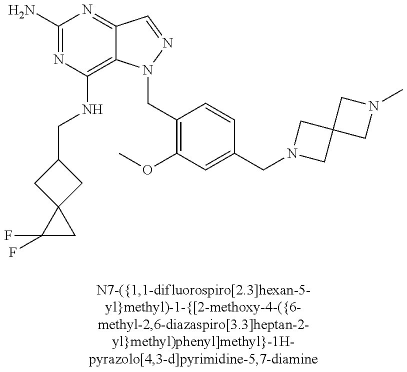 [SMILES: COc1cc(CN2CC3(CN(C)C3)C2)ccc1Cn1ncc2nc(N)nc(NCC3CC4(C3)CC4(F)F)c21] | 244.0 | | LC/MS [M + H]+ = 525.1 RT (min) 1.33 (LC/MS Procedure B) δ 7.56 (d, J = 3.7 Hz, 1H), 6.89 (d, J = 2.2 Hz, 1H), 6.68 (d, J = 7.8 Hz, 1H), 6.37 (dd, J = 20.1, 7.7 Hz, 1H), 5.67 (d, J = 7.9 Hz, 2H), 5.61 (d, J = 4.0 Hz, 2H), 3.92 (s, 1H), 3.83 (d, J = 3.2 Hz, 3H), 3.52 (dt, J = 32.7, 6.4 Hz, 3H), 2.60 (d, J = 15.4 Hz, 1H), 2.17- 2.03 (m, 6H), 1.91 (s, 3H), 1.87 (d, J = 5.4 Hz, 1H), 1.85 (d, J = 5.4 Hz, 1H), 1.81 (s, 1H), 1.77 (s, 1H), 1.32 (dt, J = 25.2, 8.4 Hz, 3H) |
| 139 | 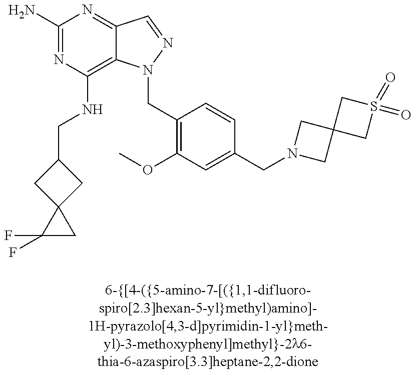 [SMILES: COc1cc(CN2CC3(C2)CS(=O)(=O)C3)ccc1Cn1ncc2nc(N)nc(NCC3CC4(C3)CC4(F)F)c21] | 381.9 | 463.8 | LC/MS [M + H]+ = 559.9 RT (min) 1.17 (LC/MS Procedure B) δ 7.56 (d, J = 3.6 Hz, 1H), 6.90 (s, 1H), 6.70 (d, J = 7.9 Hz, 1H), 6.39 (dd, J = 21.0, 7.7 Hz, 1H), 5.67 (d, J = 9.1 Hz, 2H), 5.60 (s, 2H), 4.29 (d, J = 3.0 Hz, 5H), 3.82 (d, J = 2.8 Hz, 3H), 3.55 (s, 1H), 2.69 (s, 1H), 2.09 (dt, J = 22.1, 10.7 Hz, 3H), 1.90 (s, 4H), 1.87 (d, J = 5.2 Hz, 1H), 1.86- 1.79 (m, 2H), 1.31 (dt, J = 25.0, 8.5 Hz, 3H). |
| 140 | 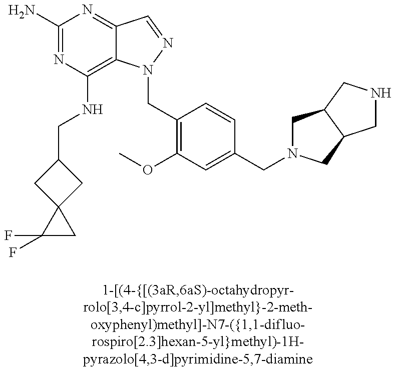 [SMILES: COc1cc(CN2C[C@H]3CNC[C@H]3C2)ccc1Cn1ncc2nc(N)nc(NCC3CC4(C3)CC4(F)F)c21] | 434.9 | | LC/MS [M + H]+ = 525.1 RT (min) 1.42 (LC/MS Procedure B) δ 8.41 (s, 1H), 7.91 (s, 1H), 7.77 (d, J = 2.5 Hz, 2H), 7.12 (s, 1H), 6.94 (s, 1H), 6.84 (d, J = 7.9 Hz, 1H), 5.74 (s, 3H), 3.77 (d, J = 4.6 Hz, 4H), 3.68 (t, J = 6.5 Hz, 2H), 3.18 (s, 1H), 2.24- 2.15 (m, 2H), 2.14 (d, J = 10.3 Hz, 2H), 2.08 (s, 1H), 2.03-1.90 (m, 5H), 1.35 (dt, J = 15.6, 8.4 Hz, 4H) |
| 141 | 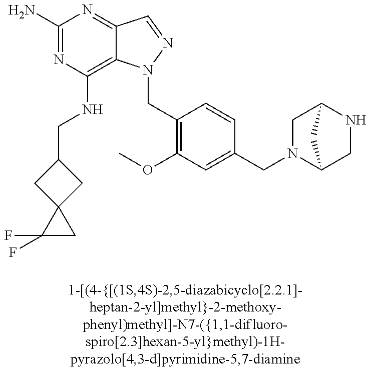 [SMILES: COc1cc(CN2C[C@@H]3C[C@H]2CN3)ccc1Cn1ncc2nc(N)nc(NCC3CC4(C3)CC4(F)F)c21] | 265.9 | 244.4 | LC/MS [M + H]⁺ = 511.1 RT (min) 1.35 (LC/MS Procedure B) δ 8.41 (s, 1H), 7.91 (s, 0H), 7.80- 7.75 (m, 1H), 7.05 (s, 1H), 6.91 (s, 1H), 6.86-6.78 (m, 1H), 5.72 (s, 2H), 4.27 (s, 1H), 3.84 (s, 0H), 3.76 (t, J = 4.4 Hz, 3H), 3.69 (s, 1H), 3.19 (d, J = 3.6 Hz, 1H), 2.94 (s, 0H), 2.19 (dd, J = 24.1, 12.5 Hz, 3H), 2.08 (d, J = 3.5 Hz, 1H), 2.01 (d, J = 7.3 Hz, 2H), 1.93 (s, 1H), 1.80 (d, J = 14.7 Hz, 1H), 1.36 (dd, J = 15.4, 8.5 Hz, 3H), 1.18 (t, J = 7.3 Hz, 1H) |
| 142 | 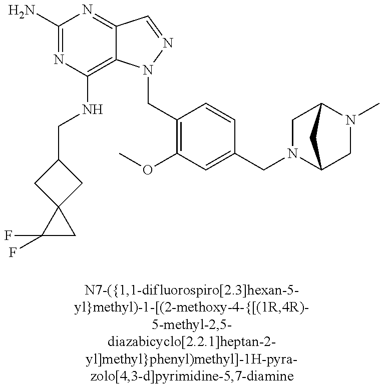 [SMILES: COc1cc(CN2C[C@H]3C[C@@H]2CN3C)ccc1Cn1ncc2nc(N)nc(NCC3CC4(C3)CC4(F)F)c21] | 49.7 | | LC/MS [M + H]+ = 525.1 RT (min) 1.13 (LC/MS Procedure B) δ 7.56 (d, J = 3.6 Hz, 1H), 7.00 (s, 1H), 6.77 (d, J = 7.8 Hz, 1H), 6.64- 6.55 (m, 1H), 6.40 (dd, J = 18.9, 7.8 Hz, 1H), 5.66 (d, J = 8.3 Hz, 2H), 5.61 (d, J = 3.7 Hz, 2H), 3.83 (d, J = 3.1 Hz, 3H), 3.63 (d, J = 13.6 Hz, 1H), 3.57 (d, J = 14.5 Hz, 1H), 3.20 (d, J = 16.6 Hz, 1H), 2.80 (d, J = 9.7 Hz, 1H), 2.69 (s, 1H), 2.60 (d, J = 9.7 Hz, 1H), 2.32 (s, 3H), 2.16-2.08 (m, 2H), 2.07 (d, J = 9.6 Hz, 2H), 1.86 (dt, J = 19.4, 15.5 Hz, 3H), 1.65 (s, 2H), 1.30 (dt, J = 22.0, 8.5 Hz, 2H) |
| 143 | 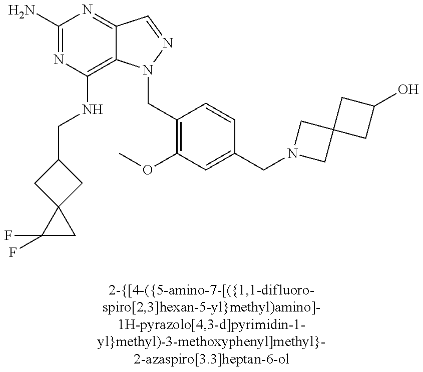 [SMILES: COc1cc(CN2CC3(CC(O)C3)C2)ccc1Cn1ncc2nc(N)nc(NCC3CC4(C3)CC4(F)F)c21] | 103.1 | | LC/MS [M + H]+ = 526.3 RT (min) 1.34 (LC/MS Procedure B) δ 7.56 (d, J = 3.7 Hz, 1H), 6.90 (s, 1H), 6.69 (d, J = 7.9 Hz, 1H), 6.36 (dd, J = 20.7, 7.7 Hz, 1H), 5.68 (d, J = 8.1 Hz, 2H), 5.60 (s, 2H), 3.90 (s, 1H), 3.82 (d, J = 3.2 Hz, 3H), 3.51 (d, J = 13.3 Hz, 8H), 3.11 (d, J = 19.4 Hz, 2H), 2.29 (d, J = 9.7 Hz, 2H), 2.15- 2.06 (m, 2H), 2.05 (d, J = 10.6 Hz, 1H), 1.91 (s, 0H), 1.84 (d, J = 10.7 Hz, 4H), 1.30 (dt, J = 24.2, 8.4 Hz, 2H). |
| 144 | 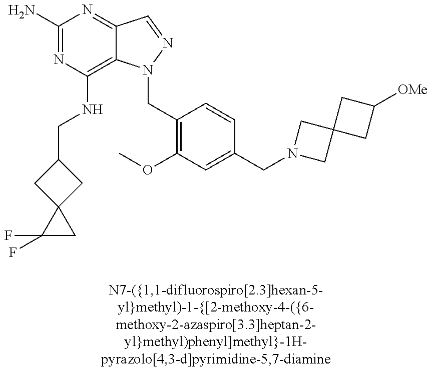 [SMILES: COc1cc(CN2CC3(CC(OC)C3)C2)ccc1Cn1ncc2nc(N)nc(NCC3CC4(C3)CC4(F)F)c21] | 69.5 | | LC/MS [M + H]+ = 540.3 RT (min) 1.52 (LC/MS Procedure B) δ 7.56 (d, J = 3.8 Hz, 1H), 6.91 (s, 1H), 6.70 (d, J = 7.4 Hz, 1H), 6.38 (dd, J = 22.8, 7.7 Hz, 1H), 5.68 (d, J = 8.5 Hz, 2H), 5.61 (d, J = 4.2 Hz, 2H), 3.82 (d, J = 3.6 Hz, 3H), 3.68 (t, J = 6.9 Hz, 1H), 3.17 (d, J = 8.7 Hz, 1H), 3.11 (s, 1H), 3.07 (s, 3H), 2.72- 2.66 (m, 1H), 2.33 (t, J = 9.5 Hz, 2H), 2.09 (dt, J = 21.7, 11.7 Hz, 2H), 1.87 (tt, J = 21.8, 11.0 Hz, 4H), 1.30 (dt, J = 24.0, 8.4 Hz, 2H). |
| 145 | 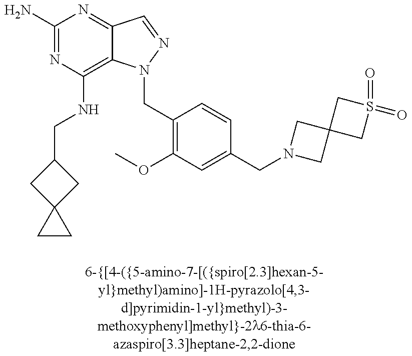 [SMILES: COc1cc(CN2CC3(C2)CS(=O)(=O)C3)ccc1Cn1ncc2nc(N)nc(NCC3CC4(CC4)C3)c21] | 179.4 | 326.2 | LC/MS [M + H]+ = 524.3 RT (min) 1.22 (LC/MS Procedure B) δ 7.56 (s, 1H), 6.91 (s, 1H), 6.71 (d, J = 7.6 Hz, 1H), 6.47-6.39 (m, 2H), 5.67 (s, 2H), 5.60 (s, 2H), 4.31 (s, 4H), 3.84 (s, 3H), 3.58-3.49 (m, 3H), 2.72-2.60 (m, 2H), 2.04- 1.95 (m, 2H), 1.92 (s, 3H), 1.75 (dd, J = 11.9, 6.0 Hz, 2H), 0.36 (s, 4H) |
| 146 | 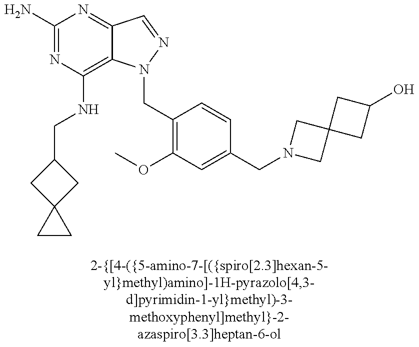 [SMILES: COc1cc(CN2CC3(CC(O)C3)C2)ccc1Cn1ncc2nc(N)nc(NCC3CC4(CC4)C3)c21] | 42.5 | 8.9 | LC/MS [M + H]+ = 490.2 RT (min) 1.2 (LC/MS Procedure B) δ 7.31 (s, 1H), 6.66 (s, 1H), 6.44 (d, J = 7.7 Hz, 1H), 6.14 (t, J = 8.0 Hz, 2H), 5.43 (s, 2H), 5.35 (s, 2H), 3.67 (d, J = 7.0 Hz, 1H), 3.59 (s, 3H), 3.29 (t, J = 6.5 Hz, 1H), 2.82 (d, J = 18.8 Hz, 3H), 2.37 (p, J = 7.5 Hz, 1H), 2.06 (ddd, J = 9.9, 7.0, 2.9 Hz, 2H), 1.77-1.65 (m, 5H), 1.61 (dd, J = 11.2, 8.6 Hz, 2H), 1.48 (dd, J = 11.8, 6.0 Hz, 2H), 0.10 (s, 4H) |
| 147 | 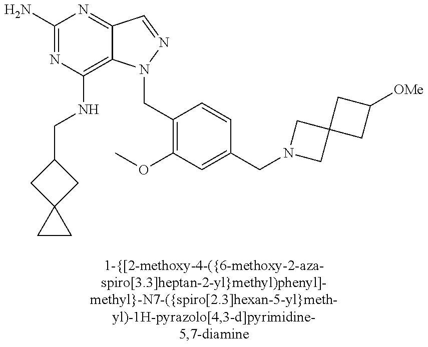 [SMILES: COc1cc(CN2CC3(CC(OC)C3)C2)ccc1Cn1ncc2nc(N)nc(NCC3CC4(CC4)C3)c21] | 40.5 | 37.0 | LC/MS [M + H]+ = 503.9 RT (min) 1.7 (LC/MS Procedure B) δ 7.31 (s, 1H), 6.67 (s, 1H), 6.45 (d, J = 7.6 Hz, 1H), 6.21-6.11 (m, 2H), 5.42 (s, 2H), 5.35 (s, 2H), 3.58 (s, 3H), 3.43 (t, J = 6.9 Hz, 1H), 2.88 (s, 2H), 2.83 (s, 5H), 2.37 (t, J = 7.5 Hz, 1H), 2.08 (ddd, J = 9.7, 6.8, 3.0 Hz, 2H), 1.76-1.60 (m, 5H), 1.48 (dd, J = 11.9, 6.1 Hz, 2H), 0.10 (s, 4H) |
| 148 | 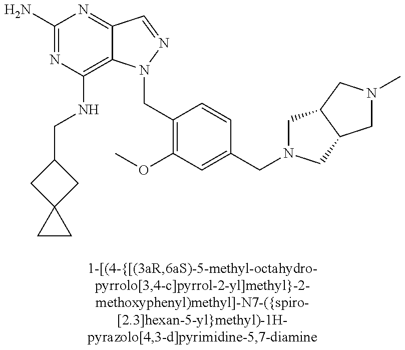 [SMILES: COc1cc(CN2C[C@H]3CN(C)C[C@H]3C2)ccc1Cn1ncc2nc(N)nc(NCC3CC4(CC4)C3)c21] | 74.2 | 9.6 | LC/MS [M + H]+ = 502.9 RT (min) 1.06 (LC/MS Procedure B) δ 7.75-7.66 (m, 2H), 7.56 (s, 1H), 6.98 (s, 1H), 6.75 (d, J = 7.9 Hz, 1H), 6.45-6.36 (m, 2H), 5.67 (s, 1H), 5.61 (s, 2H), 4.20-4.09 (m, 2H), 3.84 (s, 3H), 2.61 (s, 2H), 2.56 (s, 2H), 2.48 (d, J = 5.9 Hz, 2H), 2.34 (s, 2H), 2.26 (d, J = 20.8 Hz, 2H), 2.01- 1.90 (m, 1H), 1.72 (dd, J = 11.8, 6.1 Hz, 1H), 1.67-1.61 (m, 1H), 1.42- 1.32 (m, 2H), 1.35-1.26 (m, 2H), 0.88 (q, J = 7.0, 6.5 Hz, 2H), 0.33 (s, 4H). |
| 149 | 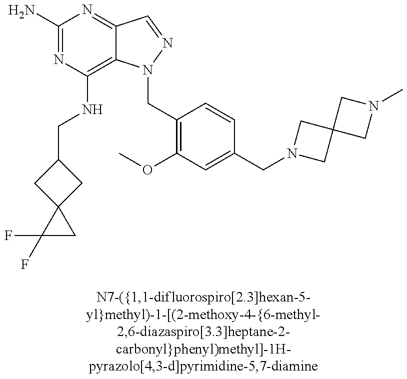 | 42.0 | 31.0 | LC/MS [M + H]⁺ = 539.2 RT (min) 1.13 (LC/MS Procedure B) δ 7.60 (d, J = 4.0 Hz, 1H), 7.22- 7.17 (m, 1H), 7.04 (d, J = 7.8 Hz, 1H), 6.38 (dd, J = 21.4, 7.9 Hz, 1H), 5.69 (t, J = 6.4 Hz, 4H), 4.28 (s, 2H), 4.07 (s, 2H), 3.87 (d, J = 3.7 Hz, 3H), 3.30 (d, J = 27.3 Hz, 2H), 2.64-2.57 (m, 1H), 2.09 (dd, J = 19.9, 10.4 Hz, 2H), 1.92 (s, 4H), 1.85 (dd, J = 12.5, 5.3 Hz, 2H), 1.80 (s, 1H), 1.30 (dt, J = 30.8, 8.4 Hz, 3H) |
| 150 | 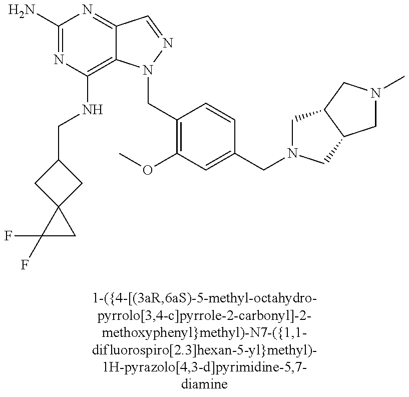 | 9.4 | 17.2 | LC/MS [M + H]⁺ = 553.2 RT (min) 1.14 (LC/MS Procedure B) δ 8.44 (d, J = 5.4 Hz, 2H), 7.79 (d, J = 1.8 Hz, 2H), 7.08 (s, 1H), 7.00 (s, 2H), 6.85-6.79 (m, 2H), 5.76 (s, 4H), 3.81-3.65 (m, 7H), 2.17 (dt, J = 34.4, 10.3 Hz, 4H), 2.02-1.90 (m, 4H), 1.40-1.30 (m, 4H) |
| 151 | 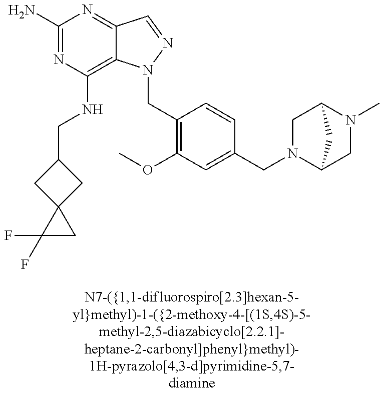 | 45.0 | 32.4 | LC/MS [M + H]+ = 539.0 RT (min) 1.13 (LC/MS Procedure B) δ 8.46 (d, J = 5.9 Hz, 1H), 7.80 (s, 1H), 7.16-7.07 (m, 2H), 7.01 (d, J = 7.6 Hz, 1H), 6.90-6.81 (m, 1H), 5.78 (s, 3H), 4.81 (s, 1H), 4.39 (s, 1H), 4.27 (s, 0H), 3.80 (s, 3H), 3.74 (t, J = 6.4 Hz, 1H), 3.68 (d, J = 13.4 Hz, 2H), 2.88 (d, J = 19.9 Hz, 4H), 2.82 (s, 1H), 2.21 (s, 1H), 2.14 (s, 2H), 2.00 (s, 1H), 1.94 (s, 1H), 1.37 (t, J = 9.3 Hz, 3H) |
| 152 | 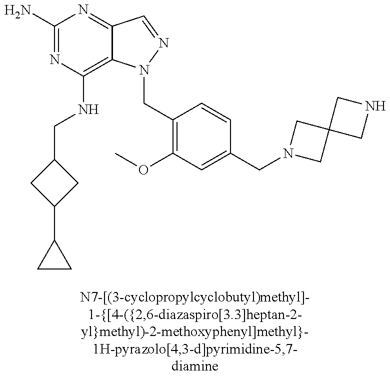 [SMILES: COc1cc(CN2CC3(CNC3)C2)ccc1Cn1ncc2nc(N)nc(NCC3CC(C4CC4)C3)c21] | 166.7 | 152.0 | LC/MS [M + H]⁺ = 489.0 RT (min) 1.19 (LC/MS Procedure B) δ 8.34 (s, 0H), 8.29 (s, 1H), 7.76 (d, J = 2.7 Hz, 1H), 7.10 (s, 1H), 6.93 (d, J = 7.8 Hz, 1H), 6.83 (dd, J = 13.0, 7.6 Hz, 1H), 5.72 (s, 2H), 4.29 (s, 2H), 4.23 (s, 4H), 4.13 (s, 4H), 3.76 (d, J = 7.7 Hz, 2H), 2.38 (d, J = 8.5 Hz, 1H), 1.94 (dd, J = 16.4, 8.4 Hz, 2H), 1.85- 1.77 (m, 1H), 1.75 (s, 1H), 1.68 (q, J = 11.4, 9.7 Hz, 1H), 1.35 (q, J = 10.0 Hz, 1H), 0.82 (s, 1H), 0.70 (s, 1H), 0.38-0.28 (m, 2H), 0.01 (s, 2H) |
| 153 | 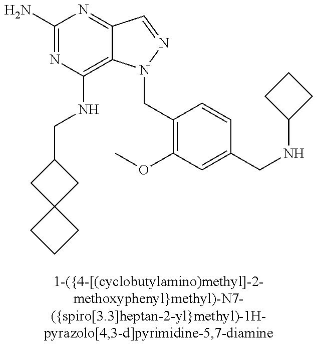 [SMILES: COc1cc(CNC2CCC2)ccc1Cn1ncc2nc(N)nc(NCC3CC4(CCC4)C3)c21] | 30.5 | 188.8 | LC/MS [M + H]⁺ = 460.0 RT (min) 1.66 (LC/MS Procedure B) δ 8.29 (s, 1H), 7.75 (s, 1H), 7.19 (d, J = 1.5 Hz, 1H), 7.02-6.96 (m, 1H), 6.87 (d, J = 7.7 Hz, 1H), 5.73 (s, 2H), 4.01 (s, 2H), 3.77 (s, 3H), 3.66 (t, J = 8.0 Hz, 1H), 3.59 (t, J = 6.4 Hz, 1H), 2.47 (d, J = 15.3 Hz, 2H), 2.20-2.11 (m, 5H), 2.03-1.86 (m, 7H), 1.84- 1.68 (m, 7H). |
| 154 | 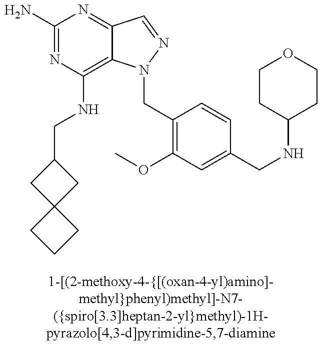 [SMILES: COc1cc(CNC2CCOCC2)ccc1Cn1ncc2nc(N)nc(NCC3CC4(CCC4)C3)c21] | 26.9 | 130.5 | LC/MS [M + H]+ = 491.9 RT (min) 1.58 (LC/MS Procedure B) δ 8.34 (t, J = 5.7 Hz, 1H), 7.75 (s, 1H), 7.22 (s, 1H), 7.03 (d, J = 7.6 Hz, 1H), 6.92 (d, J = 7.7 Hz, 1H), 5.73 (s, 2H), 4.16 (s, 2H), 3.93 (dd, J = 10.4, 4.0 Hz, 2H), 3.77 (s, 3H), 3.60 (t, J = 6.6 Hz, 1H), 3.31 (t, J = 11.7 Hz, 2H), 2.52-2.43 (m, 3H), 2.02 (s, 2H), 2.01-1.91 (m, 5H), 1.89 (t, J = 7.5 Hz, 2H), 1.80-1.68 (m, 4H), 1.61 (tt, J = 12.7, 6.3 Hz, 2H) |
| 155 | 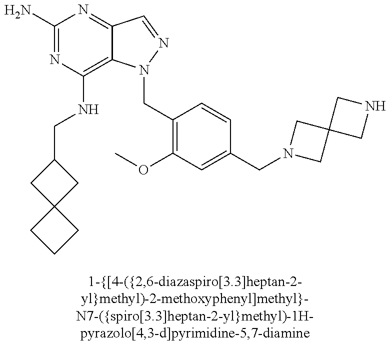 [SMILES: COc1cc(CN2CC3(CNC3)C2)ccc1Cn1ncc2nc(N)nc(NCC3CC4(CCC4)C3)c21] | 583.4 | 552.0 | LC/MS [M + H]+ = 489.1 R (min) 1.51 (LC/MS Procedure B) δ 8.26 (s, 1H), 7.76 (s, 1H), 7.10 (s, 1H), 6.94 (d, J = 7.7 Hz, 1H), 6.83 (d, J = 7.7 Hz, 1H), 5.73 (s, 2H), 4.28 (s, 2H), 4.21 (s, 3H), 4.14 (s, 4H), 3.77 (s, 3H), 2.50-2.40 (m, 2H), 1.95 (q, J = 10.0, 8.3 Hz, 5H), 1.87 (t, J = 7.4 Hz, 3H), 1.80-1.64 (m, 5H). Two protons invisible. |
| 156 | 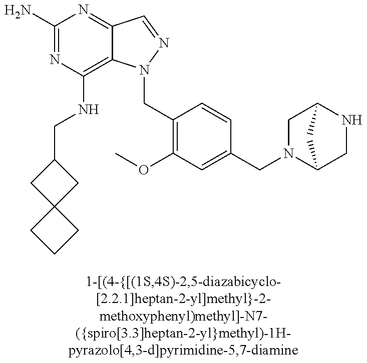 [SMILES: COc1cc(CN2C[C@@H]3C[C@H]2CN3)ccc1Cn1ncc2nc(N)nc(NCC3CC4(CCC4)C3)c21] | 76.9 | 374.3 | LC/MS [M + H]+ = 488.9 RT (min) 1.2 (LC/MS Procedure B) δ 8.28 (t, J = 5.6 Hz, 1H), 7.77 (s, 1H), 7.19 (d, J = 8.5 Hz, 1H), 7.00 (d, J = 7.7 Hz, 1H), 6.83 (d, J = 7.7 Hz, 1H), 5.73 (s, 2H), 4.41 (s, 1H), 4.24 (s, 1H), 4.17 (s, 1H), 3.78 (s, 3H), 3.59 (d, J = 6.1 Hz, 1H), 3.36-3.21 (m, 1H), 2.48-2.41 (m, 2H), 2.00- 1.90 (m, 6H), 1.87 (t, J = 7.4 Hz, 3H), 1.80-1.65 (m, 6H) |
| 157 | 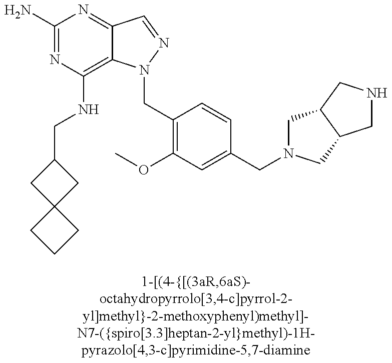 [SMILES: COc1cc(CN2C[C@H]3CNC[C@H]3C2)ccc1Cn1ncc2nc(N)nc(NCC3CC4(CCC4)C3)c21] | 65.7 | 364.2 | LC/MS [M + H]+ = 502.9 RT (min) 1.56 (LC/MS Procedure B) δ 8.30 (t, J = 5.7 Hz, 1H), 7.94 (s, 1H), 7.77 (s, 1H), 7.17 (s, 1H), 6.97 (d, J = 7.6 Hz, 1H), 6.83 (d, J = 7.7 Hz, 1H), 5.75 (s, 2H), 4.27 (s, 1H), 3.79 (s, 3H), 3.58 (t, J = 6.5 Hz, 1H), 2.50-2.40 (m, 2H), 2.00-1.84 (m, 7H), 1.80-1.63 (m, 5H) |
| 158 | 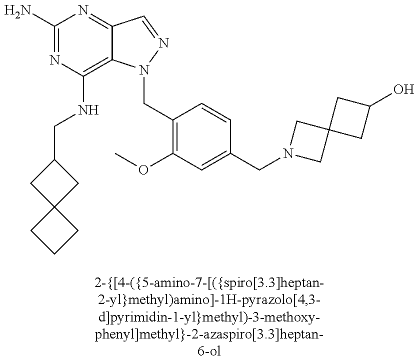 [SMILES: COc1cc(CN2CC3(CC(O)C3)C2)ccc1Cn1ncc2nc(N)nc(NCC3CC4(CCC4)C3)c21] | 38.0 | 39.1 | LC/MS [M + H]+ = 504.1 RT (min) 1.47 (LC/MS Procedure B) δ 8.26 (s, 1H), 7.76 (s, 1H), 7.12 (s, 1H), 6.95 (d, J = 7.7 Hz, 1H), 6.79 (d, J = 7.6 Hz, 1H), 5.73 (s, 2H), 4.27 (s, 2H), 4.06 (s, 2H), 3.96 (p, J = 8.0, 7.6 Hz, 3H), 3.78 (s, 3H), 3.57 (s, 1H), 3.29 (t, J = 7.1 Hz, 1H), 3.20-3.13 (m, 1H), 2.42 (d, J = 6.9 Hz, 3H), 1.95 (q, J = 9.3, 7.3 Hz, 3H), 1.87 (t, J = 7.4 Hz, 3H), 1.75 (q, J = 7.6 Hz, 3H), 1.66 (q, J = 8.9, 6.4 Hz, 4H), 1.55 (s, 1H). |
| 159 | 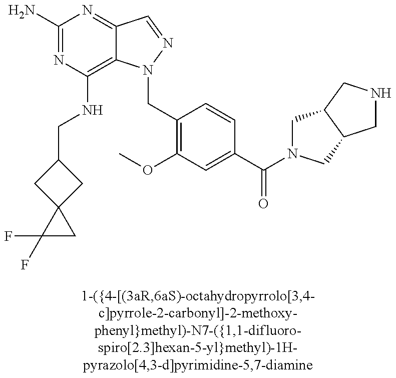 [SMILES: COc1cc(C(=O)N2C[C@H]3CNC[C@H]3C2)ccc1Cn1ncc2nc(N)nc(NCC3CC4(C3)CC4(F)F)c21] | 908.5 | 1,000.0 | LC/MS [M + H]+ = 539.1 RT (min) 1.14 (LC/MS Procedure B) δ 7.59 (d, J = 2.8 Hz, 1H), 7.10- 7.05 (m, 1H), 6.91 (d, J = 7.9 Hz, 1H), 6.41 (t, J = 8.4 Hz, 1H), 5.66 (d, J = 4.6 Hz, 4H), 3.85 (d, J = 2.2 Hz, 3H), 3.60 (d, J = 11.6 Hz, 1H), 3.49 (s, 1H), 3.38 (s, 1H), 3.17 (s, 1H), 3.05 (s, 1H), 2.96 (s, 1H), 2.77 (s, 4H), 2.69 (s, 1H), 2.61 (d, J = 7.4 Hz, 2H), 2.10 (dd, J = 24.8, 14.4 Hz, 3H), 1.28 (dt, J = 23.5, 8.3 Hz, 3H) |
| 160 | 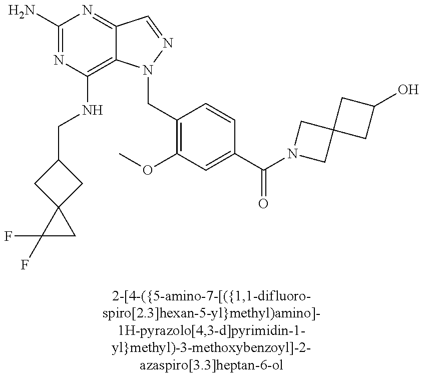 [SMILES: COc1cc(C(=O)N2CC3(CC(O)C3)C2)ccc1Cn1ncc2nc(N)nc(NCC3CC4(C3)CC4(F)F)c21] | 2,585.0 | | LC/MS [M + H]+ = 540.3 RT (min) 1.28 (LC/MS Procedure B) δ 7.71 (d, J = 4.1 Hz, 1H), 7.18 (d, J = 4.7 Hz, 1H), 7.06 (d, J = 7.8 Hz, 1H), 6.60 (s, 1H), 6.55 (d, J = 7.9 Hz, 0H), 5.73 (s, 2H), 4.20 (s, 1H), 4.15 (s, 1H), 3.99 (s, 1H), 3.92 (d, J = 17.3 Hz, 2H), 3.83 (d, J = 5.8 Hz, 3H), 3.74-3.42 (m, 3H), 3.00 (s, 1H), 2.77 (s, 1H), 2.42 (s, 2H), 2.15- 2.06 (m, 2H), 1.95 (d, J = 25.5 Hz, 4H), 1.84 (s, 1H), 1.34 (t, J = 8.5 Hz, 2H), 1.29 (d, J = 8.7 Hz, 1H). |
| 161 | 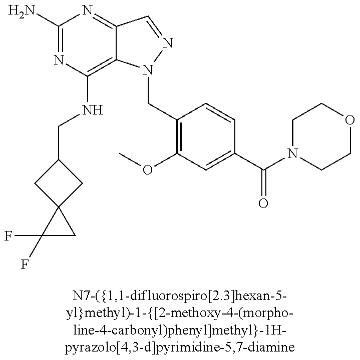 [SMILES: COc1cc(C(=O)N2CCOCC2)ccc1Cn1ncc2nc(N)nc(NCC3CC4(C3)CC4(F)F)c21] | 353.7 | | LC/MS [M + H]+ = 514.5 RT (min) 1.25 (LC/MS Procedure B) δ 7.61 (d, J = 2.9 Hz, 1H), 7.04 (dd, J = 3.7, 1.5 Hz, 1H), 6.90-6.82 (m, 2H), 6.45 (t, J = 7.5 Hz, 1H), 5.86 (s, 1H), 5.69 (d, J = 3.6 Hz, 2H), 3.86 (d, J = 3.2 Hz, 3H), 3.58 (s, 1H), 3.01 (s, 1H), 2.72 (s, 1H), 2.71-2.61 (m, 1H), 2.18-2.08 (m, 2H), 2.07 (d, J = 10.6 Hz, 2H), 1.94-1.84 (m, 3H), 1.31 (dt, J = 22.0, 8.4 Hz, 2H) |
| 162 | 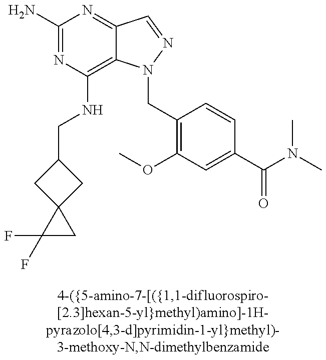 [SMILES: COc1cc(C(=O)N(C)C)ccc1Cn1ncc2nc(N)nc(NCC3CC4(C3)CC4(F)F)c21] | 497.4 | | LC/MS [M + H]+ = 472.2 RT (min) 1.11 (LC/MS Procedure B) δ 7.63 (d, J = 2.7 Hz, 1H), 7.06- 6.97 (m, 2H), 6.84 (dt, J = 7.7, 1.7 Hz, 1H), 6.48-6.41 (m, 1H), 6.03 (s, 1H), 5.70 (d, J = 2.6 Hz, 2H), 3.86 (d, J = 3.1 Hz, 3H), 3.59 (s, 1H), 3.52 (s, 0H), 2.96 (s, 3H), 2.86 (s, 3H), 2.75- 2.62 (m, 2H), 2.18-2.10 (m, 1H), 2.06 (d, J = 11.0 Hz, 1H), 1.95-1.86 (m, 2H), 1.85 (s, 1H), 1.37-1.25 (m, 2H). |
| 163 | 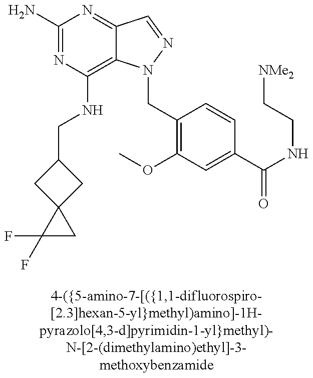 [SMILES: COc1cc(C(=O)NCCN(C)C)ccc1Cn1ncc2nc(N)nc(NCC3CC4(C3)CC4(F)F)c21] | 705.2 | 268.5 | LC/MS [M + H]+ = 515.0 RT (min) 1.22 (LC/MS Procedure B) δ 7.59 (s, 1H), 7.05 (s, 1H), 6.84 (s, 1H), 6.42 (s, 1H), 5.74 (d, J = 23.9 Hz, 1H), 5.68 (d, J = 8.0 Hz, 3H), 3.98 (s, 1H), 3.91 (d, J = 3.4 Hz, 1H), 3.86 (s, 3H), 2.94 (s, 1H), 2.85 (s, 3H), 2.73 (s, 1H), 2.33 (d, J = 5.7 Hz, 1H), 2.12 (s, 2H), 1.86 (s, 5H), 1.35- 1.26 (m, 2H) |
| 164 | 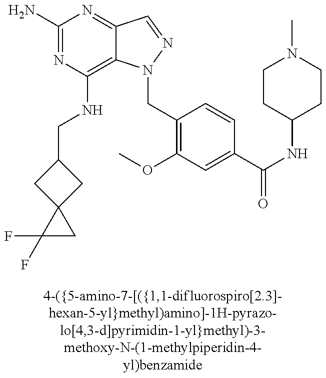 [SMILES: COc1cc(C(=O)NC2CCN(C)CC2)ccc1Cn1ncc2nc(N)nc(NCC3CC4(C3)CC4(F)F)c21] | 638.4 | | LC/MS [M + H]+ = 541.1 RT (min) 1.17 (LC/MS Procedure B) δ 8.20 (d, J = 7.7 Hz, 1H), 7.58 (d, J = 5.0 Hz, 1H), 7.42 (d, J = 5.2 Hz, 1H), 7.29 (d, J = 7.8 Hz, 1H), 6.46 (dd, J = 26.7, 7.9 Hz, 1H), 5.68 (d, J = 7.1 Hz, 3H), 3.88 (d, J = 4.6 Hz, 3H), 3.73 (s, 1H), 2.81 (d, J = 11.3 Hz, 1H), 2.70 (d, J = 8.7 Hz, 1H), 2.20 (s, 2H), 2.12 (s, 1H), 2.03 (t, J = 12.6 Hz, 3H), 1.92 (s, 4H), 1.87 (dd, J = 12.6, 5.3 Hz, 2H), 1.75 (d, J = 13.4 Hz, 2H), 1.63-1.54 (m, 2H), 1.28 (dt, J = 32.7, 8.3 Hz, 2H) |
| 165 | 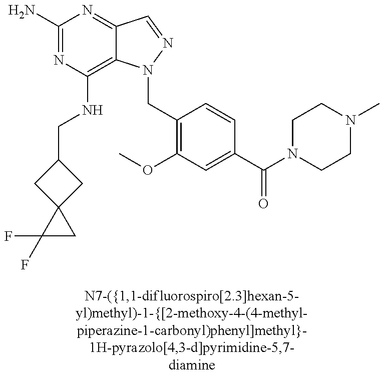 [SMILES: COc1cc(C(=O)N2CCN(C)CC2)ccc1Cn1ncc2nc(N)nc(NCC3CC4(C3)CC4(F)F)c21] | 147.0 | | LC/MS [M + H]+ = 527.2 RT (min) 0.82 (LC/MS Procedure B) δ 7.59 (d, J = 2.9 Hz, 1H), 7.00 (d, J = 3.9 Hz, 1H), 6.81 (d, J = 7.7 Hz, 1H), 6.43 (t, J = 7.8 Hz, 1H), 5.69 (dd, J = 16.4, 6.1 Hz, 4H), 3.85 (d, J = 2.8 Hz, 3H), 2.70 (d, J = 7.2 Hz, 1H), 2.66- 2.59 (m, 1H), 2.23 (s, 1H), 2.20- 2.03 (m, 7H), 1.92 (s, 3H), 1.85 (dd, J = 20.0, 8.2 Hz, 3H), 1.30 (dt, J = 22.4, 8.5 Hz, 3H). |
| 166 | 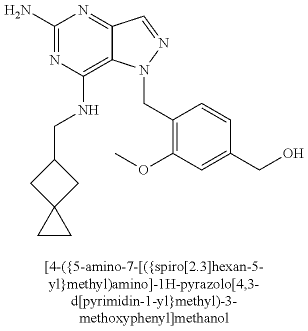 [SMILES: COc1cc(CO)ccc1Cn1ncc2nc(N)nc(NCC3CC4(CC4)C3)c21] | 261.5 | 117.2 | LC/MS [M + H]+ = 395.2 RT (min) 1.46 (LC/MS Procedure B) δ 7.71 (s, 1H), 6.97 (s, 1H), 6.82 (d, J = 7.8 Hz, 1H), 6.76 (d, J = 7.8 Hz, 1H), 5.67 (s, 2H), 4.46 (s, 2H), 3.71 (t, J = 6.6 Hz, 2H), 2.78-2.68 (m, 1H), 2.56 (s, 2H), 2.04 (dd, J = 11.5, 8.4 Hz, 2H), 1.85 (dd, J = 11.9, 6.2 Hz, 2H), 0.37 (s, 4H) |
| 167 | 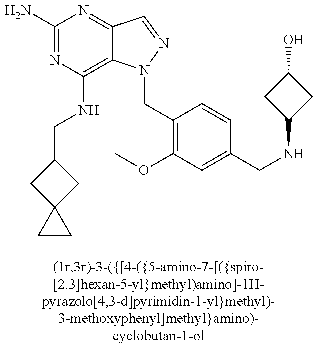 | 23.0 | 1.7 | LC/MS [M + H]+ = 464.05 RT (min) 1.25 (LC/MS Procedure B) δ 7.69 (s, 0H), 7.30 (s, 1H), 6.77 (s, 1H), 6.51 (d, J = 7.7 Hz, 1H), 6.22 (d, J = 7.6 Hz, 1H), 5.40 (s, 1H), 5.34 (s, 2H), 4.00 (t, J = 6.2 Hz, 1H), 3.36- 3.27 (m, 2H), 2.98 (d, J = 7.4 Hz, 1H), 2.65 (s, 1H), 2.37 (q, J = 7.3 Hz, 1H), 1.75 (tt, J = 8.5, 5.1 Hz, 5H), 1.65 (s, 6H), 1.50 (dd, J = 12.2, 6.0 Hz, 2H), 0.10 (s, 4H). |
| 168 | 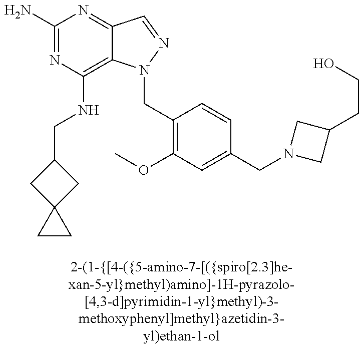 [SMILES: COc1cc(CN2CC(CCO)C2)ccc1Cn1ncc2nc(N)nc(NCC3CC4(CC4)C3)c21] | 30.4 | 3.8 | LC/MS [M + H]+ = 478.0 RT (min) 1.26 (LC/MS Procedure B) δ 7.56 (s, 1H), 6.95 (s, 1H), 6.72 (d, J = 7.7 Hz, 1H), 6.41 (d, J = 7.7 Hz, 1H), 5.66 (s, 1H), 5.59 (s, 2H), 3.82 (s, 2H), 3.63 (s, 0H), 3.35 (dt, J = 18.4, 6.9 Hz, 3H), 2.90 (s, 1H), 2.83 (t, J = 7.2 Hz, 2H), 2.74 (s, 1H), 2.60 (d, J = 14.9 Hz, 1H), 2.46 (p, J = 7.2 Hz, 2H), 1.91 (s, 5H), 1.72 (dd, J = 11.8, 6.2 Hz, 2H), 1.63 (q, J = 6.7 Hz, 2H), 0.33 (s, 4H) |
| 169 | 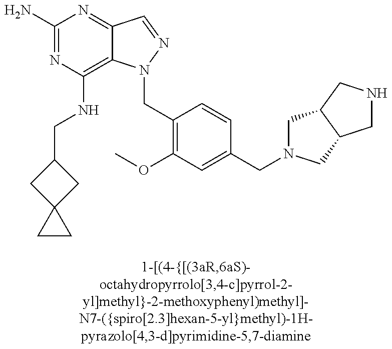 [SMILES: COc1cc(CN2C[C@H]3CNC[C@H]3C2)ccc1Cn1ncc2nc(N)nc(NCC3CC4(CC4)C3)c21] | 28.0 | 4.5 | LC/MS [M + H]+ = 489.1 RT (min) 1.46 (LC/MS Procedure B) δ 8.40 (s, 1H), 7.97 (s, 0H), 7.78 (s, 1H), 7.17 (s, 1H), 6.96 (s, 1H), 6.84 (d, J = 7.7 Hz, 1H), 5.76 (s, 2H), 4.26 (s, 1H), 3.78 (s, 3H), 3.01 (s, 0H), 2.77 (s, 1H), 2.56 (s, 14H), 2.06 (dd, J = 11.6, 8.5 Hz, 2H), 1.87 (dd, J = 1.19, 6.1 Hz, 2H), 0.38 (s, 4H) |
| 170 | 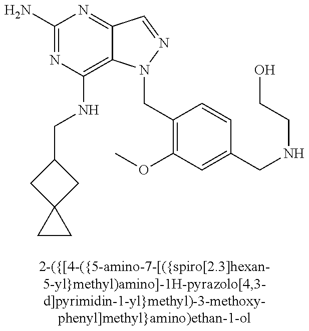 [SMILES: COc1cc(CNCCO)ccc1Cn1ncc2nc(N)nc(NCC3CC4(CC4)C3)c21] | 18.5 | 2.1 | LC/MS [M + H]+ = 438.0 RT (min) 1.24 (LC/MS Procedure B) δ 7.97 (s, 0H), 7.90 (s, 0H), 7.71 (s, 1H), 7.30 (s, 0H), 7.24 (s, 1H), 6.99 (d, J = 7.7 Hz, 1H), 6.78 (d, J = 7.7 Hz, 1H), 5.73 (s, 2H), 4.14 (s, 2H), 3.80 (s, 3H), 3.74-3.64 (m, 3H), 2.98-2.89 (m, 3H), 2.75 (s, 2H), 2.07 (dd, J = 11.5, 8.5 Hz, 2H), 1.93 (s, 0H), 1.87 (dd, J = 11.9, 6.1 Hz, 2H), 0.39 (s, 4H) |
| 171 | 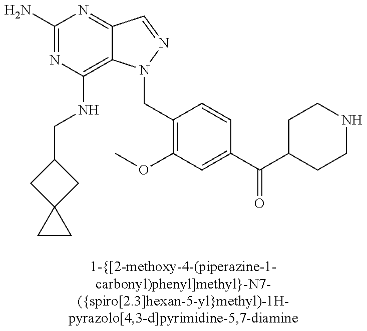 [SMILES: COc1cc(C(=O)C2CCNCC2)ccc1Cn1ncc2nc(N)nc(NCC3CC4(CC4)C3)c21] | 17.0 | 2.8 | LC/MS [M + H]+ = 477.1 RT (min) 1.21 (LC/MS Procedure B) δ 8.06 (s, 1H), 7.62 (s, 0H), 7.53 (s, 1H), 6.83 (s, 1H), 6.69 (d, J = 7.7 Hz, 1H), 6.54 (d, J = 7.7 Hz, 1H), 5.52 (s, 2H), 3.65 (s, 0H), 3.54 (d, J = 2.9 Hz, 3H), 3.48 (t, J = 6.7 Hz, 2H), 3.33 (s, 7H), 2.91 (d, J = 12.7 Hz, 2H), 2.68 (s, 1H), 2.50 (s, 0H), 2.48 (d, J = 7.2 Hz, 1H), 1.78 (t, J = 10.0 Hz, 2H), 1.59 (dd, J = 11.6, 6.2 Hz, 2H), 0.92 (t, J = 7.3 Hz, 1H), 0.12 (s, 4H). |
| 172 | 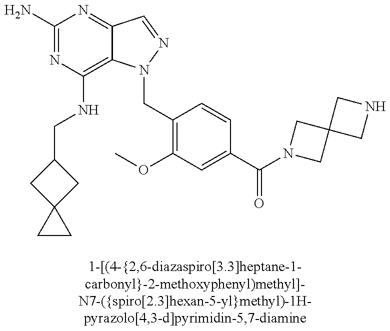 [SMILES: COc1cc(C(=O)N2CC3(CNC3)C2)ccc1Cn1ncc2nc(N)nc(NCC3CC4(CC4)C3)c21] | 784.5 | 300.1 | LC/MS [M + H]+ = 489.1 RT (min) 1.14 (LC/MS Procedure B) δ 7.59 (s, 1H), 7.17 (s, 1H), 7.02 (d, J = 7.9 Hz, 1H), 6.42 (d, J = 8.0 Hz, 1H), 5.67 (s, 4H), 4.35 (s, 2H), 4.15 (s, 2H), 3.87 (d, J = 17.1 Hz, 5H), 3.17 (s, 1H), 1.94 (t, J = 9.9 Hz, 3H), 1.90-1.85 (m, 4H), 1.72 (dd, J = 11.9, 6.0 Hz, 3H), 0.32 (s, 4H). |
| 173 | 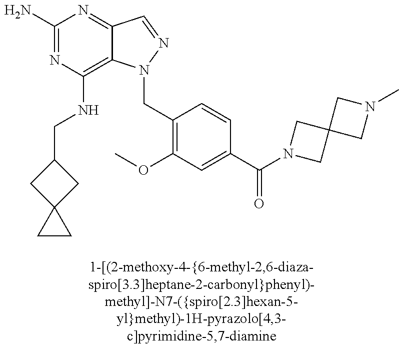 [SMILES: COc1cc(C(=O)N2CC3(CN(C)C3)C2)ccc1Cn1ncc2nc(N)nc(NCC3CC4(CC4)C3)c21] | 20.9 | 5.4 | LC/MS [M + H]+ = 503.1 RT (min) 1.33 (LC/MS Procedure B) ¹H NMR (400 MHz, DMSO-d₆) δ 8.34 (t, J = 5.5 Hz, 1H), 7.90 (s, 1H), 7.78 (s, 1H), 7.17 (d, J = 1.4 Hz, 1H), 7.09 (dd, J = 7.7, 1.5 Hz, 1H), 6.80 (d, J = 7.9 Hz, 1H), 5.78 (s, 2H), 4.43 (s, 2H), 4.35 (s, 1H), 4.20 (s, 2H), 3.80 (s, 3H), 3.73 (dd, J = 7.5, 5.6 Hz, 2H), 3.18 (s, 1H), 2.79 (s, 3H), 2.73 (q, J = 7.2 Hz, 2H), 2.09-1.98 (m, 2H), 1.90-1.80 (m, 2H), 0.37 (s, 4H) |
| 174 | 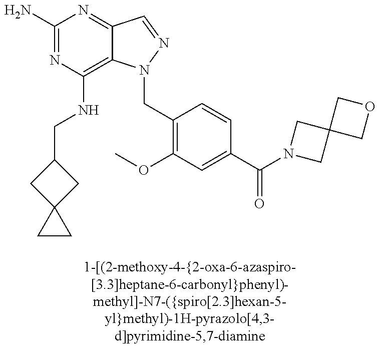 [SMILES: COc1cc(C(=O)N2CC3(COC3)C2)ccc1Cn1ncc2nc(N)nc(NCC3CC4(CC4)C3)c21] | 93.1 | 934.4 | LC/MS [M + H]+ = 490.0 RT (min) 1.45 (LC/MS Procedure B) δ 7.61 (s, 1H), 7.18 (s, 1H), 7.04 (d, J = 7.9 Hz, 1H), 6.77 (s, 1H), 6.46 (d, J = 7.7 Hz, 1H), 5.89 (s, 1H), 5.68 (s, 2H), 4.70-4.61 (m, 4H), 4.39 (s, 2H), 4.18 (s, 2H), 3.64-3.53 (m, 1H), 2.64 (d, J = 7.8 Hz, 1H), 1.95 (dd, J = 19.5, 9.8 Hz, 3H), 1.74 (dd, J = 11.9, 6.2 Hz, 2H), 0.33 (s, 4H) |
| 175 | 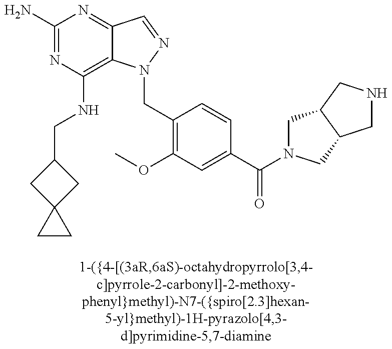 [SMILES: COc1cc(C(=O)N2C[C@H]3CNC[C@H]3C2)ccc1Cn1ncc2nc(N)nc(NCC3CC4(CC4)C3)c21] | 96.5 | 48.8 | LC/MS [M + H]+ = 503.0 RT (min) 1.3 (LC/MS Procedure B) δ 8.07 (s, 1H), 7.52 (d, J = 1.9 Hz, 1H), 6.83 (s, 1H), 6.74 (d, J = 7.7 Hz, 1H), 6.54 (d, J = 7.7 Hz, 1H), 5.50 (s, 2H), 3.53 (d, J = 2.5 Hz, 2H), 3.48 (d, J = 6.2 Hz, 1H), 3.39 (d, J = 16.4 Hz, 9H), 2.75 (s, 4H), 2.48 (t, J = 7.6 Hz, 1H), 1.78 (t, J = 9.9 Hz, 2H), 1.63- 1.55 (m, 2H), 0.11 (d, J = 2.1 Hz, 4H) |
| 176 | 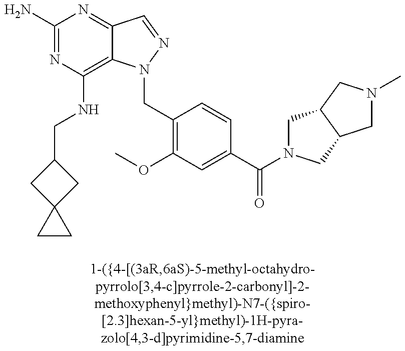 [SMILES: COc1cc(C(=O)N2C[C@H]3CN(C)C[C@H]3C2)ccc1Cn1ncc2nc(N)nc(NCC3CC4(CC4)C3)c21] | 10.8 | 3.6 | LC/MS [M + H]+ = 517.1 RT (min) 1.38 (LC/MS Procedure B) δ 7.36 (s, 1H), 6.83 (s, 1H), 6.66 (dd, J = 7.8, 1.4 Hz, 1H), 6.51 (s, 1H), 6.17 (d, J = 7.7 Hz, 1H), 5.64 (s, 1H), 5.43 (s, 2H), 3.61 (d, J = 3.2 Hz, 3H), 3.32 (s, 8H), 2.60 (s, 1H), 2.44 (s, 1H), 2.40 (d, J = 8.0 Hz, 1H), 2.16 (s, 3H), 1.76-1.65 (m, 3H), 1.50 (d, J = 5.8 Hz, 2H), 1.48 (s, 1H), 0.08 (s, 4H) |
| 177 | 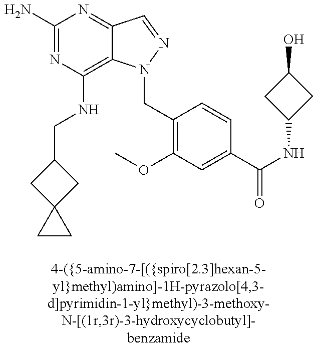 | 579.1 | 358.9 | LC/MS [M + H]+ = 478.1 RT (min) 1.38 (LC/MS Procedure B) δ 8.56 (d, J = 6.7 Hz, 1H), 7.67 (s, 1H), 7.42 (s, 1H), 7.32 (d, J = 7.8 Hz, 1H), 6.62 (d, J = 7.9 Hz, 1H), 5.71 (s, 2H), 4.41 (s, 1H), 4.30 (s, 1H), 3.18 (s, 1H), 3.00 (s, 1H), 2.68 (d, J = 14.6 Hz, 2H), 2.29-2.21 (m, 2H), 2.17 (dd, J = 8.3, 4.4 Hz, 2H), 2.01-1.90 (m, 6H), 1.77 (dd, J = 11.8, 6.2 Hz, 3H), 0.34 (s, 4H) |
| 178 | 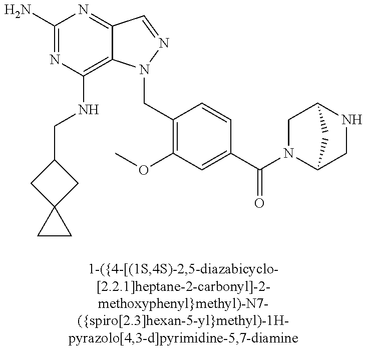 [SMILES: COc1cc(C(=O)N2C[C@@H]3C[C@H]2CN3)ccc1Cn1ncc2nc(N)nc(NCC3CC4(CC4)C3)c21] | 364.9 | 156.4 | LC/MS [M + H]+ = 489.0 RT (min) 1.14 (LC/MS Procedure B) δ 7.34 (d, J = 6.5 Hz, 1H), 6.87 (s, 1H), 6.82 (s, 1H), 6.71 (d, J = 8.1 Hz, 1H), 6.65 (d, J = 7.9 Hz, 1H), 6.36 (s, 0H), 6.17 (t, J = 8.9 Hz, 1H), 5.43 (d, J = 10.6 Hz, 4H), 4.42 (s, 0H), 3.92 (s, 1H), 3.05 (d, J = 12.1 Hz, 1H), 2.99-2.90 (m, 1H), 2.81 (d, J = 10.6 Hz, 1H), 2.75 (s, 1H), 2.66 (s, 1H), 2.39 (d, J = 9.3 Hz, 1H), 1.71 (d, J = 10.0 Hz, 2H), 1.49 (dd, J = 22.4, 11.9 Hz, 4H), 1.33 (d, J = 9.8 Hz, 1H), 0.08 (d, J = 6.1 Hz, 4H) |
| 179 | 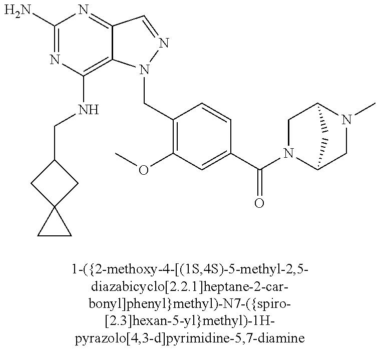 [SMILES: COc1cc(C(=O)N2C[C@@H]3C[C@H]2CN3C)ccc1Cn1ncc2nc(N)nc(NCC3CC4(CC4)C3)c21] | 36.5 | 13.1 | LC/MS [M + H]+ = 503.0 RT (min) 1.13 (LC/MS Procedure B) δ 7.40 (s, 1H), 6.91 (s, 1H), 6.85 (s, 1H), 6.77 (d, J = 8.0 Hz, 1H), 6.70 (d, J = 7.7 Hz, 1H), 6.29 (dd, J = 14.5, 8.1 Hz, 1H), 5.94 (s, 1H), 5.47 (s, 2H), 4.48 (s, 1H), 4.00 (s, 1H), 3.62 (s, 3H), 3.35 (t, J = 6.4 Hz, 1H), 2.76 (s, 2H), 2.42 (s, 4H), 1.74 (d, J = 11.1 Hz, 3H), 1.67 (d, J = 8.7 Hz, 1H), 1.53 (s, 2H), 0.11 (s, 4H) |
| 180 | 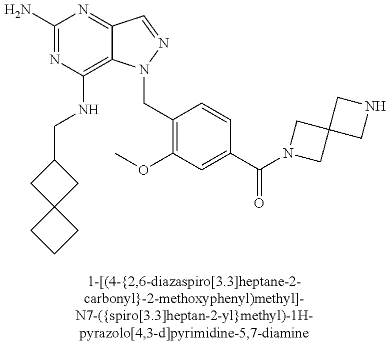 [SMILES: COc1cc(C(=O)N2CC3(CNC3)C2)ccc1Cn1ncc2nc(N)nc(NCC3CC4(CCC4)C3)c21] | 727.7 | 363.0 | LC/MS [M + H]+ = 503.3 RT (min) 1.22 (LC/MS Procedure B) δ 8.21 (s, 1H), 7.78 (s, 1H), 7.19 (d, J = 1.6 Hz, 1H), 7.13-7.07 (m, 1H), 6.79 (d, J = 7.9 Hz, 1H), 5.76 (s, 2H), 4.44 (s, 2H), 4.20 (s, 2H), 4.13 (d, J = 9.4 Hz, 3H), 3.80 (s, 3H), 2.47-2.37 (m, 1H), 1.98-1.82 (m, 7H), 1.78- 1.62 (m, 5H). |
| 181 | 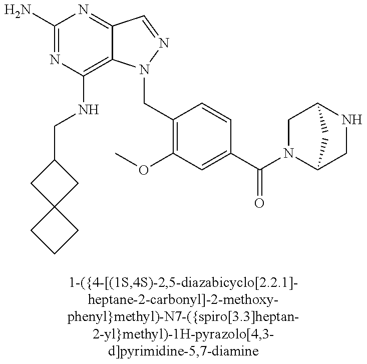 [SMILES: COc1cc(C(=O)N2C[C@@H]3C[C@H]2CN3)ccc1Cn1ncc2nc(N)nc(NCC3CC4(CCC4)C3)c21] | 253.8 | 109.8 | LC/MS [M + H]+ = 503.3 RT (min) 1.23 (LC/MS Procedure B) δ 8.23 (s, 1H), 7.78 (d, J = 10.0 Hz, 1H), 7.15 (s, 1H), 7.11 (s, 1H), 7.07 (d, J = 7.7 Hz, 1H), 7.01 (d, J = 7.9 Hz, 1H), 6.84 (d, J = 7.6 Hz, 1H), 6.77 (d, J = 7.7 Hz, 1H), 5.76 (d, J = 10.4 Hz, 3H), 4.82 (s, 1H), 4.48 (s, 1H), 4.42 (s, 1H), 4.35 (s, 1H), 3.81 (d, J = 14.2 Hz, 3H), 3.68 (s, 0H), 3.62 (s, 0H), 3.26 (dd, J = 21.6, 11.0 Hz, 1H), 2.42 (s, 1H), 2.15 (d, J = 11.2 Hz, 1H), 2.06 (d, J = 11.1 Hz, 1H), 1.97 (s, 2H), 1.87 (d, J = 7.6 Hz, 3H), 1.80 (s, 1H), 1.77-1.65 (m, 6H). |
| 182 | 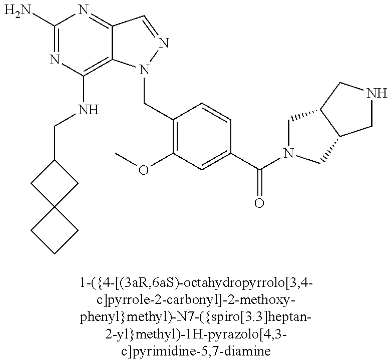 [SMILES: COc1cc(C(=O)N2C[C@H]3CNC[C@H]3C2)ccc1Cn1ncc2nc(N)nc(NCC3CC4(CCC4)C3)c21] | 394.5 | 201.9 | LC/MS [M + H]+ = 517.3 RT (min) 1.23 (LC/MS Procedure B) δ 8.29 (d, J = 6.3 Hz, 1H), 7.92 (s, 1H), 7.79 (s, 1H), 7.12 (d, J = 1.4 Hz, 1H), 7.01 (dd, J = 7.8, 1.5 Hz, 1H), 6.79 (d, J = 7.8 Hz, 1H), 5.77 (s, 2H), 3.81 (s, 3H), 3.59 (t, J = 6.4 Hz, 1H), 3.13 (s, 1H), 3.01 (s, 1H), 2.51- 2.40 (m, 2H), 2.01-1.90 (m, 5H), 1.88 (t, J = 7.4 Hz, 2H), 1.80-1.65 (m, 5H) |
| 183 | 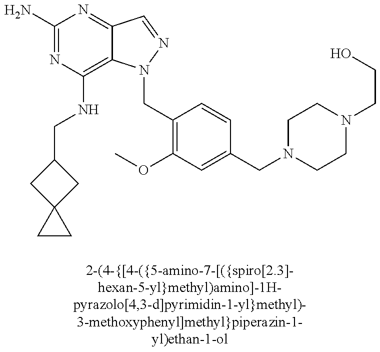 [SMILES: COc1cc(CN2CCN(CCO)CC2)ccc1Cn1ncc2nc(N)nc(NCC3CC4(CC4)C3)c21] | 31.5 | 48.5 | LC/MS [M + H]+ = 506.9 RT (min) 1.43 (LC/MS Procedure B) δ 7.32 (s, 1H), 6.72 (s, 1H), 6.50 (d, J = 7.6 Hz, 1H), 6.24-6.16 (m, 2H), 5.44 (s, 1H), 5.36 (s, 2H), 3.58 (s, 2H), 3.37 (s, 1H), 3.29 (s, 1H), 3.25 (t, J = 6.3 Hz, 1H), 3.16 (s, 1H), 2.94 (s, 0H), 2.39-2.32 (m, 4H), 2.19 (t, J = 6.1 Hz, 4H), 2.11 (s, 2H), 1.76- 1.66 (m, 4H), 1.47 (dd, J = 12.1, 6.1 Hz, 2H), 0.09 (s, 4H) |
| 184 | 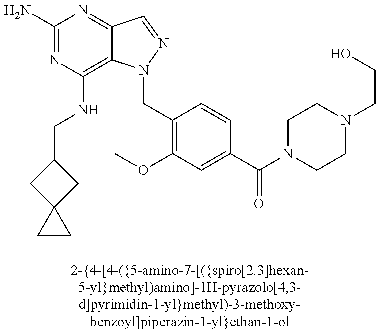 [SMILES: COc1cc(C(=O)N2CCN(CCO)CC2)ccc1Cn1ncc2nc(N)nc(NCC3CC4(CC4)C3)c21] | 67.5 | 109.6 | LC/MS [M + H]+ = 520.9 RT (min) 1.19 (LC/MS Procedure B) δ 8.35 (s, 2H), 7.79 (s, 2H), 7.07 (s, 2H), 6.96 (d, J = 7.6 Hz, 2H), 6.84 (d, J = 7.7 Hz, 2H), 5.77 (s, 4H), 3.79 (s, 3H), 2.73 (td, J = 12.9, 11.3, 5.4 Hz, 2H), 2.07-1.99 (m, 4H), 1.85 (dd, J = 12.0, 6.1 Hz, 4H), 0.37 (s, 8H) |
| 185 | 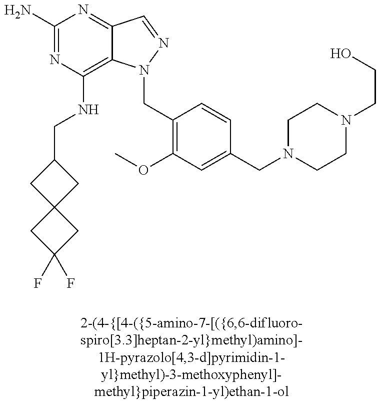 [SMILES: COc1cc(CN2CCN(CCO)CC2)ccc1Cn1ncc2nc(N)nc(NCC3CC4(C3)CC(F)(F)C4)c21] | 481.6 | 117.0 | LC/MS [M + H]+ = 557.0 RT (min) 1.09 (LC/MS Procedure B) δ 8.32 (d, J = 5.9 Hz, 1H), 7.76 (s, 1H), 7.29 (s, 1H), 7.19 (s, 1H), 7.09 (s, 1H), 7.03 (s, 1H), 6.88 (d, J = 7.9 Hz, 1H), 6.79 (d, J = 7.8 Hz, 1H), 5.71 (s, 2H), 3.76 (s, 3H), 3.75- 3.68 (m, 2H), 3.63 (d, J = 6.6 Hz, 1H), 3.18 (s, 0H), 3.13 (s, 2H), 2.61 (s, 1H), 2.58 (s, 4H), 2.08 (dd, J = 11.4, 8.6 Hz, 2H), 1.88 (dd, J = 12.0, 7.6 Hz, 2H) |
| 186 | 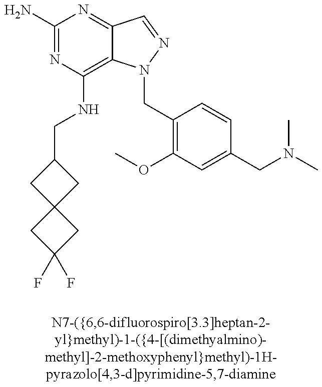 [SMILES: COc1cc(CN(C)C)ccc1Cn1ncc2nc(N)nc(NCC3CC4(C3)CC(F)(F)C4)c21] | 950.4 | 783.2 | LC/MS [M + H]+ = 472.1 RT (min) 1.18 (LC/MS Procedure B) δ 8.17 (s, 1H), 7.77 (s, 1H), 7.21 (s, 1H), 6.99 (d, J = 7.7 Hz, 1H), 6.80 (d, J = 7.8 Hz, 1H), 5.75 (s, 2H), 4.23 (s, 2H), 3.91 (s, 1H), 3.81 (s, 3H), 3.61 (s, 1H), 3.48 (s, 1H), 2.72 (s, 6H), 2.57 (s, 6H), 2.08 (t, J = 10.2 Hz, 2H), 1.91-1.83 (m, 2H). |
| 187 | 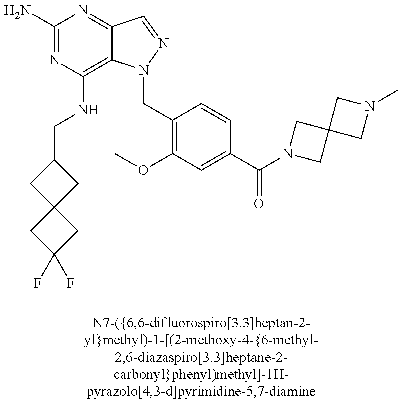 [SMILES: COc1cc(C(=O)N2CC3(CN(C)C3)C2)ccc1Cn1ncc2nc(N)nc(NCC3CC4(C3)CC(F)(F)C4)c21] | 2,306.3 | 419.5 | LC/MS [M + H]+ = 553.2 RT (min) 1.2 (LC/MS Procedure B) δ 7.60 (s, 1H), 7.20 (s, 1H), 7.04 (d, J = 7.9 Hz, 1H), 6.41 (d, J = 7.8 Hz, 1H), 5.67 (d, J = 4.7 Hz, 2H), 4.33 (s, 2H), 4.10 (s, 2H), 3.87 (s, 2H), 3.70 (d, J = 17.0 Hz, 5H), 3.51 (s, 1H), 3.49-3.40 (m, 3H), 2.46-2.39 (m, 2H), 2.30 (s, 3H), 2.00-1.92 (m, 2H), 1.91 (s, 2H), 1.73 (t, J = 9.9 Hz, 2H) |
| 188 | 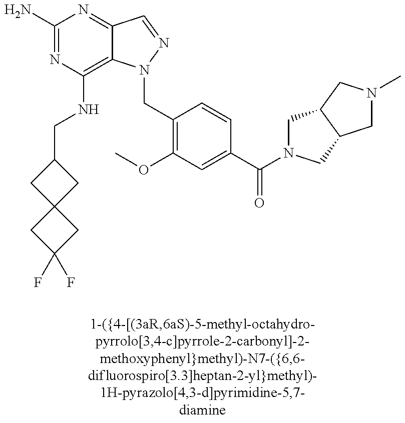 [SMILES: COc1cc(C(=O)N2C[C@H]3CN(C)C[C@H]3C2)ccc1Cn1ncc2nc(N)nc(NCC3CC4(C3)CC(F)(F)C4)c21] | 810.1 | 492.7 | LC/MS [M + H]+ = 566.9 RT (min) 1.17 (LC/MS Procedure B) NMR too noisy to analyze. |
| 189 | 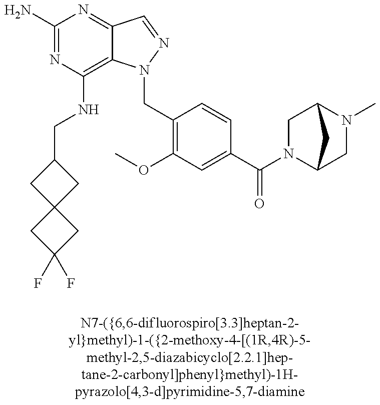 [SMILES: COc1cc(C(=O)N2C[C@H]3C[C@@H]2CN3C)ccc1Cn1ncc2nc(N)nc(NCC3CC4(C3)CC(F)(F)C4)c21] | 780.5 | 1,000.0 | LC/MS [M + H]+ = 552.9 RT (min) 1.2 (LC/MS Procedure B) δ 8.33 (s, 2H), 7.77 (d, J = 4.5 Hz, 2H), 7.31 (s, 1H), 7.21 (s, 1H), 7.16- 7.07 (m, 3H), 7.00 (d, J = 7.5 Hz, 1H), 6.91-6.83 (m, 2H), 5.75 (d, J = 4.4 Hz, 4H), 4.81 (s, 1H), 4.39 (s, 1H), 4.26 (s, 1H), 2.87 (d, J = 19.2 Hz, 6H), 2.18 (s, 1H), 2.07 (s, 4H), 1.87 (s, 3H). |
| 190 | 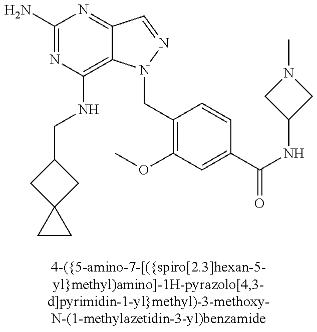 [SMILES: COc1cc(C(=O)NC2CN(C)C2)ccc1Cn1ncc2nc(N)nc(NCC3CC4(CC4)C3)c21] | 314.9 | 168.5 | LC/MS [M + H ]+ = 476.9 RT (min) 1.21 (LC/MS Procedure B) δ 8.50 (d, J = 6.8 Hz, 1H), 7.34 (s, 1H), 7.20 (s, 1H), 7.06 (d, J = 7.8 Hz, 1H), 6.33 (s, 1H), 6.23 (d, J = 7.9 Hz, 1H), 5.43 (d, J = 3.6 Hz, 4H), 4.18 (q, J = 6.9 Hz, 1H), 3.38 (d, J = 7.1 Hz, 1H), 3.30 (s, 0H), 3.20 (s, 1H) ,2.82 (d, J = 14.0 Hz, 2H), 2.42-2.35 (m, 2H), 2.06 (s, 3H), 1.75-1.65 (m, 4H), 1.53-1.46 (m, 3H), 0.08 (s, 4H). |
| 192 | 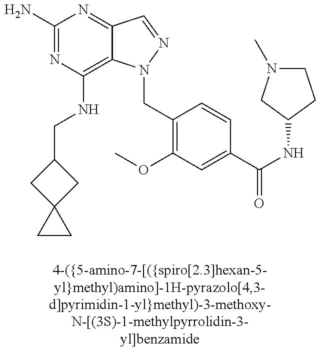 | 47.3 | 21.9 | LC/MS [M + H]+ = 491.2 RT (min) 1.2 (LC/MS Procedure B) δ 8.49 (d, J = 6.3 Hz, 1H), 7.94 (s, 1H), 7.49 (s, 1H), 7.17 (d, J = 1.9 Hz, 1H), 7.11 (d, J = 7.8 Hz, 1H), 6.58 (d, J = 7.9 Hz, 1H), 5.51 (s, 2H), 4.30 (d, J = 7.0 Hz, 1H), 3.55 (s, 3H), 3.46 (t, J = 6.6 Hz, 1H), 3.37 (s, 1H), 2.92 (s, 1H), 2.61 (s, 3H), 2.47 (p, J = 9.3, 8.6 Hz, 1H), 2.12 (s, 1H), 1.79 (dt, J = 37.0, 8.2 Hz, 3H), 1.58 (dd, J = 11.7, 6.2 Hz, 2H), 0.10 (s, 4H) |
| 193 | 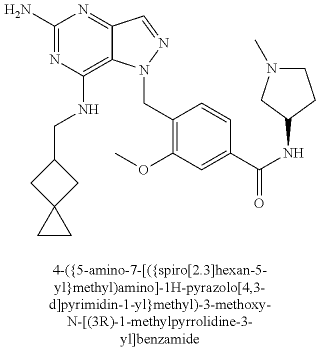 | 48.1 | 15.2 | LC/MS [M + H]+ = 490.5 RT (min) 1.22 (LC/MS Procedure B) δ 8.77 (s, 1H), 8.36 (s, 1H), 7.89 (s, 0H), 7.78 (s, 1H), 7.44 (s, 1H), 7.39 (d, J = 7.8 Hz, 1H), 6.89 (d, J = 7.8 Hz, 1H), 5.79 (s, 2H), 4.58 (s, 1H), 3.82 (s, 3H), 3.74 (t, J = 6.7 Hz, 2H), 3.51 (s, 1H), 3.47 (s, 1H), 2.89 (s, 3H), 2.79-2.72 (m, 1H), 2.04 (dd, J = 11.2, 8.9 Hz, 2H), 1.86 (dd, J = 12.0, 6.2 Hz, 2H), 0.38 (s, 4H) |
| 194 | 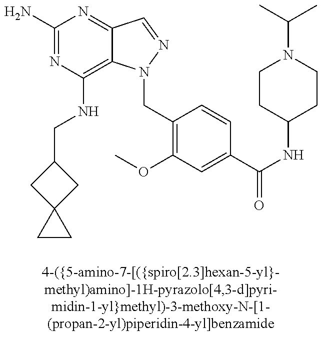 [SMILES: COc1cc(C(=O)NC2CCN(C(C)C)CC2)ccc1Cn1ncc2nc(N)nc(NCC3CC4(CC4)C3)c21] | 103.5 | 100.6 | LC/MS [M + H]+ = 533.0 RT (min) 1.37 (LC/MS Procedure B) δ 8.18 (d, J =7.8 Hz, 1H), 7.58 (s, 1H), 7.43 (s, 1H), 7.28 (d, J = 7.9 Hz, 1H), 6.55 (s, 1H), 6.46 (d, J = 7.8 Hz, 1H), 5.67 (d, J = 7.9 Hz, 4H), 3.89 (s, 3H), 2.79 (d, J = 11.3 Hz, 2H), 2.72- 2.61 (m, 3H), 2.16 (t, J = 11.5 Hz, 2H), 1.95 (t, J = 10.1 Hz, 2H), 1.88 (s, 2H), 1.80-1.69 (m, 4H), 1.53 (dd, J = 13.5, 9.9 Hz, 2H), 0.97 (d, J = 6.5 Hz, 6H), 0.33 (s, 4H). |
| 195 | 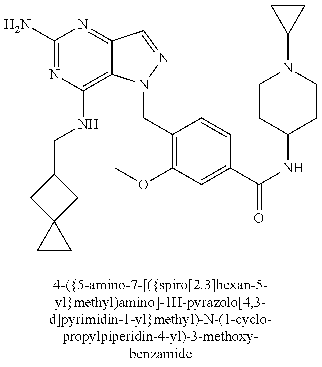 [SMILES: COc1cc(C(=O)NC2CCN(C3CC3)CC2)ccc1Cn1ncc2nc(N)nc(NCC3CC4(CC4)C3)c21] | 193.2 | 411.2 | LC/MS [M + H]+ = 531.0 RT (min) 1.27 (LC/MS Procedure B) δ 8.17 (d, J = 7.9 Hz, 1H), 7.57 (d, J = 1.6 Hz, 1H), 7.40 (s, 1H), 7.26 (d, J = 7.9 Hz, 1H), 6.54 (s, 1H), 6.47-6.41 (m, 1H), 5.65 (d, J = 7.5 Hz, 4H), 3.87 (s, 3H), 3.17 (s, 2H), 2.91 (d, J = 11.3 Hz, 2H), 2.21 (t, J = 11.7 Hz, 2H), 1.90 (s, 4H), 1.71 (dd, J = 11.9, 6.6 Hz, 4H), 1.59 (s, 1H), 1.46 (q, J = 12.2 Hz, 2H), 0.43-0.36 (m, 2H), 0.34-0.25 (m, 6H). |
| 196 | 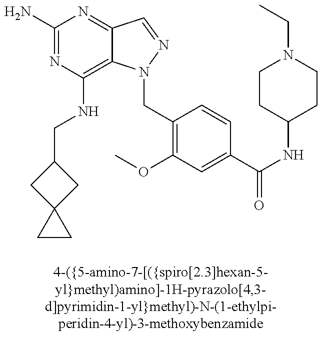 | 77.8 | 35.9 | LC/MS [M + H]+ = 591.3 RT (min) 1.24 (LC/MS Procedure B) δ 8.25 (d, J = 7.3 Hz, 1H), 8.09 (s, 1H), 7.51 (s, 1H), 7.19-7.09 (m, 2H), 6.59 (d, J = 7.9 Hz, 1H), 5.52 (s, 2H), 3.76 (s, 1H), 3.55 (s, 2H), 3.47 (d, J = 6.6 Hz, 2H), 2.92 (s, 2H), 2.84 (d, J = 7.9 Hz, 2H), 2.77 (s, 1H), 2.50- 2.45 (m, 1H), 2.44 (s, 0H), 1.76 (q, J = 13.6, 9.4 Hz, 4H), 1.55 (dt, J = 30.7, 10.1 Hz, 4H), 0.97 (t, J = 7.3 Hz, 3H), 0.10 (s, 4H) |
| 197 | 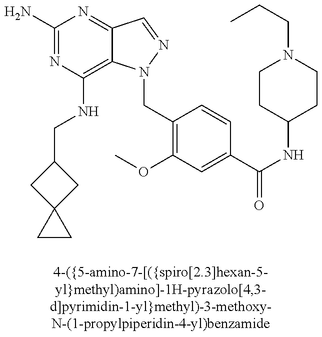 [SMILES: CCCN1CCC(NC(=O)c2ccc(Cn3ncc4nc(N)nc(NCC5CC6(CC6)C5)c43)c(OC)c2)CC1] | 32.6 | 35.5 | LC/MS [M + H]+ = 532.9 RT (min) 1.22 (LC/MS Procedure B) δ 8.24 (d, J = 7.3 Hz, 1H), 8.07 (s, 1H), 7.51 (d, J = 1.2 Hz, 1H), 7.17- 7.08 (m, 2H), 6.59 (d, J = 7.9 Hz, 1H), 5.51 (s, 2H), 3.75 (s, 0H), 3.54 (s, 2H), 3.47 (t, J = 6.6 Hz, 1H), 3.41 (s, 3H), 2.84 (s, 0H), 2.78 (s, 1H), 2.74 (d, J = 7.4 Hz, 2H), 2.46 (dd, J = 15.6, 8.5 Hz, 1H), 1.76 (q, J = 12.3, 9.6 Hz, 4H), 1.55 (dt, J = 26.2, 10.6 Hz, 4H), 1.41 (dt, J = 16.2, 7.7 Hz, 2H), 0.65 (t, J = 7.4 Hz, 3H), 0.09 (d, J = 1.9 Hz, 4H) |
| 198 | 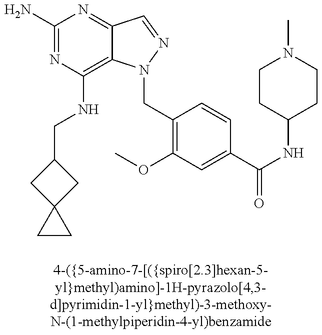 [SMILES: COc1cc(C(=O)NC2CCN(C)CC2)ccc1Cn1ncc2nc(N)nc(NCC3CC4(CC4)C3)c21] | 90.3 | 60.6 | LC-MS (ES, m/z): [M + H]⁺ 505.2 RT (min) = 1.38 (LC/MS Procedure A) δ 7.93 (br d, J = 7.9 Hz, 1H), 7.34 (s, 1H), 7.19 (s, 1H), 7.05 (br d, J = 7.6 Hz, 1H), 6.30 (br s, 1H), 6.22 (d, J = 7.9 Hz, 1H), 5.44 (br s, 4H), 3.65 (s, 3H), 3.47 (br s, 1H), 3.30 (br s, 1H), 2.55 (br d, J = 11.6 Hz, 2H), 2.50- 2.35 (m, 1H), 2.31 (br s, 2H), 1.95 (s, 3H), 1.78-1.64 (m, 4H), 1.56- 1.45 (m, 2H), 1.42-1.22 (m, 2H), 0.09 (s, 4H). |
| 199 | 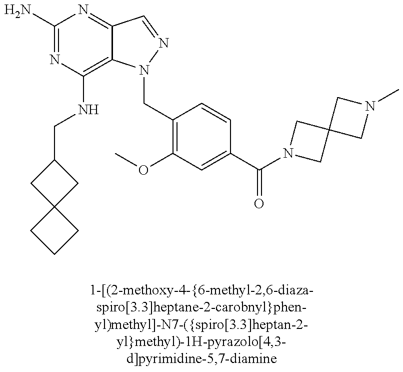 | 251.8 | 304.0 | LC/MS [M + H]⁺ = 491.1 RT (min) = 1.35 (LC/MS Procedure A) ¹H NMR (500 MHz, DMSO-d₆) δ 8.34 (br s, 1H), 8.14 (br s, 1H), 7.93 (br s, 1H), 7.80 (s, 1H), 7.10 (s, 1H), 6.94 (br d, J = 7.6 Hz, 1H), 6.79 (br d, J = 7.3 Hz, 1H), 5.79 (s, 2H), 4.08 (br s, 2H), 3.81 (s, 3H), 3.79-3.66 (m, 2H), 3.22 (br s, 2H), 2.82-2.71 (m, 1H), 2.05 (br t, J = 10.2 Hz, 2H), 1.85 (br dd, J = 11.4, 6.3 Hz, 2H), 0.38 (s, 4H) |
| 200 | 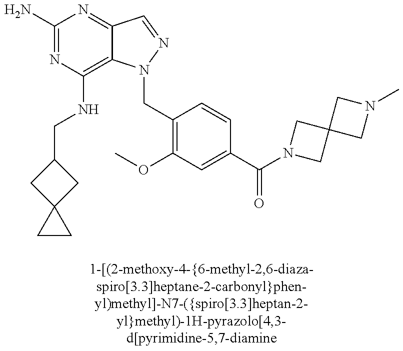 | 123.6 | 129.7 | LC/MS [M + H]+ = 517.1 RT (min) 1.2 (LC/MS Procedure B) δ 7.59 (s, 1H), 7.22 (s, 1H), 7.05 (d, J = 7.8 Hz, 1H), 6.46 (s, 0H), 6.37 (d, J = 7.8 Hz, 1H), 5.66 (d, J = 12.0 Hz, 4H), 4.31 (s, 2H), 4.08 (s, 2H), 3.89 (s, 3H), 3.53 (s, 1H), 3.38 (t, J = 6.4 Hz, 1H), 3.31 (d, J = 7.6 Hz, 1H), 3.24 (d, J = 7.5 Hz, 1H), 3.01 (d, J = 4.4 Hz, 0H), 2.31 (q, J = 7.7 Hz, 1H), 2.17 (s, 3H), 1.93-1.86 (m, 2H), 1.85 (td, J = 12.7, 11.5, 3.7 Hz, 4H), 1.80 (d, J = 7.3 Hz, 3H), 1.72 (q, J = 7.7 Hz, 2H), 1.57-1.50 (m, 2H). |
| 201 | 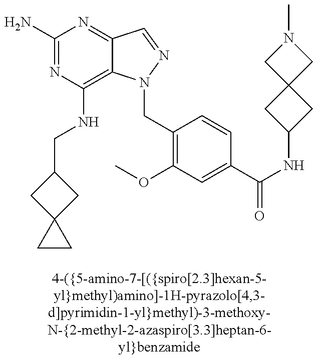 [SMILES: COc1cc(C(=O)NC2CC3(C2)CN(C)C3)ccc1Cn1ncc2nc(N)nc(NCC3CC4(CC4)C3)c21] | 841.8 | 206.0 | LC/MS [M + H]+ = 517.1 RT (min) 1.35 (LC/MS Procedure B) δ 8.65 (d, J = 7.0 Hz, 2H), 8.32 (s, 1H), 7.77 (d, J = 3.8 Hz, 1H), 7.41 (d, J = 9.8 Hz, 2H), 7.34 (d, J = 7.7 Hz, 2H), 6.86-6.78 (m, 2H), 5.77 (s, 3H), 4.32-4.23 (m, 3H), 4.14 (s, 1H), 4.03 (s, 1H), 3.96 (s, 1H), 3.81 (d, J = 9.9 Hz, 2H), 3.60 (d, J = 10.3 Hz, 1H), 2.79 (s, 3H), 2.75-2.68 (m, 1H), 2.30 (s, 2H), 2.04-1.96 (m, 2H), 1.87-1.79 (m, 2H), 0.35 (s, 4H). |
| 202 | 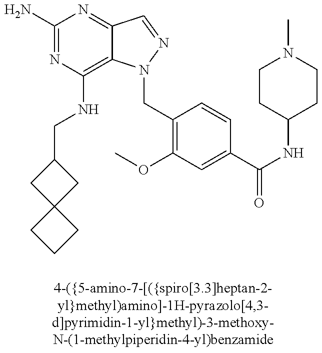 | 775.1 | 940.4 | LC/MS [M + H]+ = 491.1 RT (min) 1.23 (LC/MS Procedure B) δ 9.18 (d, J = 6.4 Hz, 1H), 8.20 (s, 1H), 7.78 (s, 1H), 7.48 (s, 1H), 7.41 (d, J = 7.9 Hz, 1H), 6.84 (d, J = 7.9 Hz, 1H), 5.79 (s, 2H), 4.77 (s, 1H), 4.17 (s, 0H), 3.84 (s, 3H), 3.57 (s, 0H), 3.47 (s, 0H), 2.91 (s, 3H), 2.45- 2.36 (m, 1H), 1.91 (d, J = 8.4 Hz, 5H), 1.86 (t, J = 7.5 Hz, 2H), 1.74 (q, J = 7.5 Hz, 2H), 1.66 (t, J = 9.9 Hz, 2H). |
| 203 | 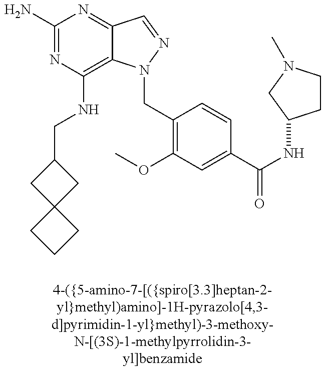 | 413.7 | 535.3 | LC/MS [M + H]⁺ = 509.1 RT (min) 1.02 (LC/MS Procedure B) δ 7.81 (s, 1H), 7.64 (br d, J = 8.5 Hz, 1H), 7.19 (s, 1H), 7.08 (br d, J = 8.1 Hz, 1H), 6.69 (d, J = 8.0 Hz, 1H), 5.80 (q, J = 16.7 Hz, 2H), 4.61-4.50 (m, 1H), 4.49-4.29 (m, 4H), 4.27-4.03 (m, 4H), 3.82 (s, 3H), 3.51-3.45 (m, 3H), 2.78 (br s, 3H), 1.78-1.67 (m, 2H), 1.60-1.45 (m, 2H), 1.19-1.06 (m, 2H), 0.80 (t, J = 7.4 Hz, 3H) |
| 204 | 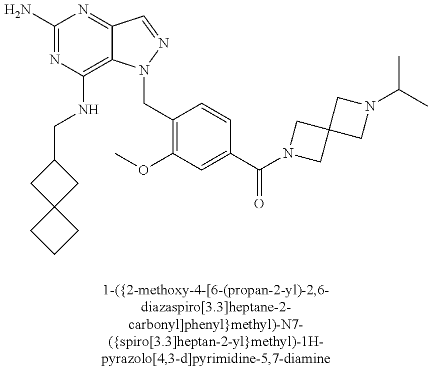 | 160.6 | 178.5 | LC-MS (ES, m/z): [M + H]⁺ 519.2 RT (min) = 1.63 (LC/MS Procedure b) δ 8.48 (br s, 1H), 8.28 (br s, 1H), 7.88-7.70 (m, 1H), 7.43 (br s, 1H), 7.39 (br s, 1H), 6.83 (br s, 2H), 5.71 (br s, 2H), 4.02 (br s, 2H), 3.82 (br s, 4H), 3.73 (br s, 2H), 2.79 (br s, 3H), 2.78-2.69 (m, 1H), 2.30 (br s, 3H), 2.03 (br s, 4H), 1.86 (br s, 2H), 1.79 (br s, 2H), 0.37 (br s, 4H). |
| 205 | 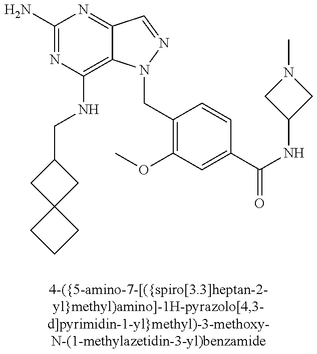 [SMILES: COc1cc(C(=O)NC2CN(C)C2)ccc1Cn1ncc2nc(N)nc(NCC3CC4(CCC4)C3)c21] | 328.3 | 313.6 | LC/MS [M + H]+ = 491.1 RT (min) 1.23 (LC/MS Procedure B) δ 9.18 (d, J = 6.4 Hz, 1H), 8.20 (s, 1H), 7.78 (s, 1H), 7.48 (s, 1H), 7.41 (d, J = 7.9 Hz, 1H), 6.84 (d, J = 7.9 Hz, 1H), 5.79 (s, 2H), 4.77 (s, 1H), 4.17 (s, 0H), 3.84 (s, 3H), 3.57 (s, 0H), 3.47 (s, 0H), 2.91 (s, 3H), 2.45- 2.36 (m, 1H), 1.91 d, J = 8.4 Hz, 5H), 1.86 (t, J = 7.5 Hz, 2H), 1.74 (q, J = 7.5 Hz, 2H), 1.66 (t, J = 9.9 Hz, 2H) |
| 206 | 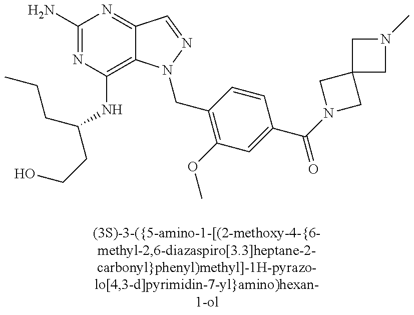 [SMILES: CCC[C@@H](CCO)Nc1nc(N)nc2cnn(Cc3ccc(C(=O)N4CC5(CN(C)C5)C4)cc3OC)c12] | 93.3 | 36.3 | LC/MS [M + H]⁺ = 509.1 RT (min) 1.02 (LC/MS Procedure B) δ 7.81 (s, 1H), 7.64 (br d, J = 8.5 Hz, 1H), 7.19 (s, 1H), 7.08 (br d, J = 8.1 Hz, 1H), 6.69 (d, J = 8.0 Hz, 1H), 5.80 (q, J = 16.7 Hz, 2H), 4.61-4.50 (m, 1H), 4.49-4.29 (m, 4H), 4.27-4.03 (m, 4H), 3.82 (s, 3H), 3.51-3.45 (m, 3H), 2.78 (br s, 3H), 1.78-1.67 (m, 2H), 1.60-1.45 (m, 2H), 1.19-1.06 (m, 2H), 0.80 (t, J = 7.4 Hz, 3H) |
| 207 | 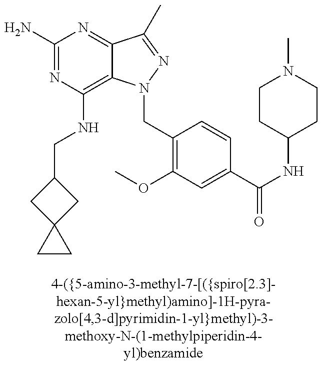 [SMILES: COc1cc(C(=O)NC2CCN(C)CC2)ccc1Cn1nc(C)c2nc(N)nc(NCC3CC4(CC4)C3)c21] | 324.8 | 93.3 | LC-MS (ES, m/z): [M + H]⁺ 519.2 RT (min) = 1.42 (LC/MS Procedure A) δ 8.48 (br s, 1H), 8.28 (br s, 1H), 7.88-7.70 (m, 1H), 7.43 (br s, 1H), 7.39 (br s, 1H), 6.83 (br s, 2H), 5.71 (br s, 2H), 4.02 (br s, 2H), 3.82 (br s, 4H), 3.73 (br s, 2H), 2.79 (br s, 3H), 2.78-2.69 (m, 1H), 2.30 (br s, 3H), 2.03 (br s, 4H), 1.86 (br s, 2H), 1.79 (br s, 2H), 0.37 (br s, 4H). |
| 208 | 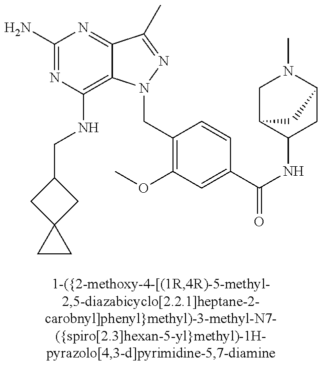 | 118.8 | 45.8 | LC/MS [M + H]+ = 517.3 RT (min) = 1.4 (LC/MS Procedure A) 1H NMR (500 MHz, DMSO-d6) δ 8.31 (br s, 1H), 7.84 (br s, 1H), 7.22- 6.94 (m, 2H), 6.88-6.69 (m, 1H), 5.72 (br s, 2H), 4.82 (br s, 1H), 4.41 (br s, 1H), 4.29 (br s, 1H), 3.81 (br d, J = 7.9 Hz, 3H), 3.77-3.62 (m, 2H), 3.01-2.82 (m, 3H), 2.75 (br s, 1H), 2.31 (br s, 3H), 2.24-1.97 (m, 4H), 1.87 (br s, 2H), 0.38 (s, 4H) |
| 209 | 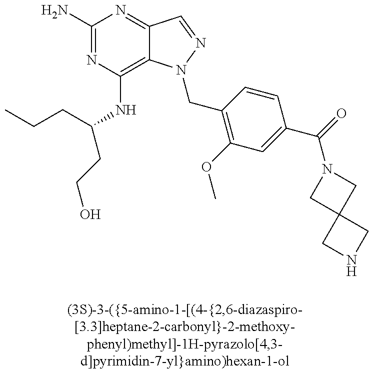 [SMILES: CCC[C@@H](CCO)Nc1nc(N)nc2cnn(Cc3ccc(C(=O)N4CC5(CNC5)C4)cc3OC)c12] | 242.1 | 195.0 | LC/MS [M + H]⁺ = 495.1 RT (min) 1.02 (LC/MS Procedure B) δ 7.80 (s, 1H), 7.64 (br d, J = 8.5 Hz, 1H), 7.31-6.94 (m, 4H), 6.69 (d, J = 7.9 Hz, 1H), 5.80 (q, J = 16.8 Hz, 2H), 4.53 (br dd, J = 13.2, 6.6 Hz, 1H), 4.43 (br s, 2H), 4.19 (br s, 2H), 4.16- 4.04 (m, 4H), 3.82 (s, 3H), 3.56- 3.45 (m, 4H), 1.72 (q, J = 6.1 Hz, 2H), 1.59-1.42 (m, 2H), 1.19-1.10 (m, 2H), 0.80 (t, J = 7.3 Hz, 3H) |
| 210 | 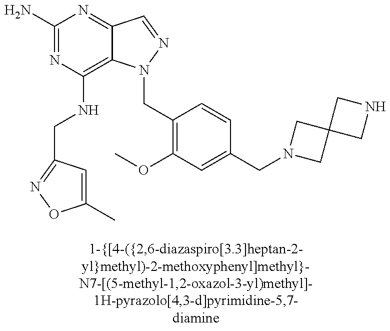 | 227.6 | 18.4 | LC/MS [M + H]+ = 491.1 RT (min) 1.09 (LC/MS Procedure B) δ 7.63-7.58 (m, 1H), 6.87 (s, 1H), 6.70 (s, 1H), 6.47 (s, 1H), 6.01 (s, 1H), 5.77 (s, 2H), 5.61 (s, 2H), 4.65 (s, 2H), 3.85-3.80 (m, 3H), 3.79- 3.74 (m, 3H), 3.24 (d, J = 4.5 Hz, 2H), 3.04-2.99 (m, 1H), 2.36 (d, J = 2.9 Hz, 3H), 1.94-1.88 (m, 2H) |
| 211 | 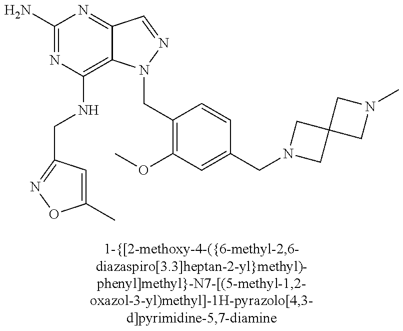 [SMILES: COc1cc(CN2CC3(CN(C)C3)C2)ccc1Cn1ncc2nc(N)nc(NCc3cc(C)on3)c21] | 640.3 | 33.7 | LC/MS [M + H]+ = 490.3 RT (min) 0.86 (LC/MS Procedure B) Proton NMR too noisy to analyze |
| 212 | 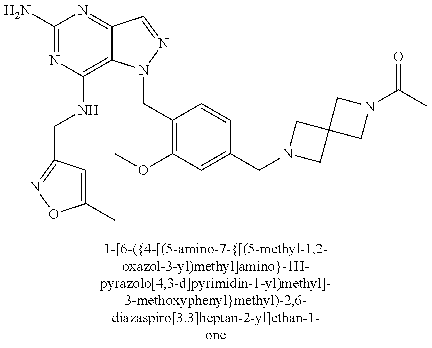 [SMILES: COc1cc(CN2CC3(C2)CN(C(C)=O)C3)ccc1Cn1ncc2nc(N)nc(NCc3cc(C)on3)c21] | 1.199.1 | 61.9 | LC/MS [M + H]+ = 518.0 RT (min) 1.07 (LC/MS Procedure B) δ 7.51 (s, 1H), 6.80 (s, 1H), 6.63 (d, J = 7.8 Hz, 1H), 6.40 (d, J = 7.7 Hz, 1H), 5.92 (s, 1H), 5.66 (s, 2H), 5.52 (s, 2H), 4.59-4.54 (m, 2H), 4.07 (s, 2H), 3.79 (s, 2H), 3.67 (d, J = 2.2 Hz, 2H), 3.16 (s, 3H), 2.26 (s, 3H), 1.84 (s, 2H), 1.63 (s, 3H) |
| 213 | 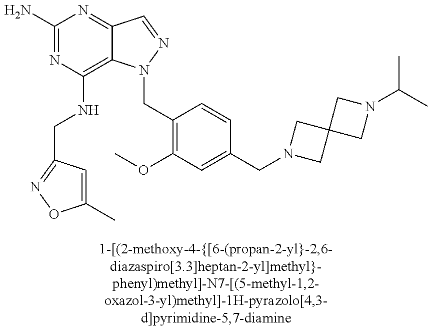 [SMILES: COc1cc(CN2CC3(C2)CN(C(C)C)C3)ccc1Cn1ncc2nc(N)nc(NCc3cc(C)on3)c21] | 53.4 | 2.3 | LC/MS [M + H]+ = 518.1 RT (min) 0.97 (LC/MS Procedure B) δ 8.94 (t, J = 5.9 Hz, 1H), 7.78 (s, 1H), 7.07 (s, 1H), 7.00-6.91 (m, 2H), 6.24 (s, 1H), 5.71 (s, 2H), 4.80 (d, J = 5.7 Hz, 2H), 4.31 (s, 2H), 4.24 (s, 4H), 3.69 (s, 1H), 3.38-3.32 (m, 1H), 2.99 (s, 0H), 2.36 (s, 3H), 1.08 (d, J = 6.4 Hz, 6H) |
| 214 | 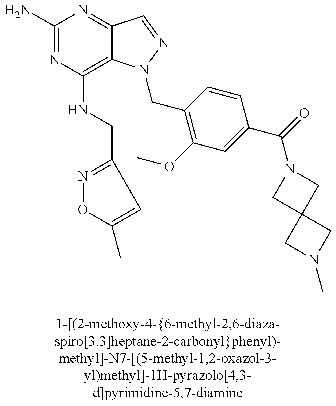 [SMILES: COc1cc(C(=O)N2CC3(CN(C)C3)C2)ccc1Cn1ncc2nc(N)nc(NCc3cc(C)on3)c21] | 67.6 | 10.9 | LC/MS [M + H]+ = 504.31 RT (min) 0.92 (LC/MS Procedure B) δ 7.63-7.60 (m, 1H), 7.34-7.28 (m, 1H), 7.16 (s, 1H), 7.04 (br d, J = 8.0 Hz, 1H), 6.45 (d, J = 7.7 Hz, 1H), 5.94 (s, 1H), 5.77 (s, 2H), 5.67 (s, 2H), 4.62 (br d, J = 4.7 Hz, 2H), 4.31 (br s, 2H), 4.08 (br s, 2H), 3.81 (s, 3H), 2.31 (s, 3H), 2.25 (s, 3H) |
| 215 | 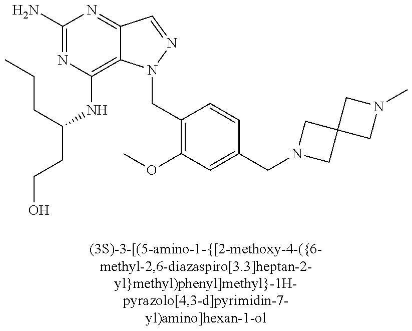 [SMILES: CCC[C@@H](CCO)Nc1nc(N)nc2cnn(Cc3ccc(CN4CC5(CN(C)C5)C4)cc3OC)c12] | 371.8 | 104.3 | LC/MS [M + H]+ = 495.1 RT (min) = 1.0 (LC/MS Procedure B) ¹H NMR (400 MHz, DMSO-d₆) δ 7.84 (s, 1H), 7.79 (s ,1H), 7.64 (d, J = 8.5 Hz, 1H), 7.12 (s, 1H), 6.92 (d, J = 7.8 Hz, 1H), 6.74 (d, J = 7.7 Hz, 1H), 5.85-5.69 (m, 2H), 4.54 (q, J = 7.2 Hz, 2H), 4.31 (s, 2H), 4.24 (s, 8H), 3.80 (s, 3H), 3.18 (s, 2H), 1.73 (q, J = 6.6 Hz, 2H), 1.54 (dt, J = 10.4, 5.4 Hz, 2H), 1.17 (q, J = 7.5 Hz, 2H), 1.10 (d, J = 6.4 Hz, 7H), 0.83 (t, J = 7.3 Hz, 3H) |
| 216 | 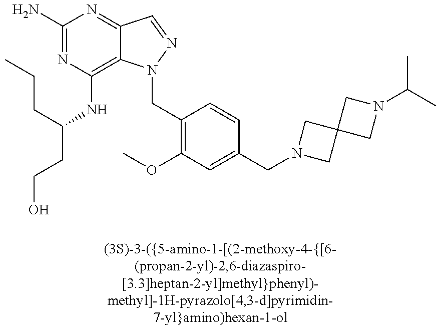 [SMILES: CCC[C@@H](CCO)Nc1nc(N)nc2cnn(Cc3ccc(CN4CC5(C4)CN(C(C)C)C5)cc3OC)c12] | 7.3 | 1.3 | LC/MS [M + H]+ = 261.9 RT (min) = 0.95 (LC/MS Procedure B δ 7.78 (s, 1H), 7.63 (d, J = 8.3 Hz, 1 H), 7.11 (s, 1H), 6.92 (d, J = 7.8 Hz, 1H), 6.72 (d, J = 7.7 Hz, 1H), 5.78 (d, J = 16.4 Hz, 1H), 5.71 (d, J = 16.6 Hz, 1H), 4.53 (d, J = 7.5 Hz, 1H), 4.29 (s, 2H), 4.23 (s, 3H), 3.78 (s, 3H), 3.37 (dt, J = 23.9, 6.6 Hz, 2H), 2.98 (s, 1H), 2.91 (d, J = 8.8 Hz, 2H), 1.72 (s, 2H), 1.52 (s, 2H), 1.15 (q, J = 6.7, 6.3 Hz, 5H), 1.08 (d, J = 6.4 Hz, 8H), 0.80 (t, J = 7.3 Hz, 4H). |
| 217 | 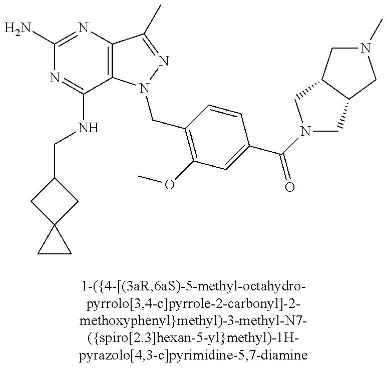 [SMILES: COc1cc(C(=O)N2C[C@H]3CN(C)C[C@H]3C2)ccc1Cn1nc(C)c2nc(N)nc(NCC3CC4(CC4)C3)c21] | 114.9 | 10.8 | LC-MS (ES, m/z): [M + H]⁺ 531.1 RT (min) 0.94 (LC/MS Procedure B) δ 6.81 (s, 1H), 6.63 (d, J = 7.7 Hz, 1H), 6.29 (br s, 1H), 6.12 (d, J = 7.6 Hz, 1H), 5.46 (br s, 2H), 5.34 (s, 2H), 3.61 (s, 3H), 3.38-3.19 (m, 2H), 2.43-2.32 (m, 2H), 2.30 (s, 3H), 1.99 (s, 3H), 1.73-1.68 (m, 2H), 1.48 (dd, J = 11.9, 6.1 Hz, 2H), 0.07 (s, 4H) |
| 218 | 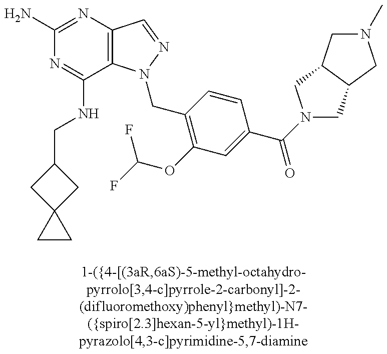 [SMILES: CN1C[C@@H]2CN(C(=O)c3ccc(Cn4ncc5nc(N)nc(NCC6CC7(CC7)C6)c54)c(OC(F)F)c3)C[C@@H]2C1] | 125.1 | 35.0 | LC-MS (ES, m/z): [M + H]⁺ 553.2 RT (min) 1.23 (LC/MS Procedure B) δ 7.37 (s, 1H), 7.18-6.82 (m, 3H), 6.52-6.41 (m, 1H), 6.22 (d, J = 7.8 Hz, 1H), 5.51 (s, 2H), 5.45 (br s, 2H), 3.28 (br s, 2H), 2.95-2.83 (m, 1H), 2.52 (br s, 2H), 2.44-2.33 (m, 1H), 2.19 (br s, 2H), 2.06 (br s, 1H), 1.96 (s, 3H), 1.73-1.61 (m, 3H), 1.45 (br dd, J = 11.3, 5.9 Hz, 2H), 0.05 (s, 4H) |
| 219 | 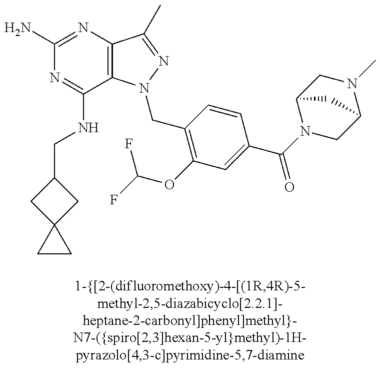 | 187.7 | 37.7 | LC-MS (ES, m/z): [M + H]⁺ 539.0 RT (min) 1.23 (LC/MS Procedure B) δ 7.61 (s, 1H), 7.48-7.07 (m, 3H), 6.79-6.66 (m, 1H), 6.48 (dd, J = 16.6, 8.2 Hz, 1H), 5.76 (s, 2H), 5.70 (s, 2H), 4.11-3.84 (m, 1H), 3.57-3.19 (m, 4H), 2.83-2.57 (m, 4H), 2.33 (m, 3H), 2.05-1.81 (m, 3H), 1.77-1.59 (m, 3H), 0.30 (br s, 4H). |
| 220 | 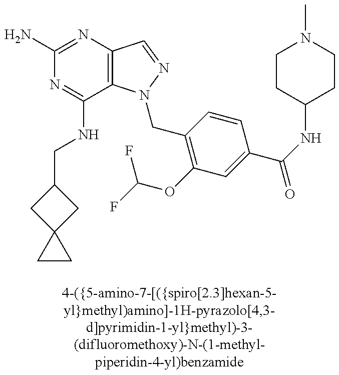 [SMILES: CN1CCC(NC(=O)c2ccc(Cn3ncc4nc(N)nc(NCC5CC6(CC6)C5)c43)c(OC(F)F)c2)CC1] | 1,236.8 | 116.9 | LC-MS (ES, m/z): [M + H]⁺ 541.2 RT (min) 1.25 (LC/MS Procedure B) δ 8.09 (br d, J = 7.7 Hz, 1H), 7.38- 7.33 (m, 2H), 7.30 (d, J = 8.0 Hz, 1H), 6.97 (t, J = 73.4 Hz, 1H), 6.51-6.42 (m, J = 4.6 Hz, 1H), 6.29 (d, J = 8.0 Hz, 1H), 5.51 (s, 2H), 5.43 (s, 2H), 3.27 (br t, J = 6.2 Hz, 2H), 2.58 (br d, J = 10.9 Hz, 2H), 2.42-2.32 (m, 1H), 1.97 (s, 3H), 1.85 (br t, J = 11.4 Hz, 2H), 1.71-1.58 (m, 3H), 1.55-1.42 (m, 4H), 1.39-1.27 (m, 2H), 0.04 (s, 4H) |
| 221 | 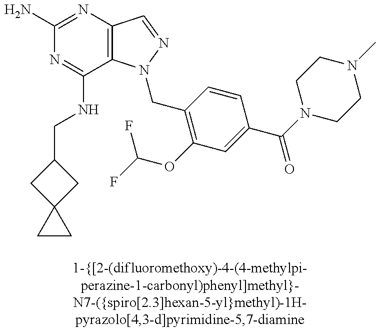 [SMILES: CN1CCN(C(=O)c2ccc(Cn3ncc4nc(N)nc(NCC5CC6(CC6)C5)c43)c(OC(F)F)c2)CC1] | 247.1 | 50.6 | LC-MS (ES, m/z): [M + H]⁺ 527.2 RT (min) 1.23 (LC/MS Procedure B) δ 7.61 (s, 1H), 7.26 (s, 1H), 7.19 (s, 1H) ,7.14-7.08 (m, J = 8.0 Hz, 1H), 6.74 (br s, 1H), 6.50 (d, J = 7.9 Hz, 1H), 5.75 (s, 2H), 5.72 (s, 2H), 3.58- 3.47 (m, 2H), 2.68-2.58 (m, 2H), 2.55 (s, 3H), 2.40-2.28 (m, 2H), 2.21 (br s, 2H), 2.16 (s, 3H), 2.05- 1.89 (m, 2H), 1.70 (br dd, J = 11.7, 6.0 Hz, 2H), 0.30 (s, 4H). |
| 222 | 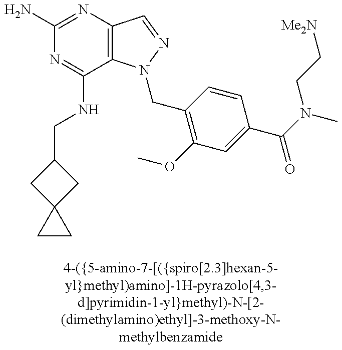 [SMILES: COc1cc(C(=O)N(C)CCN(C)C)ccc1Cn1ncc2nc(N)nc(NCC3CC4(CC4)C3)c21] | 16.0 | 2.4 | LCMS [M⁺ + H] = 493.4 RT 1.28 min (LC/MS Procedure C). ¹H NMR (400 MHz, DMSO-d₆) δ = 7.60-7.54 (m, 1H), 7.01-6.96 (m, 1H), 6.81-6.74 (m, 1H), 6.60-6.50 (m, 1H), 6.37 (d, J = 7.8 Hz, 1H), 5.67 (d, J = 11.2 Hz, 4H), 3.85 (s, 3H), 3.56- 3.51 (m, 4H), 3.23-3.17 (m, 2H), 2.93-2.78 (m, 3H), 2.68-2.56 (m, 1H), 2.19 (br s, 2H), 1.95 (br s, 6H), 1.78-1.65 (m, 2H), 0.35-0.28 (m, 4H). |
| 223 | 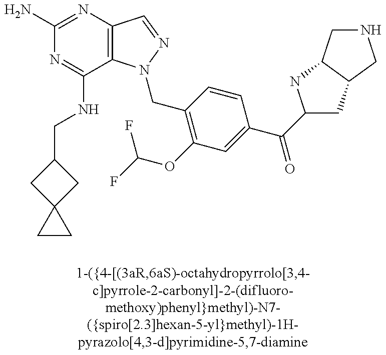 | 2116 | 58 | LC-MS (ES, m/z): [M + H]⁺ 497.2 RT (min) 1.09 (LC/MS Procedure B) δ 8.38 (br s, 1H), 7.63 (s, 1H), 7.53- 7.16 (m, 2H), 6.75-6.64 (m, 1H), 6.50 (br d, J = 8.2 Hz, 1H), 5.79 (s, 2H), 5.70 (s, 2H), 4.38 (br s, 4H), 4.16 (br s, 4H), 3.87 (br d, J = 8.2 Hz, 2H), 1.57-1.35 (m, 2H), 1.28-1.07 (m, 2H), 0.83 (br t, J = 7.3 Hz, 3H). |
| 224 | 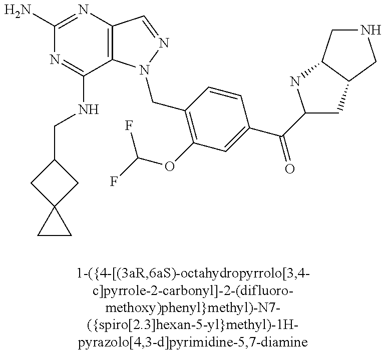 | 1,402.1 | 520.4 | LC-MS (ES, m/z): [M + H]⁺ 539.2 RT (min) 1.24 (LC/MS Procedure B) δ 8.40-8.28 (m, 1H), 7.65 (s, 1H), 7.55-7.09 (m, 3H), 6.81-6.70 (m, 1H), 6.50 (br d, J = 7.9 Hz, 1H), 5.80 (s, 2H), 5.75 (br s, 2H), 3.83-3.51 (m, 1H), 3.26-3.01 (m, 2H), 2.89 (br s, 3H), 2.81-2.63 (m, 2H), 2.03- 1.95 (m, 2H), 1.76 (br dd, J = 11.7, 6.3 Hz, 2H), 0.35 (s, 3H) |
| 225 | 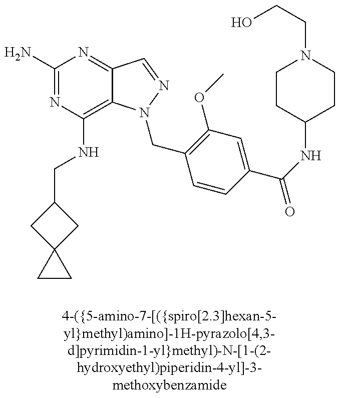 [SMILES: COc1cc(C(=O)NC2CCN(CCO)CC2)ccc1Cn1ncc2nc(N)nc(NCC3CC4(CC4)C3)c21] | 1,223.8 | 217.9 | LCMS [M⁺ + H] = 535.3 RT 1.21 min (LC/MS Procedure C). ¹H NMR (400 MHz, DMSO-d6) δ = 8.15 (d, J = 7.8 Hz, 1H), 7.56 (s, 1H), 7.41 (d, J = 1.5 Hz, 1H), 7.26 (dd, J = 1.5, 7.8 Hz, 1H), 6.57-6.48 (m, 1H), 6.43 (d, J = 7.8 Hz, 1H), 5.67 (d, J = 2.4 Hz, 4H), 3.88 (s, 3H), 3.73-3.68 (m, 1H), 3.54-3.48 (m, 2H), 2.90- 2.81 (m, 2H), 2.65-2.58 (m, 1H), 2.37 (t, J = 6.4 Hz, 2H), 2.06-1.88 (m, 6H), 1.72 (br dd, J = 6.1, 12.2 Hz, 4H), 1.53 (dq, J = 3.4, 11.9 Hz, 2H), 0.32 (s, 4H). |
| 226 | 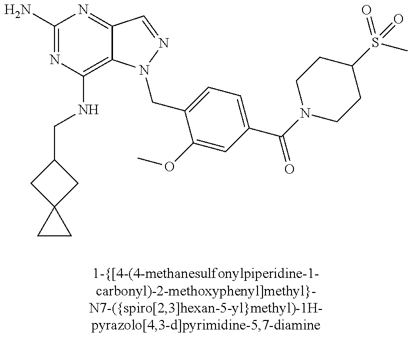 [SMILES: COc1cc(C(=O)N2CCC(S(C)(=O)=O)CC2)ccc1Cn1ncc2nc(N)nc(NCC3CC4(CC4)C3)c21] | 425.2 | 555.1 | LCMS [M⁺ + H] = 554.3 RT 1.35 min (LC/MS Procedure D). ¹H NMR (400 MHz, DMSO-d₆) δ = 7.58 (s, 1H), 7.03 (d, J = 1.5 Hz, 1H), 6.82 (dd, J = 1.5, 7.6 Hz, 1H), 6.56 (t, J = 5.4 Hz, 1H), 6.39 (d, J = 7.8 Hz, 1H), 5.67 (d, J = 5.9 Hz, 4H), 4.63-4.51 (m, 1H), 3.86 (s, 3H), 3 .72-3.59 (m, 1H), 3.53 (br dd, J = 5.6, 7.3 Hz, 3H), 3.12-2.98 (m, 1H), 2.92 (s, 3H), 2.85-2.74 (m, 1H), 2.69-2.60 (m, 1H), 2.21-2.04 (m, 1H), 2.03-1.83 (m, 3H), 1.72 (dd, J = 6.0, 12.3 Hz, 2H), 1.63-1.41 (m, 2H), 0.33 (s, 4H). |
| 227 | 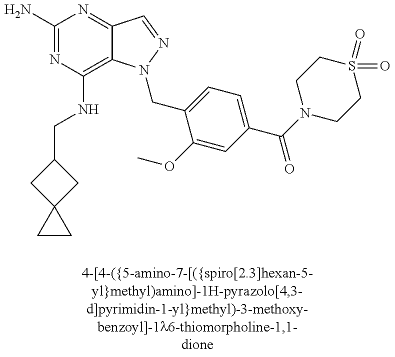 [SMILES: COc1cc(C(=O)N2CCS(=O)(=O)CC2)ccc1Cn1ncc2nc(N)nc(NCC3CC4(CC4)C3)c21] | 506.9 | 125.0 | LCMS [M⁺ + H] = 526.3; RT 1.36 min (LC/MS Procedure D). ¹H NMR (400 MHz, DMSO-d₆) δ = 7.58 (s, 1H), 7.15 (d, J = 1.2 Hz, 1H), 6.90 (dd, J = 1.1, 7.7 Hz, 1H), 6.62 (t, J = 5.5 Hz, 1H), 6.45 (d, J = 7.8 Hz, 1H), 5.68 (d, J = 17.9 Hz, 4H), 4.06- 3.91 (m, 2H), 3.86 (s, 3H), 3.71-3.59 (m, 2H), 3.55 (dd, J = 6.0, 7.0 Hz, 2H), 3.21 (br dd, J = 1.3, 2.3 Hz, 4H), 2.68- 2.60 (m, 1H), 1.99 (dd, J = 8.3, 12.0 Hz, 2H), 1.75 (dd, J = 6.2, 12.1 Hz, 2H), 0.34 (s, 4H). |
| 228 | 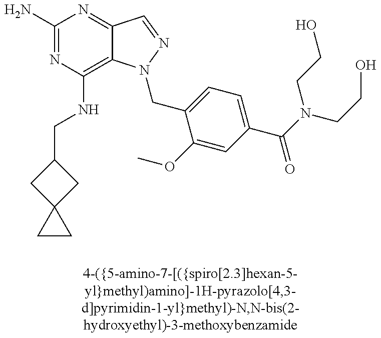 [SMILES: COc1cc(C(=O)N(CCO)CCO)ccc1Cn1ncc2nc(N)nc(NCC3CC4(CC4)C3)c21] | 893.9 | 552.7 | LCMS [M⁺ + H] = 496.3; RT 1.19 min (LC/MS Procedure D). ¹H NMR (400 MHz, DMSO-d₆) δ = 7.57 (s, 1H), 7.06 (d, J = 1.5 Hz, 1H), 6.82 (dd, J = 1.5, 7.8 Hz, 1H), 6.55 (t, J = 5.7 Hz, 1H), 6.42 (d, J = 7.6 Hz, 1H), 5.66 (d, J = 11.2 Hz, 4H), 4.84-4.66 (m, 2H), 3.85 (s, 3H), 3.54 (br dd, J = 5.6, 7.6 Hz, 4H), 3.51-3.43 (m, 4H), 3.27-3.24 (m, 2H), 2.66-2.58 (m, 1H), 2.05-1.94 (m, 2H), 1.75 (dd, J = 6.1, 12.5 Hz, 2H), 0.34 (s, 4H). |
| 229 | 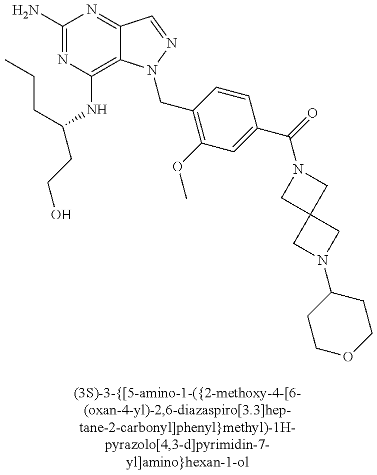 [SMILES: CCC[C@@H](CCO)Nc1nc(N)nc2cnn(Cc3ccc(C(=O)N4CC5(C4)CN(C4CCOCC4)C5)cc3OC)c12] | 37.9 | 37.6 | LC-MS (ES, m/z): [M + H]⁺ 579.3 RT (min) 1.04 (LC/MS Procedure B) δ 7.62 (s, 1H), 7.24 (s, 1H), 7.07 (br d, J = 7.9 Hz, 1H), 6.38 (d, J = 7.9 Hz, 1H), 5.91 (br d, J = 8.9 Hz, 1H), 5.81- 5.64 (m, 3H), 4.33 (br s, 3H), 4.10 (br s, 2H), 3.91 (s, 3H), 3.80 (br d, J = 11.3 Hz, 2H), 3.37-3.30 (m, 1H), 3.25 (br t, J = 10.4 Hz, 2H), 2.20 (br dd, J = 9.9, 2.3 Hz, 1H), 1.72-1.62 (m, 1H), 1.61-1.50 (m, 3H), 1.41 (dt, J = 15.4, 7.6 Hz, 2H), 1.18-0.97 (m, 4H), 0.76 (br t, J = 7.3 Hz, 3H) |
| 230 | 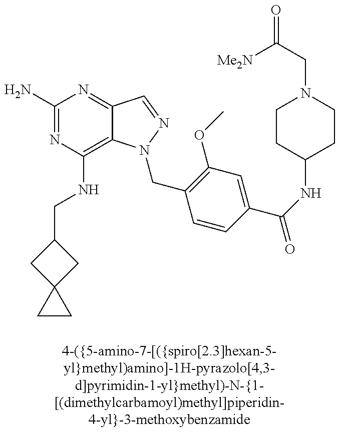 [SMILES: COc1cc(C(=O)NC2CCN(CC(=O)N(C)C)CC2)ccc1Cn1ncc2nc(N)nc(NCC3CC4(CC4)C3)c21] | 275.6 | 196.6 | LCMS [M⁺ + H] = 576.4 RT 1.34 min (LC/MS Procedure C). ¹H NMR (400 MHz, DMSO-d6) δ = 8.53-8.22 (m, 2H), 7.93 (br s, 2H), 7.76 (s, 1H), 7.42-7.40 (m, 1H), 7.39-7.37 (m, 1H), 6.84 (br d, J = 8.3 Hz, 1H), 5.77 (s, 2H), 4.23 (s, 2H), 4.04-3.93 (m, 1H), 3.80 (s, 3H), 3.72 (br t, J = 6.5 Hz, 2H), 3.57-3.46 (m, 2H), 3.18-3.06 (m, 2H), 2.92 (d, J = 13.0 Hz, 6H), 2.78-2.69 (m, 1H), 2.07-1.97 (m, 4H), 1.94-1.76 (m, 4H), 0.36 (s, 4H). |
| 231 | 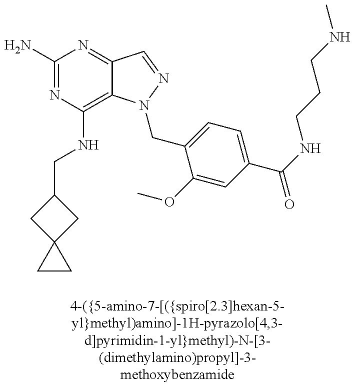 | 165.9 | 32.4 | LCMS [M⁺ + H] = 493.3 RT = 1.17 min (LC/MS Procedure D). ¹H NMR (400 MHz, DMSO-d₆) δ = 8.46 (t, J = 5.6 Hz, 1H), 7.57 (s, 1H), 7.43 (d, J = 1.5 Hz, 1H), 7.26 (dd, J = 1.5, 7.8 Hz, 1H), 6.54 (t, J = 5.4 Hz, 1H), 6.43 (d, J = 8.1 Hz, 1H), 5.67 (d, J = 4.9 Hz, 4H), 3.88 (s, 3H), 3.54- 3.52 (m, 2H), 3.30-3.20 (m, 2H), 2.69-2.58 (m, 1H), 2.26 (t, J = 7.2 Hz, 2H), 2.14 (s, 6H), 1.99-1.91 (m, 2H), 1.72 (dd, J = 6.1, 12.5 Hz, 2H), 1.67-1.57 (m, 2H), 0.32 (s, 4H). |
| 232 | 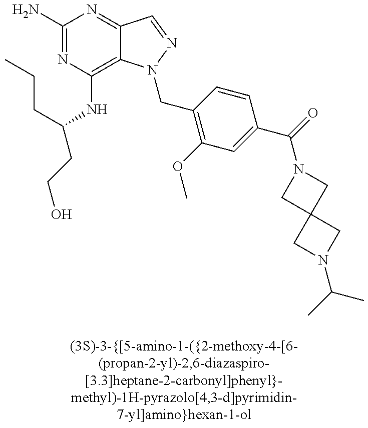 [SMILES: CCC[C@@H](CCO)Nc1nc(N)nc2cnn(Cc3ccc(C(=O)N4CC5(C4)CN(C(C)C)C5)cc3OC)c12] | 37.1 | 32.9 | LC/MS [M + H]⁺ = 537.0 RT (min) = 1.05 (LC/MS Procedure B δ 7.62 (s, 1H), 7.24 (s, 1H), 7.07 (br d, J = 7.9 Hz, 1H), 6.37 (br d, J = 7.9 Hz, 1H), 5.85-5.55 (m, 4H), 4.32 (br d, J = 0.9 Hz, 3H), 4.10 (br s, 2H), 3.97-3.82 (m, 4H), 3.33 (br d, J = 5.5 Hz, 2H), 2.43-2.36 (m, 1H), 1.72- 1.61 (m, 1H), 1.60-1.50 (m, 1H), 1.48-1.34 (m, 2H), 1.10-0.99 (m, 2H), 0.87 (br d, J = 6.1 Hz, 6H), 0.75 (br t, J = 7.3 Hz, 3H) |
| 233 | 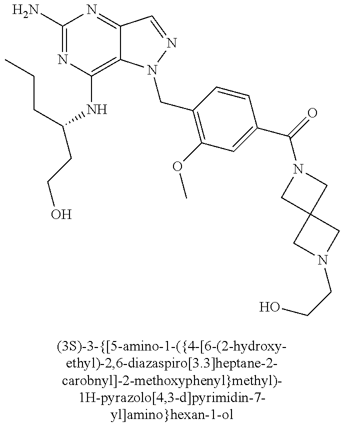 [SMILES: CCC[C@@H](CCO)Nc1nc(N)nc2cnn(Cc3ccc(C(=O)N4CC5(CN(CCO)C5)C4)cc3OC)c12] | 412.1 | 265.1 | LC/MS [M + H]⁺ = 539.0 RT (min) 1.02 (LC/MS Procedure B δ 7.62 (s, 1H), 7.23 (s, 1H), 7.06 (br d, J = 7.9 Hz, 1H), 6.37 (br d, J = 7.6 Hz, 1H), 5.88 (br d, J = 8.5 Hz, 1H), 5.83-5.54 (m, 4H), 4.33 (br s, 3H), 4.10 (br s, 2H), 3.90 (s, 3H), 3.40- 3.28 (m, 1H), 1.74-1.59 (m, 2H), 1.60-1.50 (m, 1H), 1.48-1.31 (m, 2H), 1.12-0.97 (m, 2H), 0.75 (br t, J = 7.2 Hz, 3H) |
| 234 | 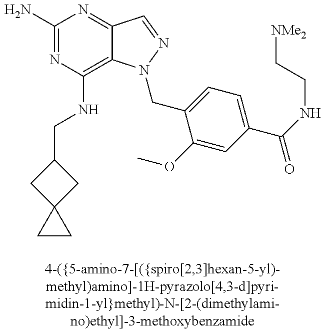 [SMILES: COc1cc(C(=O)NCCN(C)C)ccc1Cn1ncc2nc(N)nc(NCC3CC4(CC4)C3)c21] | 93.2 | 10.2 | LCMS [M⁺ + H] = 479.3 RT 1.21 min [LC/MS Procedure D). ¹H NMR (400 MHz, DMSO-d₆) δ = 8.71-8.63 (m, 1H), 7.72 (s, 1H), 7.44 (d, J = 0.7 Hz, 1H), 7.37 (br d, J = 8.3 Hz, 1H), 6.86-6.77 (m, 1H), 5.75 (s, 2H), 3.81 (s, 3H), 3.73-3.67 (m, 2H), 3.60-3.55 (m, 2H), 3.24- 3.17 (m, 2H), 2.82 (s, 6H), 2.75-2.60 (m, 1H), 2.08-1.98 (m, 2H), 1.89- 1.79 (m, 2H), 0.36 (s, 4H). |
| 235 | 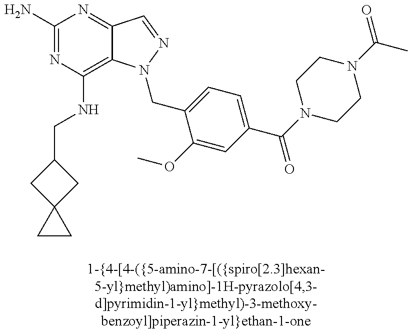 [SMILES: COc1cc(C(=O)N2CCN(C(C)=O)CC2)ccc1Cn1ncc2nc(N)nc(NCC3CC4(CC4)C3)c21] | 146.2 | 223.3 | LCMS [M⁺ + H] = 519.3 RT 1.30 min (LC/MS Procedure D). ¹H NMR (400 MHz, DMSO-d₆) δ = 7.58 (s, 1H), 7.05 (s, 1H), 6.84 (dd, J = 1.2, 7.8 Hz, 1H), 6.58 (br t, J = 5.1 Hz, 1H), 6.40 (d, J = 7.6 Hz, 1H), 5.68 (d, J = 10.3 Hz, 4H), 3.86 (s, 3H), 3.63- 3.45 (m, 8H), 3.27-3.14 (m, 2H), 2.69-2.58 (m, 1H), 2.08-1.90 (m, 5H), 1.73 (dd, J = 6.0, 11.9 Hz, 2H), 0.33 (s, 4H). |
| 236 | 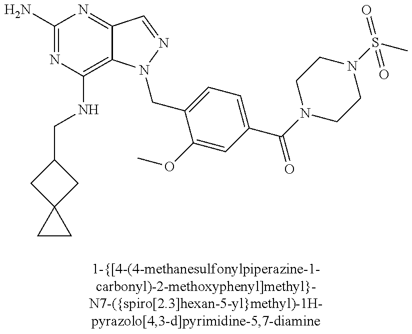 | 199.1 | 284.2 | LCMS [M⁺ + H] = 55.3 RT 1.43 min (LC/MS Procedure D). ¹H NMR (400 MHz, DMSO-d₆) δ = 7.58 (s, 1H), 7.05 (d, J = 1.2 Hz, 1H), 6.85 (dd, J = 1.2, 7.8 Hz, 1H), 6.59 (t, J = 5.6 Hz, 1H), 6.40 (d, J = 7.6 Hz, 1H), 5.68 (d, J = 8.1 Hz, 4H), 3.87 (s, 3H), 3.76-3.60 (m, 2H), 3.54 (dd, J = 5.7, 7.2 Hz, 2H), 3.40-3.30 (m, 2H), 3.23-2.97 (m, 4H), 2.88 (s, 3H), 2.68- 2.59 (m, 1H), 1.97 (dd, J = 8.6, 12.2 Hz, 2H), 1.74 (dd, J = 6.2, 12.2 Hz, 2H), 0.33 (s, 4H). |
| 237 | 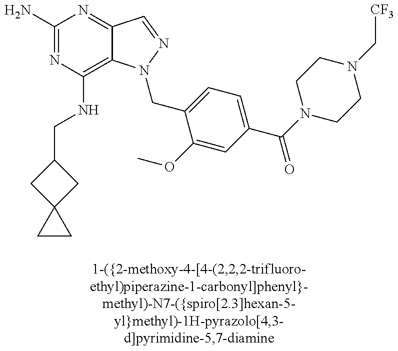 [SMILES: COc1cc(C(=O)N2CCN(CC(F)(F)F)CC2)ccc1Cn1ncc2nc(N)nc(NCC3CC4(CC4)C3)c21] | 589.0 | 957.7 | LCMS [M⁺ + H] = 559.3 RT 1.77 min (LC/MS Procedure D). ¹H NMR (400 MHz, DMSO-d₆) δ = 7.57 (s, 1H), 7.01 (s, 1H), 6.80 (d, J = 7.8 Hz, 1H), 6.55 (t, J = 5.5 Hz, 1H), 6.38 (d, J = 7.8 Hz, 1H), 5.67 (d, J = 8.8 Hz, 4H), 3.86 (s, 3H), 3.65- 3.49 (m, 4H), 3.30-3.14 (m, 4H), 2.72-2.53 (m, 5H), 1.96 (dd, J = 8.7, 11.6 Hz, 2H), 1.72 (dd, J = 6.1, 12.0 Hz, 2H), 0.32 (s, 4H). |
| 238 | 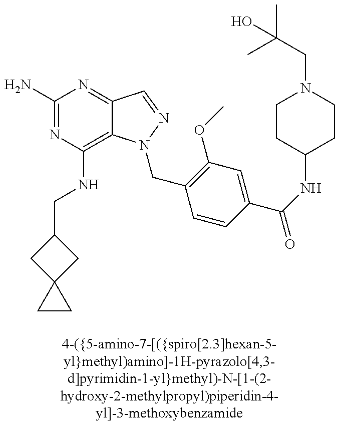 [SMILES: COc1cc(C(=O)NC2CCN(CC(C)(C)O)CC2)ccc1Cn1ncc2nc(N)nc(NCC3CC4(CC4)C3)c21] | 86.0 | 35.2 | LCMS [M⁺ + H] = 563.5 RT 1.37 min (LC/MS Procedure C). ¹H NMR (400 MHz, DMSO-d6) δ = 8.81 (br d, J = 6.4 Hz, 1H), 8.48-8.29 (m, 2H), 7.84 (dddd, J = 2.0, 3.8, 5.2, 6.9 Hz, 1H), 7.76 (s, 1H), 7.45-7.29 (m, 2H), 6.90-6.80 (m, 1H), 5.77 (s, 2H), 5.27-5.18 (m, 1H), 4.12-3.93 (m, 2H), 3.81 (s, 4H), 3.72 (br t, J = 6.2 Hz, 3H), 3.68-3.58 (m, 2H), 3.22-3.04 (m, 4H), 2.10-1.90 (m, 2H), 1.88-1.79 (m, 2H), 1.25 (s, 8H), 0.36 (s, 4H). |
| 239 | 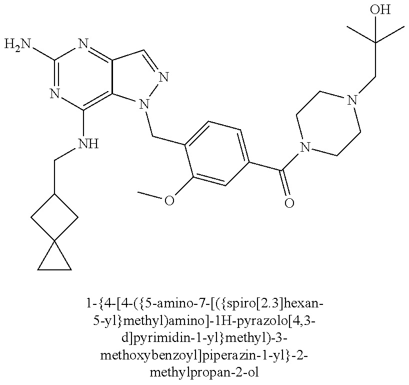 [SMILES: COc1cc(C(=O)N2CCN(CC(C)(C)O)CC2)ccc1Cn1ncc2nc(N)nc(NCC3CC4(CC4)C3)c21] | 39.0 | 109.7 | LCMS [M⁺ + H] = 549.4 RT 1.51 min (LC/MS Procedure D). ¹H NMR (400 MHz, DMSO-d₆) δ = 7.59 (s, 1H), 7.00 (d, J = 1.0 Hz, 1H), 6.79 (dd, J = 1.2, 7.8 Hz, 1H), 6.69- 6.57 (m, 1H), 6.39 (d, J = 7.8 Hz, 1H), 5.83 (br s, 2H), 5.66 (s, 2H), 4.12 (s, 1H), 3.85 (s, 3H), 3.65-3.48 (m, 4H), 3.25 (ddd, J = 3.7, 6.1, 7.8 Hz, 2H), 2.66 (s, 3H), 2.46-2.34 (m, 2H), 2.19 (s, 2H), 1.96 (dd, J = 8.6, 12.2 Hz, 2H), 1.72 (dd, J = 6.1, 12.2 Hz, 2H), 1.07 (s, 6H), 0.33 (s, 4H). |
| 240 | 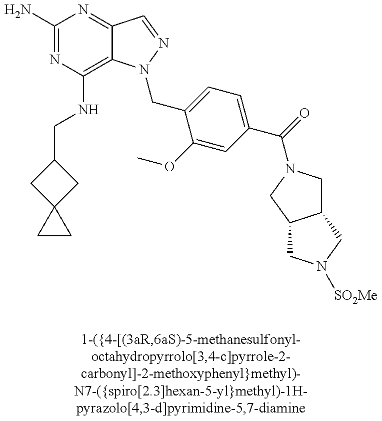 [SMILES: COc1cc(C(=O)N2C[C@@H]3CN(S(C)(=O)=O)C[C@@H]3C2)ccc1Cn1ncc2nc(N)nc(NCC3CC4(CC4)C3)c21] | 290.9 | 844.9 | LCMS [M⁺ + H] = 581.3 RT 1.37 min (LC/MS Procedure D). ¹H NMR (400 MHz, DMSO-d₆) δ = 7.58 (d, J = 0.7 Hz, 1H), 7.13 (s, 1H), 6.94 (d, J = 7.6 Hz, 1H), 6.57 (t, J = 5.6 Hz, 1H), 6.38 (d, J = 7.8 Hz, 1H), 5.67 (d, J = 7.3 Hz, 4H), 3.86 (s, 3H), 3.76- 3.69 (m, 1H), 3.54 (br t, J = 6.5 Hz, 3H), 3.47-3.39 (m, 2H), 3.26-3.17 (m, 2H), 3.06-2.99 (m, 1H), 2.98- 2.81 (m, 6H), 2.68-2.60 (m, 1H), 2.01-1.89 (m, 2H), 1.73 (dd, J = 6.0, 11.9 Hz, 2H), 0.33 (s, 4H). |
| 241 | 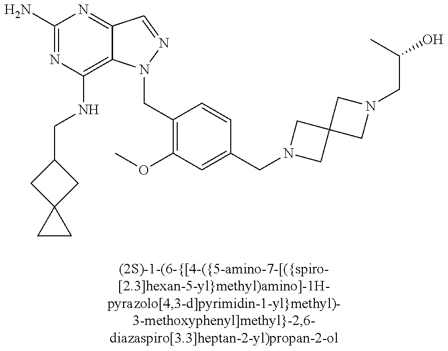 [SMILES: COc1cc(CN2CC3(C2)CN(C[C@H](C)O)C3)ccc1Cn1ncc2nc(N)nc(NCC3CC4(CC4)C3)c21] | 72.9 | 14 | LC/MS [M + H]+ = 532.9 RT (min) 1.38 (LC/MS Procedure B) δ 7.54 (s, 1H), 6.88 (d, J = 1.4 Hz, 1H), 6.68 (d, J = 7.8 Hz, 1H), 6.45- 6.39 (m, 1H), 5.61 (s, 1H), 5.56 (s, 2H), 3.95-3.73 (m, 5H), 3.51 (dd, J = 7.3, 5.3 Hz, 2H), 3.43 (d, J = 6.0 Hz, 4H), 3.18 (d, J = 3.6 Hz, 3H), 2.45 (s, 2H), 1.98-1.90 (m, 2H), 1.83 (s, 2H), 1.70 (dd, J = 11.9, 6.1 Hz, 2H), 1.00-0.95 (m, 3H), 0.32 (p, J = 3.0 Hz, 4H) |
| 242 | 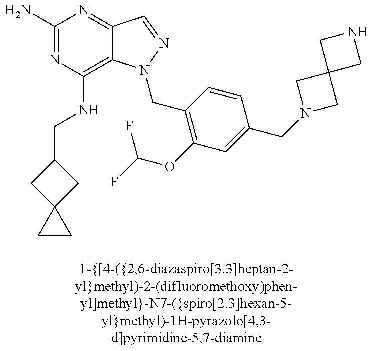 [SMILES: Nc1nc(NCC2CC3(CC3)C2)c2c(cnn2Cc2ccc(CN3CC4(CNC4)C3)cc2OC(F)F)n1] | 829.0 | 587.6 | LC-MS (ES, m/z): [M + H]⁺ 511.1, RT (min) 1.15 (LC/MS Procedure B) δ 8.47 (br s, 1H), 7.80 (s, 1H), 7.20- 7.20 (m, 1H), 7.36-7.19 (m, 1H), 7.13 (t, J = 73.0 Hz, 1H), 7.08-6.95 (m, 1H), 5.84 (s, 2H), 4.32 (br s, 2H), 4.22 (br s, 3H), 4.14 (br s, 4H), 3.81- 3.60 (m, 1H), 3.55-3.38 (m, 1H), 2.77 (br d, J = 7.3 Hz, 1H), 2.37-2.25 (m, 1H), 2.14-1.96 (m, 2H), 1.95- 1.78 (m, 2H), 1.44 (br s, 1H), 0.95 (s, 1H), 0.38 (s, 3H) |
| 243 | 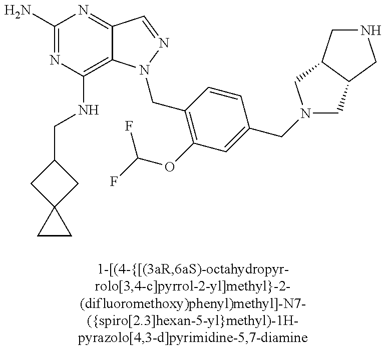 [SMILES: Nc1nc(NCC2CC3(CC3)C2)c2c(cnn2Cc2ccc(CN3C[C@H]4CNC[C@H]4C3)cc2OC(F)F)n1] | 283.2 | 193.4 | LC-MS (ES, m/z): [M + H]⁺ 525.2 RT (min) 1.15 (LC/MS Procedure B) δ 7.61 (s, 1H), 7.17 (s, 4H), 6.43 (br d, J = 7.6 Hz, 1H), 5.78-5.65 (m, 4H), 3.61-3.40 (m, 1H), 2.98 (br s, 2H), 2.67-2.60 (m, 4H), 2.45-2.32 (m, 4H), 2.08 (s, 1H), 2.02-1.91 (m, 2H), 1.73 (br dd, J = 11.7, 6.0 Hz, 2H), 1.42 (br s, 1H), 0.96-0.85 (m, 1H), 0.33 (s, 3H) |
| 244 | 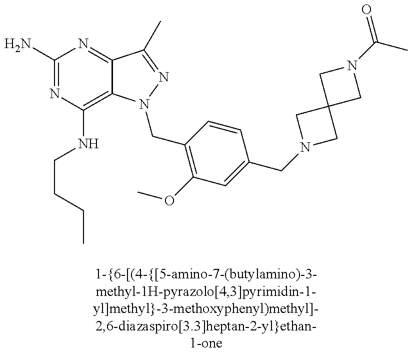 [SMILES: CCCCNc1nc(N)nc2c(C)nn(Cc3ccc(CN4CC5(C4)CN(C(C)=O)C5)cc3OC)c12] | 137.8 | 31.1 | LC/MS (M + H) 492.93 UPLC RT = 1.37 min (Procedure F) δ 6.93 (s, 1H), 6.73 (br d, J = 7.9 Hz, 1H), 6.46 (br d, J = 7.3 Hz, 2H), 5.67 (br s, 1H), 5.52 (s, 2H), 4.23-4.11 (m, 2H), 3.91-3.79 (m, 3H), 3.62- 3.50 (m, 1H), 3.46-3.36 (m, 1H), 3.30 (br s, 2H), 2.25 (s, 3H), 1.93 (s, 2H), 1.72 (s, 3H), 1.48 (quin, J = 7.2 Hz, 2H), 1.32-1.10 (m, 2H), 0.85 (t, J = 7.3 Hz, 3H) |
| 245 | 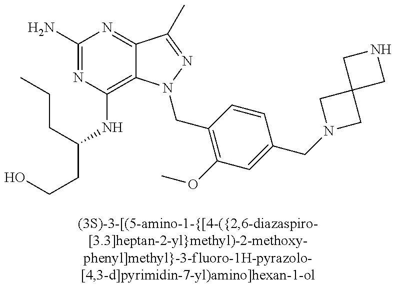 | 40.6 | 1.2 | LC-MS [M + H]⁺ = 499.0 RT (min) = 1.22 (LC/MS Procedure A) δ 6.90 (s, 1H), 6.73 (br d, J = 7.3 Hz, 1H), 6.53 (br d, J = 7.6 Hz, 1H), 6.05 (br d, J = 7.9 Hz, 1H), 5.83 (s, 2H), 5.51 (br d, J = 17.1 Hz, 1H), 5.39 (br d, J = 16.8 Hz, 1H), 4.34 (br s, 1H), 3.84-3.75 (m, 4H), 3.55 (br s, 2H), 3.45 (s, 1H), 3.35 (br t, J = 6.3 Hz, 2H), 3.27-3.17 (m, 3H), 1.82 (s, 4H), 1.74-1.61 (m, 1H), 1.57 (br d, J = 5.8 Hz, 1H), 1.50-1.30 (m, 2H), 1.17-0.98 (m, 2H), 0.79 (br t, J = 7.3 Hz, 3H) |
| 246 | 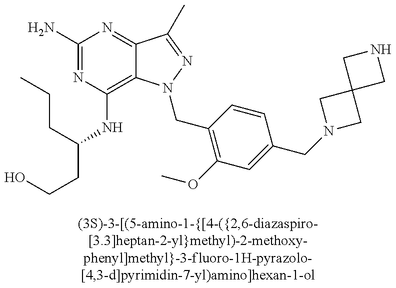 [SMILES: CCC[C@@H](CCO)Nc1nc(N)nc2c(C)nn(Cc3ccc(CN4CC5(CNC5)C4)cc3OC)c12] | 180.3 | 11.1 | LC-MS [M + H]⁺ = 495.2 RT (min) = 1.05 (Method B) NMR: broad signals making interpretation difficult. |
| 247 | 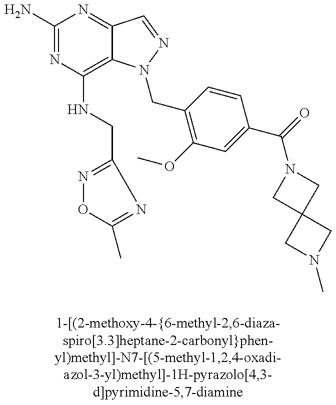 [SMILES: COc1cc(C(=O)N2CC3(CN(C)C3)C2)ccc1Cn1ncc2nc(N)nc(NCc3noc(C)n3)c21] | 196.9 | 37.1 | LC-MS [M + H]⁺ = 505.1 RT (min) = 0.91/Method B δ 7.80 (br s, 1H), 7.16-7.14 (m, 1H), 7.10 (br d, J = 7.8 Hz, 1H), 6.98 (br d, J = 7.6 Hz, 1H), 5.76 (s, 2H), 4.91 (br d, J = 4.6 Hz, 2H), 4.52-4.29 (m, 4H), 4.24-4.15 (m, 3H), 4.16- 3.98 (m, 1H), 3.78 (s, 3H), 2.78 (br s, 3H), 2.58-2.56 (m, 3H) |
| 248 | 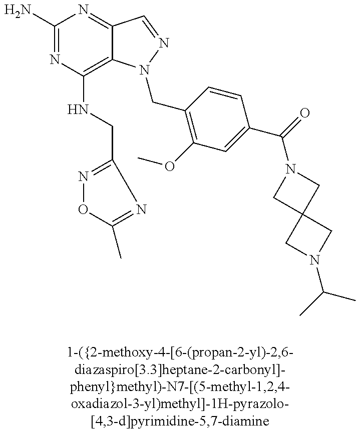 [SMILES: COc1cc(C(=O)N2CC3(C2)CN(C(C)C)C3)ccc1Cn1ncc2nc(N)nc(NCc3noc(C)n3)c21] | 73.0 | 25.4 | LC-MS [M + H]⁺ = 533.3 RT (min) = 0.97/Method B δ 7.79 (s, 1H), 7.17-7.14 (m, 1H), 7.10 (br d, J = 7.6 Hz, 1H), 6.99 (d, J = 7.8 Hz, 1H), 5.75 (s, 2H), 4.91 (br d, J = 3.9 Hz, 2H), 4.55-4.36 (m, 2H), 4.29-4.10 (m, 6H), 3.77 (s, 3H), 2.57 (s, 3H), 1.08 (d, J = 6.5 Hz, 6H). |
| 249 | 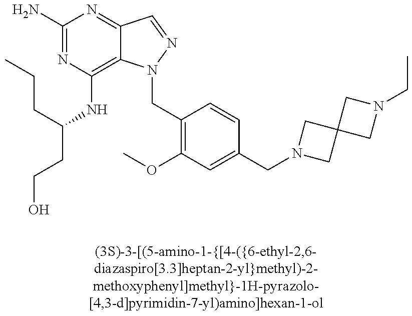 [SMILES: CCC[C@@H](CCO)Nc1nc(N)nc2cnn(Cc3ccc(CN4CC5(CN(CC)C5)C4)cc3OC)c12] | 55.6 | 1.6 | LC/MS [M + H]⁺ = 509.4 RT (min) = 0.97 (LC/MS Procedure A) δ 7.79 (s, 1H), 7.11 (s, 1H), 6.91 (s, 1H), 6.71 (s, 1H), 5.77 (q, J = 16.3 Hz, 2H), 4.54 (s, 1H), 4.28 (s, 2H), 3.82 (s, 3H), 3.13 (s, 1H), 3.01 (s, 0H), 2.57 (s, 4H), 1.73 (s, 4H), 1.54 (s, 4H), 1.26 (s, 0H), 1.17 (s, 4H), 1.05 (t, J = 7.2 Hz, 3H), 0.83 (t, J = 7.4 Hz, 3H). |
| 250 | 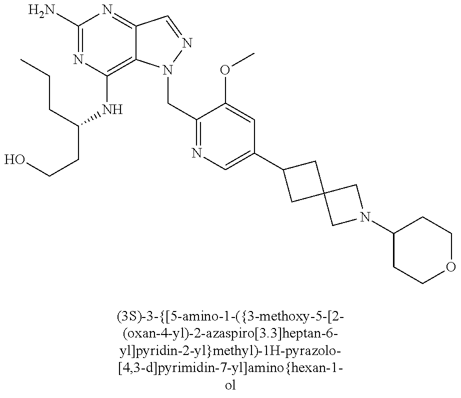 | 45.3 | 1.1 | LC/MS [M + H]+ = 536.9 RT (min) = 1.33 (LC/MS Procedure A) δ 7.92 (s, 1H), 7.82 (d, J = 8.1 Hz, 1H), 7.47 (s, 1H), 7.38 (s, 1H), 5.63 (dd, J = 22.4, 15.2 Hz, 4H), 4.41 (s, 1H), 3.89 (s, 3H), 3.82 (d, J = 11.3 Hz, 2H), 3.37 (s, 1H), 3.25 (t, J = 11.2 Hz, 2H), 3.17 (s, 2H), 3.00 (s, 3H), 2.56 (s, 4H), 2.48 (t, J = 9.9 Hz, 2H), 2.25 (t, J = 10.8 Hz, 2H), 1.82- 1.76 (m, 1H), 1.72 (s, 1H), 1.59 (d, J = 14.5 Hz, 3H), 1.32-1.25 (m, 2H), 1.14 (d, J = 11.8 Hz, 2H), 0.87 (t, J = 7.3 Hz, 3H). |
| 251 | 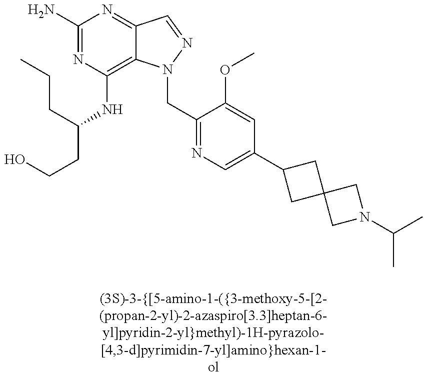 [SMILES: CCC[C@@H](CCO)Nc1nc(N)nc2cnn(Cc3ncc(C4CC5(C4)CN(C(C)C)C5)cc3OC)c12] | 134.4 | 1.8 | LC/MS [M + H]+ = 509.4 RT (min) = 1.08 (LC/MS Procedure A) δ 7.92 (s, 1H), 7.75 (d, J = 8.1 Hz, 1H), 7.45 (s, 1H), 7.38 (d, J = 1.6 Hz, 1H), 5.69-5.54 (m, 5H), 4.41 (s, 1H), 3.90 (s, 3H), 3.19 (s, 1H), 3.00 (s, 2H), 2.43 (dd, J = 11.8, 8.3 Hz, 3H), 2.19 (ddd, J = 18.6, 12.3, 7.7 Hz, 3H), 1.90 (s, 2H), 1.81-1.75 (m, 1H), 1.74-1.68 (m, 1H), 1.57 (dd, J = 15.1, 8.2 Hz, 3H), 1.29 (td, J = 18.5, 17.3, 9.2 Hz, 3H), 0.85 (dd, J = 27.7, 6.7 Hz, 8H). |
| 252 | 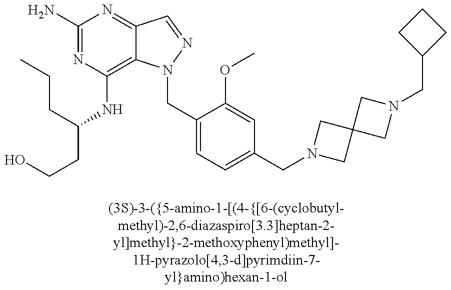 [SMILES: CCC[C@@H](CCO)Nc1nc(N)nc2cnn(Cc3ccc(CN4CC5(C4)CN(CC4CCC4)C5)cc3OC)c12] | 6.1 | 0.4 | LC/MS [M + H]⁺ = 549.2 RT (min) = 1.4 (LC/MS Procedure A) δ 7.58 (s, 1H), 6.93 (s, 1H), 6.71 (d, J = 7.9 Hz, 1H), 6.37 (d, J = 7.8 Hz, 1H), 5.71-5.61 (m, 4H), 5.55 (d, J = 17.0 Hz, 1H), 4.32 (s, 1H), 3.85 (s, 2H), 3.32 (s, 2H), 3.23 (s, 2H), 3.18 (s, 3H), 2.41 (d, J = 7.2 Hz, 2H), 2.28- 2.20 (m, 2H), 1.93 (d, J =7.7 Hz, 3H), 1.86-1.71 (m, 4H), 1.61 (dt, J = 18.6, 9.0 Hz, 3H), 1.54-1.48 (m, 1H), 1.44-1.31 (m, 1H), 1.07- 1.02 (m, 2H), 0.77 (t, J = 7.3 Hz, 3H). |
| 253 | 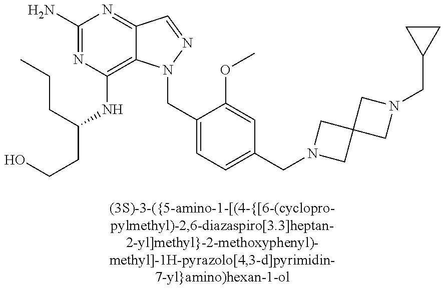 [SMILES: CCC[C@@H](CCO)Nc1nc(N)nc2cnn(Cc3ccc(CN4CC5(C4)CN(CC4CC4)C5)cc3OC)c12] | 14.5 | 1.3 | LC/MS [M + H]⁺ = 535.2 RT (min) = 1.3 (LC/MS Procedure A) δ 7.36 (s, 2H), 6.71 (s, 2H), 6.49 (d, J = 8.0 Hz, 2H), 6.16 (d, J = 7.7 Hz, 2H), 5.59 (d, J = 8.5 Hz, 1H), 5.52 (s, 3H), 5.44 (d, J = 16.9 Hz, 2H), 5.33 (d, J = 16.9 Hz, 2H), 4.10 (s, 2H), 3.31 (s, 1H), 3.10 (s, 2H), 2.55 (d, J = 7.2 Hz, 2H), 2.33 (s, 3H), 1.69 (s, 2H), 1.43 (dt, J = 13.6, 6.5 Hz, 2H), 1.33-1.27 (m, 1H), 1.23-1.11 (m, 1H), 1.12 (s, 1H), 0.82 (q, J = 7.5 Hz, 1H), 0.61 (s, 2H), 0.54 (t, J = 7.3 Hz, 3H), 0.27 (d, J = 7.7 Hz, 1H) |
| 254 | 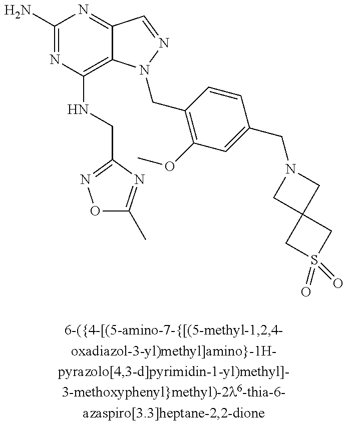 [SMILES: COc1cc(CN2CC3(C2)CS(=O)(=O)C3)ccc1Cn1ncc2nc(N)nc(NCc3noc(C)n3)c21] | 243.8 | 18.7 | LC-MS [M + H]⁺ = 526.3 RT (min) = 094/Method B δ 7.81 (s, 1H), 7.14-7.10 (m, 1H), 7.04-7.00 (m, 1H), 7.00-6.96 (m, 1H), 5.75 (s, 2H), 4.94 (br d, J = 5.5 Hz, 2H), 4.50 (s, 4H), 4.44-4.18 (m, 6H), 3.79 (s, 3H), 2.61 (s, 3H) |
| 255 | 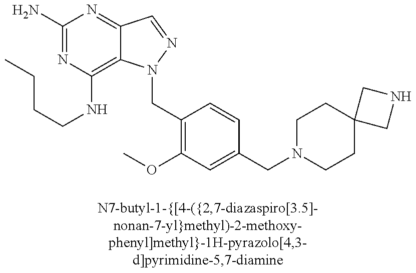 [SMILES: CCCCNc1nc(N)nc2cnn(Cc3ccc(CN4CCC5(CC4)CNC5)cc3OC)c12] | 175.1 | 5.7 | LC/MS [M + H]⁺ = 465.1 RT (min) = 1.24 (LC/MS Procedure A) δ 7.60 (s, 1H), 7.56 (s, 1H), 7.19 (s, 1H), 7.01-6.93 (m, 1H), 6.75 (d, J = 7.5 Hz, 1H), 6.58 (s, 1H), 6.50 (d, J = 7.8 Hz, 1H), 6.45-6.36 (m, 1H), 5.65 (dd, J = 26.9, 12.1 Hz, 4H), 4.57 (s, 1H), 3.91 (s, 1H), 3.57 (d, J = 18.3 Hz, 1H), 3.17 (s, 0H), 2.94 (s, 0H), 2.78 (s, 0H), 2.69 (d, J = 9.8 Hz, 0H), 2.56 (s, 1H), 2.42 (s, 1H), 2.24 (s, 3H), 1.98 (s, 1H), 1.92 (s, 1H), 1.74 (s, 1H), 1.66 (s, 3H), 1.61 (s, 0H), 1.48 (dt, J = 15.1, 7.6 Hz, 2H), 1.23- 1.15 (m, 3H), 0.84 (td, J = 7.5, 3.6 Hz, 3H). |
| 256 | 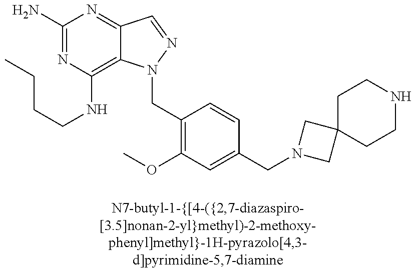 [SMILES: CCCCNc1nc(N)nc2cnn(Cc3ccc(CN4CC5(CCNCC5)C4)cc3OC)c12] | 267.2 | 10.7 | LC/MS [M + H]⁺ = 465.1 RT (min) = 1.23 (LC/MS Procedure A) δ 7.56 (s, 1H), 7.05 (s, 1H), 6.93 (s, 1H), 6.80 (d, J = 7.8 Hz, 1H), 6.73 (d, J = 7.8 Hz, 0H), 6.45 (dd, J = 17.3, 7.6 Hz, 2H), 5.62 (d, J = 12.8 Hz, 4H), 3.70 (s, 1H), 3.53 (s, 0H), 3.10 (s, 1H), 2.95 (s, 1H), 2.91 (s, 2H), 2.73-2.62 (m, 3H), 2.56 (s, 0H), 1.92 (s, 1H), 1.76 (d, J = 15.3 Hz, 3H), 1.49 (q, J = 7.2 Hz, 2H), 1.42 (s, 3H), 1.21 (p, J = 7.2 Hz, 2H), 0.86 (t, J = 7.3 Hz, 3H). |
| 257 | 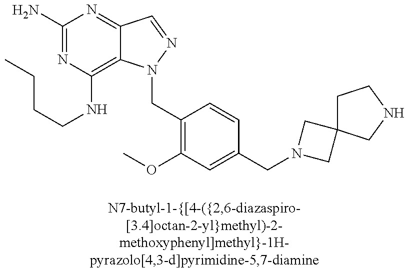 [SMILES: CCCCNc1nc(N)nc2cnn(Cc3ccc(CN4CC5(CCNC5)C4)cc3OC)c12] | 197.6 | 4.8 | LC/MS [M + H]⁺ = 450.9 RT (min) = 1.10 (LC/MS Procedure A) δ 7.56 (s, 1H), 6.93 (s, 1H), 6.73 (d, J = 7.7 Hz, 1H), 6.43 (d, J = 7.7 Hz, 1H), 5.62 (d, J = 18.1 Hz, 3H), 3.84 (s, 2H), 3.12-3.03 (m, 4H), 2.98 (s, 1H), 2.86 (s, 2H), 2.69 (d, J = 9.4 Hz, 0H), 2.56 (s, 4H), 1.87 (d, J = 4.6 Hz, 6H), 1.51-1.44 (m, 2H), 1.20 (q, J = 7.4 Hz, 2H), 0.85 (t, J = 7.3 Hz, 3H). |
| 258 | 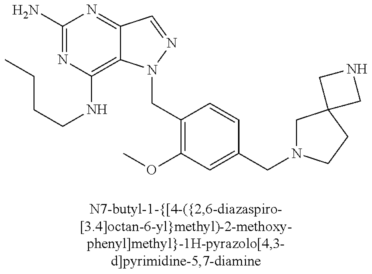 [SMILES: CCCCNc1nc(N)nc2cnn(Cc3ccc(CN4CCC5(CNC5)C4)cc3OC)c12] | 146.5 | 6.5 | LC/MS [M + H]⁺ = 451.1 RT (min) = 1.27 (LC/MS Procedure A) δ 7.57 (s, 1H), 6.97 (d, J = 9.1 Hz, 1H), 6.77 (d, J = 7.8 Hz, 1H), 6.51- 6.42 (m, 1H), 5.74 (d, J = 17.2 Hz, 1H), 5.62 (s, 2H), 3.99 (s, 1H), 3.91 (d, J = 10.4 Hz, 1H), 3.87-3.78 (m, 3H), 3.72 (s, 2H), 3.10 (s, 1H), 2.72- 2.64 (m, 1H), 2.56 (s, 2H), 2.51- 2.44 (m, 2H), 2.02 (dd, J = 13.2, 6.7 Hz, 1H), 1.92 (s, 0H), 1.88 (s, 0H), 1.72 (s, 1H), 1.59 (s, 0H), 1.48 (t, J = 8.9 Hz, 3H), 1.27-1.15 (m, 2H), 0.85 (q, J = 7.4, 6.9 Hz, 3H). |
| 259 | 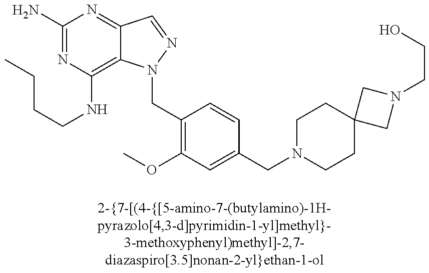 [SMILES: CCCCNc1nc(N)nc2cnn(Cc3ccc(CN4CCC5(CC4)CN(CCO)C5)cc3OC)c12] | 62.5 | 13.6 | LC/MS [M + H]⁺ = 509.0 RT (min) = 1.19 (LC/MS Procedure A) δ 8.33 (d, J = 18.7 Hz, 1H), 7.93 (s, 1H), 7.79 (d, J = 6.3 Hz, 1H), 7.30 (s, 1H), 7.19 (d, J = 10.1 Hz, 1H), 7.10 (s, 1H), 7.05 (s, 1H), 6.97 (d, J = 7.6 Hz, 1H), 6.83 (d, J = 7.8 Hz, 1H), 5.77 (d, J = 10.6 Hz, 2H), 4.64 (d, J = 9.9 Hz, 2H), 4.22 (s, 1H), 3.96 (s, 3H), 3.82 (d, J = 18.9 Hz, 2H), 3.60 (s, 1H), 3.46 (s, 2H), 3.20 (d, J = 15.6 Hz, 3H), 2.52 (s, 2H), 1.89 (s, 2H), 1.61-1.54 (m, 2H), 1.25 (q, J = 7.4 Hz, 2H), 0.88 (t, J = 7.3 Hz, 3H). |
| 260 | 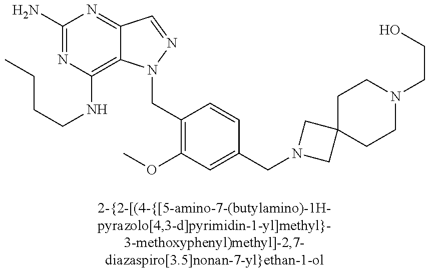 [SMILES: CCCCNc1nc(N)nc2cnn(Cc3ccc(CN4CC5(CCN(CCO)CC5)C4)cc3OC)c12] | 188.2 | 16.3 | LC/MS [M + H]⁺ = 507.1 RT (min) = 1.12 (LC/MS Procedure A) δ 7.55 (s, 1H), 6.92 (s, 1H), 6.71 (d, J = 8.1 Hz, 1H), 6.45-6.34 (m, 1H), 5.61 (d, J = 16.6 Hz, 4H), 3.83 (s, 3H), 2.92 (s, 1H), 2.87 (s, 3H), 2.77 (s, 1H), 2.66 (s, 0H), 2.55 (s, 6H), 2.30 (t, J = 6.3 Hz, 1H), 1.89 (s, 3H), 1.69 (s, 2H), 1.62 (d, J = 5.6 Hz, 3H), 1.46 (q, J = 7.1 Hz, 2H), 1.18 (q, J = 7.1 Hz, 3H), 0.84 (t, J = 7.2 Hz, 4H). |
| 261 | 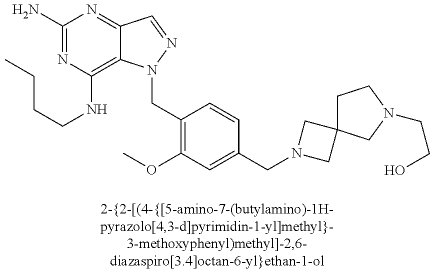 [SMILES: CCCCNc1nc(N)nc2cnn(Cc3ccc(CN4CC5(CCN(CCO)C5)C4)cc3OC)c12] | 120.0 | 8.0 | LC/MS [M + H]⁺ = 494.9 RT (min) = 1.2 (LC/MS Procedure A) δ 7.55 (s, 1H), 6.93 (s, 1H), 6.72 (d, J = 7.8 Hz, 1H), 6.43 (d, J = 7.7 Hz, 1H), 5.61 (d, J = 14.9 Hz, 4H), 3.49- 3.42 (m, 0H), 3.39 (d, J = 6.5 Hz, 1H), 3.12-2.99 (m, 4H), 2.63 (s, 2H), 2.56 (s, 8H), 2.44 (td, J = 6.8, 6.4, 2.9 Hz, 3H), 1.88 (d, J = 11.8 Hz, 5H), 1.46 (t, J = 7.5 Hz, 2H), 1.19 (q, J = 7.5 Hz, 2H), 0.84 (t, J = 7.4 Hz, 3H) |
| 262 | 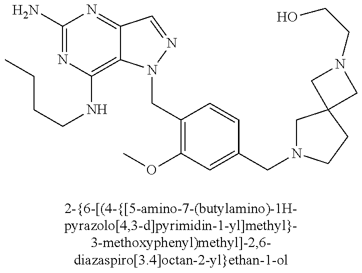 [SMILES: CCCCNc1nc(N)nc2cnn(Cc3ccc(CN4CCC5(C4)CN(CCO)C5)cc3OC)c12] | 64.5 | 4.4 | LC/MS [M + H]+ = 495.0 RT (min) = 1.23 (LC/MS Procedure A) δ 7.57 (s, 1H), 6.97 (s, 1H), 6.76 (d, J = 7.7 Hz, 1H), 6.44 (d, J = 7.6 Hz, 1H), 6.40 (s, 0H), 5.62 (d, J = 10.1 Hz, 3H), 3.91 (s, 0H), 3.40 (d, J = 6.2 Hz, 1H), 3.31 (t, J = 6.2 Hz, 1H), 3.10 (d, J = 6.9 Hz, 2H), 3.04 (d, J = 6.9 Hz, 2H), 2.53 (t, J = 2.0 Hz, 6H), 2.42 (dt, J = 8.8, 6.6 Hz, 4H), 1.89 (d, J = 6.6 Hz, 1H), 1.88 (s, 6H), 1.47 (p, J = 7.1 Hz, 2H), 1.27-1.15 (m, 2H), 0.85 (t, J = 7.3 Hz, 3H). |
| 263 | 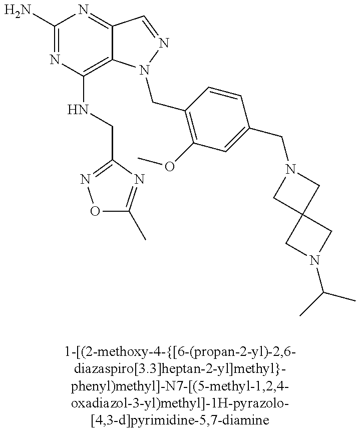 [SMILES: COc1cc(CN2CC3(C2)CN(C(C)C)C3)ccc1Cn1ncc2nc(N)nc(NCc3noc(C)n3)c21] | 68.7 | 12.7 | LC-MS [M + H]⁺ = 519.2 RT (min) = 0.90/Method B δ 7.58-7.56 (m, 1H), 7.35-7.27 (m, 1H), 6.86-6.85 (m, 1H), 6.69 (br d, J = 7.7 Hz, 1H), 6.61 (d, J = 7.7 Hz, 1H), 5.66 (s, 2H), 5.57 (s, 2H), 4.77 (br s, 2H), 3.77 (s, 3H), 3.14 (br d, J = 5.0 Hz, 8H), 2.56-2.54 (m, 3H), 2.20 (dt, J = 12.6, 6.2 Hz, 1H), 0.80 (d, J = 6.1 Hz, 6H) |
| 264 | 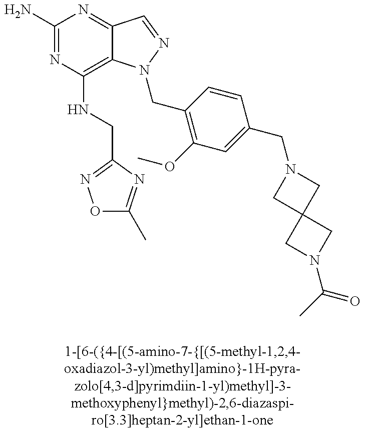 [SMILES: COc1cc(CN2CC3(C2)CN(C(C)=O)C3)ccc1Cn1ncc2nc(N)nc(NCc3noc(C)n3)c21] | 916.1 | 78.2 | LC-MS [M + H]⁺ = 519.4 RT (min) = 0.94/Method B δ 7.57 (s, 1H), 7.32 (br t, J = 5.0 Hz, 1H), 6.87-6.85 (m, 1H), 6.70 (br d, J = 7.4 Hz, 1H), 6.62 (d, J = 8.0 Hz, 1H), 5.66 (s, 2H), 5.58 (s, 2H), 4.77 (br d, J = 4.1 Hz, 2H), 4.13 (s, 2H), 3.85 (s, 2H), 3.78 (s, 3H), 3.22 (s, 4H), 2.55 (br s, 3H), 1.70 (s, 3H) |
| 265 | 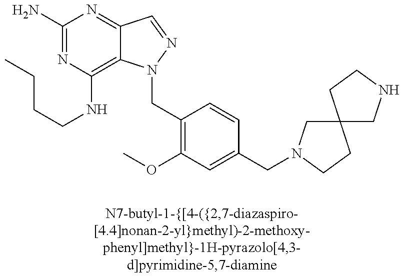 [SMILES: CCCCNc1nc(N)nc2cnn(Cc3ccc(CN4CCC5(CCNC5)C4)cc3OC)c12] | 177.4 | 33.1 | LC/MS [M + H]⁺ = 465.2 RT (min) = 1.20 (Procedure A) δ 7.56 (s, 1H), 6.98 (s, 1H), 6.77 (d, J = 7.8 Hz, 1H), 6.45 (d, J = 7.8 Hz, 1H), 5.63 (d, J = 15.2 Hz, 3H), 3.59- 3.48 (m, 2H), 3.03 (d, J = 7.7 Hz, 0H), 3.00 (d, J = 7.7 Hz, 0H), 2.94 (d, J = 11.2 Hz, 1H), 2.86 (d, J = 11.0 Hz, 1H), 2.60 (t, J = 7.5 Hz, 1H), 2.56 (s, 4H), 2.49 (d, J = 9.7 Hz, 2H), 2.31 (d, J = 9.0 Hz, 1H), 1.88 (s, 4H), 1.78 (dq, J = 21.5, 8.4, 7.5 Hz, 4H), 1.52- 1.45 (m, 2H), 1.20 (q, J = 7.5 Hz, 2H), 0.85 (t, J = 7.3 Hz, 3H). |
| 266 | 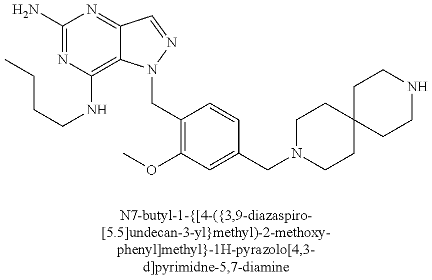 [SMILES: CCCCNc1nc(N)nc2cnn(Cc3ccc(CN4CCC5(CCNCC5)CC4)cc3OC)c12] | 140.9 | 9.3 | LC/MS [M + H]⁺ = 492.9 RT (min) = 1.33 (Procedure A) δ 8.60 (s, 2H), 8.31 (d, J = 5.9 Hz, 1H), 7.92 (s, 1H), 7.77 (s, 1H), 7.29 (s, 0H), 7.20 (s, 1H), 7.08 (s, 0H), 6.97 (d, J = 7.7 Hz, 1H), 6.83 (d, J = 7.7 Hz, 1H), 5.75 (s, 2H), 4.28 (s, 2H), 3.82 (s, 1H), 3.79 (s, 3H), 3.76 (d, J = 9.1 Hz, 1H), 3.57 (q, J = 6.7 Hz, 1H), 3.43 (s, 0H), 3.13 (s, 2H), 2.58 (s, 1H), 2.55 (s, 2H), 2.47 (t, J = 2.0 Hz, 1H), 1.84 (s, 2H), 1.76 (s, 4H), 1.58 (q, J = 7.5 Hz, 2H), 1.53 (d, J = 13.0 Hz, 3H), 1.25 (h, J = 7.4 Hz, 2H), 0.88 (q, J = 7.4, 6.8 Hz, 3H) |
| 267 | 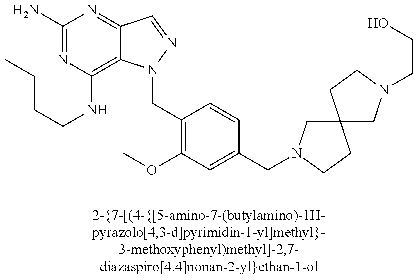 [SMILES: CCCCNc1nc(N)nc2cnn(Cc3ccc(CN4CCC5(CCN(CCO)C5)C4)cc3OC)c12] | 58.5 | 1.3 | LC/MS [M + H]⁺ = 509.21 RT (min) = 1.13 (Procedure A) δ 7.55 (d, J = 2.8 Hz, 1H), 6.98 (s, 1H), 6.79-6.72 (m, 1H), 6.47- 6.36 (m, 2H), 5.62 (d, J = 15.5 Hz, 6H), 3.53 (d, J = 18.6 Hz, 1H), 3.39 (d, J = 6.4 Hz, 0H), 3.05 (d, J = 11.5 Hz, 0H), 3.01-2.91 (m, 0H), 2.60- 2.54 (m, 1H), 2.55 (s, 5H), 2.51- 2.42 (m, 3H), 2.30 (t, J = 8.9 Hz, 1H), 1.91 (s, 3H), 1.78 (dt, J = 13.9, 7.2 Hz, 1H), 1.72 (dt, J = 13.8, 7.0 Hz, 3H), 1.46 (p, J = 7.2 Hz, 3H), 1.18 (h, J = 7.1 Hz, 3H), 0.84 (td, J = 7.4, 3.0 Hz, 4H). |
| 268 | 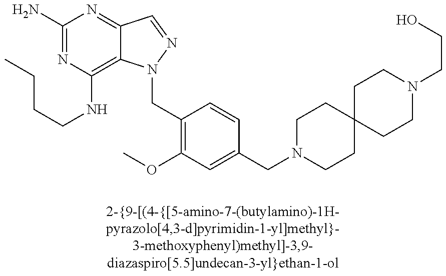 [SMILES: CCCCNc1nc(N)nc2cnn(Cc3ccc(CN4CCC5(CCN(CCO)CC5)CC4)cc3OC)c12] | 147.4 | 38.2 | LC/MS [M + H]⁺ = 535.2 RT (min) = 11.8 (LC/MS Procedure A) δ 7.56 (s, 1H), 6.97 (s, 1H), 6.75 (d, J = 7.9 Hz, 1H), 6.42 (d, J = 7.8 Hz, 1H), 5.63 (d, J = 19.1 Hz, 3H), 3.92 (s, 1H), 3.84 (s, 2H), 3.19 (s, 1H), 3.00 (s, 0H), 2.56 (s, 8H), 2.31 (s, 3H), 1.92 (s, 4H), 1.54 (s, 0H), 1.50- 1.39 (m, 8H), 1.18 (q, J = 7.4 Hz, 2H), 0.84 (t, J = 7.4 Hz, 3H). |
| 269 | 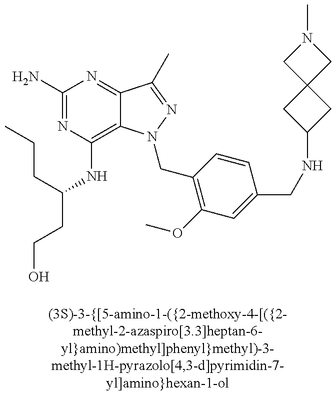 [SMILES: CCC[C@@H](CCO)Nc1nc(N)nc2c(C)nn(Cc3ccc(CNC4CC5(C4)CN(C)C5)cc3OC)c12] | 69.9 | 3.7 | LC-MS [M + H]⁺ = 523.4 RT (min) = 1.12 (Procedure A) (¹H NMR (400 MHz, DMSO-d₆) δ = 6.99-6.95 (m, 1H), 6.77-6.72 (m, 1H), 6.39-6.33 (m, 1H), 5.66-5.59 (m, 4H), 5.57-5.53 (m, 1H), 5.49- 5.42 (m, 1H), 4.33-4.25 (m, 1H), 4.17-4.01 (m, 1H), 3.84 (s, 3H), 3.70-3.68 (m, 1H), 3.40 (br s, 6H), 3.17 (br d, J = 2.7 Hz, 3H), 2.85-2.80 (m, 2H), 2.58 (s, 2H), 2.25 (s, 4H), 2.14-2.10 (m, 3H), 2.07 (s, 1H), 1.86 (s, 1H), 1.64 (dt, J = 1.8, 3.2 Hz, 2H), 1.31-1.27 (m, 2H), 1.24-1.22 (m, 1H), 1.09-0.98 (m, 2H), 0.71 (br d, J = 1.5 Hz, 3H) |
| 270 | 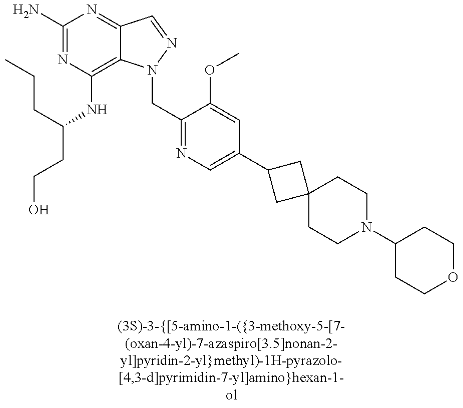 [SMILES: CCC[C@@H](CCO)Nc1nc(N)nc2cnn(Cc3ncc(C4CC5(CCN(C6CCOCC6)CC5)C4)cc3OC)c12] | 11.5 | 0.2 | LC/MS [M + H]+ = 579.3 RT (min) = 1.23 (LC/MS Procedure A) δ 7.93 (s, 1H), 7.82 (s, 1H), 7.50- 7.43 (m, 1H), 7.36 (s, 1H), 5.64- 5.54 (m, 2H), 4.41 (s, 1H), 3.92- 3.83 (m, 4H), 3.24 (t, J = 11.3 Hz, 1H), 2.61-2.52 (m, 2H), 2.55 (s, 8H), 2.46 (s, 0H), 2.34 (d, J = 12.2 Hz, 3H), 2.19 (t, J = 9.8 Hz, 1H), 1.83 (s, 5H), 1.82-1.75 (m, 0H), 1.69- 1.61 (m, 3H), 1.56 (d, J = 7.5 Hz, 1H), 1.50 (d, J = 5.4 Hz, 1H), 1.42- 1.36 (m, 1H), 1.29 (s, 2H), 0.86 (t, J = 7.4 Hz, 3H). |
| 271 | 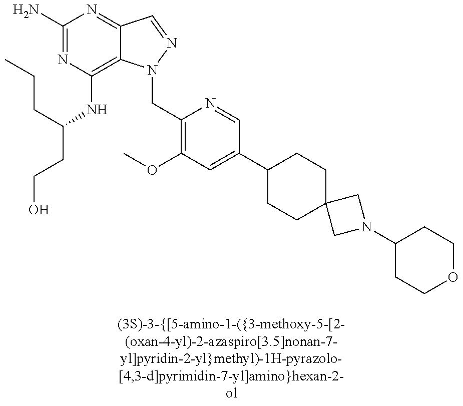 [SMILES: CCC[C@@H](CCO)Nc1nc(N)nc2cnn(Cc3ncc(C4CCC5(CC4)CN(C4CCOCC4)C5)cc3OC)c12] | 13.3 | 0.6 | LC/MS [M + H]+ = 579.2 RT (min) = 1.21 (LC/MS Procedure A) δ 7.95 (d, J = 1.7 Hz, 1H), 7.86 (d, J = 8.1 Hz, 1H), 7.47 (s, 1H), 7.40 (s, 1H), 5.68-5.56 (m, 3H), 4.42 (s, 1H), 3.84 (d, J = 11.4 Hz, 2H), 3.28 (t, J = 11.0 Hz, 1H), 3.13 (s, 1H), 3.00 (s, 1H), 2.56 (s, 7H), 2.40 (s, 1H), 1.92 (s, 6H), 1.84-1.76 (m, 1H), 1.70 (d, J = 10.6 Hz, 2H), 1.66- 1.53 (m, 3H), 1.51 (d, J = 10.8 Hz, 3H), 1.31 (tt, J = 13.9, 7.0 Hz, 2H), 1.17 (d, J = 11.3 Hz, 2H), 0.88 (t, J = 7.4 Hz, 3H). |
| 272 | 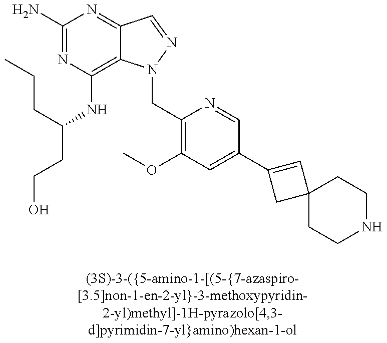 [SMILES: CCC[C@@H](CCO)Nc1nc(N)nc2cnn(Cc3ncc(C4=CC5(CCNCC5)C4)cc3OC)c12] | 667.8 | 31.9 | LC/MS [M + H]⁺ = 492.9 RT (min) = 1.21 (LC/MS Procedure A) δ 8.03 (d, J = 1.9 Hz, 1H), 7.48 (s, 2H), 6.85 (s, 1H), 5.67 (q, J = 15.9 Hz, 2H), 5.58 (s, 1H), 4.41 (s, 1H), 3.91 (s, 2H), 2.96 (s, 2H), 2.84 (s, 2H), 2.55 (d, J = 11.3 Hz, 5H), 1.85 (s, 4H), 1.81-1.74 (m, 1H), 1.70 (t, J = 6.5 Hz, 1H), 1.65 (s, 3H), 1.58- 1.50 (m, 2H), 1.25 (d, J = 7.6 Hz, 2H), 0.85 (t, J = 7.3 Hz, 3H). |
| 273 | 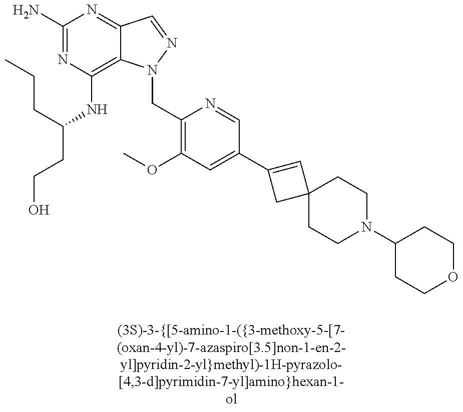 [SMILES: CCC[C@@H](CCO)Nc1nc(N)nc2cnn(Cc3ncc(C4=CC5(CCN(C6CCOCC6)CC5)C4)cc3OC)c12] | 47.5 | 1.5 | LC/MS [M + H]+ = 577.3 RT (min) = 1.3 (LC/MS Procedure A) δ 8.03 (d, J = 1.6 Hz, 1H), 7.49 (d, J = 7.4 Hz, 3H), 6.82 (s, 1H), 5.70 (d, J = 15.8 Hz, 1H), 5.64 (d, J = 15.9 Hz, 1H), 5.58 (s, 2H), 4.41 (s, 1H), 3.48 (s, 0H), 3.29 (t, J = 11.4 Hz, 1H), 3.19 (s, 0H), 2.65 (s, 2H), 2.56 (s, 6H), 2.45 (d, J = 32.7 Hz, 4H), 1.93 (s, 4H), 1.78 (t, J = 6.5 Hz, 1H), 1.68 (t, J = 11.8 Hz, 4H), 1.57 (dd, J = 15.3, 8.7 Hz, 4H), 1.47 (dd, J = 13.2, 9.2 Hz, 2H), 1.26 (d, J = 7.8 Hz, 2H), 0.86 (t, J = 7.4 Hz, 3H). |
| 274 | 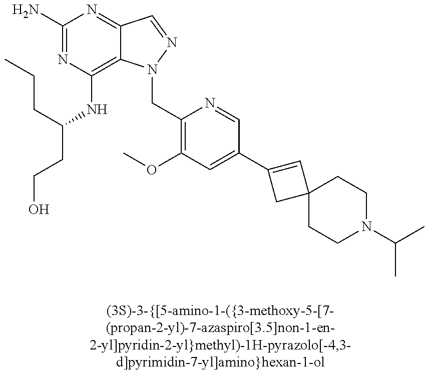 [SMILES: CCC[C@@H](CCO)Nc1nc(N)nc2cnn(Cc3ncc(C4=CC5(CCN(C(C)C)CC5)C4)cc3OC)c12] | 82.3 | 1.1 | LC/MS [M + H]+ = 535.9 RT (min) = 1.26 (LC/MS Procedure A) δ 8.03 (d, J = 1.8 Hz, 1H), 7.50 (d, J = 5.0 Hz, 2H), 6.86 (s, 1H), 5.75 (s, 2H), 5.73-5.63 (m, 2H), 3.92 (s, 3H), 2.56 (s, 9H), 1.92 (s, 3H), 1.79 (s, 5H), 1.79-1.67 (m, 1H), 1.56 (dt, J = 14.8, 7.5 Hz, 2H), 1.27 (s, 2H), 1.18 (d, J = 6.5 Hz, 6H), 0.86 (t, J = 7.3 Hz, 3H) |
| 275 | 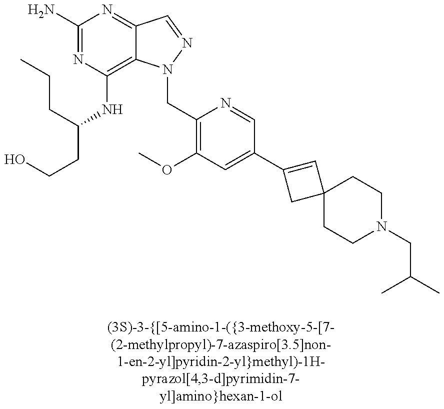 [SMILES: CCC[C@@H](CCO)Nc1nc(N)nc2cnn(Cc3ncc(C4=CC5(CCN(CC(C)C)CC5)C4)cc3OC)c12] | 11.0 | 1.2 | LC/MS [M + H]+ = 549.2 RT (min) = 1.31 (LC/MS Procedure A) δ 8.88 (d, J = 8.3 Hz, 1H), 7.85 (s, 1H), 7.80-7.69 (m, 1H), 7.54 (s, 1H), 7.49 (s, 1H), 5.88-5.77 (m, 2H), 4.56 (d, J = 7.6 Hz, 1H), 3.49 (dd, J = 16.5, 12.0 Hz, 3H), 2.96 (s, 3H), 2.55 (s, 8H), 2.50-2.39 (m, 1H), 2.10 (t, J = 6.8 Hz, 1H), 2.06 (s, 3H), 1.80 (q, J = 6.4 Hz, 2H), 1.70 (d, J = 14.7 Hz, 1H), 1.60 (q, J = 7.8 Hz, 2H), 1.25 (s, 2H), 0.95 (dd, J = 17.6, 6.6 Hz, 7H), 0.92-0.79 (m, 3H) |
| 276 | 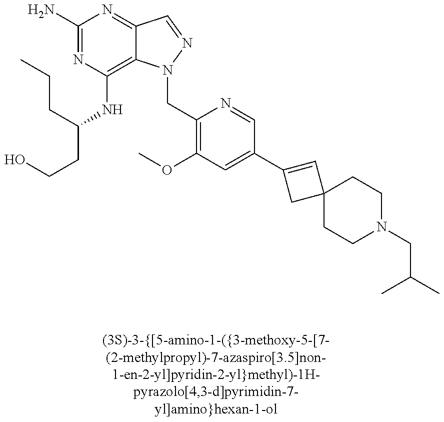 [SMILES: CCC[C@@H](CCO)Nc1nc(N)nc2cnn(Cc3ncc(C4=CC5(CCN(CC(C)C)CC5)C4)cc3OC)c12] | 49.8 | 8.4 | LC/MS [M + H]+ = 547.3 RT (min) = 1.37 (LC/MS Procedure A) δ 8.83 (s, 1H), 8.01 (s, 1H), 7.75 (d, J = 14.5 Hz, 2H), 7.52 (d, J = 18.6 Hz, 1H), 7.07 (s, 1H), 6.74 (s, 1H), 5.88- 5.80 (m, 1H), 4.56 (d, J = 9.2 Hz, 1H), 4.00 (s, 1H), 3.94 (s, 2H), 3.54 (s, 1H), 3.49 (s, 1H), 3.03 (s, 3H), 2.62 (s, 2H), 2.55 (s, 1H), 2.01 (s, 2H), 1.84-1.73 (m, 4H), 1.63- 1.56 (m, 1H), 1.27 (d, J = 7.3 Hz, 0H), 1.25 (s, 2H), 1.08 (s, 1H), 0.86 (t, J = 7.3 Hz, 2H), 0.67 (d, J = 1.7 Hz, 1H), 0.65 (s, 2H), 0.38 (s, 4H). |
| 277 | 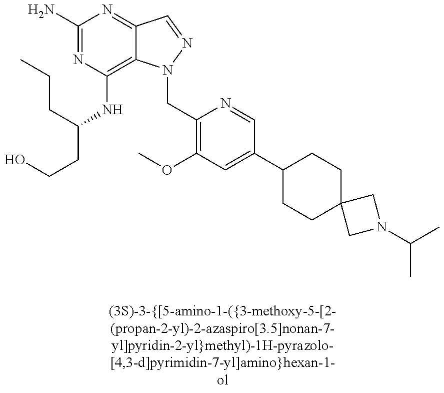 [SMILES: CCC[C@@H](CCO)Nc1nc(N)nc2cnn(Cc3ncc(C4CCC5(CC4)CN(C(C)C)C5)cc3OC)c12] | 19.7 | 0.3 | LC/MS [M + H]+ = 536.9 RT (min) = 1.33 (LC/MS Procedure A) δ 7.94 (s, 1H), 7.86 (d, J = 8.3 Hz, 1H), 7.47 (s, 1H), 7.39 (s, 1H), 5.62 (dd, J = 22.5, 14.8 Hz, 3H), 4.42 (s, 1H), 3.89 (s, 3H), 3.05 (s, 2H), 2.92 (s, 2H), 2.52 (t, J = 2.3 Hz, 6H), 2.43- 2.36 (m, 1H), 1.90 (s, 4H), 1.80 (dd, J = 13.3, 6.0 Hz, 1H), 1.73 (t, J = 6.9 Hz, 1H), 1.68 (s, 2H), 1.58 (d, J = 6.5 Hz, 2H), 1.49 (d, J = 9.0 Hz, 4H), 1.31 (dt, J = 14.9, 7.5 Hz, 2H), 0.88 (dd, J = 6.9, 4.1 Hz, 6H) |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### Industrial Applicability and Uses

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, CP- | CTLA-4 |
| | 675, 206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and analytical (LC/MS) liquid chromatography methods were used:

LCMS procedure A: Column: Waters XBridge C18, 2.1 mm×50 mm, 1.7 μm particles; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0% B to 100% B over 3 min, then a 0.50 min hold at 100% B; Flow: 1 mL/min; Detection: MS and UV (220 nm).

LCMS procedure B: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3 min, then a 0.50 min hold at 100% B; Flow: 1 mL/min; Detection: MS and UV (220 nm).

LCMS procedure C: Column: Waters XBridge BEH C18 XP (50×2.1 mm) 2.5 μm; Mobile Phase A: 5:95 acetonitrile: water with 10 mM NH₄OAc; Mobile Phase B: 95:5 acetonitrile: water with 10 mM NH₄OAc; Temperature: 50° C.; Gradient: 0-100% B over 3 minutes; Flow: 1.1 mL/min

LCMS procedure D: Column: Ascentis Express C18 (50×2.1 mm) 2.7 am; Mobile Phase A: 5:95 acetonitrile: water with 10 mM NH₄OAc; Mobile Phase B: 95:5 acetonitrile: water with 10 mM NH₄OAc; Temperature: 50° C.; Gradient: 0-100% B over 3 minutes; Flow: 1.1 mL/min.

LCMS Procedure E. Column: BEH C18 2.1×50 mm; Mobile Phase A: water with 0.05% TFA; Mobile Phase B: acetonitrile with 0.05% TFA; Temperature: 50° C.; Gradient: 2-98% B over 1.7 min; Flow: 0.8 mL/min.

LCMS Procedure F. Column: Waters XBridge C18, 2.1 mm×50 mm, 1.7 am particles; Mobile Phase A: 5:95 acetonitrile:water with 10 mM NH₄OAc; Mobile Phase B: 95:5 acetonitrile:water with 10 mM NH₄OAc; Temperature:50° C.; Gradient: 0% B to 100% B over 3 min, then a 0.50 min hold at 100% B; Flow: 1 mL/min; Detection: MS and UV (220 nm). This method is a Ultra-Performance Liquid Chromatography (UPLC™) method.

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown for convenience, but it is to be understood that they are present in the initial product mixture and separated at a later time, for example by preparative HPLC.

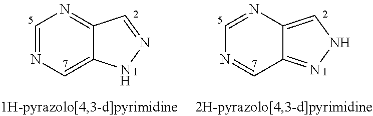 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a 15 later stage, as desired.

The compounds of the present disclosure can be prepared by a number of methods well known to one skilled in the art of synthetic organic chemistry. These methods include those described below, or variations thereof. Preferred methods include, but are not limited to, those described below in the Schemes below.

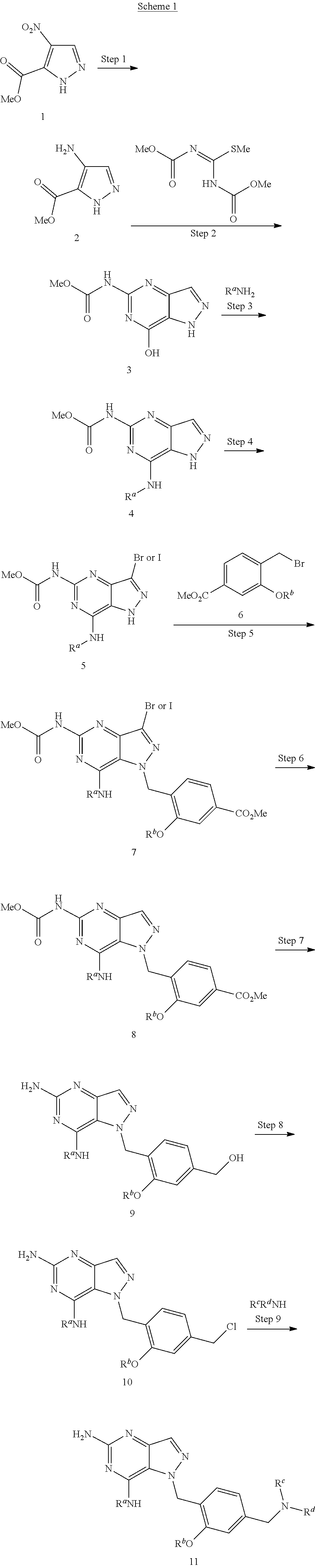

R^{a} can be, in Scheme 1 and other occurrences thereof, for example,

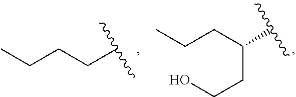

or other suitable moiety. R^{b} is, in Scheme 1 and other occurrences thereof, for example, C₁-C₃ alkyl. R^{c}NHR^{d} is, in Scheme 1 and other occurrences thereof, a primary or secondary amine. R^{a}, R^{b}, R^{c}, and/or R^{d} can have functional groups masked by a protecting group that is removed at the appropriate time during the synthetic process.

Compound 11 can be prepared by the synthetic sequence outlined in Scheme 1 above. Reduction of nitropyrazole 1 to afford compound 2 followed by cyclization with 1,3-bis(methoxycarbonyl)-2-methyl-2-thiopseudourea gives the hydroxypyrazolopyrimidine 3. The amine R^{a}NH₂ is introduced using BOP/DBU coupling conditions, and the subsequent bromination using NBS or iodination using NIS(Step 4) gives the bromo or lodo-pyrazolopyrimidine 5. Alkylation using a benzyl halide 6 gives a mixture of N1 and N2 products, which are separated, giving N1 intermediate 7. Catalytic hydrogenation (step 6) followed by a one-pot LiAIH₄ reduction and carbamate hydrolysis gives the intermediate alcohol 9. Conversion of alcohol 9 to benzyl chloride followed by displacement of it with suitable amines give compound 11. (Alkylation of brominated intermediate 5 in Step 5 gives a better ratio of N1/N2 product, compared to alkylation of unbrominated intermediate 4).

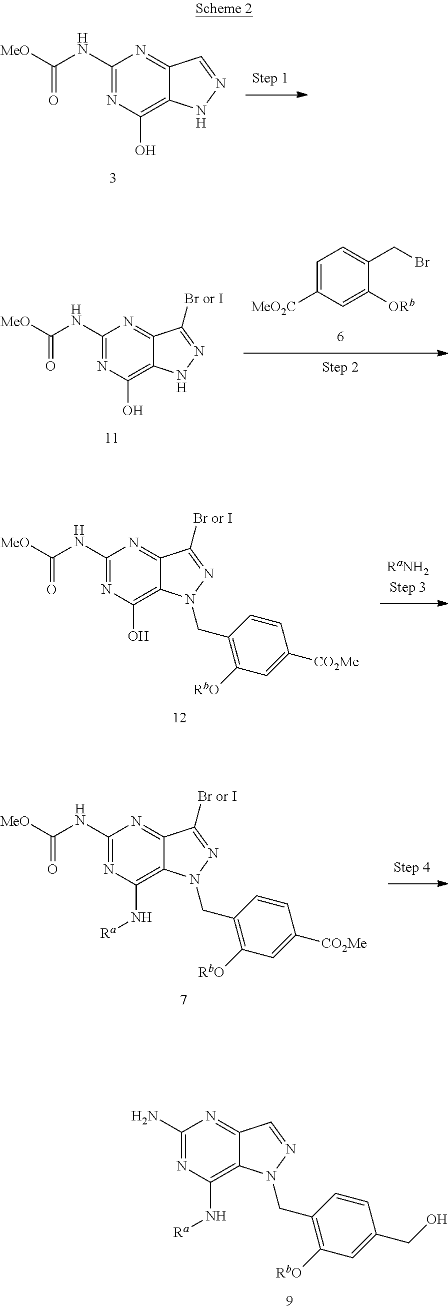

Alternatively, intermediate 9 may be accessed using the route described in Scheme 2 above. Intermediate 3 is brominated or iodinated using NBS or NIS, then alkylated to give the intermediate ester 12. Amination then follows, using BOP coupling conditions to give intermediate 7. Catalytic hydrogenation followed by LiAIH₄ reduction to alcohol and methyl carbamate deprotection gives intermediate 9.

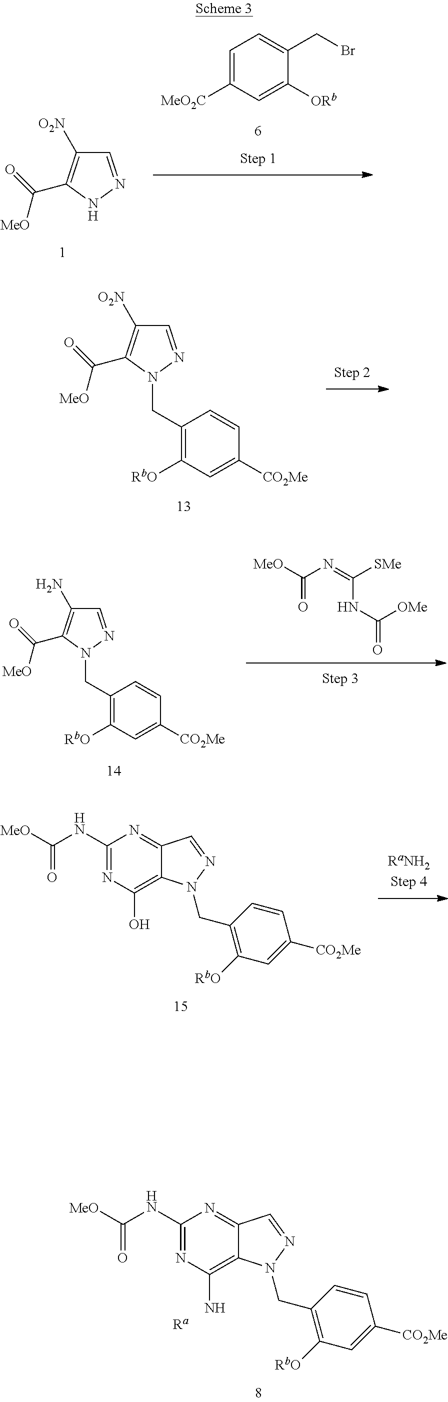

An alternative route to intermediate 8 begins with the alkylation of nitropyrazole 1 with benzyl halide 6, giving the benzyl pyrazole 13. Reduction of the nitro group followed by cyclisation with 1,3-bis(methoxycarbonyl)-2-methyl-2-thiopseudourea gives the hydroxypyrazolopyrimidine 15, which is converted to the appropriate amine derivative 8 using BOP/DBU conditions. This is illustrated in scheme 3 above.

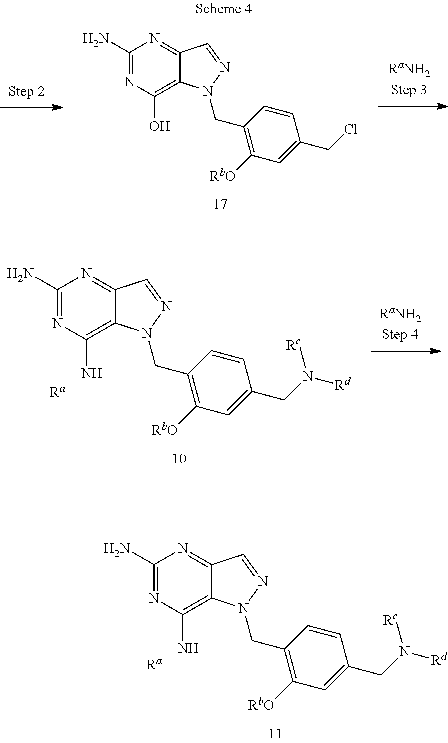

Another alternative route to the target compounds is shown in scheme 4 above. From intermediate 15, the ester group is reduced and the methyl carbamate removed using NaOH, giving the alcohol 16. Conversion of alcohol 16 to chloride followed by displacement with suitable amine gives 17, and subsequent amination using BOP/DBU conditions gives the target molecule 11.

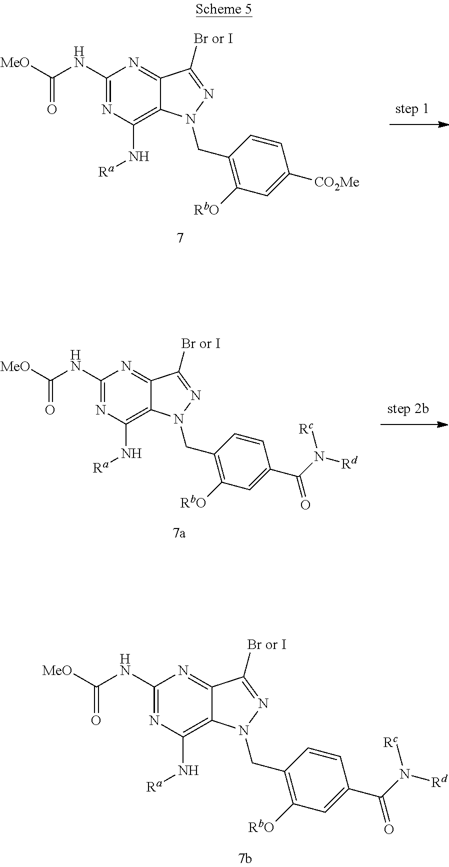

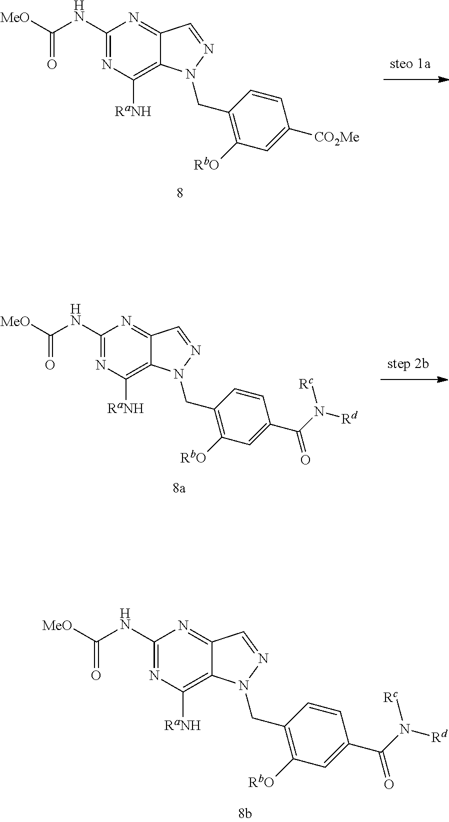

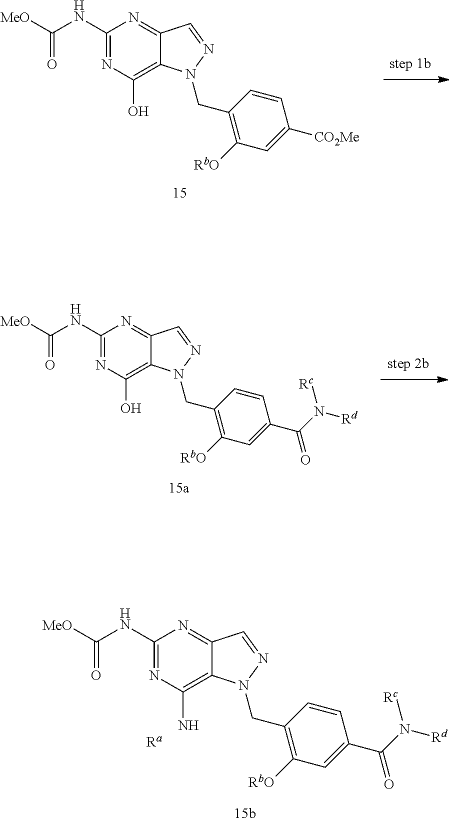

In Scheme 5 sbovr, hydrolysis of methyl ester in 7/8 or 15 followed by amide formation can give corresponding amidnes 7a/8a or 15a. Catalytic hydrogenation of 7a followed by carbamate deprotection produces compound 7b. Carbamate deprotection on 8a gives compound 8b. Finally, amine installation on 15a followed carbamate deprotection gives compound 15b.

### SYNTHESIS—SPECIFIC EXAMPLES

To further illustrate the foregoing, the following non-limiting, the following exemplary synthetic schemes are included. Variations of these examples within the scope of the the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 100 and higher can be found in Table A.

### Example 1—Compound 101

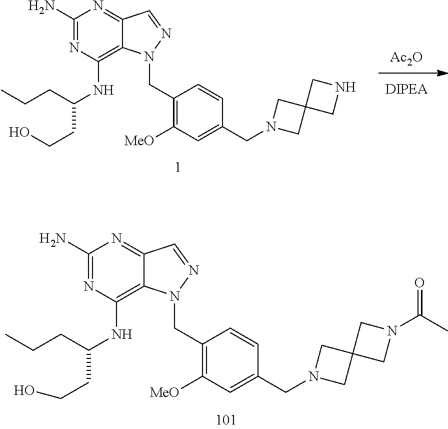 [SMILES: "Ac2O" ; CCC[C@@H](CCO)Nc1nc(N)nc2cnn(Cc3ccc(CN4CC5(CNC5)C4)cc3OC)c12 ; CCC[C@@H](CCO)Nc1nc(N)nc2cnn(Cc3ccc(CN4CC5(C4)CN(C(C)=O)C5)cc3OC)c12]

A solution of (S)-3-((1-(4-((2,6-diazaspiro[3.3]heptan-2-yl)methyl)-2-methoxybenzyl)-5-amino-1H-pyrazolo[4,3-d]pyrimidin-7-yl)amino)hexan-1-ol 1 (US 2020/0038403 A1; 31 mg, 0.065 mmol) in DMF (1 mL) was treated with Ac₂O (6.09 μL, 0.065 mmol) and stirred at RT for 1 h. The solvent was evaporated and the residue was dissolved in DMF (1 mL). The crude residue was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 4% B, 4-44% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to obtain 5 mg of Compound 101.

The following compounds were analogously prepared: Compound 106, Compound 107, Compound 215 (made by reductive amination of compound 1 with formaldehyde), and Compound 216 (made by reductive amination of Compound 1 with acetone).

### Example 2—Compound 110

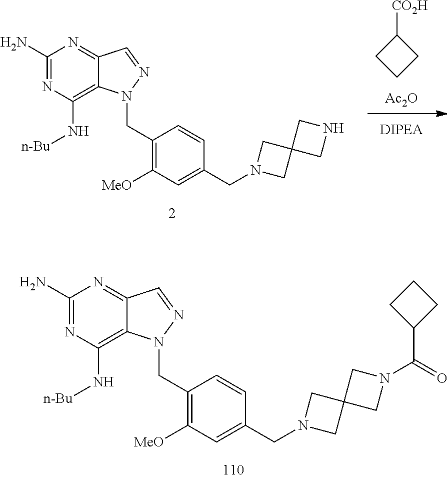

A solution of 1-(4-((2,6-diazaspiro[3.3]heptan-2-yl)methyl)-2-methoxybenzyl)-N7-butyl-1H-pyrazolo[4,3-d]pyrimidine-5,7-diamine 2 (US 2020/0038403 A1; 32 mg, 0.073 mmol) and cyclobutanecarboxylic acid (7.01 μl, 0.073 mmol) in DMF (0.5 mL) was treated with Hunig's base (0.064 mL, 0.366 mmol) and HATU (33.4 mg, 0.088 mmol) and stirred for 30 min. The base was evaporated and syringe-filtered. The crude material was purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 12% B, 12-52% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation, to yield Compound 110.

The following compounds were analogously prepared: Compound 104, Compound 105, and Compound 111.

### Example 3—Compound 102

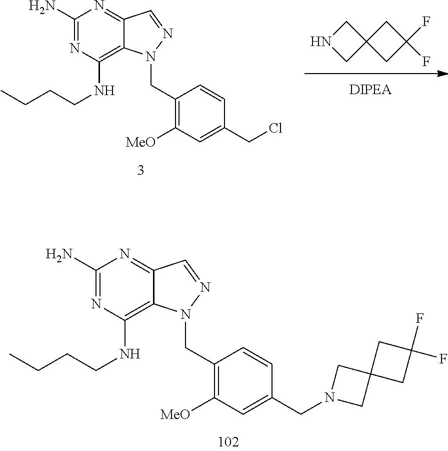 [SMILES: CCCCNc1nc(N)nc2cnn(Cc3ccc(CCl)cc3OC)c12 ; FC1(F)CC2(CNC2)C1 ; CCCCNc1nc(N)nc2cnn(Cc3ccc(CN4CC5(C4)CC(F)(F)C5)cc3OC)c12]

A solution of N7-butyl-1-(4-(chloromethyl)-2-methoxybenzyl)-1H-pyrazolo[4,3-d]pyrimidine-5,7-diamine 3 (US 2020/0038403 A1; 15 mg, 0.04 mmol) in 2 ml DMF was treated with 6,6-difluoro-2-azaspiro[3.3]heptane (10.6 mg, 0.08 mml) and heated 80° C. for 1 h. LCMS showed completion of the reaction. The reaction was syringe filtered. The crude material was purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 21% B, 21-61% B over 20 minutes, then a 4-minute hold at 100% B; Flow Rate: 20 mL/min; Column Temperature: 25° C. Fraction collection was triggered by MS and UV signals. Fractions containing the desired product were combined and dried via centrifugal evaporation, to yield compound 102.

Compound 103 was Analogously Prepared.

### Example 4—Compound 112

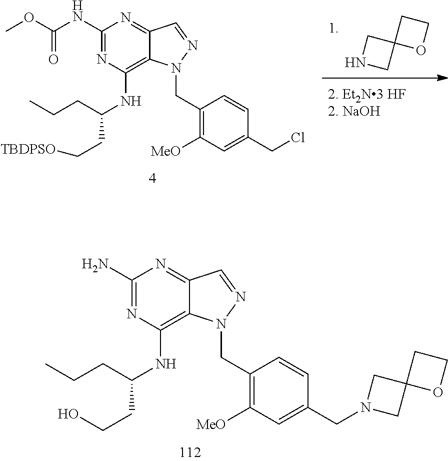

A solution of methyl (S)-(7-((1-((tert-butyldiphenylsilyl)oxy)hexan-3-yl)amino)-1-(4-(chloromethyl)-2-methoxybenzyl)-1H-pyrazolo[4,3-d]pyrimidin-5-yl)carbamate 4 (US 2020/0038403; 20 mg, 0.028 mmol) in 1 mL DMF was treated with 1-oxa-6-azaspiro[3.3]-heptane (13 mf, 0.14 mmol) and heated at 80° C. for 1 h. The reaction mixture was treated with triethylamine-trihydrofluoride (23 μl, 0.14 mmol) and stirred at RT for 3 h. The crude product was treated with NaOH (112 μl, 0.559 mmol) and heated at 80° C. for 2 h. The reaction mixture was neutralized to pH 7 with aqueous 6M HCl. The solvent was evaporated in a rotary evaporator. The residue was dissolved in 1 mL DMF and the crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 9% B, 9-49% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to yield Compound 112.

Compound 113 and Compound 114 were analogously prepared.

### Example 5—Compound 108

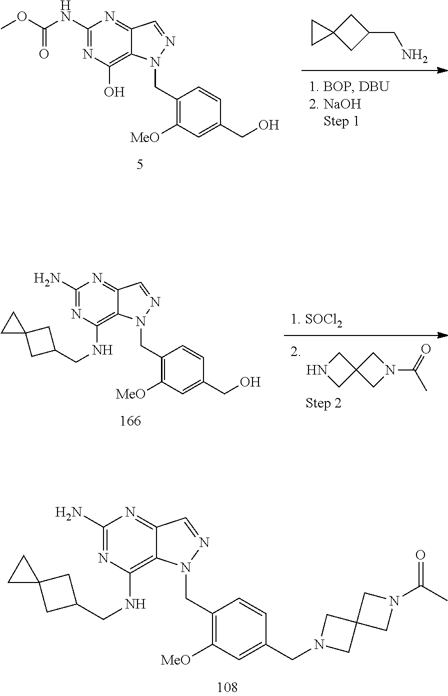

Step 1. A solution of methyl (7-hydroxy-1-(4-(hydroxymethyl)-2-methoxybenzyl)-1H-pyrazolo[4,3-d]pyrimidin-5-yl)carbamate 5 (US 2020/0038403 A1; 300 mg, 0.835 mmol), spiro[2.3]hexan-5-ylmethanamine hydrochloride (139 mg, 1.252 mmol) in DMSO (2 mL) was treated with DBU (0.378 mL, 2.505 mmol). BOP (554 mg, 1.252 mmol) was added. The reaction mixture was heated at 40° C. for 1 h. The reaction mixture was treated with NaOH (0.835 mL, 4.17 mmol) and heated at 80° C. for 2 h. The product was directly purified on reverse phase ISCO using 50 g C-18 column eluting with 0-50% water/MeCN (0.05% TFA) and fractions lyophilized to yield compound 166 as a white solid.

Step 2. A solution of (4-((5-amino-7-((spiro[2.3]hexan-5-ylmethyl)amino)-1H-pyrazolo[4,3-d]pyrimidin-1-yl)methyl)-3-methoxyphenyl)methanol 166 (300 mg, 0.760 mmol) in THF (2 mL) was treated with SOCl₂ (0.111 mL, 1.521 mmol) and stirred at RT for 30 min. The 20 solvent was evaporated in a V-10 evaporator and 30 mg of the crude chloride was dissolved in DMSO (0.5 mL) and treated with 1-(2,6-diazaspiro[3.3]heptan-2-yl)ethan-1-one (51 mg, 0.363 mmol) and Hunig's base (0.127 mL), 0.727 mmol). The reaction mixture was heated at 80° C. for 3 h. Excess base was evaporated and the crude product was was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 12% B, 12-52% B over 20 minutes, then a 0-minute hold 5 at 100% B; Flow Rate: 20 mL/min; Column Temperature: 25° C. Fraction collection was triggered by MS and UV signals. Fractions containing Compound 109 were combined and dried via centrifugal evaporation, to yield Compound 108.

The following compounds were analogously prepared: Compound 109 (spiro[2.2]pentan-1-ylmethanamine was used instead of spiro[2.3]hexan-5-ylmethanamine in Step 1), Compound 129, Compound 130, Compound 131, Compound 132, Compound 133, Compound 134, Compound 135, Compound 145, Compound 146, Compound 147, Compound 148, Compound 152 ((3-cyclopropylcyclobutyl)methanamine was used instead of spiro[2.3]hexan-5-ylmethanamine in Step 1), Compound 167, Compound 168, Compound 169, Compound 170, Compound 183, and Compound 241.

### Example 6—Compound 115

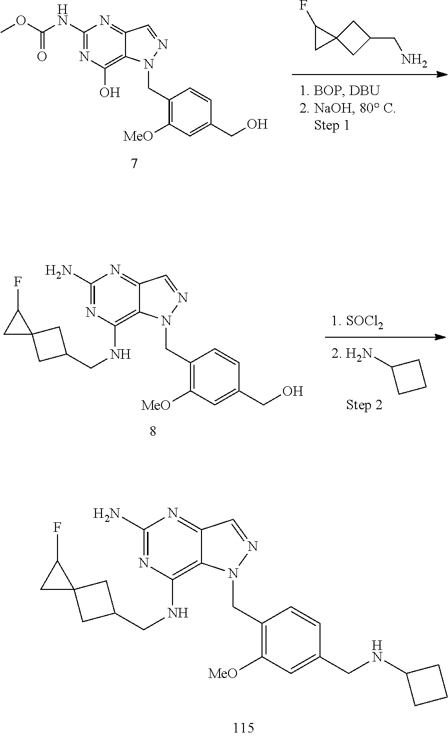 [SMILES: COC(=O)Nc1nc(O)c2c(cnn2Cc2ccc(CO)cc2OC)n1 ; NCC1CC2(C1)CC2F ; COc1cc(CO)ccc1Cn1ncc2nc(N)nc(NCC3CC4(C3)CC4F)c21 ; NC1CCC1 ; COc1cc(CNC2CCC2)ccc1Cn1ncc2nc(N)nc(NCC3CC4(C3)CC4F)c21]

Step 1. A solution of methyl (7-hydroxy-1-(4-(hydroxymethyl)-2-methoxybenzyl)-1H-pyrazolo[4,3-d]pyrimidin-5-yl)carbamate 7 (US 2020/0038403 A1; 100 mg, 0.278 mmol), (1-fluorospiro[2.3]hexan-5-yl)methanamine (71.9 mg, 0.557 mmol) in DMSO (2 mL) was treated with DBU (0.126 mL, 0.835 mmol). BOP (185 mg, 0.417 mmol) was added. The reaction mixture was heared at 40° C. for 1 h. The reaction mixture was treated with NaOH (0.278 mL, 1.391 mmol) and heated at 80° C. for 2 h. The product was directly purified on reverse phase ISC using 50 g C-18 column eluting with 0-50% water/MeCN (0.05% TFA) and desired fractions lyophilized to yield 84 mg of compound 8 as white solid as a mixture of diastereomers. LC/MS [M+H]⁺=469.1 ¹H NMR (400 MHz, DMSO-d₆) δ 8.34 (s, 1H), 7.88 (s, 1H), 7.75 (d, J=1.8 Hz, 1H), 6.98 (s, 1H), 6.86-6.74 (m, 2H), 5.71 (s, 2H), 4.67-4.58 (m, 1H), 4.45 (d, J=3.6 Hz, 3H), 3.75 (d, J=3.2 Hz, 5H), 2.80 (s, 1H), 2.18 (q, J=9.1 Hz, 1H), 2.05-1.90 (m, 2H), 1.85-1.76 (m, 1H), 0.74 (ddd, J=21.0, 11.2, 5.9 Hz, 2H).

Step 2. SOCl₂ (0.030 mL, 0.407 mmol) was added to a solution of compound 8 (84 mg, 0.204 mmol) in THF (1 mL). The reaction mixture was stirred at RT for 1 h. The solvent was evaporated in a V-10 evaporator to yield the crude chloride, which was taken to the next step without further purification. A solution of 12 mg of the chloride and cyclobutylamine (3.96 mg, 0.056 mmol) in 0.5 mL DMF in a 20 mL sealed vial was heated at 70° C. for 1 h. The excess of base was evaporated and the crude material was purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 2% B, 2-42% B over 23 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide 6 mg of Compound 115 as mixture of diastereomers.

The following compounds were analogously prepared: Compound 116, Compound 117, Compound 118, Compound 119, Compound 120, Compound 121, Compound 124, Compound 125, Compound 126, Compound 127, and Compound 128.

### Example 7- Compound 136

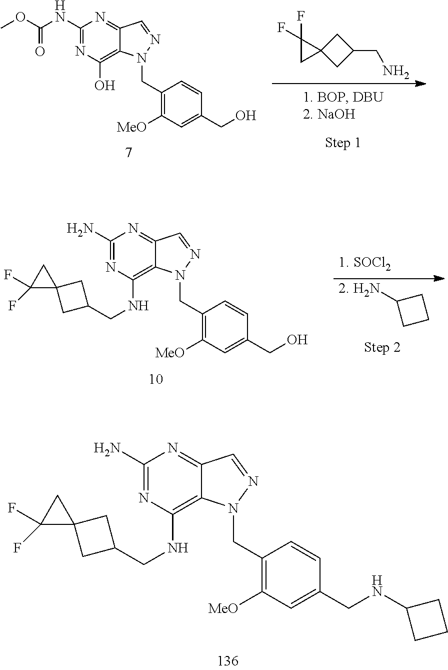

Step 1. A solution of compound 7 (200 mg, 0.557 mmol), (1,1-difluorospiro[2.3]-hexan-5-yl)methanamine (164 mg, 1.113 mmol) in DMSO (2 mL) was treated with DBU (0.252 mL, 1.670 mmol). BOP (369 mg, 0.835 mmol) was added. The reaction mixture was heated at 40° C. for 1 h. The reaction mixture was treated with NaOH (0.557 mL, 2.78 mmol) and heated at 80° C. for 2 h. The reaction was directly purified on reverse phase ISC using 50 g C-18 column eluting with 0-50% water/acetonitrile and fractions lyophilized to yield desired product as a white solid.

LC/MS expected for C₂₁H₂₄F₂N₆O₂=431.4 Observed [M+H]⁺=431.2

Step 2. A solution of compound 10 (142 mg, 0.330 mmol) in tetrahydrofuran (2 mL) was treated with SOCl₂ (0.048 mL, 0.660 mmol) and stirred for 1 h. The solvent was evaporated in a V-10 evaporator and the crude product was taken to next step. A mixture of the crude chloride and cyclobutylamine (11.8 mg, 0.167 mmol) in 0.5 mL DMF was heated at 80° C. for 1 h. Excess amine was evaporated and the crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 15% B, 15-55% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to yield 4.2 mg of Compound 136, isolated as a mixture of diastereomers.

The following compounds were analogously prepared: Compound 122, Compound 123, Compound 137, Compound 138, Compound 139, Compound 140, Compound 141, Compound 142, Compound 143, and Compound 144.

### Example 8—Compound 173

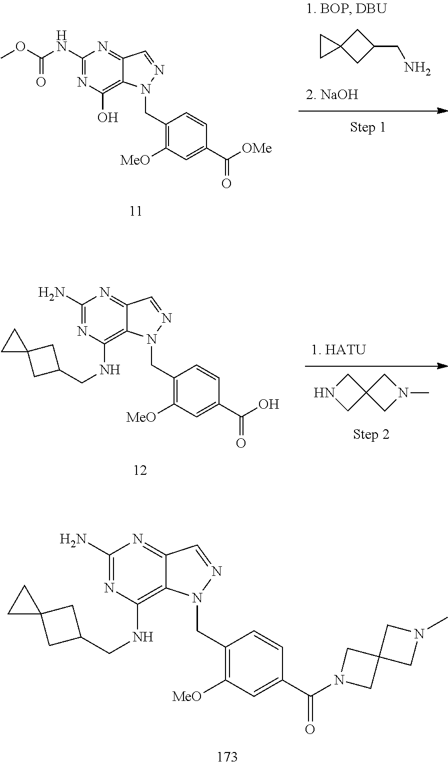

Step 1. A solution of compound 11 (US 2020/0038403 A1; 350 mg, 0.904 mmol), spiro[2.3]hexan-5-ylmethanamine hydrochloride (151 mg, 1.355 mmol) in DMSO (2 mL) was treated with DBU (0.409 mL, 2.71 mmol). BOP (599 mg, 1.355 mmol) was added. The reaction mixture was heated at 40° C. for 1 h. The reaction mixture was treated with NaOH (0.904 mL, 4.52 mmol) and heated at 80° C. for 2 h. The reaction was directly purified on reverse phase ISCO using 50 g C-18 column eluting with 0-50% water/acetonitrile (0.05% TFA) and fractions lyophilized to yield compound 12 as a white solid.

### LC/MS [M+H]⁺=395.2.

¹H NMR (400 MHz, DMSO-d₆) 12.34 (s, 1H), 8.32 (t, J=5.7 Hz, 1H), 7.86 (s, 1H), 7.80 (s, 1H), 7.53-7.43 (m, 2H), 6.79 (d, J=7.9 Hz, 1H), 5.81 (s, 2H), 3.84 (s, 3H), 3.72 (t, J=6.5 Hz, 3H), 2.77-2.65 (m, 1H), 1.82 (dd, J=12.0, 6.3 Hz, 3H), 1.66 (s, 1H), 0.36 (s, 4H).

Step 2. A solution of compound 12 (40 mg, 0.098 mmol) and 2-methyl-2,6-diazaspiro[3.3]heptane (11 mg, 0.098 mmol) in 0.5 mL DMF was treated with Hunig's base (1 microliter, 0.294 mmol) and HATU (44 mg, 0.118 mmol). The reaction mixture was stirred at RT for 30 min. Excess base was evaporated and the crude product was purified by reverse phase ISCO using 50 g C-18 column eluting with 0-50% water/acetonitrile (0.05% TFA) and fractions were lyophilized to yield Compound 173 as a white solid.

The following compounds were analogously prepared: Compound 171, Compound 172, Compound 174, Compound 175, Compound 176, Compound 177, Compound 178, Compound 179, Compound 184, Compound 190, Compound 192, Compound 193, Compound 194, Compound 195, Compound 196, Compound 197, Compound 201, Compound 222, Compound 225, Compound 226, Compound 227, Compound 228, Compound 230, Compound 231, Compound 234, Compound 235, Compound 236, Compound 237, Compound 238, Compound 239, and Compound 240.

### Example 9—Compound 149

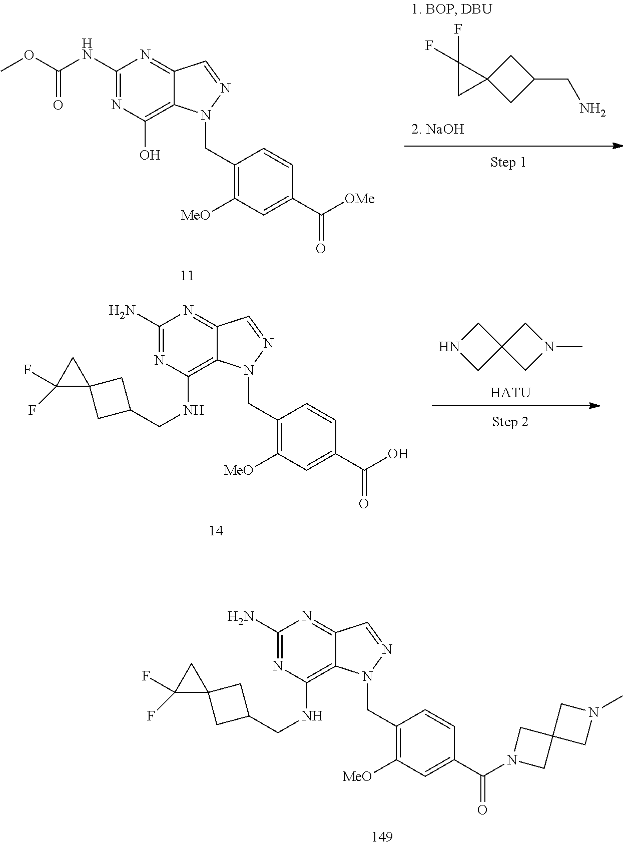

Step 1. A solution of compound 11 (100 mg, 0.258 mmol), (1,1-difluorospiro[2.3]-hexan-5-yl)methanamine (76 mg, 0.516 mmol) in DMSO (2 mL) was treated with DBU (0.117 mL, 0.774 mmol). BOP (171 mg, 0.387 mmol) was added. The reaction mixture was heated at 40° C. for 1 h, treated with NaOH (0.258 mL, 1.291 mmol), heated at 80° C. for 2 h and directly purified on reverse phase ISC using 50 g C-18 column eluting with 0-50% water/acetonitrile (0.05% TFA) and fractions lyophilized to yield 91 mg of compound 14 as a white solid. LC/MS [M−H]⁺=443.2.

Step 2. A solution of compound 14 (15 mg, 0.034 mmol) and 2-methyl-2,6-diazaspiro[3.3]heptane (3.8 mg, 0.034 mmol) in 0.5 mL DMF was treated with Hunig's base (18 microliter, 0.1 mmol) and HATU (15.4 mg, 0.041 mmol). The reaction was stirred at RT for 20 min. Excess amine was evaporated and the crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 8% B, 8-48% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to obtain Compound 149 as a white solid.

The following compounds were analogously prepared: Compound 150, Compound 151, Compound 159, Compound 160, Compound 161, Compound 162, Compound 163, Compound 164, and Compound 165.

### Example 10—Compound 153

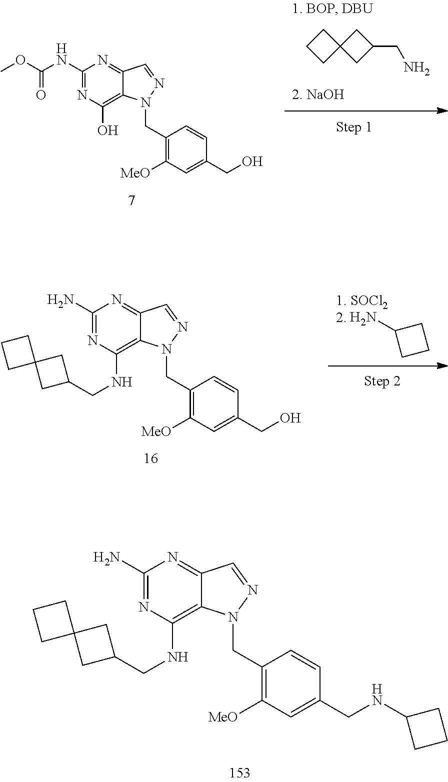 [SMILES: COC(=O)Nc1nc(O)c2c(cnn2Cc2ccc(CO)cc2OC)n1 ; NCC1CC2(CCC2)C1 ; COc1cc(CO)ccc1Cn1ncc2nc(N)nc(NCC3CC4(CCC4)C3)c21 ; NC1CCC1 ; COc1cc(CNC2CCC2)ccc1Cn1ncc2nc(N)nc(NCC3CC4(CCC4)C3)c21]

Step 1. A solution of compound 7 (100 mg, 0.278 mmol), spiro[3.3]heptan-2-ylmethanamine (69.7 mg, 0.557 mmol) in DMSO (2 mL) was treated with DBU (0.126 mL, 0.835 mmol). BOP (185 mg, 0.417 mmol) was added. The reaction mixture was heated at 40° C. for 1 h, treated with NaOH (0.278 mL, 1.391 mmol), heated at 80° C. for 2 h, and directly purified on reverse phase ISC using 50 g C-18 column eluting with 0-50% water/MeCN (0,05% TFA) and fractions lyophilized to yield compound 16 as a white solid. LC/MS [M+H]⁺=409.3

Step 2. A solution of compound 16 (190 mg, 0.465 mmol) in THF (1 mL) was treated with SOCl₂ (0.068 mL, 0.930 mmol) and stirred for 30 min. The solvent was evaporated and the crude chloride was taken to the next step. A solution of the chloride (15 mg, 0.035 mmol) and cyclobutylamine (12 mg, 0.176 mmol) was dissolved in 0.5 mL of DMF and heated at 70° for 1 h. The cyclobutylamine was evaporated and the crude material was purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 9% B, 9-49% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide 7.9 mg of Compound 153.

The following compounds were analogously prepared per this Example: Compound 154, Compound 155, Compound 156, Compound 157, Compound 158, Compound 185, and Compound 186. In the instance of Compound 185 and Compound 186, (6,6-difluorospiro[3.3]-heptan-2-yl)methanamine was used instead of spiro[3.3]heptan-2-ylmethanamine in Step 1).

### Example 11—Compound 200

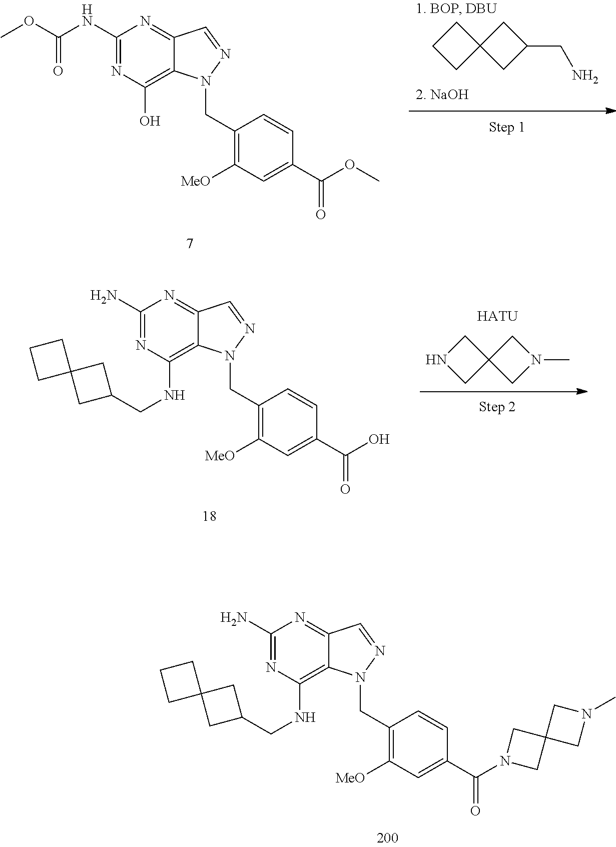

Step 1. A solution of compound 7 (100 mg, 0.258 mmol), spiro[3.3]heptan-2-ylmethanamine (48.5 mg, 0.387 mmol) in DMSO (2 mL) was treated with DBU (0.117 mL, 0.774 mmol). BOP (171 mg, 0.387 mmol) was added. The reaction mixture was heated at 40° C. for 1 h, treated with NaOH (0.258 mL, 1.291 mmol), and heated at 80° C. for 2 h. The reaction product was directly purified on reverse phase ISC using 50 g C-18 column eluting with 0-50% water/acetonitrile (0.05% TFA) and fractions lyophilized to yield compound 18 as white solid. LC/MS [M+H]⁺=422.3

¹H NMR (500 MHz, DMSO-d₆) δ 7.59 (s, 1H), 7.22 (s, 1H), 7.05 (d, J=7.8 Hz, 1H), 6.46 (s, OH), 6.37 (d, J=7.8 Hz, 1H), 5.66 (d, J=12.0 Hz, 4H), 4.31 (s, 2H), 4.08 (s, 2H), 3.89 (s, 3H), 3.53 (s, 1H), 3.38 (t, J=6.4 Hz, 1H), 3.31 (d, J=7.6 Hz, 1H), 3.24 (d, J=7.5 Hz, 1H), 3.01 (d, J=4.4 Hz, OH), 2.31 (q, J=7.7 Hz, 1H), 2.17 (s, 3H), 1.93-1.86 (m, 2H), 1.85 (td, J=12.7, 11.5, 3.7 Hz, 4H), 1.80 (d, J=7.3 Hz, 3H), 1.72 (q, J=7.7 Hz, 2H), 1.57-1.50 (m, 2H).

Step 2. A solution of compound 18 (20 mg, 0.047 mmol) in DMF (0.5 mL) was treated with 2-methyl-2,6-diazaspiro[3.3]heptane (5.31 mg, 0.047 mmol) followed by HATU (21.60 mg, 0.057 mmol) and Hunig's base (0.025 mL, 0.142 mmol). LCMS after 30 min showed completion of the reaction. The crude material was purified via preparative LC/MS with the following conditions: Column: XBridge C18, 200 mm×19 mm, 5-?m particles; Mobile Phase A: 5:95 acetonitrile: water with NH₄OAc; Mobile Phase B: 95:5 acetonitrile: water with NH₄OAc; Gradient: a 0-minute hold at 11% B, 11-51% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to yield Compound 200.

The following compounds were analogously prepared: Compound 180, Compound 181, Compound 182, Compound 187, Compound 188, Compound 189, Compound 202, Compound 203, Compound 204, and Compound 205. In the instance of Compound 187, (6,6-difluorospiro[3.3]heptan-2-yl)methanamine was used instead of spiro[3.3]heptan-2-ylmethanamine in Step 1.

### Example 12—Compound 210

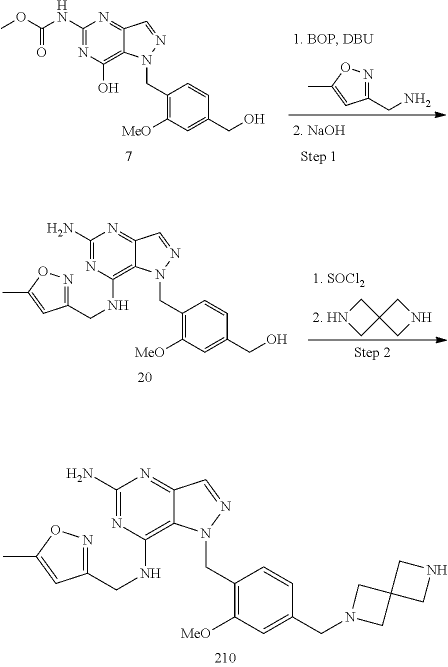

Step 1. A solution of compound 7 (100 mg, 0.278 mmol) and (5-methylisoxazol-3-yl)methanamine (62 mg, 0.557 mmol) in DMSO (2 mL) was treated with DBU (0.210 mL, 1.391 mmol). BOP (185 mg, 0.417 mmol). The reaction mixture was heated at 40° C. for 1 h, treated with NaOH (0.278 mL, 1.391 mmol), and heated at 80° C. for 2 h. The reaction mixture was directly purified on reverse phase ISC using 50 g C-18 column eluting with 0-50% water/acetonitrile (0.05% TFA). Fractions were lyophilized to yield compound 20 (white solid).

### LC/MS [M+H]+=396.1.

¹H NMR (400 MHz, DMSO-d₆) δ 8.80 (t, J=5.9 Hz, 1H), 7.84 (s, 1H), 7.70 (s, 1H), 6.88 (s, 1H), 6.81-6.71 (m, 2H), 6.11 (s, 1H), 5.62 (s, 2H), 4.73 (d, J=5.8 Hz, 2H), 4.39 (s, 2H), 4.13 (d, J=5.9 Hz, OH), 3.61 (s, 3H), 2.29 (d, J=4.0 Hz, 3H), 1.56-1.43 (m, 1H), 0.59-0.50 (m, 1H).

Step 2. A solution of compound 20 (70 mg, 0.177 mmol) in THF (0.5 mL) was treated with SOCl₂ (0.026 mL, 0.354 mmol) and stirred at RT for 30 min. The solvent was evaporated in a V-10 evaporator and the crude chloride was taken to next step. The crude chloride (18 mg, 0.043 mmol) and 2,6-diazaspiro[3.3]heptane (21 mg, 0.217 mmol) was mixed in 0.5 mL of DMSO and the reaction mixture heated at 80° C. for 1 h. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 5% B, 5-45% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide 7.4 mg of Compound 210 as a white solid.

The following compounds were analogously prepared: Compound 211, Compound 212, and Compound 213.

### Example 13—Compound 214

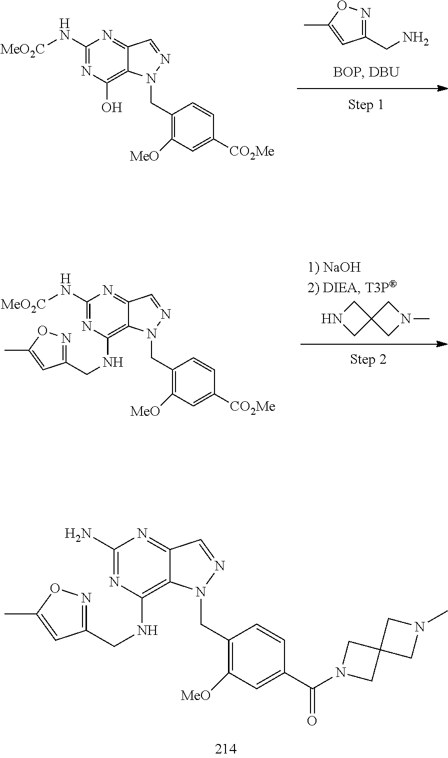

Step 1. A solution of methyl 4-((7-hydroxy-5-((methoxycarbonyl)amino)-1H-pyrazolo[4,3-d]pyrimidin-1-yl)methyl)-3-methoxybenzoate (300 mg, 0.774 mmol) in DMSO (3.9 mL) was treated with (5-methylisoxazol-3-yl)methanamine (174 mg, 1.55 mmol), BOP (411 mg, 0.929 mmol) and DBU (233 μL, 1.549 mmol). The reaction mixture was stirred at RT for 2 h, diluted with EtOAc, and washed with H₂O (3×). The organic layer was dried over Na₂SO₄, filtered and concentrated in vacuo to give methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (353 mg, 95% yield).

¹H NMR (400 MHz, DMSO-d₆) δ 9.80 (s, 1H), 7.99-7.93 (m, 1H), 7.77 (t, J=5.9 Hz, 1H), 7.49 (d, J=1.5 Hz, 1H), 7.45 (dd, J=7.8, 1.5 Hz, 1H), 6.62 (d, J=7.9 Hz, 1H), 6.10 (d, J=0.9 Hz, 1H), 5.80 (s, 2H), 4.73 (d, J=5.9 Hz, 2H), 3.84 (s, 3H), 3.82 (s, 3H), 3.64 (s, 3H), 2.31 (s, 3H). LC/MS conditions: Column: Aquity UPLC BEH C18, 2.1 mm×50 mm, 1.7 μm particles; Mobile Phase A: 100% water with 0.05% TFA; Mobile Phase B: 100% acetonitrile with 0.05% TFA; Gradient: 2% B to 98% B over 1 min, then a 0.50 min hold at 98% B; Flow: 0.8 mL/min. LC RT: 0.67 min. LC/MS (M+H) 482.3.

Step 2. A solution of methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (125 mg, 0.260 mmol) in dioxane (1.3 mL) was treated with NaOH (10 M aq soln, 0.2 mL, 2.0 mmol) and heated to 75° C. After 2 h, the reaction mixture was cooled to RT and treated with HCl (4 M in dioxane, 0.52 mL, 2.1 mmol). The resulting solution was concentrated in vacuo. The residue was redissolved in MeOH/DCM and concentrated in vacuo again to give the crude 4-((5-amino-7-(((5-methylisoxazol-3-yl)methyl)amino)-1H-pyrazolo[4,3-d]pyrimidin-1-yl)methyl)-3-methoxybenzoic acid. A solution of this crude product (40 mg) in DMF (469 μL) was treated with 2-methyl-2,6-diazaspiro[3.3]heptane •2 HCl (17 mg, 0.094 mmol), DIEA (57 μl, 0.33 mmol) and 2,4,6-tripropyl-1,3,5,2,4,6-trioxatriphosphorinane-2,4,6-trioxide (50% solution in EtOAc, 55.8 μL, 0.094 mmol). The reaction mixture was stirred at RT for 1 h. The reaction mixture was diluted with DMF (1 mL) and H₂O (0.2 mL) and filtered through a PTFE frit. The crude material was purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 5% B, 5-45% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 214 (13.7 mg, 58% yield).

### Example 14—Compound 198

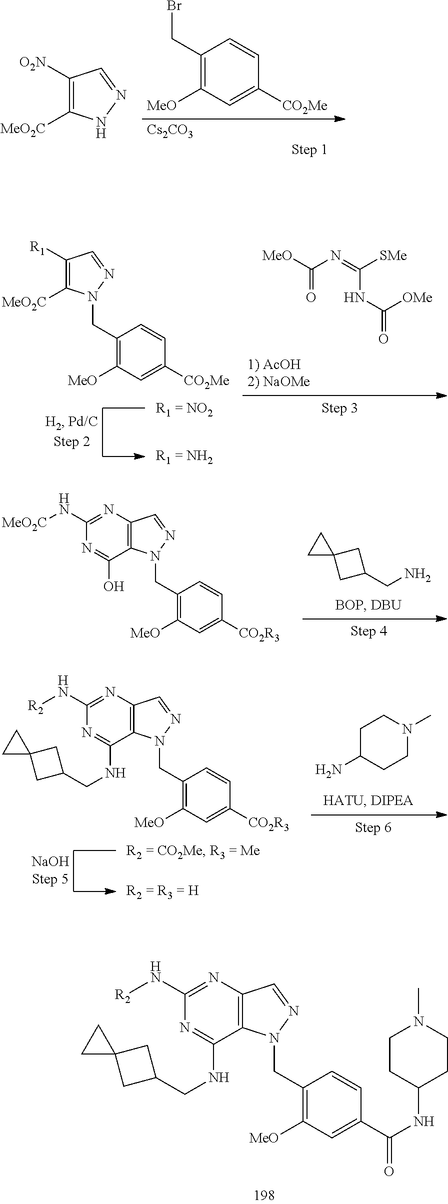

Step 1. To a stirred solution of methyl 4-nitro-1H-pyrazole-5-carboxylate (5 g, 29.2 mmol) in DMF (30 mL) was added Cs₂CO₃ (11.42 g, 35.1 mmol). After cooling in an ice bath, a solution of methyl 4-(bromomethyl)-3-methoxybenzoate (7.57 g, 29.2 mmol) in DMF (20 mL) was added portionwise over 5 minutes. The reaction was allowed to warm slowly to RT, stirred overnight, poured into water (150 mL), and extracted with EtOAc (3×70 mL). The combined organic phases were washed with brine (4×50 mL), dried (MgSO₄), filtered and concentrated. Flash chromatography (220 g SiO₂ column, 0 to 50% EtOAc in hexanes) gave methyl 1-(2-methoxy-4-(methoxycarbonyl)benzyl)-4-nitro-1H-pyrazole-5-carboxylate (1.012 g, 2.90 mmol, 9.92% yield), as a solid.

LC-MS (ES, m/z): [M+H]⁺ 350.1.

¹H NMR (400 MHz, DMSO-d₆) δ 8.40 (s, 1H), 7.57 (d, J=7.6 Hz, 1H), 7.50 (s, 1H), 7.27 (d, J=7.9 Hz, 1H), 5.53 (s, 2H), 3.96 (s, 3H), 3.86 (s, 3H), 3.82 (s, 3H).

Step 2. Methyl 1-(2-methoxy-4-(methoxycarbonyl)benzyl)-4-nitro-1H-pyrazole-5-carboxylate (2 g, 5.73 mmol) was suspended in ethanol (100 mL). 10% palladium on carbon (100 mg) was added, and the reaction vessel was evacuated and purged six times with hydrogen. The reaction mixture was stirred overnight under a hydrogen atmosphere, and filtered through CELITE™, with washing with EtOH (100 mL). The filtrated was evaporated to dryness, giving methyl 4-amino-1-(2-methoxy-4-(methoxycarbonyl)benzyl)-1H-pyrazole-5-carboxylate (1.764 g, 5.52 mmol, 96% yield) as a solid.

LC-MS (ES, m/z): [M+H]⁺ 320.1.

¹H NMR (400 MHz, DMSO-d₆) δ 7.50 (s, 1H), 7.46 (d, J=7.7 Hz, 1H), 7.18 (s, 1H), 6.42 (d, J=7.9 Hz, 1H), 5.55 (s, 2H), 5.14 (s, 2H), 3.91 (s, 3H), 3.84 (s, 3H), 3.70 (s, 3H)

Step 3. Methyl 4-amino-1-(2-methoxy-4-(methoxycarbonyl)benzyl)-1H-pyrazole-5-carboxylate (1.75 g, 5.48 mmol) was suspended in MeOH (60 mL). 1,3-Bis(methoxycarbonyl)-2-methyl-2-thiopseudourea (1.243 g, 6.03 mmol) was added, followed by HOAc (1.882 mL, 32.9 mmol). The reaction mixture was stirred for 1 h at RT. 2 mL of TFA was added, and the reaction mixture was stirred overnight. NaOMe (23.69 g, 110 mmol, 25% by wt.) was added, followed by 4 h stirring at RT. The precipitate was filtered off, and suspended in MeOH (50 mL). NaOMe (3 g, 13.88 mmol, 25% by weight) was added and the reaction stirred at RT for 1 hour. The reaction mixture was acidified with AcOH, and after stirring for 10 min, the product was filtered off, washing with MeOH, to give solid methyl 4-((7-hydroxy-5-((methoxycarbonyl)amino)-1H-pyrazolo[4,3-d]pyrimidin-1-yl)methyl)-3-methoxybenzoate (670 mg, 1.730 mmol, 32% yield).

LC-MS (ES, m/z): [M+H]⁺ 388.1. ¹

### H NMR (400 MHz, DMSO-d₆) δ 7.92 (s, 1H), 7.52 (s, 1H), 7.47 (d, J=7.6 Hz, 1H), 6.70 (d, J=7.7 Hz, 1H), 5.76 (s, 2H), 3.90 (s, 3H), 3.85 (s, 3H), 3.76 (s, 3H).

Step 4. A 20 mL scintillation vial was charged with methyl 4-((7-hydroxy-5-((methoxycarbonyl)amino)-1H-pyrazolo[4,3-d]pyrimidin-1-yl)methyl)-3-methoxybenzoate (180 mg, 0.465 mmol), spiro[2.3]hexan-5-ylmethanamine hydrochloride (103 mg, 0.697 mmol), BOP (308 mg, 0.697 mmol) and DMSO (1 mL). DBU (0.245 mL, 1.626 mmol) was added. The reaction mixture was stirred at 60° C. for 2 h, cooled, filtered and purified using reverse-phase flash chromatography (50 g C₁₈ column, 0 to 65% MeCN in water containing 0.05% formic acid), giving methyl 3-methoxy-4-((5-((methoxycarbonyl)amino)-7-((spiro[2.3]hexan-5-ylmethyl)amino)-1H-pyrazolo[4,3-d]pyrimidin-1-yl)methyl)benzoate (165 mg, 0.343 mmol, 73.9% yield, white solid). LC-MS (ES, m/z): [M+H]⁺ 481.2.

Step 5. Methyl 3-methoxy-4-((5-((methoxycarbonyl)amino)-7-((spiro[2.3]hexan-5-ylmethyl)amino)-1H-pyrazolo[4,3-d]pyrimidin-1-yl)methyl)benzoate (165 mg, 0.343 mmol) was dissolved in dioxane (4 mL). NaOH (1.030 mL, 5.15 mmol) was added, and the reaction heated at 80° C. for 2 hours. After cooling, the reaction mixture was acidified with HCl and evaporated to dryness, then the product used without purification.

LC-MS (ES, m/z): [M+H]⁺ 409.3

Step 6. A 20 mL scintillation vial was charged with 4-((5-amino-7-((spiro[2.3]hexan5-ylmethyl)amino)-1H-pyrazolo[4,3-d]pyrimidin-1-yl)methyl)-3-methoxybenzoic acid (100 mg, 0.086 mmol), HBTU (39.0 mg, 0.103 mmol), 1-methylpiperidin-4-amine (19.57 mg, 0.171 mmol) and DMF (2 mL). DIPEA (0.045 mL, 0.257 mmol) was added. The reaction mixture was stirred at RT for 1 h, filtered, and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 0% B, 0-40% B over 25 minutes, then a 0-minute hold at 100% B; Flow Rate: 20 mL/min; Column Temperature: 25° C. Fraction collection was triggered by MS and UV signals. FractionCompound 198 (16.4 mg, 0. 032 mmol, 38% yield).

Compound 199 was analogously prepared.

### Example 15—Compound 207, Ditrifluoroacetate

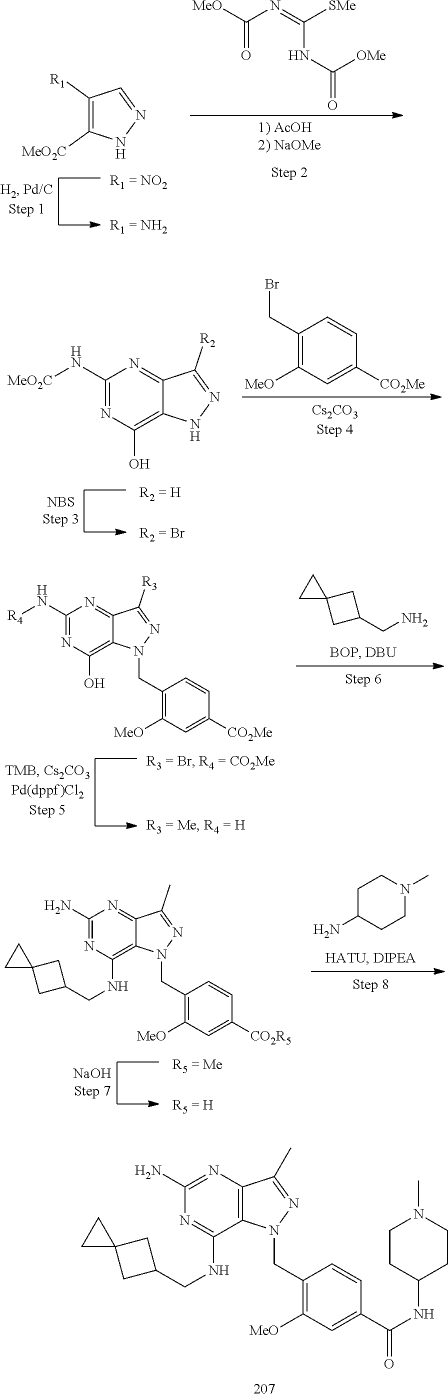

Step 1. To a stirred solution of methyl 4-nitro-1H-pyrazole-5-carboxylate (10 g, 58.4 mmol) in EtOH (100 mL) was added 10% palladium on carbon (0.622 g, 0.584 mmol). The reaction was evacuated and purged with hydrogen six times, then stirred under a hydrogen atmosphere for 2 days. The reaction mixture was filtered through CELITE™, washing with EtOH (100 mL). The filtrate was evaporated to dryness and triturated with ether/hexanes to give methyl 4-amino-1H-pyrazole-5-carboxylate (8.012 g, 56.8 mmol, 97% yield) as a solid. LC-MS (ES, m/z): [M+H]⁺ 142.1.

Step 2. Methyl 4-amino-1H-pyrazole-5-carboxylate (4 g, 28.3 mmol) was dissolved in MeOH (75 mL), and 1,3-bis(methoxycarbonyl)-2-methyl-2-thiopseudourea (6.43 g, 31.2 mmol) was added, followed by acetic acid (6.49 mL, 113 mmol). The reaction mixture was stirred at RT for 5 hours. NaOMe (36.7 g, 170 mmol, 25% by weight) was added. The reaction mixture was stirred at RT overnight, acidified with AcOH, and filtered, washing with water (100 mL), THF (100 mL) and ether (100 mL), to give methyl (7-hydroxy-1H-pyrazolo[4,3-d]pyrimidin-5-yl)carbamate (5.098 g, 24.37 mmol, 86% yield) as a solid. LC-MS (ES, m/z): [M+H]⁺ 210.0.

Step 3. Methyl (7-hydroxy-1H-pyrazolo[4,3-d]pyrimidin-5-yl)carbamate (5.1 g, 24.38 mmol) was suspended in DMF (100 mL). NBS (4.34 g, 24.38 mmol) was added, and the reaction stirred at RT for 1 hour. The reaction mixture was quenched with water (100 mL), stirred for 10 minutes, then filtered, washing with water (100 mL), THF (2×50 mL) and ether (2×50 mL), giving methyl (3-bromo-7-hydroxy-1H-pyrazolo[4,3-d]pyrimidin-5-yl)carbamate (8.32 g, 23.11 mmol, 95% yield) as a solid.

LC-MS (ES, m/z): [M+H]⁺ 288.0, 290.0.

Step 4. To a stirred solution of methyl (3-bromo-7-hydroxy-1H-pyrazolo[4,3-d]pyrimidin-5-yl)carbamate (2.50 g, 8.68 mmol) in DMF (35 mL) was added Cs₂CO₃ (3.11 g, 9.55 mmol) followed by a stirred solution of methyl 4-(bromomethyl)-3-methoxybenzoate (2.249 g, 8.68 mmol) in DMF (15 mL). The reaction mixture was stirred at RT overnight, quenched with water (400 mL), and extracted with EtOAc (3×150 mL). The combined organic phases were washed with brine (4×100 mL), dried (MgSO₄), filtered and concentrated. Trituration using DCM/ether/hexanes gave methyl 4-((3-bromo-7-hydroxy-5-((methoxycarbonyl)amino)-1H-pyrazolo[4,3-d]pyrimidin-1-yl)methyl)-3-methoxybenzoate (1.791 g, 3.07 mmol, 35.4% yield), which was 80% pure by LCMS (the other 20% being the N2-regioisomer). LC-MS (ES, m/z): [M+H]⁺ 466.1, 468.1.

Step 5. A 20 mL microwave vial was charged with methyl 4-((3-bromo-7-hydroxy-5-((methoxycarbonyl)amino)-1H-pyrazolo[4,3-d]pyrimidin-1-yl)methyl)-3-methoxybenzoate (500 mg, 1.072 mmol) (ca. 80% pure contaminated with the N2-regioisomer), 2,4,6-trimethyl-1,3,5,2,4,6-trioxatriborinane (TMB, 269 mg, 2.145 mmol), [1,1′-bis(diphenylphosphino)-ferrocene]dichloropalladium(II) (235 mg, 0.322 mmol), K₂CO₃ (296 mg, 2.145 mmol), dioxane (12 mL) and water (3 mL). The reaction mixture was heated in a microwave oven at 120° C. for 1 h and evaporated to dryness. DMSO (3 mL) was added to the residue. The mixture was filtered and purified using reverse-phase flash chromatography (100 g C₁₈ column, 0 to 50% acetonitrile in water containing 0.05 TFA) gave methyl 4-((5-amino-7-hydroxy-3-methyl-1H-pyrazolo[4,3-d]pyrimidin-1-yl)methyl)-3-methoxybenzoate (117 mg, 0.341 mmol, 31.8% yield) as an off-white solid.

LC-MS (ES, m/z): [M+H]⁺ 344.1.

Step 6. A 20 mL scintillation vial was charged with methyl 4-((5-amino-7-hydroxy-3-methyl-1H-pyrazolo[4,3-d]pyrimidin-1-yl)methyl)-3-methoxybenzoate (130 mg, 0.379 mmol), spiro[2.3]hexan-5-ylmethanamine hydrochloride (84 mg, 0.568 mmol), BOP (251 mg, 0.568 mmol) and DMSO (2 mL). DBU (0.200 mL, 1.325 mmol) was added. The reaction mixture stirred at 50° C. for 1 hour, cooled, diluted with water (1 mL), filtered and purified using reverse-phase flash chromatography (50 g C₁ column, 0 to 60% acetonitrile in water containing 0.05% TFA), giving methyl 4-((5-amino-3-methyl-7-((spiro[2.3]hexan-5-ylmethyl)amino)-1H-pyrazolo[4,3-d]pyrimidin-1-yl)methyl)-3-methoxybenzoate (80 mg, 0.183 mmol, 48.4% yield) as a solid.

LC-MS (ES, m/z): [M+H]⁺ 437.3.

¹H NMR (400 MHz, DMSO-d₆) δ 8.29 (br t, J=5.6 Hz, 1H), 7.80 (br s, 2H), 7.53-7.46 (m, 2H), 6.79 (d, J=7.7 Hz, 1H), 5.74 (s, 2H), 3.85 (s, 6H), 3.71 (br t, J=6.5 Hz, 2H), 2.78-2.64 (m, 1H), 2.31 (s, 3H), 2.03-1.93 (m, 2H), 1.82 (dd, J=12.1, 6.4 Hz, 2H), 0.35 (s, 4H).

Step 7. A 20 mL scintillation vial was charged with methyl 4-((5-amino-3-methyl-7-((spiro[2.3]hexan-5-ylmethyl)amino)-1H-pyrazolo[4,3-d]pyrimidin-1-yl)methyl)-3-methoxybenzoate (75 mg, 0.172 mmol), dioxane (2 mL) and NaOH (0.412 mL, 2.062 mmol). The reaction mixture was heated to 80° C. for 2 h, cooled, neutralized with 5N HCl, and evaporated to dryness, giving 4-((5-amino-3-methyl-7-((spiro[2.3]hexan-5-ylmethyl)amino)-1H-pyrazolo[4,3-d]pyrimidin-1-yl)methyl)-3-methoxybenzoic acid (190 mg, 0.157 mmol, 92% yield) as a solid which was used without purification.

LC-MS (ES, m/z): [M+H]⁺ 423.3.

Step 8. A 20 mL scintillation vial was charged with 4-((5-amino-3-methyl-7-((spiro-[2.3]hexan-5-ylmethyl)amino)-1H-pyrazolo[4,3-d]pyrimidin-1-yl)methyl)-3-methoxybenzoic acid (100 mg, 0.083 mmol, 35% pure), HATU (37.8 mg, 0.099 mmol), 1-methylpiperidin-4-amine (18.92 mg, 0.166 mmol) and DMF (2 mL). DIPEA (0.043 mL, 0.249 mmol) was added. The reaction mixture was stirred at RT for 1 h, filtered, and purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 9% B, 9-49% B over 20 minutes, then a 0-minute hold at 100% B; Flow Rate: 20 mL/min; Column Temperature: 25° C. Fraction collection was triggered by MS and UV signals. Fractions containing the product were combined and dried via centrifugal evaporation, giving Compound 207 (43.3 mg, 0.058 mmol, 70% yield).

Compound 208 and Compound 217 were analogously prepared.

### Example 16—Compound 218

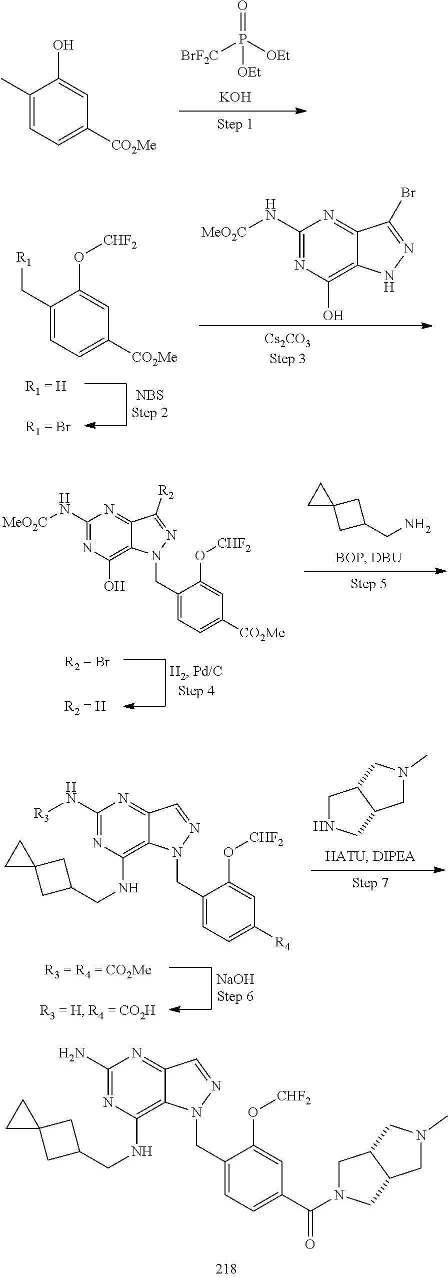

Step 1. A solution of potassium hydroxide (5N, 24.07 mL, 120 mmol) in water was added to a cooled (ice bath) solution of methyl 3-hydroxy-4-methylbenzoate (4 g, 24.07 mmol) in acetonitrile (150 mL). After stirring at 0° C. for 5 min, diethyl (bromodifluoromethyl)phospho-nate (12.85 g, 48.1 mmol) was added. The reaction mixture was allowed to warm slowly to RT and stirred for 16 h. More KOH solution (5N, 16 mL, 80 mmol) was added. The reaction mixture was stirred at RT for a further 30 min, diluted with water (200 mL) and extracted with EtOAc (3×50 mL). The combined organic phases washed with brine (2×50 mL), dried (MgSO₄), filtered and concentrated. Flash chromatography (SiO₂ column, 0 to 10% EtOAc in hexanes) gave methyl 3-(difluoromethoxy)-4-methylbenzoate (2.552 g, 11.80 mmol, 49.0% yield) as an oil.

LC-MS (ES, m/z): [M+H]⁺ 217.1.

¹H NMR (400 MHz, DMSO-d₆) δ 7.76 (dd, J=7.8, 1.7 Hz, 1H), 7.68 (br. s, 1H), 7.51-7.10 (m, 2H), 3.87 (s, 3H), 2.31 (s, 3H).

Step 2. NBS (1.811 g, 10.18 mmol) and benzoyl peroxide (0.448 g, 1.850 mmol) were added to a stirred solution of methyl 3-(difluoromethoxy)-4-methylbenzoate (2 g, 9.25 mmol) in carbon tetrachloride (20 mL). The reaction was stirred at 75° C. for 4 h, then at RT overnight. The reaction mixture was evaporated to dryness and purified using flash chromatography (SiO₂ column, 0 to 15% EtOAc in hexanes), giving methyl 4-(bromomethyl)-3-(difluoromethoxy)-benzoate (1.561 g, 5.29 mmol, 57.2% yield) as an oil.

LC-MS (ES, m/z): [M+H]⁺ 295.0, 297.0.

¹H NMR (400 MHz, CDCl₃) δ 7.88 (dd, J=8.1, 1.5 Hz, 1H), 7.80 (s, 1H), 7.52 (d, J=8.1 Hz, 1H), 6.64 (t, J=73.0 Hz, 1H), 4.57-4.51 (m, 2H), 3.98-3.90 (m, 3H).

Step 3. A stirred suspension of methyl (3-bromo-7-hydroxy-1H-pyrazolo[4,3-d]pyrimidin-5-yl)carbamate (1.269 g, 4.41 mmol) and Cs₂CO₃ (1.579 g, 4.85 mmol) in DMF (30 mL) was cooled in an ice bath. A solution of methyl 4-(bromomethyl)-3-(difluoromethoxy)-benzoate (1.3 g, 4.41 mmol) in DMF (5 mL) was added. The reaction mixture was allowed to warm slowly to RT and stirred for 3 h. The reaction mixture was poured into water (400 mL), and extracted with EtOAc (3×150 mL). The combined organic phases were washed with brine (4×80 mL), dried (MgSO₄), filtered and concentrated, giving methyl 4-((3-bromo-7-hydroxy-5-((methoxycarbonyl)amino)-1H-pyrazolo[4,3-d]pyrimidin-1-yl)methyl)-3-(difluoromethoxy)-benzoate (1.69 g, 3.37 mmol, 76% yield) as a solid.

LC-MS (ES, m/z): [M+H]⁺ 502.1, 504.0.

¹H NMR (400 MHz, DMSO-d₆) δ 11.72 (br s, 1H), 11.45 (br s, 1H), 7.80 (dd, J=7.9, 1.3 Hz, 1H), 7.74 (s, 1H), 7.35 (t, J=73.2 Hz, 1H), 7.26-7.18 (m, 1H), 5.82 (s, 2H), 3.87 (s, 3H), 3.76 (s, 3H).

Step 4. To a stirred suspension of methyl 4-((3-bromo-7-hydroxy-5-((methoxyc-arbonyl)amino)-1H-pyrazolo[4,3-d]pyrimidin-1-yl)methyl)-3-(difluoromethoxy)benzoate (1.6 g, 3.19 mmol) in ethanol (150 mL) was added 10% palladium on carbon (0.16 g). The reaction mixture was evacuated and purged with hydrogen six times, stirred under a hydrogen atmosphere for 24 h, and filtered through CELITE™. Most of the product was stuck on the CELITE™ with the palladium, so all the solid material was scraped off the CELITE™ into water (100 mL), and extracted with EtOAc (3×70 mL). The combined organic phases were washed with brine (2×50 mL) and filtered again through the CELITE™. The filtrate was combined with the original filtrate, dried (MgSO₄), filtered and concentrated, giving methyl 3-(difluoromethoxy)-4-((7-hydroxy-5-((methoxycarbonyl)amino)-1H-pyrazolo[4,3-d]pyrimidin-1-yl)methyl)benzoate (1.2 g, 2.83 mmol, 89% yield) as an off-white solid.

LC-MS (ES, m/z): [M+H]⁺ 424.1.

¹H NMR (400 MHz, DMSO-d₆) δ 11.16 (br s, 1H), 7.93 (s, 1H), 7.77 (d, J=8.5 Hz, 1H), 7.73 (s, 1H), 7.36 (t, J=73.2 Hz, 1H), 7.04 (d, J=7.9 Hz, 1H), 5.84 (s, 2H), 3.87 (s, 3H), 3.76 (s, 3H).

Step 7. A 20 mL scintillation vial was charged with methyl 3-(difluoromethoxy)-4-((7-hydroxy-5-((methoxycarbonyl)amino)-1H-pyrazolo[4,3-d]pyrimidin-1-yl)methyl)benzoate (1.250 g, 2.95 mmol), spiro[2.3]hexan-5-ylmethanamine hydrochloride (0.654 g, 4.43 mmol), BOP (1.959 g, 4.43 mmol) and DMSO (15 mL). DBU (1.558 mL, 10.33 mmol) was added, and the reaction stirred at 50° C. for 3 h. The reaction mixture was poured into saturated NaHCO₃ solution (100 mL) and extracted with EtOAc (3×50 mL). The combined organic phases were washed with brine (4×50 mL), dried (MgSO₄), filtered and concentrated. Flash chromatography (80 g SiO₂ column, loaded on CELITE™, 0 to 100% EtOAc in hexanes) gave methyl 3-(difluoromethoxy)-4-((5-((methoxycarbonyl)amino)-7-((spiro[2.3]hexan-5-ylmethyl)amino)-1H-pyrazolo[4,3-d]pyrimidin-1-yl)methyl)benzoate (338 mg, 0.654 mmol, 22.16% yield) as a solid.

LC-MS (ES, m/z): [M+H]⁺ 517.3.

Step 6. To a stirred solution of methyl 3-(difluoromethoxy)-4-((5-((methoxycarbonyl)amino)-7-((spiro[2.3]hexan-5-ylmethyl)amino)-1H-pyrazolo[4,3-d]pyrimidin-1-yl)methyl)benzoate (330 mg, 0.639 mmol) in dioxane (3600 μL) was added NaOH (1278 μL, 6.39 mmol). The reaction was stirred for 2 h at 80° C. After cooling, the reaction mixture was neutralized using 5N HCl (1.28 mL) and evaporated to dryness. The residue was suspended in DMSO (2 mL), water (35 mL) was added, and the product filtered off, washing with water (30 mL) giving 4-((5-amino-7-((spiro[2.3]hexan-5-ylmethyl)amino)-1H-pyrazolo[4,3-d]pyrimidin-1-yl)methyl)-3-(difluoromethoxy)benzoic acid (126 mg, 0.284 mmol, 44.4% yield) as a solid.

LC-MS (ES, m/z): [M+H]⁺ 445.2.

¹H NMR (400 MHz, DMSO-d₆) δ 7.71 (s, 1H), 7.67 (d, J=7.9 Hz, 1H), 7.63 (s, 1H), 7.37 (t, J=73.5 Hz, 1H), 6.80 (br t, J=5.4 Hz, 1H), 6.55 (d, J=7.9 Hz, 1H), 5.92 (br s, 2H), 5.81 (s, 2H), 3.57-3.51 (m, 2H), 2.72-2.57 (m, 1H), 1.98-1.88 (m, 2H), 1.77-1.69 (m, 2H), 0.37-0.25 (m, 4H).

Step 7. A 20 mL scintillation vial was charged with 4-((5-amino-7-((spiro[2.3]hexan-5-ylmethyl)amino)-1H-pyrazolo[4,3-d]pyrimidin-1-yl)methyl)-3-(difluoromethoxy)benzoic acid (30 mg, 0.068 mmol), HATU (30.8 mg, 0.081 mmol), (3aR,6aS)-2-methyloctahydropyrrolo[3,4-c]pyrrole (12.78 mg, 0.101 mmol) and DMF (2 mL). DIPEA (0.035 mL, 0.203 mmol) was added. The reaction stirred at RT overnight, filtered, and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 10% B, 10-50% B over 25 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 218 (30.3 mg, 81% yield).

The following compounds were analogously prepared: Compound 219, Compound 220, Compound 221, and Compound 224.

### Example 17- Compound 223

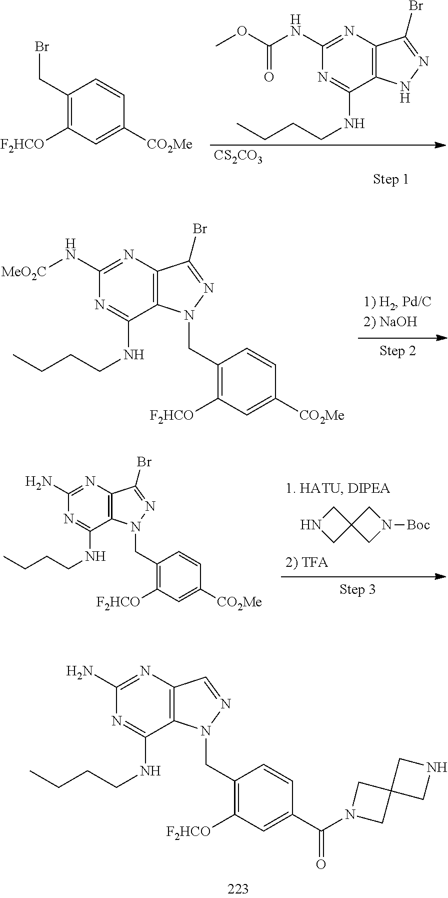

Step 1. Cs₂CO₃ (1329 mg, 4.08 mmol) was added to a stirred solution of methyl (3-bromo-7-(butylamino)-1H-pyrazolo[4,3-d]pyrimidin-5-yl)carbamate (700 mg, 2.040 mmol) in DMF (5 mL). After cooling in an ice bath, a solution of methyl 4-(bromomethyl)-3-(difluoro-methoxy)benzoate (572 mg, 1.938 mmol) in DMF (2 mL) was added. The reaction mixture was allowed to warm to RT and stirred for 3 h. Water (20 mL) was added, and the reaction mixture extracted with EtOAc (3×5 mL). The combined organic phases were washed with brine (4×10 mL), dried (MgSO₄), filtered and concentrated. Flash chromatography (SiO₂ column, loaded in DCM, 0 to 60% EtOAc in hexanes) gave methyl 4-((3-bromo-7-(butylamino)-5-((methoxy-carbonyl)amino)-1H-pyrazolo[4,3-d]pyrimidin-1-yl)methyl)-3-(difluoromethoxy)benzoate (275 mg, 0.493 mmol, 24.19% yield) as a solid.

LC-MS (ES, m/z): [M+H]⁺ 557.1, 559.1.

¹H NMR (400 MHz, DMSO-d₆) δ 9.89 (s, 1H), 7.82-7.69 (m, 2H), 7.61-7.14 (m, 2H), 6.87 (d, J=7.9 Hz, 1H), 5.88 (s, 2H), 3.87 (s, 3H), 3.64 (s, 3H), 3.54-3.45 (m, 2H), 1.58-1.46 (m, 2H), 1.19 (dq, J=15.0, 7.4 Hz, 2H), 0.83 (t, J=7.3 Hz, 3H).

Step 2. Methyl 4-((3-bromo-7-(butylamino)-5-((methoxycarbonyl)amino)-1H-pyrazolo[4,3-d]pyrimidin-1-yl)methyl)-3-(difluoromethoxy)benzoate (275 mg, 0.493 mmol) was dissolved in ethanol (15 mL). 10% Pd/C (27 mg) was added. The reaction vessel was evacuated and purged six times, with hydrogen. The reaction mixture was stirred under a H₂ atmosphere for 2 h, filtered and evaporated to dryness. The residue was dissolved in dioxane (2 mL). NaOH (0.564 mL, 2.82 mmol) was added. The reaction mixture was stirred at 80° C. for 2 h, cooled, neutralized with 5N HCl, and evaporated to dryness. The residue was dissolved in MeOH/water (1:1, 8 mL). The methanol was removed by evaporation. The residual aqueous suspension filtered, washing with water, to give 4-((5-amino-7-(butylamino)-1H-pyrazolo[4,3-d]pyrimidin-1-yl)methyl)-3-(difluoromethoxy)benzoic acid (54 mg, 0.133 mmol, 27% yield) as a solid.

LC-MS (ES, m/z): [M+H]⁺=407.22.

¹H NMR (400 MHz, DMSO-d₆) δ 8.50 (br s, 1H), 7.84 (s, 2H), 7.79-7.68 (m, 2H), 7.63-7.05 (t, J=73.2 Hz 1H), 6.97 (d, J=7.9 Hz, 1H), 5.94 (s, 2H), 3.54 (q, J=6.4 Hz, 2H), 1.54 (quin, J=7.2 Hz, 2H), 1.19 (dq, J=14.9, 7.3 Hz, 2H), 0.84 (t, J=7.3 Hz, 3H).

Step 3. A 20 mL scintillation vial was charged with 4-((5-amino-7-(butylamino)-1H-pyrazolo[4,3-d]pyrimidin-1-yl)methyl)-3-(difluoromethoxy)benzoic acid (50 mg, 0.123 mmol), HATU (56.1 mg, 0.148 mmol), tert-butyl 2,6-diazaspiro[3.3]heptane-2-carboxylate (24.39 mg, 0.123 mmol) and DMF (2 mL). DIPEA (0.064 mL, 0.369 mmol) was added. The reaction mixture was stirred at RT for 1 h, quenched with saturated NaHCO₃ solution (10 mL), and extracted with EtOAc (3×5 mL). The combined organic phases were washed with brine (4×5 mL), dried (MgSO₄), filtered and concentrated. The residue was dissolved in DCM (1.5 mL) and TFA (0.5 mL) was added. The reaction was stirred at RT for 30 minutes then evaporated to dryness. The crude material was dissolved in DMF (2 mL), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 0% B, 0-37% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation, giving Compound 223 (20.9 mg, 0.043 mmol, 35% yield).

### Example 18—Compound 242, tri-TFA salt

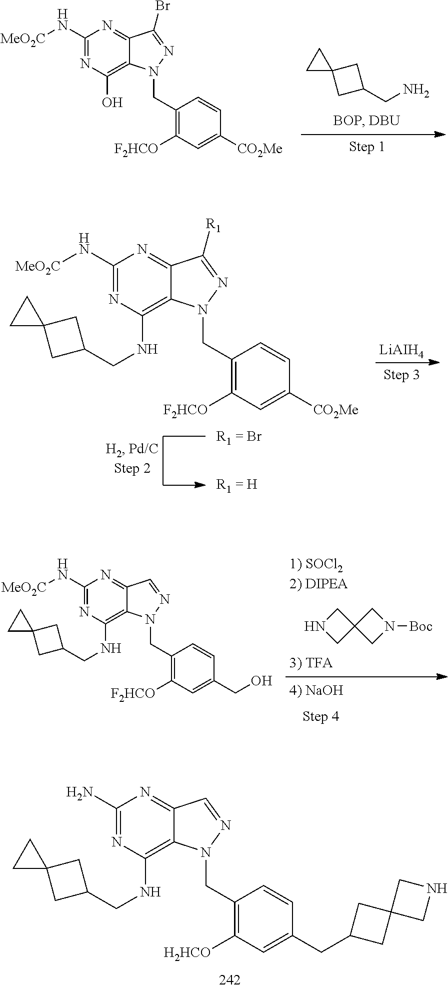

Step 1. A 20 mL scintillation vial was charged with methyl 4-((3-bromo-7-hydroxy-5-((methoxycarbonyl)amino)-1H-pyrazolo[4,3-d]pyrimidin-1-yl)methyl)-3-(difluoromethoxy)-benzoate (750 mg, 1.493 mmol), spiro[2.3]hexan-5-ylmethanamine hydrochloride (500 mg, 2.370 mmol), BOP (991 mg, 2.240 mmol) and DMSO (7.5 mL). DBU (0.788 mL, 5.23 mmol) was added. The reaction mixture was stirred at 50° C. overnight, poured into saturated NaHCO₃ solution (100 mL), and extracted with EtOAc (3×50 mL). The combined organic phases were washed with brine (4×50 mL), dried (MgSO₄), filtered and concentrated. Flash chromatography (80 g SiO₂ column, 0 to 60% EtOAc in hexanes) gave methyl 4-((3-bromo-5-((methoxycarbonyl)amino)-7-((spiro[2.3]hexan-5-ylmethyl)amino)-1H-pyrazolo[4,3-d]pyrimidin-1-yl)methyl)-3-(difluoromethoxy)benzoate (286 mg, 0.480 mmol, 32.2% yield) as a solid.

LC-MS (ES, m/z): [M+H]⁺ 595.1, 597.1.

¹H NMR (400 MHz, DMSO-d₆) δ 9.91 (s, 1H), 7.78-7.71 (m, 2H), 7.44 (t, J=5.4 Hz, 1H), 7.38 (t, J=73.2 Hz, 1H), 6.86 (d, J=7.9 Hz, 1H), 5.89 (s, 2H), 3.86 (s, 3H), 3.70-3.59 (m, 5H), 2.76 (br t, J=7.2 Hz, 1H), 2.15-2.03 (m, 2H), 1.80 (dd, J=12.1, 6.4 Hz, 2H), 0.32 (s, 4H).

Step 2. To a stirred solution of methyl 4-((3-bromo-5-((methoxycarbonyl)amino)-7-((spiro[2.3]hexan-5-ylmethyl)amino)-1H-pyrazolo[4,3-d]pyrimidin-1-yl)methyl)-3-(difluoromethoxy)benzoate (286 mg, 0.480 mmol) in ethanol (15 mL) was added 10% palladium on carbon (28 mg). The reaction mixture was evacuated and purged with hydrogen six times, then stirred under a hydrogen atmosphere for 1 hour. The reaction mixture was filtered and evaporated to dryness, giving methyl 3-(difluoromethoxy)-4-((5-((methoxycarbonyl)amino)-7-((spiro[2.3]hexan-5-ylmethyl)amino)-1H-pyrazolo[4,3-d]pyrimidin-1-yl)methyl)benzoate (290 mg, 0.477 mmol, 99% yield) as a white solid.

LC-MS (ES, m/z): [M+H]⁺ 517.3.

¹H NMR (400 MHz, DMSO-d₆) δ 11.93 (br s, 1H), 8.92 (br s, 1H), 8.17 (s, 1H), 7.80 (d, J=7.9 Hz, 1H), 7.77 (s, 1H), 7.42 (t, J=73.0 Hz, 1H), 7.11 (d, J=7.9 Hz, 1H), 6.04 (s, 2H), 3.92 (s, 3H), 3.90 (s, 3H), 3.82 (br t, J=6.5 Hz, 2H), 2.89-2.75 (m, 1H), 2.03 (dd, J=12.1, 8.4 Hz, 2H), 1.92 (dd, J=12.2, 6.7 Hz, 2H), 0.40 (s, 4H).

Step 3. To a stirred solution of methyl 3-(difluoromethoxy)-4-((5-((methoxy-carbonyl)amino)-7-((spiro[2.3]hexan-5-ylmethyl)amino)-1H-pyrazolo[4,3-d]pyrimidin-1-yl)methyl)benzoate (250 mg, 0.484 mmol) in THF (10 mL) at 0° C. was added LiAlH₄ (1.065 mL, 1.065 mmol), portionwise over 10 minutes. The reaction mixture was stirred for 30 minutes at 0° C. and then quenched with Rochelle's salt (10 mL, 20 w/v). After stirring for 10 minutes the reaction mixture was transferred to a separating funnel containing 50 mL water and extracted with EtOAc (3×30 mL). The combined organics were washed with brine (3×30 mL), dried (MgSO₄), filtered and concentrated. Flash chromatography (24 g SiO₂ column, loaded in DCM, 0 to 10% MeOH in DCM) gave methyl (1-(2-(difluoromethoxy)-4-(hydroxymethyl)benzyl)-7-((spiro[2.3]hexan-5-ylmethyl)amino)-1H-pyrazolo[4,3-d]pyrimidin-5-yl)carbamate (117 mg, 0.240 mmol, 49.5% yield) as a solid.

LC-MS (ES, m/z): [M+H]⁺ 489.2.

¹H NMR (400 MHz, DMSO-d₆) δ 9.65 (s, 1H), 7.88 (s, 1H), 7.43-6.98 (m, 4H), 6.62 (d, J=7.9 Hz, 1H), 5.79 (s, 2H), 5.29 (t, J=5.6 Hz, 1H), 4.46 (d, J=5.5 Hz, 2H), 3.68-3.59 (m, 5H), 2.78 (dt, J=15.0, 7.3 Hz, 1H), 2.00 (dd, J=12.0, 8.5 Hz, 2H), 1.83 (dd, J=12.2, 6.3 Hz, 2H), 0.35 (s, 4H).

Step 4. Methyl (1-(2-(difluoromethoxy)-4-(hydroxymethyl)benzyl)-7-((spiro[2.3]hexan-5-ylmethyl)amino)-1H-pyrazolo[4,3-d]pyrimidin-5-yl)carbamate (55 mg, 0.113 mmol) was dissolved in DCM (2 mL), and SOCl₂ (0.025 mL, 0.338 mmol) added. The reaction mixture was stirred at RT for 30 minutes, then evaporated to dryness. The residue was dissolved in DMF (2 mL), and tert-butyl 2,6-diazaspiro[3.3]heptane-2-carboxylate (33.5 mg, 0.169 mmol) was added, followed by DIPEA (0.059 mL, 0.338 mmol). The reaction mixture was stirred at 50° C. for 4 h, then at RT overnight, s quenched with saturated NaHCO₃ solution (10 mL), and extracted with EtOAc (3×4 mL). The combined organic phases were washed with brine (3×5 mL), dried (MgSO₄), filtered and concentrated. The residue was dissolved in DCM (2 mL), and TFA (0.4 mL) was added. The reaction was stirred at RTfor 1 h, then evaporated to dryness and redissolved in dioxane (2 mL). NaOH (0.338 mL, 1.689 mmol, 5N) was added, and the reaction stirred at 80° C. for 1 hour, cooled, neutralized using 5N HCl, and evaporated to dryness. The residue was dissolved in DMF (2 mL), filtered and purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 2% B, 2-42% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 242, 3 TFA salt (21.1 mg, 0.025 mmol, 21.7% yield).

Compound 243 was Analogously Prepared.

### Example 19—Compound 206

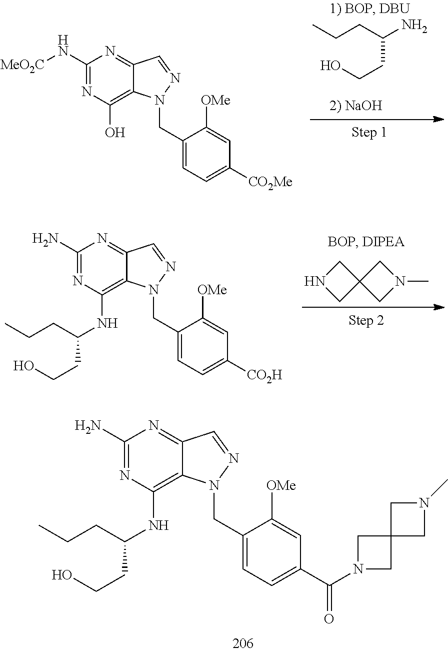

Step 1. DBU (0.856 mL, 5.68 mmol) was added to a suspension of methyl 4-((7-hydroxy-5-((methoxycarbonyl)amino)-1H-pyrazolo[4,3-d]pyrimidin-1-yl)methyl)-3-methoxybenzoate (550 mg, 1.420 mmol; see Step 6 of Example 2 before NaOH treatment) and 15 (S)-3-aminohexan-1-ol hydrochloride 2 (327 mg, 2.130 mmol) in DMSO (5 mL). The reaction mixture was stirred at RT for 10 min, when it became a clear solution. BOP (1256 mg, 2.84 mmol) was added and the reaction mixture was stirred at 70° C. for 2 h, after which LCMS did not detect any starting material. This solution was treated with 5M NaOH (5 mL, 25.00 mmol) solution and stirred at 70° C. for 0.5 h. After cooling, the reaction mixture filtered through a 20 syringe filter disc. The filtrate was purified on preparative reverse C18 column (150 g), eluted with acetonitrile:water (with 0.05% TFA modifier)=0-50%, the desired fraction was freezed and lyophilized to afford (S)-4-((5-amino-7-((1-hydroxyhexan-3-yl)amino)-1H-pyrazolo[4,3-d]pyrimidin-1-yl)methyl)-3-methoxybenzoic acid (860.8 mg, 1.246 mmol, 88% yield). LCMS ESI: calculated for C₂₀H₂₇N₆O₄=415.2 (M+H⁺), found 415.2(M+H⁺).

Step 2. A mixture of (S)-4-((5-amino-7-((1-hydroxyhexan-3-yl)amino)-1H-pyrazolo[4,3-d]pyrimidin-1-yl)methyl)-3-methoxybenzoic acid (60 mg, 0.145 mmol), 2-methyl-2,6-diazaspiro[3.3]heptane, 2 HCl (53.6 mg, 0.290 mmol) in DMF (1 mL) was treated with Hunig's base (0.126 mL, 0.724 mmol), followed by BOP (96 mg, 0.217 mmol). The reaction mixture was stirred at RT for 3 h. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 5% B, 5-45% B over 25 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation and yield Compound 206 (15.5 mg, 0.030 mmol, 20.88% yield).

The following compounds were analogously prepared: Compound 209, Compound 229, Compound 232, and Compound 233.

### Example 20—Compound 244

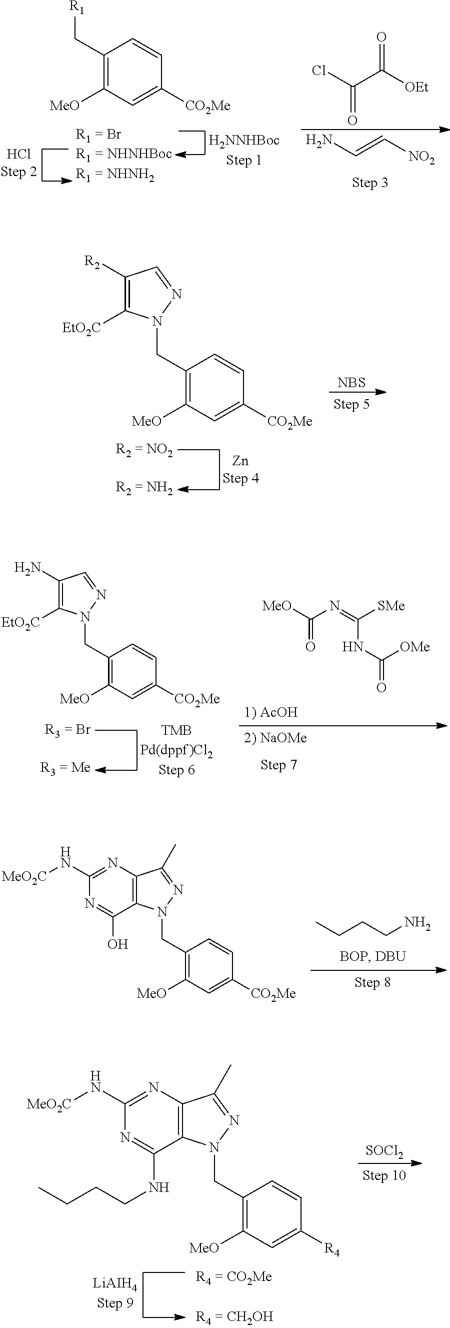

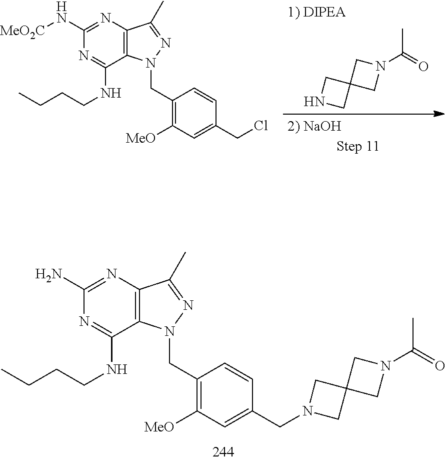

Step 1. A solution of tert-butyl hydrazinecarboxylate (12.75 g, 96 mmol) and DIPEA in DMF (24 mL) at RT was treated with the dropwise addition of methyl 4-(bromomethyl)-3-methoxybenzoate (5 g, 19.30 mmol) in 24 mL of DMF via an addition funnel over 1 hour. The reaction mixture was stirred at RT overnight. EtOAc (135 mL) and H₂O (75 mL) were added and the biphasic mixture was stirred for 30 minutes. The reaction mixture was poured into a separatory funnel and the aqueous layer was removed. The organic layer was washed with 2 additional portions of H₂O (75 mL), 2 portions of 10% LiCl solution (75 mL), ried over Na₂SO₄ and concentrated. Column chromatography (Isco, 220 g SiO2, 0% CH₂Cl₂ (5 min) then 15% EtOAc-CH₂Cl₂) provided tert-butyl 2-(2-methoxy-4-(methoxycarbonyl)benzyl)hydrazine-1-carboxylate as as clear oil (3.85 g).

### LC/MS (M+H) 311.2; LC RT=0.80 min (Procedure E).

¹H NMR (400 MHz, CHLOROFORM-d) δ 7.64 (dd, J=7.7, 1.5 Hz, 1H), 7.56 (d, J=1.5 Hz, 1H), 7.37 (d, J=7.7 Hz, 1H), 6.08-5.87 (m, 1H), 4.07 (s, 2H), 3.94 (d, J=4.6 Hz, 6H), 1.50-1.40 (m, 9H).

Step 2. tert-Butyl 2-(2-methoxy-4-(methoxycarbonyl)benzyl)hydrazine-1-carboxylate (25.4 g, 82 mmol) was dissolved in MeOH (164 mL) at RT. 4 N HCl-dioxane (123 ml, 59.5 mmol) was added and the reaction was stirred at RT overnight. The white precipitate was collected by filtration and dried to afford methyl 4-(hydrazineylmethyl)-3-methoxybenzoate, dihydrochloride (20 g).

### LC/MS (M+H) 211.1; LC RT=0.51 min (Procedure F).

¹H NMR (400 MHz, DMSO-d6) δ 9.12 (br s), 7.62-7.55 (m, 1H), 7.53-7.47 (m, 2H), 4.10 (s, 2H), 3.88 (s, 3H), 3.87 (s, 3H).

Step 3. A solution of (E)-N,N-dimethyl-2-nitroethen-1-amine (46.4 g, 400 mmol) and pyridine (420 ml, 5195 mmol) in CH₂Cl₂ (799 ml) was cooled to −10° C. and slowly treated with ethyl 2-chloro-2-oxoacetate (51.4 ml, 460 mmol). The reaction mixture was allow to warm to 25° C. over 2 h and stirred overnight. The CH₂Cl₂ was removed by rotary evaporation and methyl 4-(hydrazineylmethyl)-3-methoxybenzoate dihydrochloride (31.7 g, 112 mmol) was added in one portion. The solution was stirred for 2 h at RT and the solvent was removed under vacuum. The residue was washed with water, 1N aqueous HCl soln and extracted with EtOAc. The organic layers were dried over Na₂SO₄, and concentrated. The residue was dissolved in CH₂Cl₂, passed through a short silica gel column and recrystallized from ethanol to afford ethyl 1-(2-methoxy-4-(methoxycarbonyl)benzyl)-4-nitro-1H-pyrazole-5-carboxylate (29.4 g).

### LC/MS (M+Na) 386.0; LC RT=0.98 min (Procedure F).

¹H NMR (400 MHz, CHLOROFORM-d) δ 8.06 (s, 1H), 7.64 (dd, J=7.9, 1.5 Hz, 1H), 7.56 (d, J=1.5 Hz, 1H), 7.13 (d, J=7.8 Hz, 1H), 5.53 (s, 2H), 4.45 (q, J=7.2 Hz, 2H), 3.94 (s, 3H), 3.88 (s, 3H), 1.37 (t, J=7.2 Hz, 3H).

Step 4. Ammonium formate (1.41 g, 22.4 mmol) and zinc (0.915 g, 14.0 mmol) were added to a solution of ethyl 1-(2-methoxy-4-(methoxycarbonyl)benzyl)-4-nitro-1H-pyrazole-5-carboxylate (2.03 g, 5.60 mmol) in THF (4.67 ml)/MeOH (4.7 ml) at RT. The reaction was stirred at RT for 2 h and additional portions of ammonium formate (0.353 g, 5.60 mmol) and zinc (0.229 g, 4.67 mmol) were added. After 1 h, the reaction mixture filtered through a pad of CELITE™, and the filtrate was concentrated in vacuo to afford a white solid. The solid was suspended in EtOAc, stirred for 30 minutes and filtered. The organic filtrate was then concentrated in vacuo to give ethyl 4-amino-1-(2-methoxy-4-(methoxycarbonyl)benzyl)-1H-pyrazole-5-carboxylate (1.83 g, 98%).

¹H NMR (400 MHz, DMSO-d₆) δ 7.50-7.49 (m, 1H), 7.48-7.44 (m, 1H), 7.18 (s, 1H), 6.39 (d, J=7.8 Hz, 1H), 5.53 (s, 2H), 5.10 (s, 2H), 4.14 (q, J=7.1 Hz, 2H), 3.90 (s, 3H), 3.83 (s, 3H), 1.13 (t, J=7.1 Hz, 3H).

LC/MS conditions: Column: Aquity UPLC BEH C18, 2.1 mm×50 mm, 1.7 μm particles; Mobile Phase A: 100% water with 0.05% TFA; Mobile Phase B: 100% acetonitrile with 0.05% TFA; Gradient: 2% B to 98% B over 1 min, then a 0.50 min hold at 98% B; Flow: 0.8 mL/min. LC RT: 0.86 min. LCMS (M+H)=334.2.

Step 5. Ethyl 4-amino-1-(2-methoxy-4-(methoxycarbonyl)benzyl)-1H-pyrazole-5-carboxylate (1.65 g, 4.95 mmol) was dissolved in CHCl₃ (49.5 ml) and cooled to 0° C. NBS (0.925 g, 5.20 mmol) was added to the mixture in one portion. After 15 minutes, the reaction was diluted with CHCl₃ and vigorously stirred with 10% aqueous Na₂S₂O₃ solution for 10 minutes. The organic phase was separated, washed with H₂O, dried over MgSO₄ and concentrated. The crude product was purified by column chromatography (80 g SiO2, 0 to 50% EtOAc-hexane gradient elution) to afford ethyl 4-amino-3-bromo-1-(2-methoxy-4-(methoxycarbonyl)benzyl)-1H-pyrazole-5-carboxylate (1.32 g) as a white solid.

### LC/MS (M+H) 412.2/414.2; LC RT=1.02 min (Procedure E).

¹H NMR (400 MHz, DMSO-d6) δ 7.61-7.41 (m, 2H), 6.55 (d, J=8.3 Hz, 1H), 5.56 (s, 2H), 5.02 (s, 2H), 4.20 (q, J=7.1 Hz, 2H), 3.90 (s, 3H), 3.85 (s, 3H), 1.15 (t, J=7.1 Hz, 3H).

Step 6. Ethyl 4-amino-1-(2-methoxy-4-(methoxycarbonyl)benzyl)-3-methyl-1H-pyrazole-5-carboxylate (741.2 mg, 67.1% yield), K₂CO₃ (1.098 g, 7.94 mmol) and 2,4,6-trimethyl-1,3,5,2,4,6-trioxatriborinane (3.5 M in THF) (1.816 ml, 6.36 mmol) were suspended in dioxane (26.5 ml):Water (5.30 ml) (5:1). A stream of N₂ was bubbled through the reaction mixture for 5 min before the addition of PdCl₂(dppf)-CH₂Cl₂ adduct (0.052 g, 0.064 mmol) and continued for another 4 min before sealing the reaction and heating to 90° C. After 3 h, additional portions of 2,4,6-trimethyl-1,3,5,2,4,6-trioxatriborinane (3.5 M in THF) (0.908 ml, 3.18 mmol) and PdCl₂(dppf)-CH₂Cl₂ adduct (0.052 g, 0.064 mmol) were added and the reaction was stirred at 100° C. for 16 hours. The cooled reaction mixture was diluted with 100 mL of EtOAc and filtered through CELITE™, washing with additional EtOAc. The crude product was concentrated onto 4 g CELITE™. Column chromatography (80 g SiO2, 0 to 30% EtOAc-CH₂Cl₂ gradient elution) afforded the expected product, ethyl 4-amino-1-(2-methoxy-4-(methoxy-carbonyl)benzyl)-3-methyl-1H-pyrazole-5-carboxylate (741 mg) as a cream colored solid. LC/MS (M+H) 348.2; LC RT=0.89 min (Procedure E).

¹H NMR (400 MHz, DMSO-d6) δ 7.49 (d, J=1.5 Hz, 1H), 7.46 (dd, J=7.9, 1.5 Hz, 1H), 6.40 (d, J=7.8 Hz, 1H), 5.48 (s, 2H), 4.94-4.86 (m, 2H), 4.14 (q, J=7.0 Hz, 2H), 3.90 (s, 3H), 3.84 (s, 3H), 2.10 (s, 3H), 1.15-1.08 (m, 3H).

Step 7. Ethyl 4-amino-1-(2-methoxy-4-(methoxycarbonyl)benzyl)-3-methyl-1H-pyrazole-5-carboxylate (742 mg, 2.136 mmol) was suspended in MeOH (10.800 mL) and heated gently with vigorous stirring to solubilize the material. 1,3-bis-(Methoxycarbonyl)-2-methyl-2-thiopseudourea (661 mg, 3.20 mmol) was added followed by AcOH (0.611 mL, 10.68 mmol). The reaction mixture was stirred at RT for 16 h. An additional portion of AcOH was added (0.049 mL, 0.854 mmol) followed by stirring at RT for another 72 h before the addition of NaOMe (25% wt in MeOH) (5.69 mL, 25.6 mmol). After stirring for 3 h, the reaction mixture was re-acidified with AcOH. The product was collected by filtration, air-dried for 10 minutes and thoroughly dried in a chem-dry oven to afford methyl 4-((7-hydroxy-5-((methoxycarbonyl)-amino)-3-methyl-1H-pyrazolo[4,3-d]pyrimidin-1-yl)methyl)-3-methoxybenzoate (722.0 mg) as a cream colored solid.

### LC/MS (M+H) 402.3; LC RT=0.86 min (Procedure E).

¹H NMR (400 MHz, DMSO-d6) δ 11.58-11.17 (m, 2H), 7.51 (d, J=1.4 Hz, 1H), 7.49-7.42 (m, 1H), 6.67 (d, J=7.9 Hz, 1H), 5.67 (s, 2H), 3.90 (s, 3H), 3.84 (s, 3H), 3.71 (s, 3H), 2.31 (s, 3H).

Step 8. Methyl 4-((7-hydroxy-5-((methoxycarbonyl)amino)-3-methyl-1H-pyrazolo[4,3-d]pyrimidin-1-yl)methyl)-3-methoxybenzoate (200 mg, 0.498 mmol) and BOP (331 mg, 0.747 mmol) were suspended in DMF (2491 μl) at RT. Butan-1-amine (64.0 μl, 0.648 mmol) was added followed by DBU (3 eq) (225 μl, 1.495 mmol) after which the reaction mixture became homogeneous. The reaction mixture was stirred at 40° C. for 16 h. Additional butan-1-amine (64.0 μl, 0.648 mmol), BOP (331 mg, 0.747 mmol) and DBU (3 eq) (225 μl, 1.495 mmol) were added to the reaction and it was stirred at 40° C. for 2 h before cooling to RT. The reaction mixture was partitioned between EtOAc and saturated NaHCO₃. The organic layer was removed and the aqueous phase was extracted with three additional portions of EtOAc. The combined organics were washed with 10% LiCl solution, dried over Na₂SO₄ and concentrated. The crude product was purified by column chromatography (24 g SiO₂, 0 to 80% EtOAc-hexane gradient elution) to afford methyl 4-((7-(butylamino)-5-((methoxycarbonyl)amino)-3-methyl-1H-pyrazolo[4,3-d]pyrimidin-1-yl)methyl)-3-methoxybenzoate (117.4 mg).

### LC/MS (M+H) 457.4; LC RT=0.84 min (Procedure E).

¹H NMR (400 MHz, CHLOROFORM-d) δ 7.66 (d, J=1.3 Hz, 1H), 7.64 (dd, J=8.0, 1.4 Hz, 1H), 7.11 (d, J=7.8 Hz, 1H), 5.64 (s, 2H), 4.04 (s, 3H), 3.94 (s, 3H), 3.86 (s, 3H), 3.54-3.44 (m, 2H), 2.43 (s, 3H), 1.50 (quin, J=7.3 Hz, 2H), 1.32-1.19 (m, 2H), 0.94-0.87 (m, 3H).

Step 9. Methyl 4-((7-(butylamino)-5-((methoxycarbonyl)amino)-3-methyl-1H-pyrazolo[4,3-d]pyrimidin-1-yl)methyl)-3-methoxybenzoate (117 mg, 0.256 mmol) was dissolved in THF (854 μl) at RT. LiAIH₄ (1M in THF) (256 μl, 0.256 mmol) was added dropwise and the reaction was stirred at RT for 20 min. Additional LiAIH₄ (1M in THF) (256 μl, 0.256 mmol) was added and the reaction was stirred for another 20 min. The reaction mixture was quenched with MeOH, diluted with Rochelle's salts and EtOAc and stirred for 16 h. The organic layer was separated and the aqueous phase was extractect with three additional portions of EtOAc. The combined organic layers were dried over Na₂SO₄ and concentrated to afford methyl (7-(butylamino)-1-(4-(hydroxymethyl)-2-methoxybenzyl)-3-methyl-1H-pyrazolo[4,3-d]pyrimidin-5-yl)carbamate (86.6 mg).

### LC/MS (M+H) 429.4; LC RT=0.74 min (Procedure E).

¹H NMR (400 MHz, CHLOROFORM-d) δ 7.04 (s, 1H), 6.99 (d, J=7.8 Hz, 1H), 6.91-6.86 (m, 1H), 5.58 (s, 3H), 4.70 (s, 2H), 3.97 (s, 3H), 3.81 (s, 3H), 3.50 (td, J=6.9, 5.6 Hz, 2H), 2.54 (s, 3H), 1.54-1.43 (m, 2H), 1.31-1.22 (m, 2H), 0.94-0.88 (m, 3H).

Step 10. Methyl (7-(butylamino)-1-(4-(hydroxymethyl)-2-methoxybenzyl)-3-methyl-1H-pyrazolo[4,3-d]pyrimidin-5-yl)carbamate (86 mg, 0.201 mmol) was dissolved in THF (1004 μl) at RT. SOCl₂ (73.2 μl, 1.004 mmol) was added. The reaction mixture was stirred at RT for 1 h and concentrated to afford methyl (7-(butylamino)-1-(4-(chloromethyl)-2-methoxybenzyl)-3-methyl-1H-pyrazolo[4,3-d]pyrimidin-5-yl)carbamate (57.1 mg).

### LC/MS (M+H) 447.4; LC RT=0.89 min (Procedure E).

Step 11. Methyl (7-(butylamino)-1-(4-(chloromethyl)-2-methoxybenzyl)-3-methyl-1H-pyrazolo[4,3-d]pyrimidin-5-yl)carbamate (28 mg, 0.063 mmol) and 1-(2,6-diazaspiro[3.3]heptan-2-yl)ethan-1-one hydrochloride (33.2 mg, 0.188 mmol) were dissolved in acetonitrile (626 μl) at RT. DIPEA (32.8 μl, 0.188 mmol was added and the reaction mixture was heated to 50° C. for 16 h. The reaction mixture was concentrated and the residue was redissolved in dioxane (0.7 mL) to which NaOH solution (10 M, 125 μl, 1.253 mmol) was added. The reaction mixture was heated to 80° C. for 3 h, cooled to RT and concentrated. The residue was dissolved in DMF:H₂O:AcOH (6:2:2, 1 mL), filtered through a PTFE frit and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 10% B, 10-50% B over 25 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to afford Compound 244 (4.7 mg) containing 0.8 eq of AcOH.

### Example 21—Compound 269

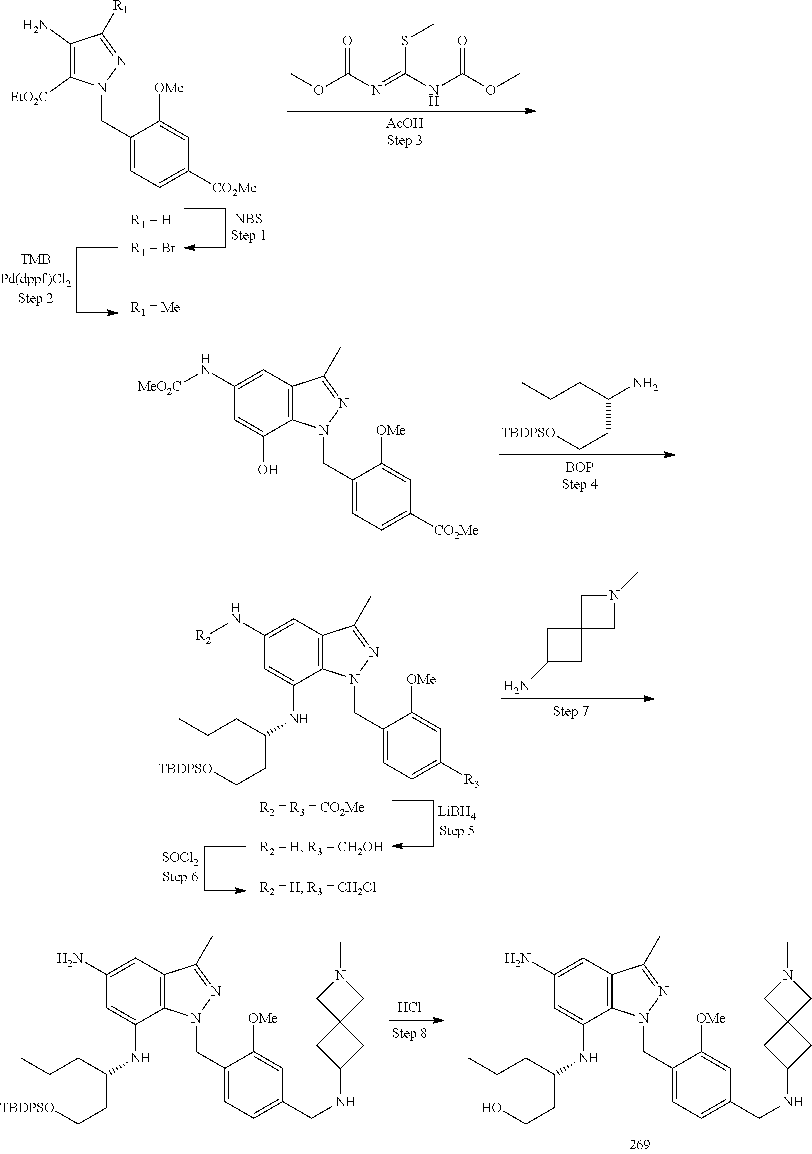

Step 1. Ethyl 4-amino-1-(2-methoxy-4-(methoxycarbonyl)benzyl)-1H-pyrazole-5-carboxylate (1.65 g, 4.95 mmol) was dissolved in CHCl₃ (49.5 ml) and cooled to 0° C. NBS (0.925 g, 5.20 mmol) was added in one portion. After 15 minutes, the reaction was diluted with CHCl₃ and vigorously stirred with 10% aqueous Na₂S₂O₃ solution for 10 minutes. The organic phase was separated, washed with H₂O, dried over MgSO₄ and concentrated. The crude product was purified by column chromatography (80 g SiO2, 0 to 50% EtOAc-hexane gradient elution) to afford ethyl 4-amino-3-bromo-1-(2-methoxy-4-(methoxycarbonyl)benzyl)-1H-pyrazole-5-carboxylate (1.32 g) as a white solid.

### LC/MS (M+H) 412.2/414.2; LC RT=1.02 min (Method A).

¹H NMR (400 MHz, DMSO-d6) δ 7.61-7.41 (m, 2H), 6.55 (d, J=8.3 Hz, 1H), 5.56 (s, 2H), 5.02 (s, 2H), 4.20 (q, J=7.1 Hz, 2H), 3.90 (s, 3H), 3.85 (s, 3H), 1.15 (t, J=7.1 Hz, 3H).

Step 2. Ethyl 4-amino-1-(2-methoxy-4-(methoxycarbonyl)benzyl)-3-methyl-1H-pyrazole-5-carboxylate (741.2 mg, 67.1% yield), K₂CO₃ (1.098 g, 7.94 mmol) and 2,4,6-trimethyl-1,3,5,2,4,6-trioxatriborinane (3.5 M in THF) (1.816 ml, 6.36 mmol) were suspended in dioxane (26.5 ml):water (5.30 ml) (5:1). A stream of N₂ was bubbled through the reaction mixture for 5 min before the addition of PdCl₂(dppf)-CH₂Cl₂ adduct (0.052 g, 0.064 mmol) and continued for another 4 min before sealing the reaction vessel and heating to 90° C. After 3 h, additional portions of 2,4,6-trimethyl-1,3,5,2,4,6-trioxatriborinane (TMB, 3.5 M in THF; 0.908 ml, 3.18 mmol) and PdCl₂(dppf)-CH₂Cl₂ adduct (0.052 g, 0.064 mmol) were added and the reaction mixture was stirred at 100° C. for 16 h. The cooled reaction mixture was diluted with 100 mL of EtOAc and filtered through CELITE™, washing with additional EtOAc. The crude product was concentrated onto 4 g CELITE™. Column chromatography (80 g SiO2, 0 to 30% EtOAc-CH₂Cl₂ gradient elution) afforded the expected product, ethyl 4-amino-1-(2-methoxy-4-(methoxycarbonyl)benzyl)-3-methyl-1H-pyrazole-5-carboxylate (741 mg) as a cream solid.

### LC/MS (M+H) 348.2; LC RT=0.89 min (Method A).

¹H NMR (400 MHz, DMSO-d6) δ 7.49 (d, J=1.5 Hz, 1H), 7.46 (dd, J=7.9, 1.5 Hz, 1H), 6.40 (d, J=7.8 Hz, 1H), 5.48 (s, 2H), 4.94-4.86 (m, 2H), 4.14 (q, J=7.0 Hz, 2H), 3.90 (s, 3H), 3.84 (s, 3H), 2.10 (s, 3H), 1.15-1.08 (m, 3H).

Step 3. Ethyl 4-amino-1-(2-methoxy-4-(methoxycarbonyl)benzyl)-3-methyl-1H-pyrazole-5-carboxylate (742 mg, 2.136 mmol) was suspended in MeOH (10.800 mL) and heated gently with vigorous stirring to solubilize the material. 1,3-bis-(Methoxycarbonyl)-2-methyl-2-thiopseudourea (661 mg, 3.20 mmol), was added followed by AcOH (0.611 mL, 10.68 mmol). 30 The reaction mixture was stirred at RT for 16 h. An additional portion of AcOH was added (0.049 mL, 0.854 mmol) and the reaction was stirred at RT for another 72 h before the addition of NaOMe (25% wt in MeOH) (5.69 mL, 25.6 mmol). After stirring for 3 h, the reaction mixture was reacidified with AcOH until acidic. The product was collected by filtration, air-dried for 10 minutes and thoroughly dried in a chem-dry oven to afford methyl 4-((7-hydroxy-5-((methoxycarbonyl)amino)-3-methyl-1H-pyrazolo[4,3-d]pyrimidin-1-yl)methyl)-3-methoxybenzoate (722.0 mg) as a cream solid.

### LC/MS (M+H) 402.3; LC RT=0.86 min (Method A).

¹H NMR (400 MHz, DMSO-d6) δ 11.58-11.17 (m, 2H), 7.51 (d, J=1.4 Hz, 1H), 7.49-7.42 (m, 1H), 6.67 (d, J=7.9 Hz, 1H), 5.67 (s, 2H), 3.90 (s, 3H), 3.84 (s, 3H), 3.71 (s, 3H), 2.31 (s, 3H).

Step 4. Methyl 4-((7-hydroxy-5-((methoxycarbonyl)amino)-3-methyl-1H-pyrazolo[4,3-d]pyrimidin-1-yl)methyl)-3-methoxybenzoate (300 mg, 0.747 mmol), (S)-1-((tert-butyldiphenylsilyl)oxy)hexan-3-amine, HCl (381 mg, 0.972 mmol) and BOP (496 mg, 1.121 mmol) were suspended in DMF (3737 μl) at RT. After the addition of DBU (4 eq) (451 μl, 2.99 mmol), the reaction mixture became homogenous and was heated to 40° C. After 15 min, an additional portion of DBU (2 eq) (225 μl, 1.495 mmol) was added and the reaction was stirred at 40° C. for 16 h. (S)-1-((tert-butyldiphenylsilyl)oxy)hexan-3-amine, HCl (381 mg, 0.972 mmol), BOP (496 mg, 1.121) and DBU (4 eq) (451 μl, 2.99 mmol) was added and the reaction was stirred for an additional 48 h. The reaction mixture was diluted with EtOAc and washed with H₂O (2×), and 10% LiCl solution (1×). The organic phase was dried over Na₂SO₄ and concentrated. The crude product was purified by column chromatography (24 g SiO₂, 0 to 80% EtOAc-hexane gradient elution) then further purified (12 g SiO₂, 0 to 70% EtOAc-hexane gradient elutionto provide methyl (S)-4-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-3-methoxybenzoate (270.6 mg).

### LC/MS (M+H) 739.7; LC RT=1.04 min (Method A).

Step 5. To a solution of methyl (S)-4-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-3-methoxybenzoate (500 mg, 0.677 mmol) in dry THF (10 mL) and MeOH (3 mL) was added LiBH₄ (1.692 mL, 3.38 mmol) under nitrogen atmosphere. The reaction mixture was heated at 45° C. for 24 h. The reaction mixture was partitioned between aqueous NH₄Cl solution and EtOAc. The organic layer was washed with water and brine, dried over anhydrous Na₂SO₄ and concentrated. The crude product was purified by CombiFlash chromatography (60-120 silica gel; 10-100% ethyl acetate in petroleum ether to provide (S)-(4-((5-amino-7-((1-((tert-butyldiphenylsilyl)oxy)hexan-3-yl)amino)-3-methyl-1H-pyrazolo[4,3-d]pyrimidin-1-yl)methyl)-3-methoxyphenyl)methanol (150 mg, 0.230 mmol, 34.0% yield) as a light yellow solid.

### LC/MS (M+H) 653.4

Step 6: To a stirred solution of (S)-(4-((5-amino-7-((1-((tert-butyldiphenylsilyl)oxy)hexan-3-yl)amino)-3-methyl-1H-pyrazolo[4,3-d]pyrimidin-1-yl)methyl)-3-methoxyphenyl)methanol (150 mg, 0.230 mmol) in THF (0.5 mL) was added SOCl₂ (0.1 ml, 1.370 mmol). The reaction mixture was stirred at 0° C. for 1 h under nitrogen atmosphere and subsequently concentrated in vacuo to provide (S)-N7-(1-((tert-butyldiphenylsilyl)oxy)hexan-3-yl)-1-(4-(chloromethyl)-2-methoxybenzyl)-3-methyl-1H-pyrazolo[4,3-d]pyrimidine-5,7-diamine as a light yellow solid, which was taken for next step without further purification.

### LC/MS (M+H) 671.4

Step 7: To a stirred solution of (S)-N7-(1-((tert-butyldiphenylsilyl)oxy)hexan-3-yl)-1-(4-(chloromethyl)-2-methoxybenzyl)-3-methyl-1H-pyrazolo[4,3-d]pyrimidine-5,7-diamine (150 mg, 0.223 mmol) in DMF (2 mL) were added 2-methyl-2-azaspiro[3.3]heptan-6-amine, HCl (72.7 mg, 0.447 mmol) and K₂CO₃ (61.8 mg, 0.447 mmol). The reaction mixture was stirred at 50° C. for 3 h and subsequently filtered. The filtrate was concentrated in vacuo to provide (S)-N7-(1-((tert-butyldiphenylsilyl)oxy)hexan-3-yl)-1-(2-methoxy-4-(((2-methyl-2-azaspiro[3.3]heptan-6-yl)amino)methyl)benzyl)-3-methyl-1H-pyrazolo[4,3-d]pyrimidine-5,7-diamine as a light brownish oil, which was taken for next step without further purification. LC/MS (M+H) 761.5

Step 8:To a stirred solution of (S)-N7-(1-((tert-butyldiphenylsilyl)oxy)hexan-3-yl)-1-(2-methoxy-4-(((2-methyl-2-azaspiro[3.3]heptan-6-yl)amino)methyl)benzyl)-3-methyl-1H-pyrazolo[4,3-d]pyrimidine-5,7-diamine (150 mg, 0.197 mmol) in MeOH (3 mL), was added HCl (0.3 mL, 9.87 mmol). The reaction mixture was stirred at 0° C. to RT for 2 h under nitrogen atmosphere and subsequently concentrated in vacuo. The residue was purified by prep-HPLC with the following condition (Column:Column: Ascentis Express C18(50×2.1 mm),2.7 μm; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0-100% B over 3 minutes; Flow: 1.1 ml/min. Injection 2 conditions: Column: Ascentis Express C18(50×2.1 mm),2.7 μm; Mobile Phase A: 5:95 acetonitrile:water with 0.1% TFA; Mobile Phase B: 95:5 acetonitrile:water with 0.1% TFA; Temperature:50° C.; Gradient:0-100% B over 3 minutes; Flow: 1.1 ml/min.) to provide Compound 269 (14.6 mg, 0.027 mmol, 13.75% yield).

### Example 22—Compound 245

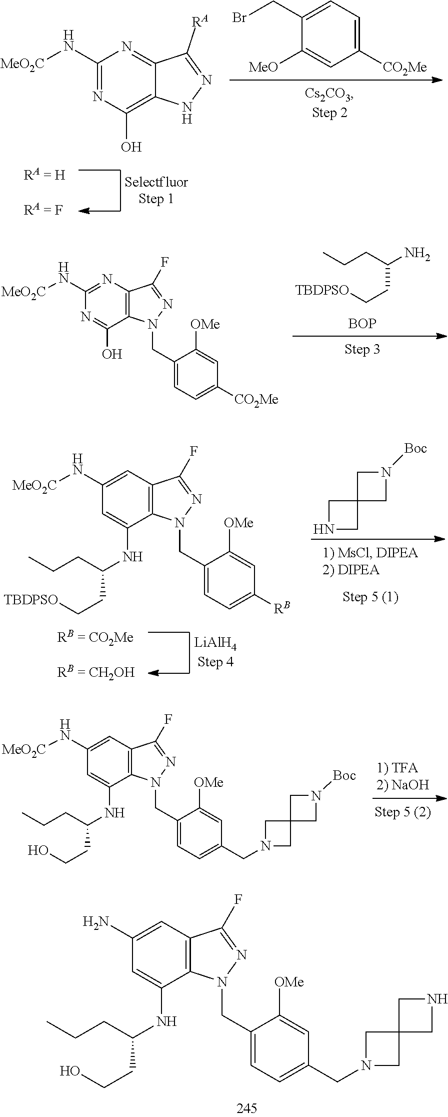

Step 1. Methyl (7-hydroxy-1H-pyrazolo[4,3-d]pyrimidin-5-yl)carbamate (2 g, 9.56 mmol) and Selectfluor™ (10.16 g, 28.7 mmol) were suspended in MeCN (20 mL). Acetic Acid (2 mL) was added. The reaction mixture stirred at 70° C. for 24 hours, cooled, and poured into water (100 mL). The resulting mixture was left to stand in the freezer (−20° C.) for 30 minutes. The precipitated the product was collected by filtration, washing with water (40 mL), giving methyl (3-fluoro-7-hydroxy-1H-pyrazolo[4,3-d]pyrimidin-5-yl)carbamate (1311 mg, 5.77 mmol, 60.4% yield) as a solid.

LC-MS (ES, m/z): [M+H]⁺=228.2.

¹H NMR (400 MHz, DMSO-d₆) δ 13.69 (s, 1H), 11.63 (s, 1H), 11.26 (s, 1H), 3.76 (s, 3H).

Step 2. A stirred suspension of methyl (3-fluoro-7-hydroxy-1H-pyrazolo[4,3-d]pyrimidin-5-yl)carbamate (1.311 g, 5.77 mmol) and Cs₂CO₃ (2.257 g, 6.93 mmol) in DMF (5 mL) was cooled in an ice bath. A solution of methyl 4-(bromomethyl)-3-methoxybenzoate (1.495 g, 5.77 mmol) in DMF (5 mL) was added. The reaction mixture allowed to warm slowly to RT, stirred overnight and filtered. The filtrate evaporated in a Genevac apparatus. The precipitate was washed with THF (100 mL) and water (100 mL), and the filtrates collected separately. The final precipitate was combined with the dried material from the DMF filtrate and the THF filtrate, and evaporated onto silica, then purified using flash chromatography (80 g SiO₂ column, 0 to 10% MeOH in DCM), giving methyl 4-((3-fluoro-7-hydroxy-5-((methoxycarbonyl)amino)-1H-pyrazolo[4,3-d]pyrimidin-1-yl)methyl)-3-methoxybenzoate (1.03 g, 2.54 mmol, 44.0% yield) as a solid.

LC-MS (ES, m/z): [M+H]⁺=406.1.

Step 3. A 40 mL scintillation vial was charged with methyl 4-((3-fluoro-7-hydroxy-5-((methoxycarbonyl)amino)-1H-pyrazolo[4,3-d]pyrimidin-1-yl)methyl)-3-methoxybenzoate (1013 mg, 2.499 mmol), (S)-1-((tert-butyldiphenylsilyl)oxy)hexan-3-amine (1333 mg, 3.75 mmol), BOP (1658 mg, 3.75 mmol), DBU (1.13 mL, 7.5 mmol) and DMSO (10 mL). The reaction mixture was stirred at 60° C. for 2 h, cooled, poured into saturated NaHCO₃ solution (150 mL) and extracted into EtOAc (3×60 mL). The combined organic phases were washed with brine (4×50 mL), dried (MgSO₄), filtered and concentrated. The crude material was purified using flash chromatography (80 g SiO₂ column, 0 to 70% EtOAc in hexanes), giving methyl (S)-4-((7-((1-((tert-butyldiphenylsilyl)oxy)hexan-3-yl)amino)-3-fluoro-5-((methoxycarbonyl)amino)-1H-pyrazolo-[4,3-d]pyrimidin-1-yl)methyl)-3-methoxybenzoate (493 mg, 0.664 mmol, 26.6% yield) as an oil. LC-MS (ES, m/z): [M+H]⁺=743.3.

Step 4. A solution of methyl (S)-4-((7-((1-((tert-butyldiphenylsilyl)oxy)hexan-3-yl)amino)-3-fluoro-5-((methoxycarbonyl)amino)-1H-pyrazolo[4,3-d]pyrimidin-1-yl)methyl)-3-methoxybenzoate (493 mg, 0.664 mmol) in THF (50 mL) was cooled in an ice bath. LiAIH₄ (0.697 mL, 1.394 mmol) was added. The reaction mixture was stirred at 0° C. for 15 min. Rochelle's salt (20 mL, 20 w/v) was added, and after stirring for 15 minutes at RT the reaction mixture was poured into water (100 mL) and extracted into EtOAc (3×50 mL). The combined organic phases were washed with brine (3×40 mL), dried (MgSO₄), filtered and concentrated. The crude material was purified using flash chromatography (24 g SiO₂ column, 0 to 100% EtOAc in hexanes), giving methyl (S)-(3-fluoro-7-((1-hydroxyhexan-3-yl)amino)-1-(4-(hydroxymethyl)-2-methoxybenzyl)-1H-pyrazolo[4,3-d]pyrimidin-5-yl)carbamate (125 mg, 0.262 mmol, 39.5% yield), as a solid.

LC-MS (ES, m/z): [M+H]⁺=477.2.

Step 5. To a stirred solution of methyl (S)-(3-fluoro-7-((1-hydroxyhexan-3-yl)amino)-1-(4-(hydroxymethyl)-2-methoxybenzyl)-1H-pyrazolo[4,3-d]pyrimidin-5-yl)carbamate (40 mg, 0.084 mmol) in DCM (2 mL) was added DIPEA (0.044 mL, 0.252 mmol) and methanesulfonyl chloride (0.013 mL, 0.168 mmol). The reaction mixture was stirred at RT for 30 min and then evaporated to dryness. The residue was dissolved in DMF (2 mL), and tert-butyl 2,6-diazaspiro[3.3]heptane-2-carboxylate (33.3 mg, 0.168 mmol) and DIPEA (0.044 mL, 0.252 mmol) were added. The reaction mixture was stirred at 80° C. for 2 h, cooled, quenched with saturated NaHCO₃ solution (10 mL), and extracted into EtOAc (3×5 mL). The combined organic phases were washed with brine (4×5 mL), dried (MgSO₄), filtered and concentrated. The residue was dissolved in DCM (2 mL), and TFA (0.4 mL) was added. The reaction mixture was stirred overnight at RT and then evaporated to dryness. The residue was then dissolved in dioxane (2 mL). NaOH (0.420 mL, 2.099 mmol) was added. The reaction mixture stirred at 80° C. for 2 h, cooled, acidified with 5N HCl, and evaporated to dryness. The crude material was dissolved in DMF (2 mL), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 4% B, 4-44% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 245 (6.1 mg, 0.012 mmol, 14.14% yield).

### Example 23—Compound 246

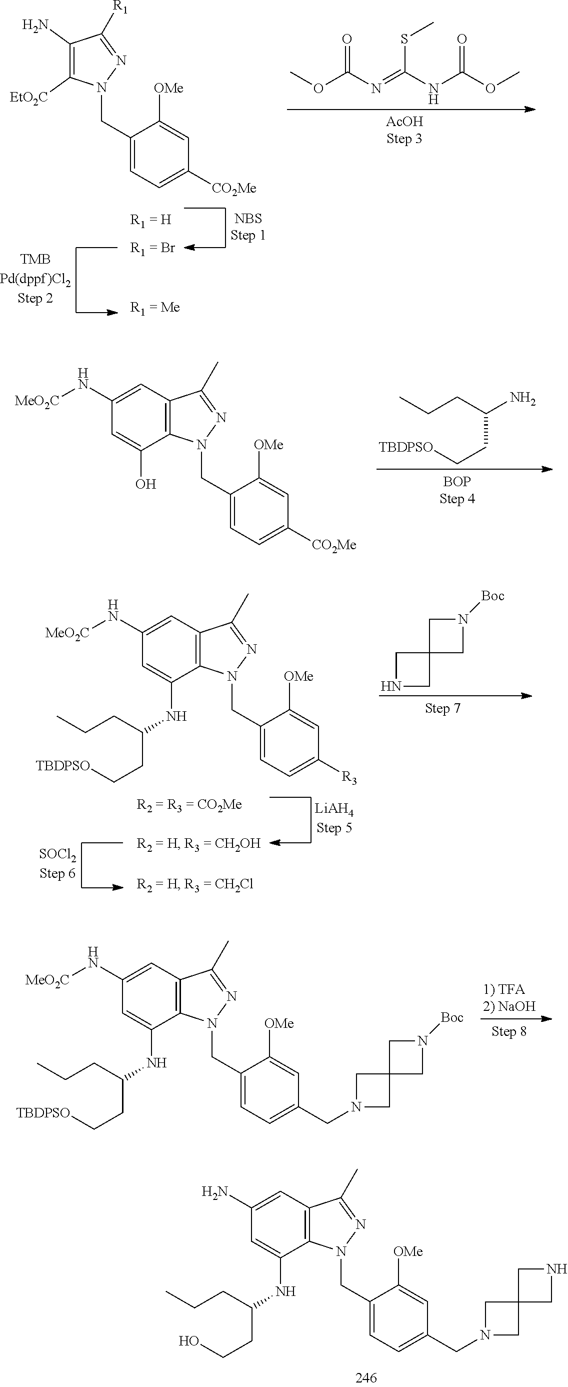

Step 1. Ethyl 4-amino-1-(2-methoxy-4-(methoxycarbonyl)benzyl)-1H-pyrazole-5-carboxylate (1.65 g, 4.95 mmol) was dissolved in CHCl₃ (49.5 ml) and cooled to 0° C. NBS (0.925 g, 5.20 mmol) was added to the reaction mixture in one portion. After 15 minutes, the reaction was diluted with CHCl₃ and vigorously stirred with 10% aqueous Na₂S₂O₃ solution for 10 minutes. The organic phase was separated, washed with H₂O, dried over MgSO₄ and concentrated. The crude product was purified by column chromatography (80 g SiO2, 0 to 50% EtOAc-hexane gradient elution) to afford ethyl 4-amino-3-bromo-1-(2-methoxy-4-(methoxycarbonyl)benzyl)-1H-pyrazole-5-carboxylate (1.32 g) as a white solid. LC/MS (M+H) 412.2/414.2; LC RT=1.02 min (Method A).

¹H NMR (400 MHz, DMSO-d6) δ 7.61-7.41 (m, 2H), 6.55 (d, J=8.3 Hz, 1H), 5.56 (s, 2H), 5.02 (s, 2H), 4.20 (q, J=7.1 Hz, 2H), 3.90 (s, 3H), 3.85 (s, 3H), 1.15 (t, J=7.1 Hz, 3H).

Step 2. Ethyl 4-amino-1-(2-methoxy-4-(methoxycarbonyl)benzyl)-3-methyl-1H-pyrazole-5-carboxylate (741.2 mg, 67.1% yield), K₂CO₃ (1.098 g, 7.94 mmol) and 2,4,6-trimethyl-1,3,5,2,4,6-trioxatriborinane (3.5 M in THF) (1.816 ml, 6.36 mmol) were suspended in dioxane (26.5 ml):water (5.30 ml) (5:1). A stream of N₂ was bubbled through the reaction mixture for 5 min before the addition of PdCl₂(dppf)-CH₂Cl₂ adduct (0.052 g, 0.064 mmol) and continued for another 4 min before sealing the reaction vessel and heating to 90° C. After 3 h, additional portions of 2,4,6-trimethyl-1,3,5,2,4,6-trioxatriborinane (3.5 M in THF) (0.908 ml, 3.18 mmol) and PdCl₂(dppf)-CH₂Cl₂ adduct (0.052 g, 0.064 mmol) were added and the reaction mixture was stirred at 100° C. for 16 hours. The cooled reaction mixture was diluted with 100 mL of EtOAc and filtered through CELITE™, washing with additional EtOAc. The crude product was concentrated onto 4 g CELITE™. Column chromatography (80 g SiO2, 0 to 30% EtOAc-CH₂Cl₂ gradient elution) afforded the expected product, ethyl 4-amino-1-(2-methoxy-4-(methoxycarbonyl)benzyl)-3-methyl-1H-pyrazole-5-carboxylate (741 mg) as a cream solid.

### LC/MS (M+H) 348.2; LC RT=0.89 min (Method A).

¹H NMR (400 MHz, DMSO-d6) δ 7.49 (d, J=1.5 Hz, 1H), 7.46 (dd, J=7.9, 1.5 Hz, 1H), 6.40 (d, J=7.8 Hz, 1H), 5.48 (s, 2H), 4.94-4.86 (m, 2H), 4.14 (q, J=7.0 Hz, 2H), 3.90 (s, 3H), 3.84 (s, 3H), 2.10 (s, 3H), 1.15-1.08 (m, 3H).

Step 3. Ethyl 4-amino-1-(2-methoxy-4-(methoxycarbonyl)benzyl)-3-methyl-1H-pyrazole-5-carboxylate (742 mg, 2.136 mmol) was suspended in MeOH (10.800 mL) and heated gently with vigorous stirring to solubilize the material. 1,3-bis-(Methoxycarbonyl)-2-methyl-2-thiopseudourea (661 mg, 3.20 mmol), was added followed by AcOH (0.611 mL, 10.68 mmol). The reaction mixture was stirred at RT for 16 h. An additional portion of AcOH was added (0.049 mL, 0.854 mmol) and the reaction was stirred at RT for another 72 h before the addition of NaOMe (25% wt in MeOH) (5.69 mL, 25.6 mmol). After stirring for 3 h, the reaction mixture was reacidified with AcOH until acidic. The product was collected by filtration, air-dried for 10 minutes and thoroughly dried in a chem-dry oven to afford methyl 4-((7-hydroxy-5-((methoxycarbonyl)amino)-3-methyl-1H-pyrazolo[4,3-d]pyrimidin-1-yl)methyl)-3-methoxybenzoate (722.0 mg) as a cream solid.

### LC/MS (M+H) 402.3; LC RT=0.86 min (Method A).

¹H NMR (400 MHz, DMSO-d6) δ 11.58-11.17 (m, 2H), 7.51 (d, J=1.4 Hz, 1H), 7.49-7.42 (m, 1H), 6.67 (d, J=7.9 Hz, 1H), 5.67 (s, 2H), 3.90 (s, 3H), 3.84 (s, 3H), 3.71 (s, 3H), 2.31 (s, 3H).

Step 4. Methyl 4-((7-hydroxy-5-((methoxycarbonyl)amino)-3-methyl-1H-pyrazolo[4,3-d]pyrimidin-1-yl)methyl)-3-methoxybenzoate (300 mg, 0.747 mmol), (S)-1-((tert-butyldiphenylsilyl)oxy)hexan-3-amine, HCl (381 mg, 0.972 mmol) and BOP (496 mg, 1.121 mmol) were suspended in DMF (3737 pI) at RT. After the addition of DBU (4 eq) (451 pI, 2.99 mmol), the reaction mixture became homogenous and was heated to 40° C. After 15 min, an additional portion of DBU (2 eq) (225 μl, 1.495 mmol) was added and the reaction was stirred at 40° C. for 16 h. (S)-1-((tert-butyldiphenylsilyl)oxy)hexan-3-amine, HCl (381 mg, 0.972 mmol), BOP (496 mg, 1.121) and DBU (4 eq) (451 μl, 2.99 mmol) was added and the reaction was stirred for an additional 48 h. The reaction mixture was diluted with EtOAc and washed with H₂O (2×), and 10% LiCl solution (1×). The organic phase was dried over Na₂SO₄ and concentrated. The crude product was purified by column chromatography (24 g SiO₂, 0 to 80% EtOAc-hexane gradient elution) then further purified (12 g SiO₂, 0 to 70% EtOAc-hexane gradient elutionto provide methyl (S)-4-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-3-methoxybenzoate (270.6 mg).

### LC/MS (M+H) 739.7; LC RT=1.04 min (Method A).

Step 5. Methyl (S)-4-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-3-methoxybenzoate (270 mg, 0.365 mmol) was dissolved in THF (3654 μl) at RT. LiAIH₄ (731 μl, 0.731 mmol) was added dropwise over 5 minutes. The reaction mixture was stirred for 15 min at RT and was quenched with MeOH and Rochelle's salt. EtOAc was added and the mixture was stirred for 3 h, until the layers cleared. The organic phase was removed and the aqueous layer was extracted with three additional portions of EtOAc. The combined organic phases were washed with brine, dried over Na₂SO₄ and concentrated. Column chromatography(12 g SiO2, 0 to 100% EtOAc-hexane gradient elution, then 0 to 20% MeOH—CH₂Cl₂) provided the expected material, methyl (S)-(7-((1-((tert-butyldiphenylsilyl)oxy)hexan-3-yl)amino)-1-(4-(hydroxyl-methyl)-2-methoxybenzyl)-3-methyl-1H-pyrazolo[4,3-d]pyrimidin-5-yl)carbamate (61.7 mg).

### LC/MS (M+H) 711.4; LC RT=1.08 min (Metod A).

Step 6. Methyl (S)-(7-((1-((tert-butyldiphenylsilyl)oxy)hexan-3-yl)amino)-1-(4-(hydroxymethyl)-2-methoxybenzyl)-3-methyl-1H-pyrazolo[4,3-d]pyrimidin-5-yl)carbamate (60 mg, 0.084 mmol) was dissolved in CH₂Cl₂ (844 μl) at RT. SOCl₂ (30.8 μL, 0.422 mmol) was added and the reaction for hr. Concentration afforded the expected product, methyl (S)-(7-((1-((tert-butyldiphenylsilyl)oxy)hexan-3-yl)amino)-1-(4-(chloromethyl)-2-methoxybenzyl)-3-methyl-1H-pyrazolo[4,3-d]pyrimidin-5-yl)carbamate (56.6 mg).

### LC/MS (M+H) 729.3; LC RT=1.18 min (Method A).

Step 7. Methyl (S)-(7-((1-((tert-butyldiphenylsilyl)oxy)hexan-3-yl)amino)-1-(4-(chlo-romethyl)-2-methoxybenzyl)-3-methyl-1H-pyrazolo[4,3-d]pyrimidin-5-yl)carbamate (45 mg, 0.02 mmol) was dissolved in acetonitrile (620 L) at RT. tert-Butyl 2,6-diazaspiro[3.3]heptane-2-carboxylate, HCl (29.0 mg, 0.123 mmol) was added followed by DIPEA (21.55 μl, 0.123 mmol). The reaction mixture was heated at 80° C. for 16 h and concentrated. The residue was purified by column chromatography (4 g SiO₂, 0-5% MeOH—CH₂Cl₂ gradient). Some by-product carried through the column and partially purified tert-butyl (S)-6-(4-((7-((1-((tert-butyldiphenylsilyl)-oxy)hexan-3-yl)amino)-5-((methoxycarbonyl)amino)-3-methyl-1H-pyrazolo[4,3-d]pyrimidin-1-yl)methyl)-3-methoxybenzyl)-2,6-diazaspiro[3.3]heptane-2-carboxylate (42 mg) was obtained.

### LC/MS (M+H) 892.7; LC RT=0.995 min (Method A).

Step 8. tert-butyl (S)-6-(4-((7-((1-((tert-butyldiphenylsilyl)oxy)hexan-3-yl)amino)-5-((methoxycarbonyl)amino)-3-methyl-1H-pyrazolo[4,3-d]pyrimidin-1-yl)methyl)-3-methoxy-benzyl)-2,6-diazaspiro[3.3]heptane-2-carboxylate (42 mg, 0.047 mmol) was dissolved in CH₂Cl₂ (471 μl) at RT. TFA (100 μl) was added. After 2 h, the reaction was concentrated under a stream of N₂ and re-dissolved in dioxane (470 μL). Triethylamine trihydrofluoride (16.79 μL, 0.103 mmol) was added and the reaction was heated to 70° C. After 45 min, 10 M aqueous NaOH (61.3 μl, 0.613 mmol) was added. The reaction mixture was stirred at 70° C. for 16 h, quenched with AcOH (54 μl), concentrated under a stream of N₂, diluted with DMF-H₂O, filtered through at PTFE frit and purified via preparative LC/MS with these conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-min hold at 1% B, 1-41% B over 20 min, then a 0-min hold at 100% B; Flow Rate: 20 mL/min; Column Temperature: 25° C. Fractions containing the product—collection triggered by MS signals—were combined and dried by centrifugal evaporation to provide Compound 246 (2.9 mg).

### Example 24—Compound 247

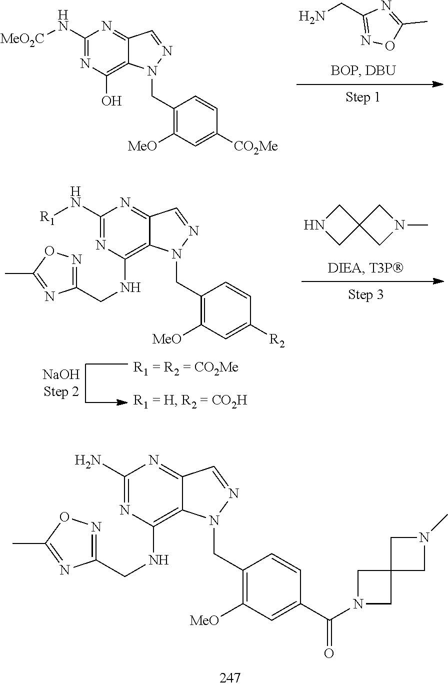

Step 1. A solution of methyl 4-((7-hydroxy-5-((methoxycarbonyl)amino)-1H-pyrazolo[4,3-d]pyrimidin-1-yl)methyl)-3-methoxybenzoate (510 mg, 1.32 mmol; US 2020/0038403 A1, FIG. 2A, compound 16) in DMSO (6.6 mL) was treated with (5-methyl-1,2,4-oxadiazol-3-yl)methanamine.HCl (236 mg, 1.58 mmol), BOP (698 mg, 1.58 mmol) and DBU (595 μL, 3.95 mmol). The reaction was stirred at RT. After 16 h, additional (5-methyl-1,2,4-oxadiazol-3-yl)methanamine.HCl (50 mg, 0.33 mmol), BOP (50 mg, 0.11 mmol) and DBU (200 μL, 1.33 mmol) were added and the reaction was stirred at RT for 2. The reaction mixture was diluted with EtOAc and washed with H₂O (4×). The organic layer was absorbed onto CELITE™ and purified via column chromatography (100 g C18 gold column; Mobile Phase A: 5:95 acetonitrile:water with 0.05% TFA; Mobile Phase B: 95:5 acetonitrile:water with 0.05% TFA; Flow Rate: 60 mL/min, 20-60% gradient). Fractions containing the desired product were combined, treated with HCl (1 M in H₂O, 2 mL, 2 mmol) and concentrated in vacuo to give methyl 3-methoxy-4-((5-((methoxycarbonyl)amino)-7-(((5-methyl-1,2,4-oxadiazol-3-yl)methyl)amino)-1H-pyrazolo[4,3-d]pyrimidin-1-yl)methyl)benzoate (382 mg, 60% yield).

¹H NMR (400 MHz, DMSO-d₆) δ 9.72-9.70 (m, 1H), 7.96-7.94 (m, 1H), 7.83-7.76 (m, 1H), 7.49 (d, J=1.4 Hz, 1H), 7.46 (dd, J=7.8, 1.5 Hz, 1H), 6.74 (d, J=7.8 Hz, 1H), 5.79 (s, 2H), 4.86 (d, J=5.8 Hz, 2H), 3.86 (s, 3H), 3.84 (s, 3H), 3.60 (s, 3H), 2.54 (s, 3H).

LC RT: 0.64 min. LC/MS [M+H]⁺ 483.3 (Method E).

Step 2. A solution of methyl 3-methoxy-4-((5-((methoxycarbonyl)amino)-7-(((5-methyl-1,2,4-oxadiazol-3-yl)methyl)amino)-1H-pyrazolo[4,3-d]pyrimidin-1-yl)methyl)benzoate (382 mg, 0.791 mmol) in Dioxane (9.0 mL) was treated with NaOH (10 M aqueous soln, 0.32 mL, 3.2 mmol) and heated to 40° C. After 30 minutes the temperature was increased to 60° C. Additional portions of NaOH (10M aqueous Soln, 450 μL, 3 mmol) and MeOH (1 mL) were added to the reaction mixture over a period of 6 h. The reaction mixture was cooled to RT, neutralized with HOAc and concentrated in vacuo. The crude product was dissolved in MeOH, filtered through a PTFE frit, and purified via preparative HPLC with the following conditions: Column: Axia C18 100 mm×30 mm, 5-μm particles; Mobile Phase A: 10:90 Methanol: water with 0.1% TFA; Mobile Phase B: 90:10 Methanol: water with 0.1% TFA; Gradient: a 0-minute hold at 15% B, 15-30% B over 10 minutes, then a 4-minute hold at 30% B; Flow Rate: 40 mL/min; UV detection at 220 nm; Column Temperature: 25° C. Fractions containing the desired product were combined, treated with HCl (1 M in H₂O, 2 mL, 2 mmol) and concentrated in vacuo to give 4-((5-amino-7-(((5-methyl-1,2,4-oxadiazol-3-yl)methyl)amino)-1H-pyrazolo[4,3-d]pyrimidin-1-yl)methyl)-3-methoxybenzoic acid-HCl (98.9 mg, 28% yield).

¹H NMR (400 MHz, DMSO-d₆) δ 13.23-12.93 (m, 1H), 12.67-12.43 (m, 1H), 9.06-8.92 (m, 1H), 8.03-7.87 (m, 2H), 7.83 (s, 1H), 7.51-7.46 (m, 2H), 6.98 (d, J=8.2 Hz, 1H), 5.80 (s, 2H), 4.91 (d, J=5.7 Hz, 2H), 3.82 (s, 3H), 2.57 (s, 3H). LC RT: 0.52 min.

### LC/MS [M+H]⁺ 411.3 (Method E).

Step 3. A solution of 4-((5-amino-7-(((5-methyl-1,2,4-oxadiazol-3-yl)methyl)amino)-1H-pyrazolo[4,3-d]pyrimidin-1-yl)methyl)-3-methoxybenzoic acid-HCl (25 mg, 0.056 mmol) in DMF (0.6 mL) was treated with 2-methyl-2,6-diazaspiro[3.3]heptane-2 HCl (20.7 mg, 0.112 mmol), DIEA (68 μL, 0.39 mmol) and 2,4,6-Tripropyl-1,3,5,2,4,6-trioxatriphosphorinane-2,4,6-trioxide (50% solution in EtOAc, 67 μL, 0.11 mmol). The reaction was stirred at RT. After 16 h the reaction mixture was diluted with DMF (1 mL) and H₂O (0.2 mL) and filtered through a PTFE frit. The crude material was purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 26 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 247 as the bis TFA salt (20.5 mg, 72% yield).

Compound 248 was analogously prepared.

### Example 25—Compound 254

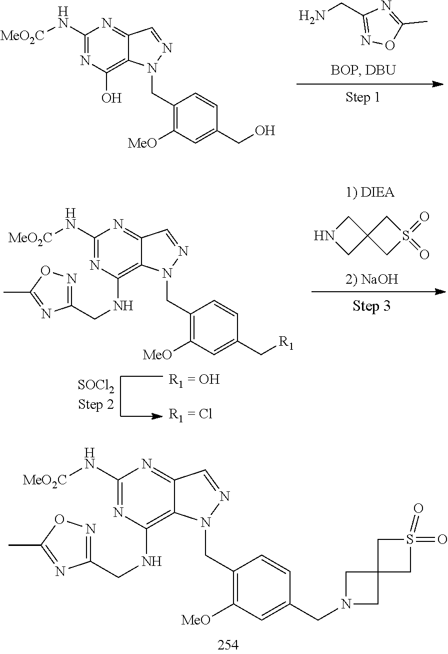

Step 1: A solution of methyl (7-hydroxy-1-(4-(hydroxymethyl)-2-methoxybenzyl)-1H-pyrazolo[4,3-d]pyrimidin-5-yl)carbamate (700 mg, 1.95 mmol; US 2020/0038403 A1; FIG. 7, compound 64) in DMSO (9.7 mL) was treated with (5-methyl-1,2,4-oxadiazol-3-yl)methan-amine-HCl (379 mg, 2.53 mmol), BOP (129 mg, 2.92 mmol) and DBU (1.0 mL, 6.8 mmol). The reaction mixture was stirred at RT for 2 h, diluted with DCM and washed with H₂O. The organic layer was washed with H₂O (6×) and dried over Na₂SO₄, filtered and concentrated in vacuo. The residue was dissolved in DCM/MeOH, absorbed onto CELITE™ and purified via column chromatography (100 g C18 gold column; Mobile Phase A: 5:95 acetonitrile:water with 0.05% TFA; Mobile Phase B: 95:5 acetonitrile:water with 0.05% TFA; Flow Rate: 60 mL/min, 10-50% gradient). The purified product was dissolved in DCM and washed with saturated aqueous NaHCO₃ soln. The organic layer was dried over Na₂SO₄, filtered and concentrated in vacuo to give methyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (372 mg, 42% yield). ¹H NMR (400 MHz, DMSO-d₆) δ 9.69-9.66 (m, 1H), 7.89 (s, 1H), 7.76 (t, J=5.8 Hz, 1H), 6.95 (s, 1H), 6.81-6.77 (m, 1H), 6.76-6.70 (m, 1H), 5.69 (s, 2H), 5.17 (t, J=5.7 Hz, 1H), 4.89 (d, J=5.7 Hz, 2H), 4.45 (d, J=5.8 Hz, 2H), 3.77 (s, 3H), 3.60 (s, 3H), 2.56 (s, 3H).

LC RT: 0.56 min. LC/MS [M+H]⁺ 455.3 (Method E)

Step 2: A solution of methyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (372 mg, 0.818 mmol) in DCM (8.2 mL) was treated with SOCl₂ (179 μL, 2.46 mmol). The reaction mixture was stirred at RT for 10 min and concentrated in vacuo. The residue was redissolved in DCM and concentrated in vacuo to give methyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (387 mg, 100%).

¹H NMR (400 MHz, DMSO-d₆) δ 11.82-11.60 (m, 1H), 9.40-9.21 (m, 1H), 8.12-8.08 (m, 1H), 7.10 (s, 1H), 7.04-6.95 (m, 2H), 5.81 (s, 2H), 5.02 (br d, J=5.3 Hz, 2H), 4.74 (s, 2H), 3.80 (s, 3H), 3.75 (s, 3H), 2.60 (s, 3H). LC RT: 0.70 min.

### LC/MS [M+H]⁺=473.3 (Method E)

Step 3: A solution of methyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (45 mg, 0.095 mmol) in DMF (1.9 mL) was treated with DIEA (83 μL, 0.48 mmol) and 2-thia-6-azaspiro-[3.3]heptane 2,2-dioxide-HCl (26.2 mg, 0.143 mmol). The reaction mixture was stirred at 60° C. for 6 h and concentrated in vacuo. The residue was dissolved in dioxane (0.7 mL) and treated with NaOH (10M aqueous soln, 76 μL, 0.76 mmol) and heated to 60° C. for 5 h. The reaction mixture was neutralized at RT with HOAc and concentrated in vacuo. The crude product was dissolved in DMF and H₂O, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 1% B, 1-41% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The isolated product was purified further via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 254 (11.7 mg, 16%).

### Example 26—Compound 263

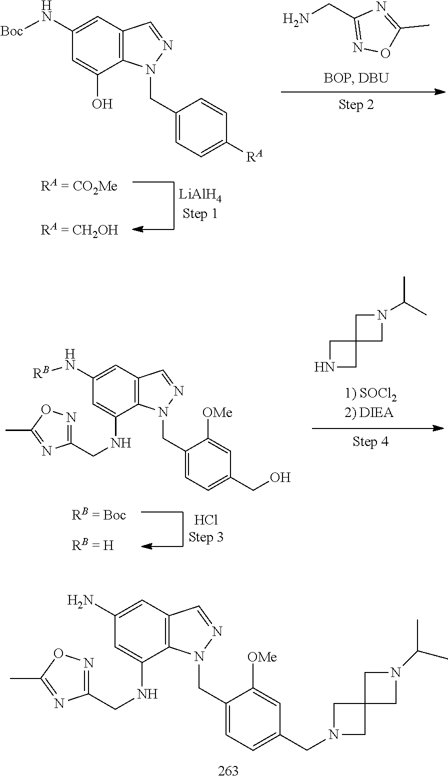

Step 1. A solution of methyl 4-((5-((tert-butoxycarbonyl)amino)-7-hydroxy-1H-pyrazolo[4,3-d]pyrimidin-1-yl)methyl)-3-methoxybenzoate (685 mg, 1.59 mmol; US 2020/0038403 A1, FIG. 8, compound 71) in THF (16 mL) was cooled to 0° C. and treated with LiAIH₄ (1 M in THF, 2.8 mL, 2.8 mmol). The reaction mixture was stirred for 15 min at 0° C., quenched with H₂O and Rochelle's salt (saturated aqueous soln), and stirred at RT for 3 h. The organic layer was absorbed onto CELITE™ and purified via column chromatography (24 g SiO₂; 0 to 20% MeOH-DCM gradient elution) to give tert-butyl (7-hydroxy-1-(4-(hydroxymethyl)-2-methoxybenzyl)-1H-pyrazolo[4,3-d]pyrimidin-5-yl)carbamate (460 mg, 72% yield). 1H (400 MHz, DMSO-d₆) δ 11.69-11.43 (m, 1H), 10.95-10.62 (m, 1H), 7.87-7.79 (m, 1H), 6.97 (s, 1H), 6.77 (d, J=7.7 Hz, 1H), 6.59 (d, J=7.8 Hz, 1H), 5.66 (s, 2H), 5.16 (t, J=5.8 Hz, 1H), 4.45 (d, J=5.8 Hz, 2H), 3.79 (s, 3H), 1.49 (s, 9H).

LC RT: 0.77 min. LC/MS [M+H]⁺=402.2 (Method E).

Step 2. A solution of tert-butyl (7-hydroxy-1-(4-(hydroxymethyl)-2-methoxybenzyl)-1H-pyrazolo[4,3-d]pyrimidin-5-yl)carbamate (460 mg, 1.15 mmol) in DMSO (5.7 mL) was treated with (5-methyl-1,2,4-oxadiazol-3-yl)methanamine-HCl (223 mg, 1.49 mmol), BOP (760 mg, 1.72 mmol) and DBU (0.69 mL, 4.6 mmol). The reaction mixture was stirred at RT for 2 h, diluted with EtOAc and washed with H₂O (2×). The organic layer was absorbed onto CELITE™ and purified via column chromatography (100 g C18 gold column; Mobile Phase A: 5:95 acetonitrile:water with 0.05% TFA; Mobile Phase B: 95:5 acetonitrile:water with 0.05% TFA; Flow Rate: 60 mL/min, 30-50% gradient). The purified product was dissolved in DCM and washed with saturated aqueous NaHCO₃ soln. The organic layer was dried over Na₂SO₄, filtered and concentrated in vacuo to give tert-butyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (190 mg, 33% yield).

¹H NMR (400 MHz, DMSO-d₆) δ 9.24-9.15 (m, 1H), 7.87 (s, 1H), 7.72 (t, J=5.8 Hz, 1H), 6.95 (s, 1H), 6.82-6.75 (m, 1H), 6.73-6.68 (m, 1H), 5.68 (s, 2H), 5.17 (t, J=5.7 Hz, 1H), 4.87 (d, J=5.7 Hz, 2H), 4.44 (d, J=5.7 Hz, 2H), 3.76 (s, 3H), 2.55 (s, 3H), 1.43 (s, 9H).

LC RT: 0.75 min. LC/MS [M+H]⁺=497.2 (Method E)

Step 3. A solution of tert-butyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (91.5 mg, 0.184 mmol) in dioxane (0.6 mL) was treated with HCl (4 M in dioxane, 0.69 mL, 2.8 mmol), stirred at 40° C. for 90 min and concentrated. The residue was dissolved in DCM and concentrated in vacuo to give (4-((5-amino-7-(((5-methyl-1,2,4-oxadiazol-3-yl)methyl)amino)-1H-pyrazolo[4,3-d]pyrimidin-1-yl)methyl)-3-methoxyphenyl)methanol (73.1 mg, 100% yield). 25 LC RT: 0.65 min. LC/MS [M+H]⁺=397.1 (Method E)

Step 4. A solution of 1-(4-(chloromethyl)-2-methoxybenzyl)-N7-((5-methyl-1,2,4-oxadiazol-3-yl)methyl)-1H-pyrazolo[4,3-d]pyrimidine-5,7-diamine (27 mg, 0.065 mmol) in DMSO (1.3 mL) was treated with DIEA (57 μL, 0.33 mmol) and 2-isopropyl-2,6-diazaspiro[3.3]-heptane (14 mg, 0.098 mmol). The reaction mixture was stirred at 65° C. for 30 min, diluted with DMSO, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 263 (13.7 mg, 35%) as the acetic acid salt.

Compound 264 was analogously prepared.

### Example 27- Compound 249

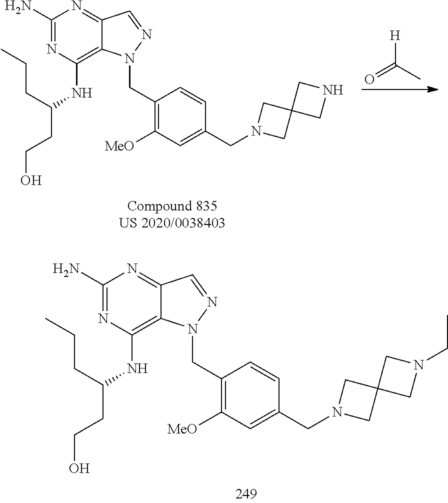

A mixture of compound 835 (20 mg, 0.042 mmol) and acetaldehyde (183 mg, 0.083 mmol) in DMF (1 mL) was treated with acetic acid (0.024 mL, 0.416 mmol) and 20 mg 4 Å molecular sieves, followed by sodium triacetoxyborohydride (35.3 mg, 0.166 mmol). The reaction mixture was stirred at RT for 1 h. The acetic acid (0.024 mL, 0.416 mmol) was evaporated. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 25 minutes, then a 0-minute hold at 100% B; Flow Rate: 20 mL/min; Column Temperature: 25° C. Fraction collection was triggered by MS signals. Fractions containing the desired compound 249 were combined and dried via centrifugal evaporation.

Compound 252 and Compound 253 were analogously prepared.

### Example 28—Compound 255

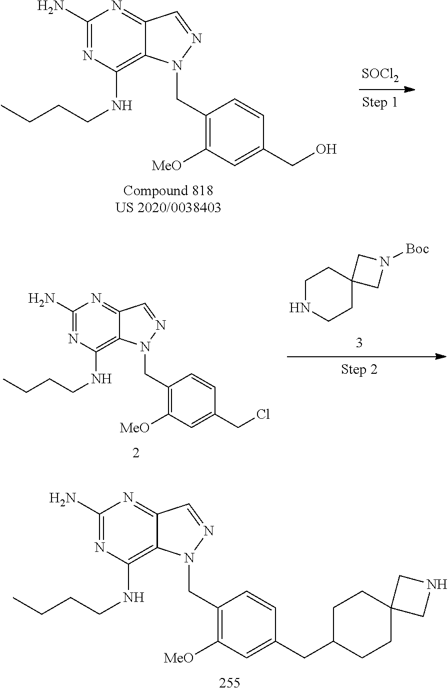

Step 1. A solution of (4-((5-amino-7-(butylamino)-1H-pyrazolo[4,3-d]pyrimidin-1-yl)methyl)-3-methoxyphenyl)methanol 818 (400 mg, 1.122 mmol) in THF (2 mL) was treated with SOCl₂ (0.164 mL, 2.244 mmol) and stirred for 1 h at RT. The solvent was evaporated and crude chloride 2 taken to next step without further purification.

Step 2. A solution of chloride 2 in DMSO was treated with amine 3 (commercially available, CAS: 236406-55-6) and heated at 80° C. for 2 h, after which LCMS showed completion of the reaction. The reaction mixture was treated with TFA and stirred for 1 h. The TFA was evaporated. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 4% B, 4-44% B over 20 minutes, then a 0-minute hold at 100% B; Flow Rate: 20 mL/min; Column Temperature: 25° C. Fraction collection was triggered by MS and UV signals. Fractions containing the desired compound 255 were combined and dried via centrifugal evaporation.

The following compounds were analogously prepared: Compound 256, Compound 257, Compound 258, Compound 265, and Compound 266.

### Example 29—Compound 259

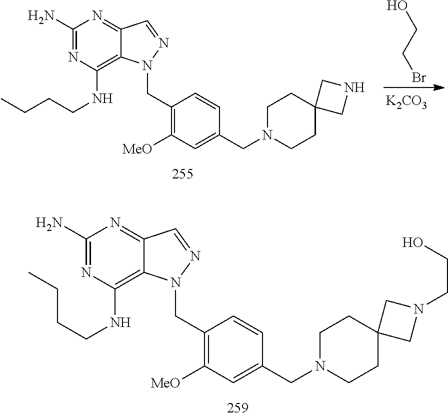

A solution of Compound 255 (18 mg, 0.039 mmol) in DMF (0.5 mL) was treated with K₂CO₃ (16.06 mg, 0.116 mmol)/2-bromoethan-1-ol (5.49 μl, 0.077 mmol) and heated at 50° C. for 2 h. The crude material was purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS and UV signals. Fractions containing the desired compound 259 were combined and dried via centrifugal evaporation.

The following compounds were analogously prepared: Compound 260, Compound 261, Compound 262, Compound 267, and Compound 268.

### Example 30—Compound 270

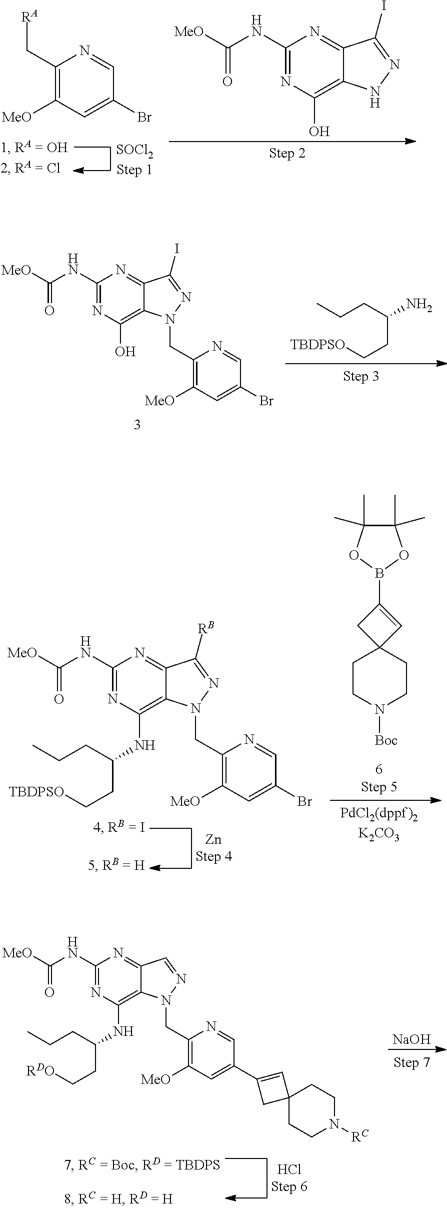

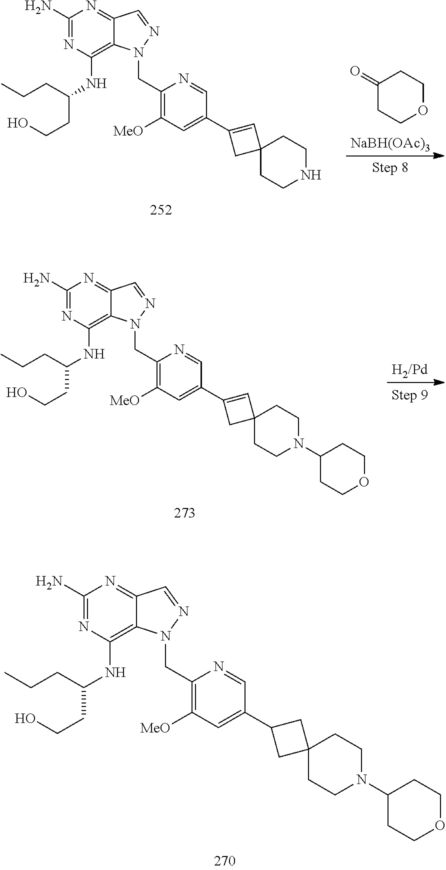 [SMILES: CCC[C@@H](CCO)Nc1nc(N)nc2cnn(Cc3ncc(C4=CC5(CCNCC5)C4)cc3OC)c12 ; O=C1CCOCC1 ; CCC[C@@H](CCO)Nc1nc(N)nc2cnn(Cc3ncc(C4=CC5(CCN(C6CCOCC6)CC5)C4)cc3OC)c12 ; "H2/Pd" ; "NaBH(OAc)3" ; CCC[C@@H](CCO)Nc1nc(N)nc2cnn(Cc3ncc(C4CC5(CCN(C6CCOCC6)CC5)C4)cc3OC)c12]

Step 1. To a 0° C. solution of (5-bromo-3-methoxypyridin-2-yl)methanol (Sigma-Aldrich) (2.462 g, 11.29 mmol) in CH₂Cl₂ (113 ml) was added SOCl₂ (1.235 ml, 16.94 mmol), dropwise. The reaction was stirred at RT for 1 h and concentrated in vacuo. The residue was mixed with CH₂Cl₂ and concentrated in vacuo (2×) to provide crude 5-bromo-2-(chloromethyl)-3-methoxypyridine. This material was used without further purification.

### LC-MS m/z 236/238 [M+H]⁺.

Step 2 To a RT suspension of methyl (7-hydroxy-3-iodo-1H-pyrazolo[4,3-d]pyrimidin-5-yl)carbamate (3.44 g, 10.26 mmol) in DMF (45.6 ml) was added Cs₂CO₃ (13.37 g, 41.0 mmol). The mixture was stirred at 0° C. for 10 min; then a solution of the crude material from Step 1 in DMF (22.80 ml) was added. The reaction mixture was stirred at 0° C. for 1 h. The cooling bath was removed and stirring was continued at RT for 20 h. The reaction mixture was added to H₂O (250 mL) and the resulting mixture was allowed to stand at RT. The solids were collected by vacuum filtration and washed with H₂O (3×15 mL), MeOH (2×15 mL), CH₂Cl₂ (15 mL), and hexanes (15 mL) to provide methyl (1-((5-bromo-3-methoxypyridin-2-yl)methyl)-7-hydroxy-3-iodo-1H-pyrazolo[4,3-d]pyrimidin-5-yl)carbamate (4.431 g, 81%).

### LC-MS m/z 535/537 [M+H]⁺.

¹H NMR (400 MHz, DMSO-d₆) δ 13.19-12.96 (m, 1H), 8.95-8.80 (m, 1H), 8.06 (s, 1H), 7.71 (s, 1H), 5.87-5.65 (m, 2H), 3.89 (s, 3H), 3.53 (br s, 3H).

Step 3. To a RT suspension of methyl (1-((5-bromo-3-methoxypyridin-2-yl)methyl)-7-hydroxy-3-iodo-1H-pyrazolo[4,3-d]pyrimidin-5-yl)carbamate (0.990 g, 1.850 mmol) in DMSO (12.33 ml) was added (S)-1-((tert-butyldiphenylsilyl)oxy)hexan-3-amine, HCl salt (0.870 g, 2.220 mmol) (US 2020/0038403 A1, FIG. 8, compound 71a), followed by 1,8-diazabicyclo[5.4.0]undec-7-ene (1.245 ml, 8.33 mmol) and (benzotriazol-1-yloxy)tris(dimethylamino)phosphonium hexafluorophosphate (0.982 g, 2.220 mmol). The reaction mixture was stirred at RT for 1 h, diluted with EtOAc (100 mL), and washed with H₂O (100 mL). The layers were separated, and the aqueous layer was extracted with EtOAc (100 mL). The combined organic layers were washed with saturated aqueous NaCl (100 mL), dried over Na₂SO₄, filtered, and concentrated in vacuo. The crude material was purified by flash chromatography (80 g silica gel; linear gradient 0-100% EtOAc-hexanes) to provide methyl (S)-(1-((5-bromo-3-methoxypyridin-2-yl)methyl)-7-((1-((tert-butyldiphenylsilyl)oxy)hexan-3-yl)amino)-3-iodo-1H-pyrazolo[4,3-d]pyrimidin-5-yl)carbamate (810 mg, 50%) as a yellow foam.

### LC-MS m/z 872/874 [M+H]⁺.

¹H NMR (400 MHz, DMSO-d₆) δ 9.69 (s, 1H), 7.92 (d, J=1.8 Hz, 1H), 7.79 (d, J=1.8 Hz, 1H), 7.57-7.53 (m, 2H), 7.50-7.46 (m, 2H), 7.42-7.31 (m, 4H), 7.25-7.20 (m, 2H), 7.12 (d, J=8.3 Hz, 1H), 5.78-5.69 (m, 2H), 4.64-4.55 (m, 1H), 3.91 (s, 3H), 3.70-3.64 (m, 2H), 3.58 (s, 3H), 1.90-1.82 (m, 2H), 1.57-1.48 (m, 2H), 1.25-1.13 (m, 2H), 0.92 (s, 9H), 0.81 (t, J=7.3 Hz, 3H).

Step 4. To a 0° C. solution of methyl (S)-(1-((5-bromo-3-methoxypyridin-2-yl)methyl)-7-((1-((tert-butyldiphenylsilyl)oxy)hexan-3-yl)amino)-3-iodo-1H-pyrazolo[4,3-d]pyrimidin-5-yl)carbamate (0.810 g, 0.928 mmol) in a mixture of MeOH (9.28 ml) and AcOH (9.28 ml) was added zinc (0.607 g, 9.28 mmol). The reaction mixture was stirred at 0° C. for 30 min and filtered through CELITE™ with washing with MeOH (10 mL) and EtOAc (50 mL). The filtrate was diluted with EtOAc (200 mL). While stirring, saturated aqueous NaHCO₃ (250 mL) was slowly added to this solution (the rate of addition adjusted to control the rate of gas evolution). The layers were separated and the organic layer was washed with saturated aqueous NaCl (250 mL), dried over Na₂SO₄, filtered, and concentrated in vacuo to provide the crude product, methyl (S)-(1-((5-bromo-3-methoxypyridin-2-yl)methyl)-7-((1-((tert-butyldiphenylsilyl)oxy)hexan-3-yl)amino)-1H-pyrazolo[4,3-d]pyrimidin-5-yl)carbamate. This material was used without further purification.

### LC-MS m/z 746/748 [M+H]⁺.

Step 5. Nitrogen gas was bubbled through a solution of compound 5 (500 mg, 0.670 mmol), compound 6 (304 mg, 0.870 mmol, CAS 2240187-78-2) and K₂CO₃ (370 mg, 2.68 mmol) in DMF (2 mL) for 2 min. PdCl₂(dppf)-CH₂Cl₂ adduct (54.7 mg, 0.067 mmol) was added and the reaction mixture was bubbled again with N₂ for 1 min. The reaction flask was sealed and heated at 70° C. for 5 h. Purification on a 50 g silica gel column eluting with 0-50% MeOH/DCM to provide 476 mg of compound 7.

### LC/MS [M+H]⁺=889.5.

¹H NMR (400 MHz, Chloroform-d) δ 8.02 (s, 5H), 7.92 (d, J=19.1 Hz, 1H), 7.63-7.50 (m, 3H), 7.37-7.23 (m, 2H), 7.22-7.14 (m, 2H), 7.04 (s, 1H), 6.62 (d, J=2.6H z, 1H), 5.64 (dd, J=14.7, 1.4 Hz, 1H), 5.39 (d, J=14.9 Hz, 1H), 4.63 (s, 1H), 3.95 (d, J=2.0 Hz, 2H), 3.82 (d, J=7.5 Hz, 3H), 3.57 (s, 1H), 3.31-3.21 (m, 1H), 2.96 (s, 7H), 2.88 (s, 7H), 2.49 (s, 1H), 1.95 (d, J=17.4 Hz, 1H), 1.47 (s, 5H), 1.52-1.36 (m, 2H), 1.26 (d, J=13.9 Hz, 5H), 1.02 (s, 3H), 1.04-0.90 (m, 2H).

Steps 6-7. Solid compound 7 (476 mg, 0.535 mmol) was treated with HCl in dioxane (1.338 mL, 5.35 mmol) with stirring at RT for 2 h, after which LC/MS showed completion of the reaction. The HCl was evaporated using a V-10 evaporator. The crude product 8 was dissolved in 1 mL dioxane and heated with aqueous NaOH solution (1.071 mL, 10.71 mmol) for 2 h, after which LC/MS showed completion of the reaction. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 20 minutes, then a 0-minute hold at 100% B; Flow Rate: 20 mL/min; Column Temperature: 25° C. Fraction collection was triggered by MS and UV signals. Fractions containing compound 272 ((3S)-3-({5-amino-1-[(5-{7-azaspiro[3.5]non-1-en-2-yl}-3-methoxypyridin-2-yl)methyl]-1H-pyrazolo[4,3-d]pyrimidin-7-yl}amino)hexan-1-ol) were combined and dried via centrifugal evaporation.

Step 8. A solution of compound 272 (40 mg, 0.081 mmol), tetrahydro-4H-pyran-4-one (37.5 μl, 0.406 mmol) in DMA (1 mL) was treated with acetic acid (46.5 μL, 0.812 mmol) followed by 50 mg of granular 4 Å molecular seives and sodium triacetoxyborohydride (86 mg, 0.406 mmol). The reaction mixture was stirred at RT overnight and syringe filtered. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 7% B, 7-47% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to provide compound 273 ((3S)-3-{[5-amino-1-({3-methoxy-5-[7-(oxan-4-yl)-7-azaspiro[3.5]non-1-en-2-yl]pyridin-2-yl}methyl)-1H-pyrazolo[4,3-d]pyrimidin-7-yl]amino}hexan-1-ol).

Step 9. Hydrogen gas was bubbled through a solution of compound 273 (18 mg, 0.026 mmol) in MeOH (1 mL) and Pd/C (2.73 mg, 0.026 mmol) for 1 min. The reaction mixture was heated at 60° C. under an atmosphere of a hydrogen balloon for 2 h. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 7% B, 7-47% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to provide compound 27.

The following compounds were analogously prepared: Compound 274, Compound 275, and Compound 278.

### Example 31—Compound 271

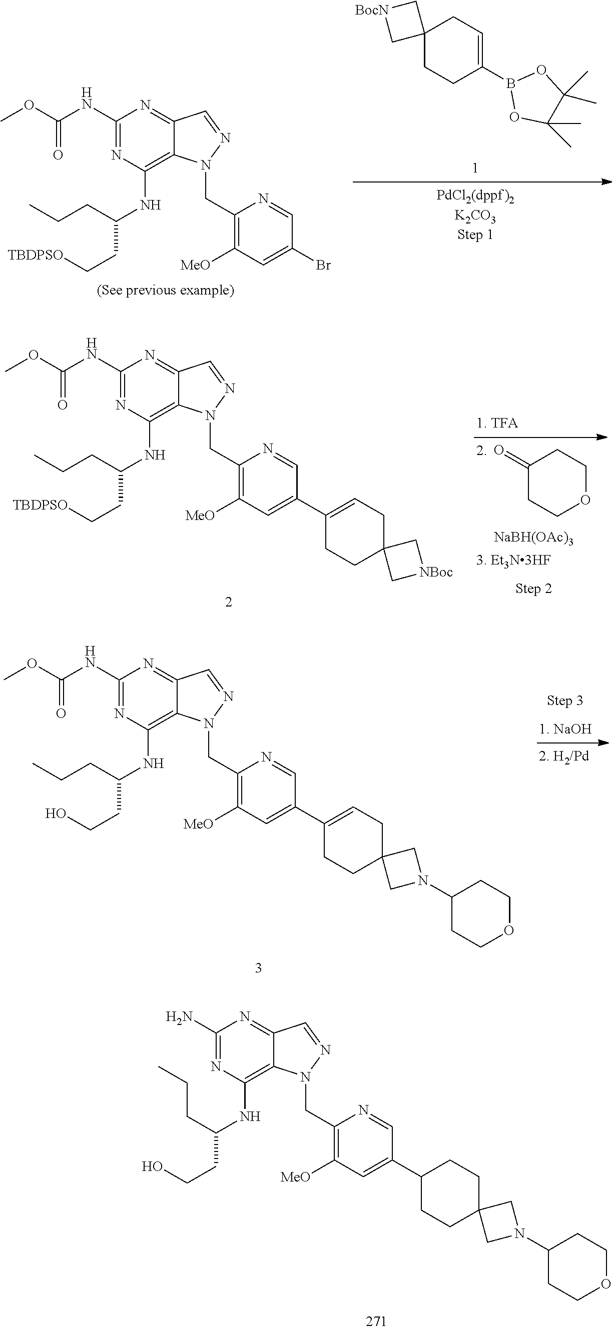

Step 1. A solution of methyl (S)-(1-((5-bromo-3-methoxypyridin-2-yl)methyl)-7-((1-((tert-butyldiphenylsilyl)oxy)hexan-3-yl)amino)-1H-pyrazolo[4,3-d]pyrimidin-5-yl)carbamate (552 mg, 0.739 mmol), tert-butyl 7-(4,4,5,5-tetramethyl-1,3,2-dioxaborolan-2-yl)-2-azaspiro[3.5]non-6-ene-2-carboxylate 1 (336 mg, 0.961 mmol; CAS 235276-13-4) and K₂CO₃ (409 mg, 2.96 mmol) in DMF (5 mL) was bubbled with N₂ for 2 min. PdCl₂(dppf)-CH₂Cl₂ adduct (60.4 mg, 0.074 mmol) was added and again N₂ was bubbled for 1 min. The reaction vessel was sealed and heated at 70° C. for 5 h. Purification on a 50 g silica gel column eluting with 0-50% MeOH/DCM to provide 477 mg of compound 2.

### LC/MS [M+H]⁺=889.5.

¹H NMR (400 MHz, Chloroform-d) δ 8.70 (d, J=8.1 Hz, 1H), 8.04-7.95 (m, 1H), 7.87 (s, 1H), 7.65-7.58 (m, 2H), 7.58-7.51 (m, 2H), 7.40-7.31 (m, 1H), 7.33-7.24 (m, 3H), 7.21-7.12 (m, 3H), 7.08 (s, 1H), 6.02 (s, 1H), 5.63 (d, J=14.9 Hz, 1H), 5.40 (d, J=15.0 Hz, 1H), 4.58 (s, 1H), 3.94 (s, 3H), 3.85-3.74 (m, 4H), 3.73-3.64 (m, 2H), 3.62 (d, J=8.3 Hz, 2H), 3.49 (s, 2H), 2.95 (s, 1H), 2.88 (s, 1H), 2.41 (d, J=4.3 Hz, 4H), 2.02 (dd, J=13.0, 6.2 Hz, OH), 1.95 (s, 1H), 1.91 (d, J=5.7 Hz, 1H), 1.45 (s, 6H), 1.45-1.36 (m, 1H), 1.24 (s, 6H), 1.04 (d, J=8.8 Hz, OH), 1.03 (s, 6H), 1.02 (s, 1H), 0.94 (t, J=7.3 Hz, 3H).

Step 2. Compound 2 (90 mg, 0.101 mmol) was treated with TFA (0.078 mL, 1.012 mmol). The reaction mixture was stirred at RT for 30 min. The TFA was evaporated in a V-10 evaporator. The residue was dissolved in DMA (0.5 mL) and treated with tetrahydro-4H-pyran-4-one (0.028 mL, 0.506 mmol), acetic acid (0.029 mL, 0.506 mmol), 50 mg 4 Å molecular sieves and finally with sodium triacetoxyborohydride (107 mg, 0.506 mmol). After stirring at RT for 1 h, the reaction mixture was treated with triethylamine trihydrofluoride (0.165 mL, 1.012 mmol) and stirred at RT for 2 h. The reaction mixture was directly purified on a 50 g reverse phase ISCO eluting with 0-50% MeCN/water (0.05% TFA) to yield compound 3 as white solid.

### LC/MS [M+H]⁺=635.3.

Step 3, Part 1. A solution of compound (58 mg, 0.091 mmol) in DMSO (0.5 mL) was treated with NaOH (0.091 mL, 0.914 mmol) and heated at 80° C. for 2 h to provide decarboylated compound 3.

### LC/MS [M+H]⁺=577.3.

Step 3, Part 2. A solution of decarbamoylated compound 3 (12 mg, 0.021 mmol) in MeOH (1 mL) containing Pd—C(2.214 mg, 0.021 mmol) was bubbled with H₂ for 1 min. The reaction mixture was heated under a hydrogen balloon atmosphere at 60° C. for 2 h. The crude product was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 9% B, 9-49% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide 4.7 mg of compound 271.

Compound 277 was analogously prepared.

### Example 32—Compound 250

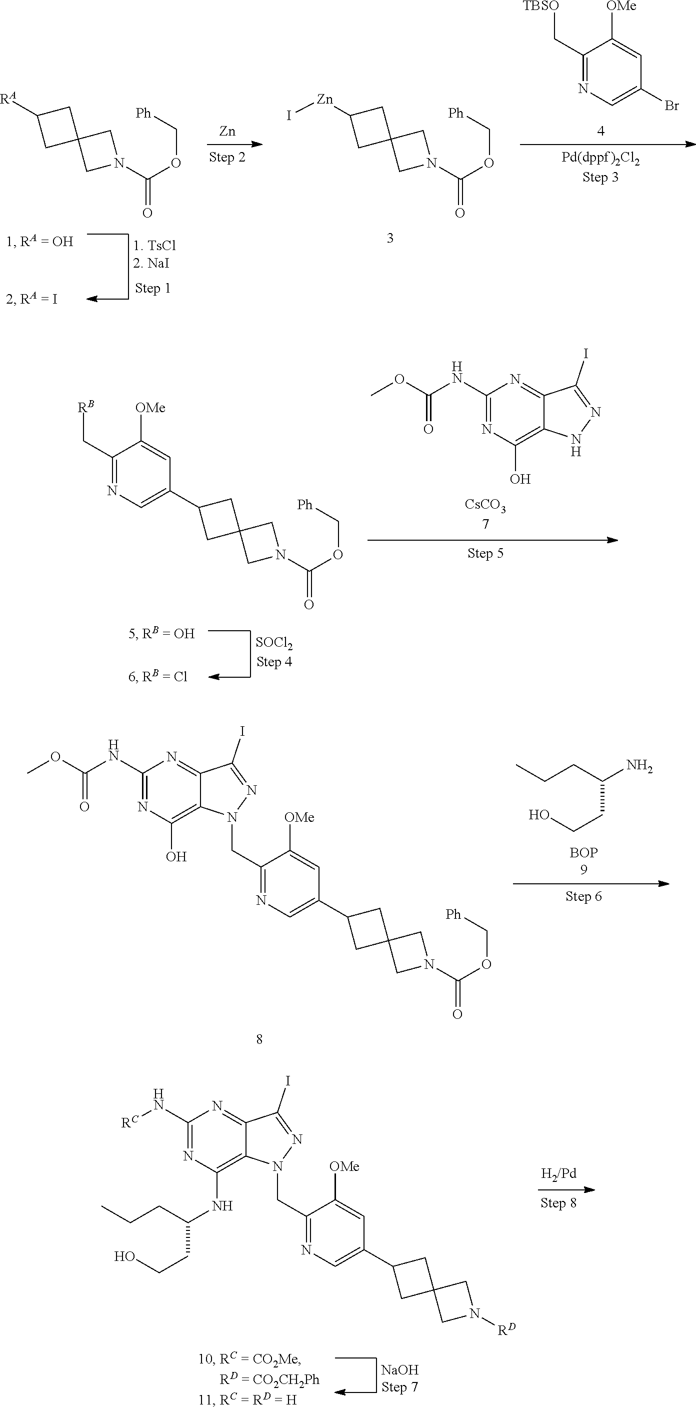

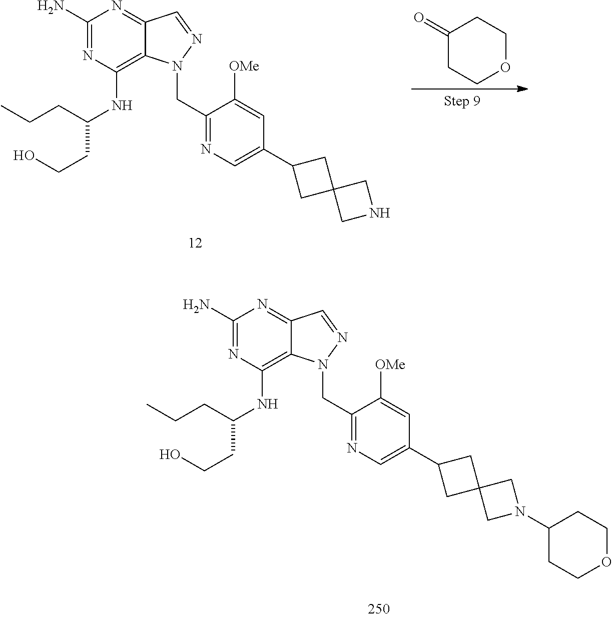 [SMILES: CCC[C@@H](CCO)Nc1nc(N)nc2cnn(Cc3ncc(C4CC5(CNC5)C4)cc3OC)c12 ; O=C1CCOCC1 ; CCC[C@@H](CCO)Nc1nc(N)nc2cnn(Cc3ncc(C4CC5(C4)CN(C4CCOCC4)C5)cc3OC)c12]

Step 1. A solution of benzyl 6-hydroxy-2-azaspiro[3.3]heptane-2-carboxylate 1 (CAS #1363383-32-7; 3 g, 12.13 mmol) in DCM (20 mL) was treated with triethylamine (2.029 mL, 14.56 mmol), DMAP (0.296 g, 2.426 mmol) and tosyl-CI (2.54 g, 13.34 mmol) at 0° C. The reaction was allowed to proceed over 2 h. The reaction was quenched with 50 mL water and washed with 50 mL 1M aqueous HCl solution, brine (50 mL) and dried over Na₂SO₄, filtered and concentrated to provide crude tosylated intermediate as a yellowish residue. This was dissolved in DMSO (20 mL) and treated with sodium iodide (5.46 g, 36.4 mmol). After heating at 120° C. over 2 h. The reaction mixture was dissolved in 50 mL EtOAc and washed with saturated aqueous Na₂S₂O₃ solution (50 mL), water (50 mL), brine (50 mL) and dried over Na₂SO₄. Filtration, concentration, and purification on an 80 g silica gel column eluting with 0-50% EtOAc/hexanes provided compound 2 as white solid.

### LC/MS [M+H]⁺=357.9.

¹H NMR (400 MHz, DMSO-d₆) δ 7.42-7.27 (m, 5H), 5.01 (s, 2H), 4.43 (p, J=7.9 Hz, 1H), 3.96 (s, 4H), 2.92 (ddd, J=10.4, 7.5, 3.1 Hz, 2H), 2.74-2.61 (m, 2H).

Step 2. A solution of compound 2 (1649 mg, 4.62 mmol) in 4 mL THF was added to Rieke zinc in THF (12.08 mL, 9.23 mmol) in an oven-dried round bottom flask under N₂. The temperature of the flask increased, indicating formation of zinc reagent 3. The reaction mixture was stirred at RT for 1 h and kept under N₂ for future use.

Step 3. A solution of 5-bromo-2-(((tert-butyldimethylsilyl)oxy)methyl)-3-methoxypyridine (1.4 g, 4.21 mmol), 1,1′-bis(diphenylphosphino)ferrocenedichloro palladium(II) dichloromethane complex (0.308 g, 0.421 mmol) and copper(I) iodide (0.160 g, 0.843 mmol) in DMF (10 mL) was bubbled with N₂ for 1 min. (2-((Benzyloxy)carbonyl)-2-azaspiro[3.3]heptan-6-yl)zinc(II) iodide 3 (17.49 mL, 5.06 mmol) was added. The reaction mixture was heated at 70° C. for h. This solution was treated with triethylamine trihydrofluoride (1.372 mL, 8.43 mmol) and stirred overnight. LCMS showed formation of compound 5 (167 mg, 0.453 mmol, 10.76% yield). The reaction as directly purified on an 150 g reverse phase C-18 column eluting with 0-50% MeCN/water (0.05% TFA) and desired fractions were collected to give compound 5 a as pale yellow solid.

### LC/MS [M+H]⁺=369.2.

¹H NMR (400 MHz, DMSO-d₆) δ 8.08 (s, 1H), 7.81 (s, 1H), 7.43-7.28 (m, 5H), 5.04 (s, 2H), 4.71 (s, 2H), 4.11 (s, 2H), 3.66-3.56 (m, 3H), 3.61-3.48 (m, 1H), 2.58 (ddt, J=10.6, 8.4, 2.5 Hz, 2H), 2.41 (td, J=9.5, 2.9 Hz, 2H), 1.84-1.70 (m, 3H).

Steps 4-5. A solution of compound 5(167 mg, 0.453 mmol) in THF (1 mL) was treated with SOCl₂ (0.066 mL, 0.907 mmol) and stirred at RT for 30 min. The solvent was evaporated with a V-10 evaporator. Crude product 6 in 1 mL DMF was added to a solution of methyl (7-hydroxy-3-iodo-1H-pyrazolo[4,3-d]pyrimidin-5-yl)carbamate 7 (152 mg, 0.453 mmol) and Cs₂CO₃ (295 mg, 0.907 mmol) in 1 mL of DMF. After heating at 60° C. for 2 h. The reaction was filtered and directly purified on a 50 g reverse phase C-18 column, eluting with 0-50% MeCN/water (0.05% TFA). The desired fractions which were lyophilized to provide compound 8 as a pale yellow solid.

### LC/MS [M+H]⁺=886.1.

Steps 6-7. A solution of compound 8 (140 mg, 0.204 mmol) and (S)-3-aminohexan-1-ol 9 (47.9 mg, 0.408 mmol) in DMSO (1 mL) was treated with DBU (0.092 mL, 0.613 mmol) followed by BOP (135 mg, 0.306 mmol). After heating at 40° C. for 1 h, LMCS showed completion of reaction to provide intermediate 10. The reaction mixture was treated with NaOH (0.204 mL, 2.042 mmol) and heated at 80° C. for 2 h. The reaction mixture was directly purified on a 50 g C-18 reverse phase column eluting with 0-50% MeCN/water (0.05% TFA). The desired fractions were lyophilized to yield compound 11 as pale yellow solid.

### LC/MS [M+H]⁺=593.1.

Step 8. A solution of compound 11 (30 mg, 0.051 mmol) in MeOH (1 mL) with Pd—C (5.39 mg, 0.051 mmol) was bubbled with H₂ (10.21 mg, 5.06 mmol) for 1 min. The reaction mixture was heated at 50° C. under a H₂ balloon for 2 h. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 6% B, 6-46% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to provide 7.6 mg of compound 12.

### LC/MS [M+H]⁺=466.9.

¹H NMR (500 MHz, DMSO-d₆) δ 7.93 (s, 2H), 7.90 (d, J=1.6 Hz, 1H), 7.51 (d, J=7.6 Hz, 1H), 7.43 (d, J=8.6 Hz, 1H), 5.96 (s, 1H), 5.71-5.59 (m, 3H), 4.43 (s, 2H), 4.11 (s, 1H), 3.90 (d, J=6.9 Hz, 3H), 2.68 (d, J=9.9 Hz, OH), 2.57 (d, J=22.7 Hz, 2H), 2.35 (d, J=14.2 Hz, 2H), 2.24 (s, 1H), 1.92 (s, 1H), 1.78 (d, J=6.3 Hz, 2H), 1.77-1.69 (m, 2H), 1.58 (s, 4H), 1.29 (s, 2H), 0.91-0.84 (m, 3H).

Step 9. A solution of compound 12 (30 mg, 0.064 mmol) and tetrahydro-4H-pyran-4-one (0.012 mL, 0.129 mmol) in DMF (0.5 mL) was treated with 2 drops of acetic acid and 50 mg 4 Å molecular sieves and sodium triacetoxyborohydride (54.5 mg, 0.257 mmol). After stirring at RT for 1 h. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 13% B, 13-53% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The material was further purified via preparative LC/MS with the following conditions: Column: XBridge Phenyl, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 9% B, 9-49% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide 1.9 mg of compound 250.

Compound 251 was analogously prepared.

### Example 33—Starting Materials and Intermediates

The Charts below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

### Chart 1

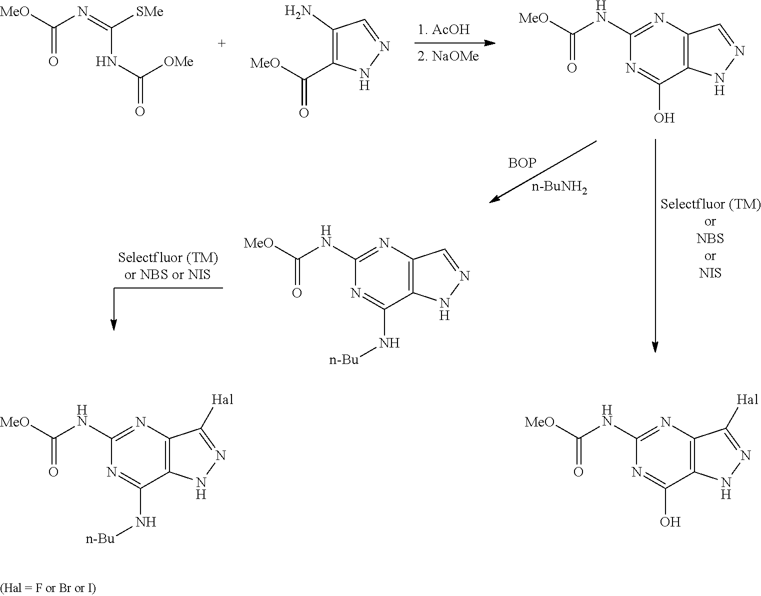

### Chart 2

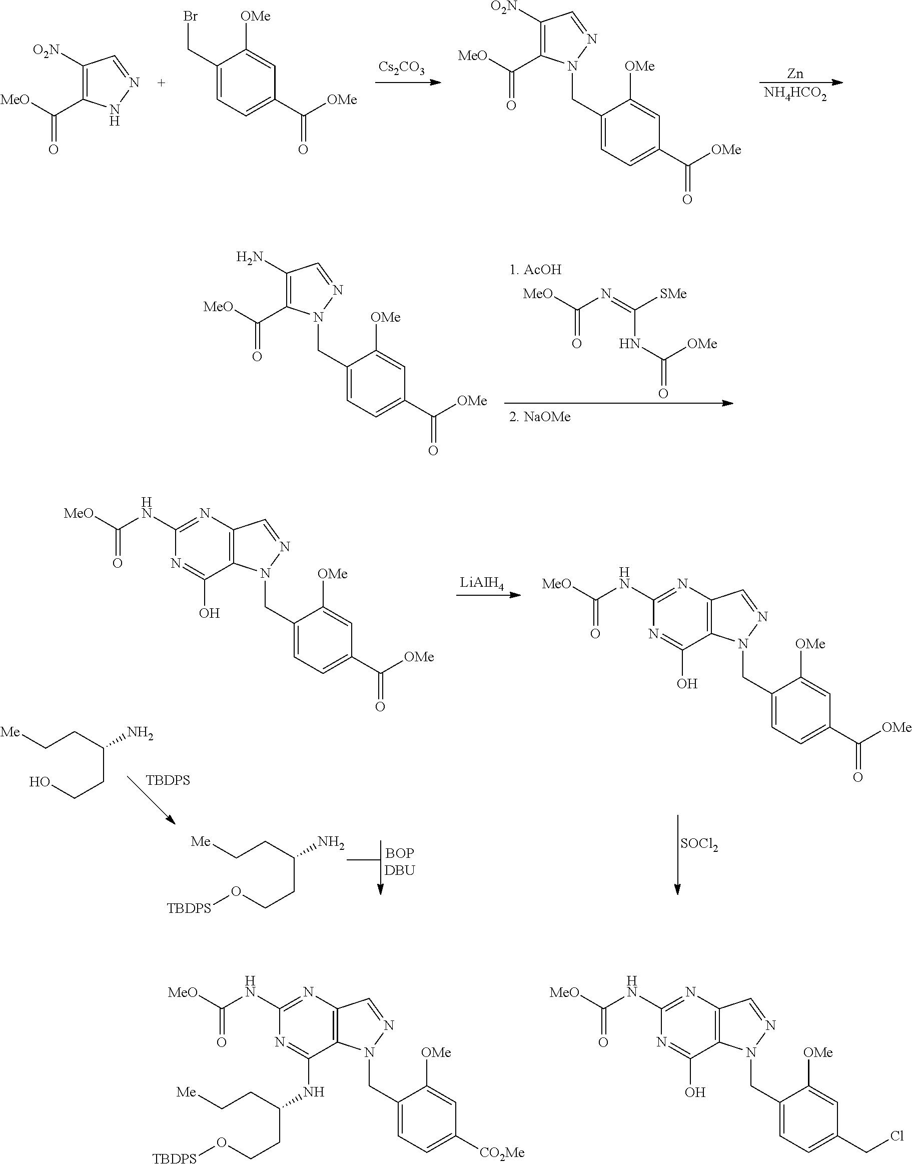

### Chart 3

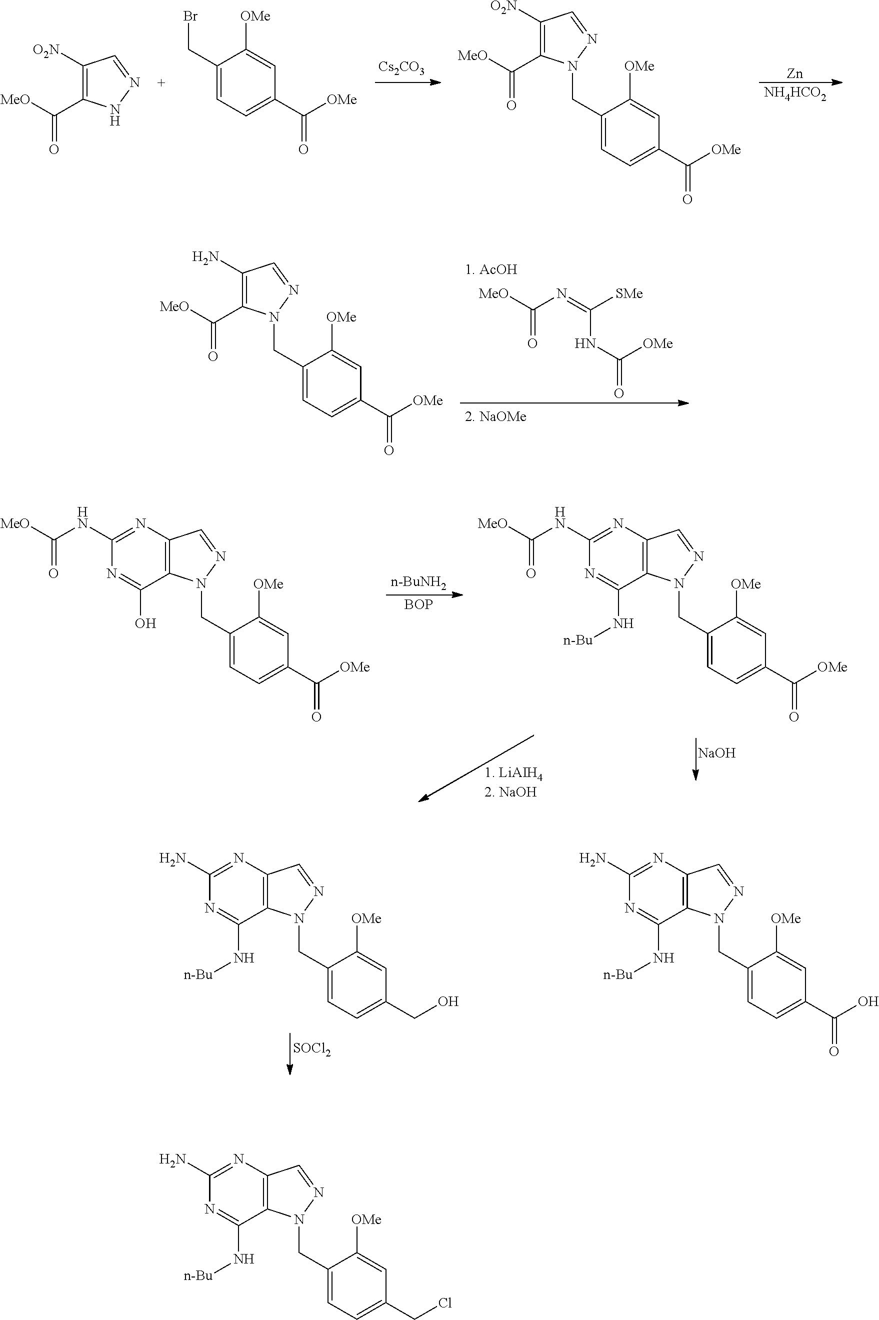

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue™ reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×->1×in H₂O, warm at 37° C.; Cat# BD 558049) and kept the perm buffer (on ice) for later use.

For surface markers staining (CD69): prepared surface Abs: 0.045 ul hCD14-FITC (ThermoFisher Cat # MHCD1401)+0.6 ul hCD19-ef450 (ThermoFisher Cat #48-0198-42)+1.5 ul hCD69-PE (cat# BD555531)+0.855 ul FACS buffer. Added 3 ul/well, spin1000 rpm for 1 min and mixed on shaker for 30 sec, put on ice for 30 mins. Stop stimulation after 30 minutes with 70 uL of prewarmed 1×fix/lysis buffer and use Feliex mate to resuspend (15 times, change tips for each plate) and incubate at 37° C. for 10 minutes.

Centrifuge at 2000 rpm for 5 minutes aspirate with HCS plate washer, mix on shaker for 30 sec and then wash with 70 uL in dPBS and pelleted 2 xs (2000 rpm for 5 min) and 50 ul wash in FACS buffer pelleted 1 xs(2000 rpm for 5 min). Mix on shaker for 30 sec. For Intracellular markers staining (MX-1): Add 50 ul of BD Perm buffer III and mix on shaker for 30 sec. Incubate on ice for 30 minutes (in the dark). Wash with 50 uL of FACS buffer 2×(spin @2300 rpm×5 min after perm) followed by mixing on shaker for 30 sec. Resuspended in 20 ul of FACS buffer containing MX1 antibody ( )(4812)-Alexa 647: Novus Biologicals #NBP2-43704AF647) 20 ul FACS bf+0.8 ul hlgG+0.04 ul MX-1. Spin 1000 rpm for 1 min, mix on shaker for 30 se and the samples were incubated at RT in the dark for 45 minutes followed by washing 2×FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% CO₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by ThermoFisher Scientific). A 70 uL volume of mRNA catcher lysis buffer (1×) with DTT from the Invitrogen mRNA Catcher Plus kit (Cat#K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat# K1570-02) according to the manufacturer's instructions. Half yield of mRNA from RNA extraction were used to synthesize cDNA in 20 μL reverse transcriptase reactions using Invitrogen SuperScript IV VILO Master Mix (Cat#11756500). TaqMan*real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the 15 number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

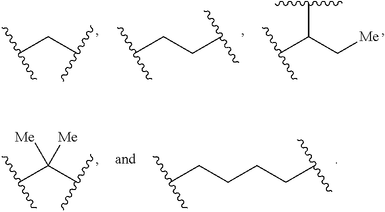

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E—(or Z—) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cycloalkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (osr “bicycloalkylene”) and “spiroalkanediyl” (or “spiroalkylene”) refer to divalent counterparts of a bicycloalkyl and spiroalkyl (or “spirocycloalkyl”) group. By way of illustration, an example of a

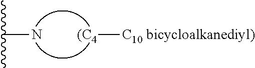

moiety is

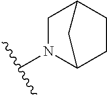 [SMILES: *N1CC2CCC1C2]

and an example of a

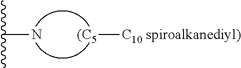

moiety is

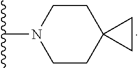 [SMILES: *N1CCC2(CC1)CC2]

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi—, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi—, or tricyclic ring system (preferably 5- to 7-membered monocyclic) wherein each ring has from 3 to 7 carbon atoms and at least one ring is an aromatic ring containing from 1 to 4 heteroatoms independently selected from from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in Ci and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond or an asterisk (*) at the end of the bond denotes a covalent attachment site. For instance, a statement that R is

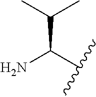 [SMILES: *[C@@H](N)C(C)C]

or that R is

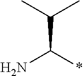 [SMILES: *[C@@H](N)C(C)C]

in the formula

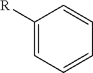

means

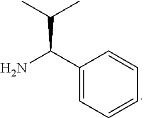

In the formulae of this specification, a bond traversing an aromatic ring between two carbons thereof means that the group attached to the bond may be located at any of the positions of the aromatic ring made available by removal of the hydrogen that is implicitly there (or explicitly there, if written out). By way of illustration:

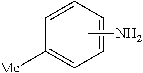

represents

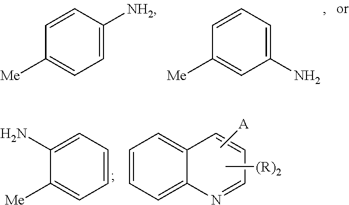

represents

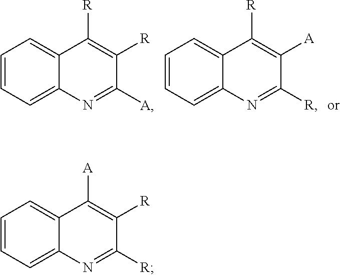

and

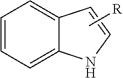

represents

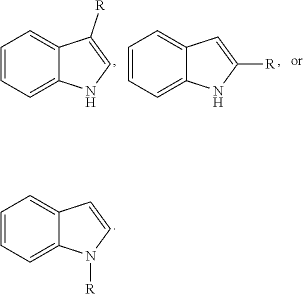

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### ACRONYMS AND ABBREVIATIONS

Table C provides a list of acronyms and abbreviations used in this specification, along with their meanings.

TABLE C

| ACRONYM OR | |
|---|---|
| ABBREVIATION | MEANING OR DEFINITION |
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hunig's base |
| DMA | N,N-Dimethylacetamide |
| DMAP | 4-(Dimethylamino) pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenylmethyloxycarbonyl |
| HATU | Hexafluorophosphate Azabenzotriazole |
| | Tetramethyl Uronium; 1-[Bis(dimethylamino)- |
| | methylene]-1H-1,2,3-triazolo[4,5-b] pyridinium |
| | 3-oxide hexafluorophosphate |
| HEPES | 4-(2-Hydroxyethyl)piperazine-1-ethanesulfonic |
| | acid, N-(2-Hydroxyethyl)piperazine-N′-(2- |
| | ethanesulfonic acid) |
| HPLC | High pressure liquid chromatography |
| Hunig's base | See DIPEA, DIEA |
| LCMS, LC-MS, | Liquid chromatography-mass spectrometry |
| LC/MS | |
| mCPBA | m-chloroperbenzoic acid |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluororethylene) |
| RT (in context of | Retention time, in min |
| liquid | |
| chromatography) | |
| RT (in the context of | Room (ambient) temperature, circa 25° C. |
| reaction conditions) | |
| Sat. | Saturated |
| Soln | Solution |
| TBDPS | tert-Butyldiphenylsilyl |
| TBS | t-Butyldimethylsily group |
| TEA | Triethylamine |
| TEAA | Triethylammonium acetate |
| TFA | Trifluoroacetic acid |
| THF | Tetrahydrofuran |

### REFERENCES

Full citations for the following references cited in abbreviated fashion by first author (or inventor) and date earlier in this specification are provided below. Each of these references is incorporated herein by reference for all purposes.

Akinbobuyi et al., Tetrahedron Lett. 2015, 56, 458, “Facile syntheses of functionalized toll-like receptor 7 agonists”.

Akinbobuyi et al., Bioorg. Med. Chem. Lett. 2016, 26, 4246, “Synthesis and immunostimulatory activity of substituted TLR7 agonists.”

Barberis et al., US 2012/0003298 A1 (2012).

Beesu et al., J. Med. Chem. 2017, 60, 2084, “Identification of High-Potency Human TLR8 and Dual TLR7/TLR8 Agonists in Pyrimidine-2,4-diamines.”

Berghöfer et al., J. Immunol. 2007, 178, 4072, “Natural and Synthetic TLR7 Ligands Inhibit CpG-A- and CpG-C-Oligodeoxynucleotide-Induced IFN-α Production.”

Bonfanti et al., US 2014/0323441 A1 (2015) [2015a].

Bonfanti et al., US 2015/0299221 A1 (2015) [2015b].

Bonfanti et al., US 2016/0304531 A1 (2016).

Carson et al., US 2013/0202629 A1 (2013).

Carson et al., U.S. Pat. No. 8,729,088 B2 (2014).

Carson et al., U.S. Pat. No. 9,050,376 B2 (2015).

Carson et al., US 2016/0199499 A1 (2016).

Chan et al., Bioconjugate Chem. 2009, 20, 1194, “Synthesis and Immunological Characterization of Toll-Like Receptor 7 Agonistic Conjugates.”

Chan et al., Bioconjugate Chem. 2011, 22, 445, “Synthesis and Characterization of PEGylated Toll Like Receptor 7 Ligands.”

Chen et al., U.S. Pat. No. 7,919,498 B2 (2011).

Coe et al., U.S. Pat. No. 9,662,336 B2 (2017).

Cortez and Va, Medicinal Chem. Rev. 2018, 53, 481, “Recent Advances in Small-Molecule TLR7 Agonists for Drug Discovery”.

Cortez et al., US 2017/0121421 A1 (2017).

Cortez et al., U.S. Pat. No. 9,944,649 B2 (2018).

Dellaria et al., WO 2007/028129 A1 (2007).

Desai et al., U.S. Pat. No. 9,127,006 B2 (2015).

Ding et al., WO 2016/107536 A1 (2016).

Ding et al., US 2017/0273983 A1 (2017) [2017a].

Ding et al., WO 2017/076346 A1 (2017) [2017b].

Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”

Graupe et al., U.S. Pat. No. 8,993,755 B2 (2015).

Embrechts et al., J. Med. Chem. 2018, 61, 6236, “2,4-Diaminoquinazolines as Dual Toll Like Receptor (TLR) 7/8 Modulators for the Treatment of Hepatitis B Virus.”

Halcomb et al., U.S. Pat. No. 9,161,934 B2 (2015).

Hashimoto et al., US 2009/0118263 A1 (2009).

He et al., U.S. Pat. No. 10,487,084 B2 (2019) [2019a].

He et al., U.S. Pat. No. 10,508,115 B2 (2019) [2019b].

Hirota et al., U.S. Pat. No. 6,028,076 (2000).

Holldack et al., US 2012/0083473 A1 (2012).

Isobe et al., U.S. Pat. No. 6,376,501 B1 (2002).

Isobe et al., JP 2004137157 (2004).

Isobe et al., J. Med. Chem. 2006, 49 (6), 2088, “Synthesis and Biological Evaluation of Novel 9-Substituted-8-Hydroxyadenine Derivatives as Potent Interferon Inducers.”

Isobe et al., U.S. Pat. No. 7,521,454 B2 (2009) [2009a].

Isobe et al., US 2009/0105212 A1 (2009) [2009b].

Isobe et al., US 2011/0028715 A1 (2011).

Isobe et al., U.S. Pat. No. 8,148,371 B2 (2012).

Jensen et al., WO 2015/036044 A1 (2015).

Jones et al., U.S. Pat. No. 7,691,877 B2 (2010).

Jones et al., US 2012/0302598 A1 (2012).

Kasibhatla et al., U.S. Pat. No. 7,241,890 B2 (2007).

Koga-Yamakawa et al.,/nt. J. Cancer 2013, 132 (3), 580, “Intratracheal and oral administration of SM-276001: A selective TLR7 agonist, leads to antitumor efficacy in primary and metastatic models of cancer.”

Li et al., U.S. Pat. No. 9,902,730 B2 (2018).

Lioux et al., U.S. Pat. No. 9,295,732 B2 (2016).

Lund et al., Proc. Nat'l Acad. Sci (USA) 2004, 101 (15), 5598, “Recognition of single-stranded RNA viruses by Toll-like receptor 7.”

Maj et al., U.S. Pat. No. 9,173,935 B2 (2015).

McGowan et al., US 2016/0168150 A1 (2016) [2016a].

McGowan et al., U.S. Pat. No. 9,499,549 B2 (2016) [2016b].

McGowan et al., J. Med. Chem. 2017, 60, 6137, “Identification and Optimization of Pyrrolo[3,2-d]pyrimidine Toll-like Receptor 7 (TLR7) Selective Agonists for the Treatment of Hepatitis B.”

Musmuca et al., J. Chem. Information & Modeling 2009, 49 (7), 1777, “Small-Molecule Interferon Inducers. Toward the Comprehension of the Molecular Determinants through Ligand-Based Approaches.”

Nakamura et al., Bioorg. Med. Chem. Lett. 2013, 13, 669, “Synthesis and evaluation of 8-oxoadenine derivatives as potent Toll-like receptor agonists with high water solubility.”

Ogita et al., US 2007/0225303 A1 (2007).

Ota et al., WO 2019/124500 A1 (2019).

Pilatte et al., WO 2017/216293 A1 (2017).

Poudel et al., U.S. Pat. No. 10,472,361 B2 (2019) [2019a].

Poudel et al., U.S. Pat. No. 10,494,370 B2 (2019) [2019b].

Poudel et al., US 2020/0038403 A1 (2020) [2020a].

Poudel et al., US 2020/0039986 A1 (2020) [2020b].

Purandare et al., WO 2019/209811 A1 (2019).

Pryde, U.S. Pat. No. 7,642,350 B2 (2010).

Sato-Kaneko et al., JCI Insight 2017, 2, e93397, “Combination Immunotherapy with TLR Agonists and Checkpoint Inhibitors Suppresses Head and Neck Cancer”.

Smits et al., The Oncologist 2008, 13, 859, “The Use of TLR7 and TLR8 Ligands for the Enhancement of Cancer Immunotherapy”.

Vasilakos and Tomai, Expert Rev. Vaccines 2013, 12, 809, “The Use of Toll-like Receptor 7/8 Agonists as Vaccine Adjuvants”.

Vernejoul et al., US 2014/0141033 A1 (2014).

Young et al., U.S. Pat. No. 10,457,681 B2 (2019).

Yu et al., PLoS One 2013, 8 (3), e56514, “Toll-Like Receptor 7 Agonists: Chemical Feature Based Pharmacophore Identification and Molecular Docking Studies.”

Zhang et al., Immunity 2016, 45, 737, “Structural Analysis Reveals that Toll-like Receptor 7 Is a Dual Receptor for Guanosine and Single-Stranded RNA.”

Zhang et al., WO 2018/095426 A1 (2018)>

Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula (I) 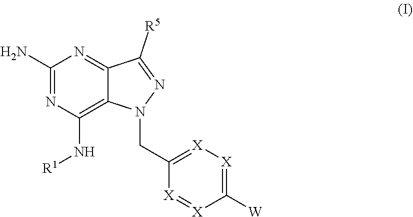
wherein
W is H, halo, C₁-C₃ alkyl, CN, (C₁-C₄ alkanediyl)OH, 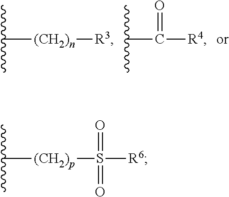
each X is independently N or CR²;
R¹ is (C₁-C₅ alkyl),
(C₁-C₅ alkenyl),
(C₁-C₅ alkanediyl)₀₋₁(C₃-C₆ cycloalkyl),
(C₁-C₅ alkanediyl)₀₋₁(C₅-C₁₀ spiroalkyl),
(C₂-C₅ alkanediyl)OH,
(C₂-C₅ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
(C₂-C₈ alkanediyl)₀₋₁(C₃-C₆ cycloalkanediyl)(C₃-C₆ cycloalkyl), or
(C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl,
O(C₃-C₄ cycloalkyl), S(C₃-C₄ cycloalkyl), SO₂(C₃-C₄ cycloalkyl), C₃-C₄ cycloalkyl, Cl, F, CN, or [C(═O)]₀₋₁NR^{x}R^{y};
R³ is H, halo, OH, CN,
NH₂,
NH[C(═O)]₀₋₁(C₁-C₅ alkyl),
N(C₁-C₅ alkyl)₂,
NH[C(═O)]₀₋₁(C₁-C₄ alkanediyl)₀₋₁(C₃-C₅ cycloalkyl),
NH[C(═O)]₀₋₁(C₁-C₄ alkanediyl)₀₋₁(C₄-C₁₀ bicycloalkyl),
NH[C(═O)]₀₋₁(C₁-C₄ alkanediyl)₀₋₁(C₅-C₁₀ spiroalkyl),
N(C₃-C₆ cycloalkyl)₂,
O(C₁-C₄ alkanediyl)₀₋₁(C₃-C₅ cycloalkyl),
O(C₁-C₄ alkanediyl)₀₋₁(C₄-C₅ bicycloalkyl),
O(C₁-C₄ alkanediyl)₀₋₁(C₅-C₁₀ spiroalkyl),
O(C₁-C₄ alkanediyl)₀₋₁(C₁-C₆ alkyl),
N[C₁-C₃ alkyl]C(═O)(C₁-C₆ alkyl),
NH(SO₂)(C₁-C₅ alkyl),
NH(SO₂)(C₁-C₄ alkanediyl)₀₋₁(C₃-C₅ cycloalkyl),
NH(SO₂)(C₁-C₄ alkanediyl)₀₋₁(C₄-C₁₀ bicycloalkyl),
NH(SO₂)(C₁-C₄ alkanediyl)₀₋₁(C₅-C₁₀ spiroalkyl),
a 6-membered aromatic or heteroaromatic moiety,
a 5-membered heteroaromatic moiety, or
a moiety having the structure 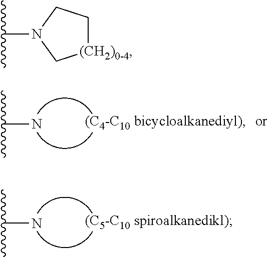
R⁴ is NH₂,
NH(C₁-C₅ alkyl),
N(C₁-C₅ alkyl)₂,
NH(C₁-C₄ alkanediyl)₀₋₁(C₃-C₅ cycloalkyl),
NH(C₁-C₄ alkanediyl)₀₋₁(C₄-C₁₀ bicycloalkyl),
NH(C₁-C₄ alkanediyl)₀₋₁(C₅-C₁₀ spiroalkyl),
N(C₃-C₆ cycloalkyl)₂,
or
a moiety having the structure 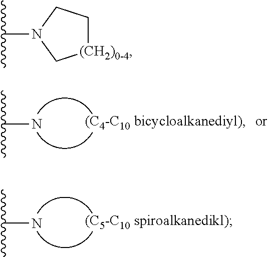
R⁵ is H, C₁-C₅ alkyl, C₂-C₅ alkenyl, C₃-C₆ cycloalkyl, halo, O(C₁-C₅ alkyl),
(C₁-C₄ alkanediyl)OH, (C₁-C₄ alkanediyl)O(C₁-C₃ alkyl), phenyl, NH(C₁-C₅ alkyl), 5 or 6 membered heteroaryl, 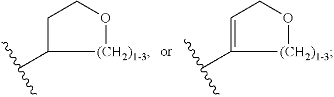
R⁶ is NH₂,
(NH)₀₋₁(C₁-C₅ alkyl),
N(C₁-C₅ alkyl)₂,
(NH)₀₋₁(C₁-C₄ alkanediyl)₀₋₁(C₃-C₅ cycloalkyl),
(NH)₀₋₁(C₁-C₄ alkanediyl)₀₋₁(C₄-C₁₀ bicycloalkyl),
(NH)₀₋₁(C₁-C₄ alkanediyl)₀₋₁(C₅-C₁₀ spiroalkyl),
N(C₃-C₆ cycloalkyl)₂,
or
a moiety having the structure 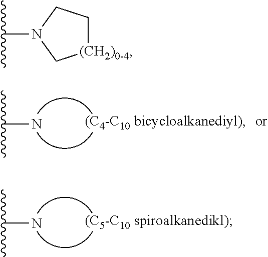
R^{x} and R^{y} are independently H or C₁-C₃ alkyl or R^{x} and R^{y} combine with the nitrogen to which they are bonded to form a 3- to 7-membered heterocycle;
n is 1, 2, or 3;
and
p is 0, 1, 2, or 3;
wherein in R¹, R², R³, R⁴, R⁵, and R⁶
an alkyl, alkenyl, cycloalkyl, alkanediyl, bicycloalkyl, spiroalkyl, cyclic amine, 6-membered aromatic or heteroaromatic moiety, 5-membered heteroaromatic moiety or a moiety of the formula 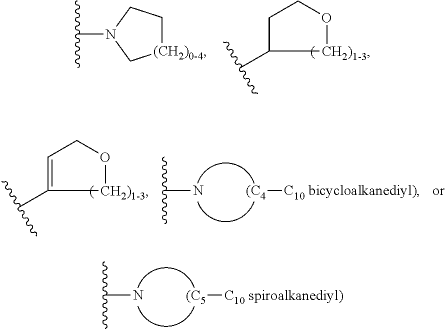
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y},
(C₁-C₄ alkanediyl)OH, (C₁-C₄ alkanediyl)O(C₁-C₃ alkyl);
and
an alkyl, alkenyl, alkanediyl, cycloalkyl, bicycloalkyl, spiroalkyl, or a moiety of the formula 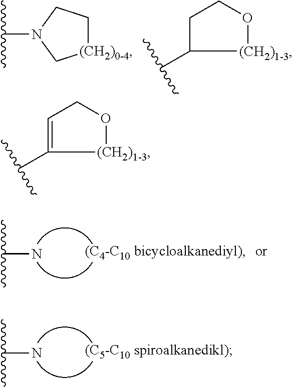
optionally may have a CH₂ group replaced by O, SO₂, CF₂, C(═O), NH,
N[C(═O)]₀₋₁(C₁-C₅ alkyl),
N[C(═O)]₀₋₁(C₁-C₄ alkanediyl)CF₃,
N[C(═O)]₀₋₁(C₂-C₄ alkanediyl)OH
N(SO₂)(C₁-C₃ alkyl),
N(C₁-C₃ alkanediyl)₀₋₁[C(═O)]NR^{x}R^{y},
or
N[C(═O)]₀₋₁(C₁-C₄ alkanediyl)₀₋₁(C₃-C₅ cycloalkyl);
with the provisos that at least one or R¹ and W comprises a spiroalkyl or spiroalkanediyl
moiety and that the compound of formula (I) is other than

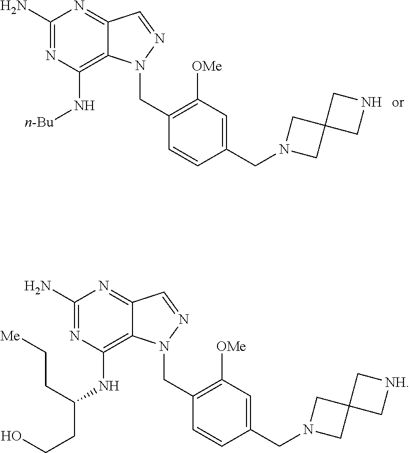

2. A compound according to claim 1, wherein W is

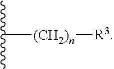

3. A compound according to claim 1, wherein W is

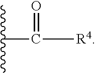

4. A compound according to claim 1, wherein each of R¹ and W comprises a spiroalkyl or spiroalkanediyl moiety.

5. A compound according R¹ comprises a spiroalkyl moiety and W comprises a bicycloalkyl or bicycloalkanediyl moiety.

6. A compound according to claim 1, wherein R¹ is selected from the group consisting of

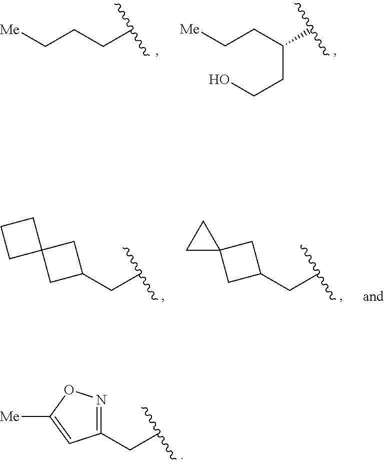 [SMILES: *CCCC ; *[C@@H](CCC)CCO ; *CC1CC2(CCC2)C1 ; *CC1CC2(CC2)C1 ; *Cc1cc(C)on1]

7. A compound according to claim 1, wherein R² is OMe or OCHF₂, preferably OMe.

8. A compound according to claim 1, wherein R⁵ is H, CH₂OH, or Me, preferably H.

9. A compound according to claim 1, having a structure according to formula (Ia)

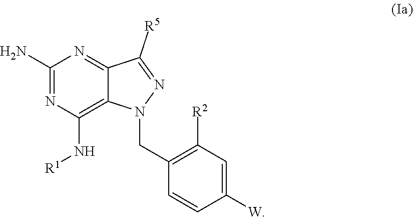

10. A compound according to claim 1, having a structure according to formula (Ib)

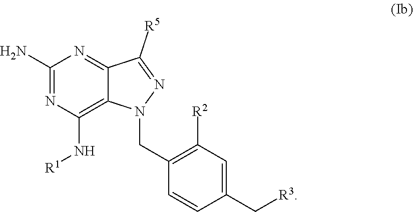

11. A compound according to claim 10, wherein 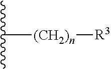
is selected from the group consisting of

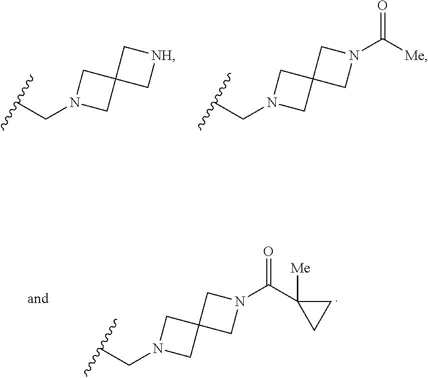 [SMILES: *CN1CC2(CNC2)C1 ; *CN1CC2(C1)CN(C(C)=O)C2 ; *CN1CC2(C1)CN(C(=O)C1(C)CC1)C2]

12. A compound according to claim 1, having a structure according to formula (Ic)

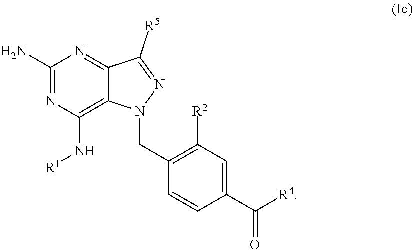

13. A compound according to claim 12, wherein 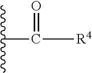
is selected from the group consisting of

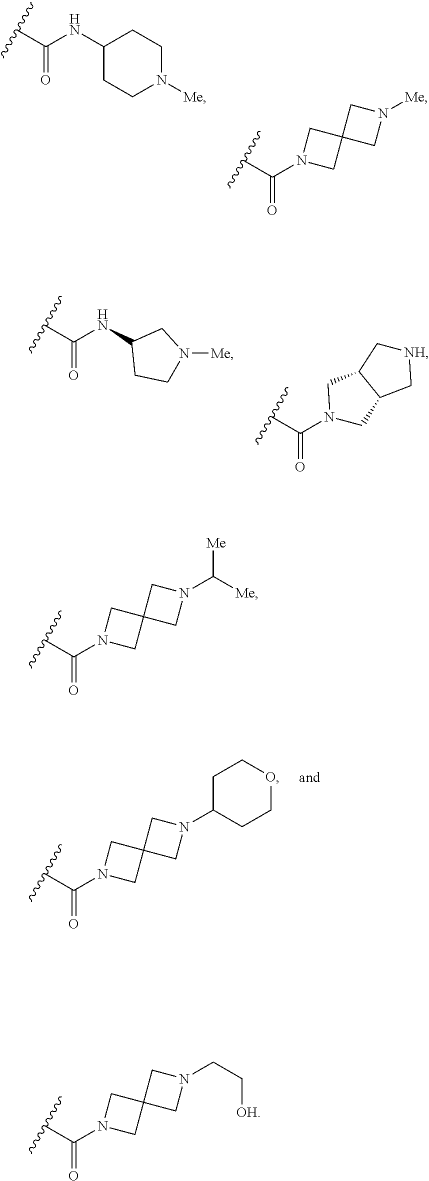

14. A compound having a structure according to formula (Id) 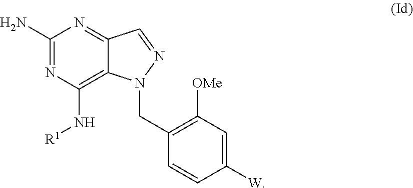
wherein
R¹ is 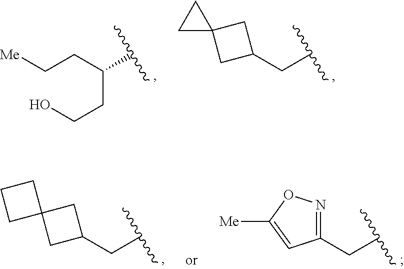 [SMILES: *[C@@H](CCC)CCO ; *CC1CC2(CC2)C1 ; *CC1CC2(CCC2)C1 ; *Cc1cc(C)on1]
and
W is

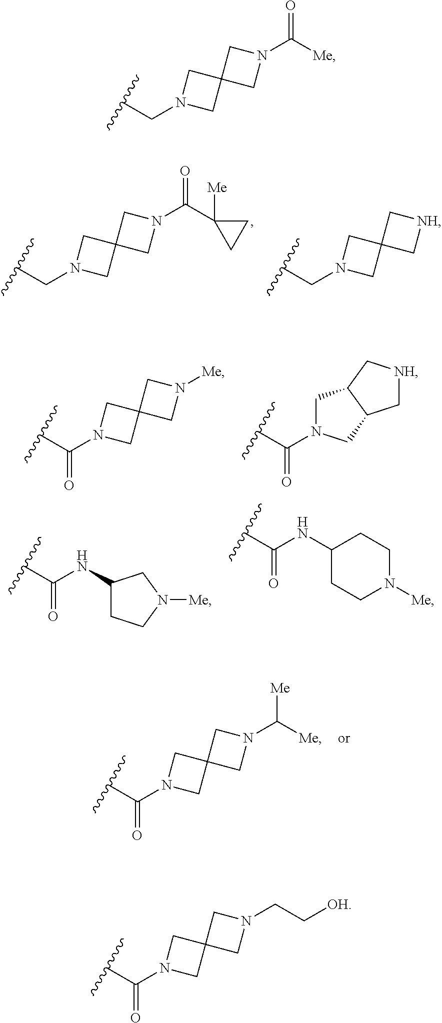

15. A method of treating a cancer, comprising administering to a patient suffering from such cancer a therapeutically effective combination of an anti-cancer immunotherapy agent and a compound according to claim 1.

16. A method according to claim 15, wherein the anti-cancer immunotherapy agent is an antagonistic anti-CTLA-4, anti-PD-1, or anti-PD-L1 antibody.

17. A method according to claim 16, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

18. A method according to claim 17, wherein the anti-cancer immunotherapy agent is ipilimumab, nivolumab, or pembrolizumab.

19. A compound having a structure according to formula (Ie) 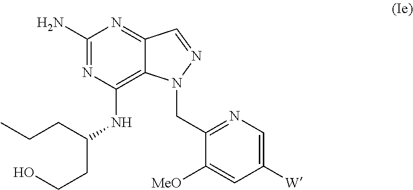
wherein W′ is 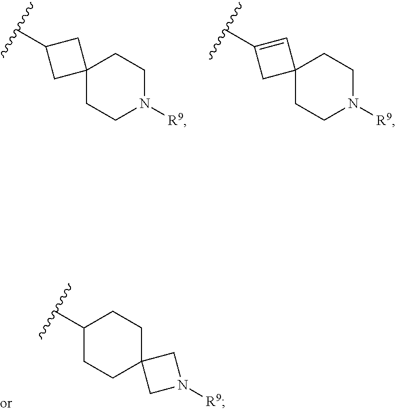
and R⁹ is H, C₁-C₅ alkyl, (CH₂)₁₋₂(C₃-C₅ cycloalkyl), or

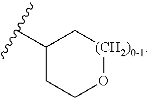

20. A method of treating a cancer, comprising administering to a patient suffering from such cancer a therapeutically effective combination of an anti-cancer immunotherapy agent and a compound according to claim 14.
